(19) United States
(12) Patent Application Publication
(10) Pub. No.: US 2022/0133740 A1
(43) Pub. Date: May 5, 2022

(54) VALPROIC ACID COMPOUNDS AND WNT AGONISTS FOR TREATING EAR DISORDERS

(71) Applicant: Frequency Therapeutics, Inc., Lexington, MA (US)

(72) Inventors: Will MCLEAN, North Haven, CT (US); Christopher LOOSE, Winchester, MA (US)

(73) Assignee: Frequency Therapeutics, Inc., Lexington, MA (US)

(21) Appl. No.: 17/429,269

(22) PCT Filed: Feb. 7, 2020
(86) PCT No.: PCT/US2020/017352

§ 371 Date: Aug. 6, 2021

Related U.S. Application Data
(60) Provisional application No. 62/803,345, filed on Feb. 8, 2019.

(51) Int. Cl. A61K 31/5517; A61P 27/16; A61K 31/4439; A61K 31/19; A61K 31/506; A61K 9/00; A61K 31/5377

(52) U.S. Cl. CPC A61K 31/5517; A61P 27/16; A61K 31/4439; A61K 31/5377; A61K 31/506; A61K 9/0046; A61K 31/19

(57) ABSTRACT

The present invention provides methods for treating diseases and disorders of the ear with valproic acid compounds and Wnt agonists. The present invention further provides kits for the same.

### CROSS-REFERENCE TO RELATED APPLICATION

This application claims priority to U.S. Provisional Application No. 62/803,345 filed Feb. 8, 2019, entitled “METHODS OF TREATMENT WITH VALPROIC ACID COMPOUNDS AND WNT AGONISTS”, the disclosures of which are incorporated by reference herein.

### FIELD OF THE INVENTION

The present disclosure relates to compositions and methods comprising a systemic valproic acid compound and a local Wnt agonist for increasing proliferation of cochlear supporting cells or vestibular supporting cells, production of an expanded population of cochlear or vestibular cells, in particular Lgr5+ cells, and related methods of treating an inner hearing or balance disorder.

### BACKGROUND OF THE INVENTION

Generation of sensory hair cells from undifferentiated cell populations is likely to provide a therapy for several inner ear hearing and balance disorders that arise from damage and loss of sensory hair cells in the inner ear. Strategies for the in situ replacement of hair cells in damaged sensory epithelium in vivo are of particular interest but are likely to require the reliable delivery of therapeutic agents to the sensory epithelium of the inner ear.

Sensorineural hearing loss (SNHL), which is largely due to the loss of sensory hair cells and their neural connections is a widespread problem. It is estimated that over one billion young people are at risk for noise-related sensorineural hearing loss. SNHL accounts for about 90% of all hearing loss (Li et al., Adv. Drug Deliv. Rev. 108, 2-12, 2017), and leading causes include advanced age, ototoxic medications, and noise exposure (Liberman & Kujkawa, Hear. Res. 349, 138-147, 2017). The majority of children and adults with SNHL are managed with hearing aids or cochlear implants, as there is currently no therapeutic option to restore function in the damaged inner ear (see, for example, Ramakers et al., Laryngoscope 125, 2584-92, 2015; Raman et al., Effectiveness of Cochlear Implants in Adults with Sensorineural Hearing Loss. Agency for Healthcare Research and Quality (US), 2011; and Roche & Hansen, Otolaryngol. Clin. North Am. 48, 1097-116, 2015). Thus, there is a need in the art for effective therapies for treating SNHL and related conditions.

The underlying pathophysiologic changes of the inner ear in these patients include damage to sensory transducers of the cochlea called hair cells. Hair cells are susceptible to damage, and although other species such as birds, fish, and amphibians can regenerate these cells throughout life, mammals lack this ability (Fujioka et al., Trends Neurosci. 38, 139-44, 2015).

Loss or damage of hair cells in the vestibular system of inner ear can lead to balance disorders (for example, dizziness and vertigo), incidences of which also increase with age. Like the cochlea, there is currently no therapeutic option to restore function in damaged vestibular epithelia, and regeneration of hair cells may also be an effective therapeutic approach for balance disorders

Several approaches are being investigated to replace damaged or absent hair cells in mammalian inner ear sensory epithelia (reviewed in Mittal et al. Front Mol Neurosci. (2017); 10: 236). These include cell-based approaches (which aim to deliver exogenous cells to the inner ear to restore the sensory epithelia) and gene-based approaches (which aim to deliver exogenous genes to the sensory epithelia and reprogram endogenous cells to generate hair cells). For example, adenovirus-mediated delivery of Atoh1 is able to stimulate cells within the sensory epithelia to differentiate into hair cells. One drawback with these approaches is the requirement to deliver cells or vectors into the inner ear of the patient, which can be challenging in the complex system of the inner car. Molecular approaches, in which the endogenous signaling pathways of inner ear cells are modulated by exogenous agents are therefore attractive, as the delivery of such agents for prolonged periods of time is more straightforward than cell-based or gene-based approaches. One issue with local administration to the ear is achieving delivery of an effective amount of a composition over the entire cochlea.

A combination of a Wnt pathway agonist (a GSK3β inhibitor) in combination with a histone deacetylase complex (HDAC) inhibitor has been found to stimulate expansion of an Lgr5+ supporting cell population in the inner ear (McLean et al. Cell Rep. 2017 Feb. 21; 18(8): 1917-1929). However, these studies did not conclusively investigate what delivery routes are available for therapeutic regeneration of hair cells in the inner ear.

There remains a need for the development of effective in vivo hair cell regeneration strategies in the inner ear, which includes strategies for improved delivery of therapeutic compositions.

### SUMMARY OF THE INVENTION

Applicants have discovered that systemic administration of a composition can result in delivery of therapeutic amounts of a compound, e.g. a valproic acid compound, to the inner ear. Furthermore, applicants have discovered that a combination of systemic and local delivery provide an improvement in delivery and distribution of such a compound.

In one embodiment, the present disclosure provides a method of treating or preventing a disease or disorder of the ear in a subject in need thereof, comprising (a) systemically administering a valproic acid compound to the subject; and (b) locally administering a Wnt agonist to the middle or inner ear of the subject, wherein steps a) and b) may occur in any order or simultaneously. The Wnt agonist can include any Wnt agonist disclosed herein, administered as described herein with any valproic acid compound as described herein.

In one embodiment, the present disclosure provides a method of treating or preventing a disease or disorder of the ear in a subject in need thereof comprising a) systemically administering a valproic acid compound to the middle or inner ear of the subject, wherein the subject has been, or will be, b) administered a Wnt agonist locally to the middle or inner ear of the subject, wherein steps a) and b) may occur in any order or simultaneously.

In one embodiment, a method of treating or preventing a disease or disorder of the ear in a subject in need thereof comprising b) locally administering a Wnt agonist to the middle or inner ear of the subject, wherein the subject has been, or will be, a) administered a valproic acid compound systemically, wherein steps a) and b) may occur in any order or simultaneously.

In one embodiment, the present disclosure provides a method of treating or preventing a disease or disorder of the ear in a subject in need thereof, locally administering a Wnt agonist to the middle or inner ear of the subject, wherein the locally administering is at a time when the subject has a systemic plasma concentration of valproic acid of about 5 μg/mL to about 5000 μg/mL.

In one embodiment, the present disclosure provides a method of treating or preventing a disease or disorder of the ear in a subject in need thereof, comprising (a) systemically administering a valproic acid compound to the subject; and (b) systemically administering a Wnt agonist to the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) systemically administering GSK-3 Inhibitor XXII, or a pharmaceutically acceptable salt thereof, to the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) systemically administering 1Y-2090314, or a pharmaceutically acceptable salt thereof, to the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) systemically administering AZD1080, or a pharmaceutically acceptable salt thereof, to the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) systemically administering a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, to the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) locally administering CHIR99021, or a pharmaceutically acceptable salt thereof, to the middle or inner ear of the subject wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) locally administering GSK-3 Inhibitor XXII, or a pharmaceutically acceptable salt thereof, to the middle or inner ear of the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) locally administering LY2090314, or a pharmaceutically acceptable salt thereof, to the middle or inner ear of the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) locally administering AZD1080, or a pharmaceutically acceptable salt thereof, to the middle or inner ear of the subject, wherein steps a) and b) may occur in any order or simultaneously.

In one embodiment, the present disclosure provides a method of treating or preventing a disease or disorder of the ear in a subject in need thereof, comprising (a) systemically administering a valproic acid compound to the subject; and (b) locally administering a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, to the middle or inner ear of the subject, wherein steps a) and b) may occur in any order or simultaneously.

In one embodiment, the present disclosure provides a valproic acid compound and a Wnt agonist for use in a method of treating or preventing a disease or disorder of the ear in a subject in need thereof, the method comprising (a) systemically administering the valproic acid compound to the subject; and (b) locally administering the Wnt agonist to the middle or inner ear of the subject, wherein steps a) and b) may occur in any order or simultaneously.

In one embodiment, the present disclosure provides a valproic acid compound for use in a method of treating or preventing a disease or disorder of the ear in a subject in need thereof, said method comprising a) systemically administering the valproic acid compound, wherein the subject has been, or will be, b) administered a Wnt agonist locally to the middle or inner ear of the subject, wherein steps a) and b) may occur in any order or simultaneously.

In one embodiment, the present disclosure provides a Wnt agonist for use in a method of treating or preventing a disease or disorder of the ear in a subject in need thereof, said method comprising b) locally administering the Wnt agonist to the middle or inner ear of the subject, wherein the subject has been, or will be, a) administered a valproic acid compound systemically, wherein steps a) and b) may occur in any order or simultaneously.

In one embodiment, the present disclosure provides a Wnt agonist for use in a method of treating or preventing a disease or disorder of the ear in a subject in need thereof, said method comprising: a) systemically administering a valproic acid compound to the subject; and b) locally administering the Wnt agonist to the middle or inner ear of the subject, wherein steps a) and b) can occur in any order or simultaneously.

In one embodiment, the present disclosure provides a valproic acid compound for use in a method of treating or preventing a disease or disorder of the ear in a subject in need thereof, said method comprising: a) systemically administering the valproic acid compound to the subject; and b) locally administering a Wnt agonist to the middle or inner ear of the subject, wherein steps a) and b) can occur in any order or simultaneously.

In one embodiment, the present disclosure provides a method of treating or preventing a disease or disorder of the ear in a subject in need thereof, comprising (a) systemically administering a valproic acid compound to the subject; and (b) locally administering a Wnt agonist to the middle or inner ear of the subject, and (c) locally administering a valproic acid compound to the middle or inner ear of the subject, wherein steps a), b), and c) may occur in any order or simultaneously. Local administration of the valproic acid compound in addition to systemic administration may further increase delivery of valproic acid to the inner ear, thereby enhancing the therapeutic effect.

In one embodiment, the present disclosure provides a kit including a first pharmaceutical composition comprising a valproic acid compound; a second pharmaceutical composition comprising a Wnt agonist; and a set of instructions directing a user to: a) systemically administer the first pharmaceutical composition to a subject; and b) locally administer the second pharmaceutical composition to the middle or inner ear of the subject, wherein steps a) and b) can occur in any order or simultaneously.

In one embodiment, the present disclosure provides a kit including a first pharmaceutical composition comprising a valproic acid compound; a second pharmaceutical composition comprising a Wnt agonist; and a set of instructions directing a user to: a) locally administer the first pharmaceutical composition to the middle or inner ear of a subject; and b) locally administer the second pharmaceutical composition to the middle or inner ear of the subject, wherein steps a) and b) can occur in any order or simultaneously.

In one embodiment, the present disclosure provides a kit including a pharmaceutical composition comprising: a valproic acid compound, and a Wnt agonist; and a set of instructions directing a user to locally administer the pharmaceutical composition to the middle or inner ear of a subject.

In one embodiment, the present disclosure provides a kit including a first pharmaceutical composition comprising a valproic acid compound; a second pharmaceutical composition comprising CHIR99021, or a pharmaceutically acceptable salt thereof; and a set of instructions directing a user to: a) systemically administer the first pharmaceutical composition to a subject; and b) locally administer the second pharmaceutical composition to the middle or inner ear of the subject, wherein steps a) and b) can occur in any order or simultaneously.

In one embodiment, the present disclosure provides a kit including a first pharmaceutical composition comprising a valproic acid compound; a second pharmaceutical composition comprising CHIR99021, or a pharmaceutically acceptable salt thereof; and a set of instructions directing a user to: a) locally administer the first pharmaceutical composition to the middle or inner ear of a subject; and b) locally administer the second pharmaceutical composition to the middle or inner ear of the subject, wherein steps a) and b) can occur in any order or simultaneously.

In one embodiment, the present disclosure provides a kit including a pharmaceutical composition comprising: a valproic acid compound, and CHIR99021, or a pharmaceutically acceptable salt thereof; and a set of instructions directing a user to locally administer the pharmaceutical composition to the middle or inner ear of a subject.

In one embodiment, the present disclosure provides a kit including a first pharmaceutical composition comprising a valproic acid compound; a second pharmaceutical composition comprising GSK-3 Inhibitor XXII, or a pharmaceutically acceptable salt thereof; and a set of instructions directing a user to: a) systemically administer the first pharmaceutical composition to a subject; and b) locally administer the second pharmaceutical composition to the middle or inner ear of the subject, wherein steps a) and b) can occur in any order or simultaneously.

In one embodiment, the present disclosure provides a kit including a first pharmaceutical composition comprising a valproic acid compound; a second pharmaceutical composition comprising GSK-3 Inhibitor XXII, or a pharmaceutically acceptable salt thereof; and a set of instructions directing a user to: a) locally administer the first pharmaceutical composition to the middle or inner ear of a subject; and b) locally administer the second pharmaceutical composition to the middle or inner ear of the subject, wherein steps a) and b) can occur in any order or simultaneously.

In one embodiment, the present disclosure provides a kit including a pharmaceutical composition comprising: a valproic acid compound, and GSK-3 Inhibitor XXII, or a pharmaceutically acceptable salt thereof; and a set of instructions directing a user to locally administer the pharmaceutical composition to the middle or inner ear of a subject.

In one embodiment, the present disclosure provides a kit including a first pharmaceutical composition comprising a valproic acid compound; a second pharmaceutical composition comprising LY2090314, or a pharmaceutically acceptable salt thereof; and a set of instructions directing a user to: a) systemically administer the first pharmaceutical composition to a subject; and b) locally administer the second pharmaceutical composition to the middle or inner ear of the subject, wherein steps a) and b) can occur in any order or simultaneously.

In one embodiment, the present disclosure provides a kit including a first pharmaceutical composition comprising a valproic acid compound; a second pharmaceutical composition comprising LY2090314, or a pharmaceutically acceptable salt thereof; and a set of instructions directing a user to: a) locally administer the first pharmaceutical composition to the middle or inner ear of a subject; and b) locally administer the second pharmaceutical composition to the middle or inner ear of the subject, wherein steps a) and b) can occur in any order or simultaneously.

In one embodiment, the present disclosure provides a kit including a pharmaceutical composition comprising: a valproic acid compound, and LY2090314, or a pharmaceutically acceptable salt thereof; and a set of instructions directing a user to locally administer the pharmaceutical composition to the middle or inner ear of a subject.

In one embodiment, the present disclosure provides a kit including a first pharmaceutical composition comprising a valproic acid compound; a second pharmaceutical composition comprising AZD1080, or a pharmaceutically acceptable salt thereof; and a set of instructions directing a user to: a) systemically administer the first pharmaceutical composition to a subject; and b) locally administer the second pharmaceutical composition to the middle or inner ear of the subject, wherein steps a) and b) can occur in any order or simultaneously.

In one embodiment, the present disclosure provides a kit including a first pharmaceutical composition comprising a valproic acid compound; a second pharmaceutical composition comprising AZD1080, or a pharmaceutically acceptable salt thereof; and a set of instructions directing a user to: a) locally administer the first pharmaceutical composition to the middle or inner ear of a subject; and b) locally administer the second pharmaceutical composition to the middle or inner ear of the subject, wherein steps a) and b) can occur in any order or simultaneously.

In one embodiment, the present disclosure provides a kit including a pharmaceutical composition comprising: a valproic acid compound, and AZD1080, or a pharmaceutically acceptable salt thereof; and a set of instructions directing a user to locally administer the pharmaceutical composition to the middle or inner ear of a subject.

In one embodiment, the present disclosure provides a kit including a first pharmaceutical composition comprising a valproic acid compound; a second pharmaceutical composition comprising a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, and a set of instructions directing a user to: a) systemically administer the first pharmaceutical composition to a subject; and b) locally administer the second pharmaceutical composition to the middle or inner ear of the subject, wherein steps a) and b) can occur in any order or simultaneously.

In one embodiment, the present disclosure provides a kit including a first pharmaceutical composition comprising a valproic acid compound; a second pharmaceutical composition comprising a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, and a set of instructions directing a user to: a) locally administer the first pharmaceutical composition to the middle or inner ear of a subject; and b) locally administer the second pharmaceutical composition to the middle or inner ear of the subject, wherein steps a) and b) can occur in any order or simultaneously.

In one embodiment, the present disclosure provides a kit including a pharmaceutical composition comprising: a valproic acid compound, and a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof; and a set of instructions directing a user to locally administer the pharmaceutical composition to the middle or inner ear of a subject.

In one embodiment, the present disclosure provides a kit including a first pharmaceutical composition comprising a valproic acid compound; a second pharmaceutical composition comprising a Wnt agonist; a third pharmaceutical composition comprising a valproic acid compound, and a set of instructions directing a user to: a) systemically administer the first pharmaceutical composition to the middle or inner ear of a subject; b) locally administer the second pharmaceutical composition to the middle or inner ear of the subject, and c) locally administer the third pharmaceutical composition to the middle or inner ear of the subject, wherein steps a), b), and c) can occur in any order or simultaneously.

In one embodiment, the present disclosure provides a kit including a first pharmaceutical composition comprising a valproic acid compound; a second pharmaceutical composition comprising a Wnt agonist and a valproic acid compound; and a set of instructions directing a user to: a) systemically administer the first pharmaceutical composition to the middle or inner ear of a subject; b) locally administer the second pharmaceutical composition to the middle or inner ear of the subject, wherein steps a), and b) can occur in any order or simultaneously.

The present methods, and advantages thereof, are further illustrated by the following non-limiting detailed description and Examples.

### BRIEF DESCRIPTION OF THE DRAWINGS

FIG. 1 is a depiction of the human ear with the cochlea schematically unwound to show that the highest frequencies are detected in the base and the lowest frequencies are detected in the apex. A drug delivered to the middle ear (black circle) closest to the entry site at the round window diffuses along the cochlea, from high frequency loci to low frequency loci, with decreasing concentration.

FIG. 2A is a graph comparing blood plasma valproic acid concentrations in guinea pigs and humans upon systemic administration of 100 mg/mL sodium valproate (FX00) after 3 hours.

FIG. 2B is a graph showing uniform perilymph concentrations in guinea pigs 3 hours after systemic administration of 100 mg/mL sodium valproate (FX00).

FIG. 3A is a graph comparing blood plasma valproic acid concentrations in guinea pigs at two different systemically administered valproic acid (FX00) concentrations after 3 hours.

FIG. 31B is a graph comparing perilymph valproic acid concentrations in guinea pigs at two different systemically administered valproic acid (FX00) concentrations after 3 hours.

FIG. 4 is a graph comparing perilymph valproic acid concentrations in guinea pigs after 3 hours upon 1) systemic valproate and local CHIR99021/valproate administration, and 2) only local CHIR99021/valproate-only administration.

FIG. 5 is graph modeling human in vivo administration of systemic valproate and local CHIR99021/valproate administration.

### DETAILED DESCRIPTION

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

The present disclosure is based upon the surprising discovery that systemic delivery of valproic acid combined with local delivery of valproic acid and/or Wnt agonists to the middle ear of a subject results in superior therapeutic concentrations of compounds in the inner ear of the subject. This combination of systemic and local administration improved the drug distribution and retention in the subject cochlea. Accordingly, the present invention provides a superior method of treating a disease or disorder of the ear in a subject in need thereof.

### Methods of Use

In a first aspect, the present disclosure provides a method of treating a disease or disorder of the ear in a subject in need thereof comprising systemically administering an effective amount of a valproic acid compound to the subject and locally administering a Wnt agonist to the middle or inner ear of the subject.

In some embodiments, the Wnt agonist may include any Wnt agonist described herein.

In some the Wnt agonist is a GSK3 inhibitor.

In some embodiments, the GSK inhibitor is LY2090314 or a pharmaceutically acceptable salt thereof.

In some embodiments, the GSK3 inhibitor is AZD1080 or a pharmaceutically acceptable salt thereof.

In some embodiments, the GSK3 inhibitor is CHIR99021 or a pharmaceutically acceptable salt thereof.

In some embodiments, the GSK3 inhibitor is GSK-3 Inhibitor XXII or a pharmaceutically acceptable salt thereof.

In some embodiments, the Wnt agonist may include is a substituted 3-imidazo[1,2-a]pyridin-3-yl-4-(1,2,3,4-tetrahydro-[1,4]diazepino-[6,7,1-hi]indol-7-yl)pyrrole-2,5-dione (Formula A), or a pharmaceutically acceptable salt thereof.

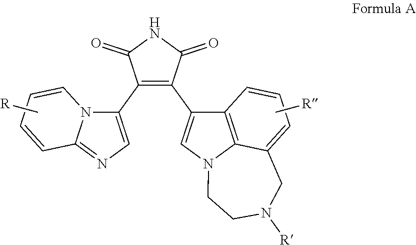

In some embodiments, the Wnt agonist may include a substituted 3-imidazo[1,2-a]pyridin-3-yl-4-(1,2,3,4-tetrahydro-[1,4]diazepino-[6,7,1-hi]indol-7-yl)pyrrole-2,5-dione selected from Compounds I-1-I-48, or a pharmaceutically acceptable salt thereof.

In some embodiments, the Wnt agonist may include a substituted 3-imidazo[1,2-a]pyridin-3-yl-4-(1,2,3,4-tetrahydro-[1,4]diazepino-[6,7,1-hi]indol-7-yl)pyrrole-2,5-dione selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof.

In various embodiments, the effective amount of the systemically administered valproic acid compound is any effective amount described herein in connection with any valproic acid compound described herein.

In various embodiments, the effective amount of the locally administered Wnt agonist is any effective amount described herein in connection with any Wnt agonist described herein.

In another aspect, the present disclosure provides for treating a disease or disorder of the ear in a subject in need thereof comprising systemically and locally administering an effective amount of a valproic acid compound to the subject, and locally administering a Wnt agonist to the middle or inner ear of the subject.

In various embodiments, the effective amount of the systemically administered valproic acid compound is any effective amount described herein in connection with any valproic acid compound described herein.

In various embodiments, the effective amount of the locally administered Wnt agonist is any effective amount described herein in connection with any Wnt agonist described herein.

In various embodiments, the effective amount of the locally administered valproic acid compound is any effective amount described herein in connection with any valproic acid compound described herein.

When a valproic acid compound is administered both systemically and locally, the locally administered valproic acid compound can be provided at a reduced amount or concentration compared to the amount or concentration that would be required to provide a substantially similar therapeutic effect, absent any systemic administration of a valproic acid compound. In some embodiments, the reduced amount or concentration of locally administered valproic acid compound would be considered a sub-therapeutic amount or concentration if administered in the absence of the systemically administered valproic acid compound, as it would not, by itself, produce the desired physiological effect (e.g. clinically relevant reduction in symptoms of tinnitus, hyperacusis, vertigo and Meniere's disease, improvement in hearing, regrowth of sensory hair cells, etc., as described herein). In other embodiments, the systemically administered amount or concentration of valproic acid compound would be considered a sub-therapeutic amount or concentration if administered in the absence of the locally administered valproic acid compound, as it would, by itself, not produce the desired physiological effect (e.g. clinically relevant reduction in symptoms of dizziness due to diseases of the inner ear area, Meniere's disease, tinnitus, and hearing loss due to antibiotics and cytostatics and other drugs, hyperacusis, vertigo improvement in hearing, regrowth of sensory hair cells, etc., as described herein). In still other embodiments, both the systemically administered amount or concentration of valproic acid compound would be considered a sub-therapeutic amount or concentration if administered individually, but in combination are therapeutically effective and provide e.g. a clinically relevant reduction in symptoms of dizziness due to diseases of the inner ear area, Meniere's disease, tinnitus, and hearing loss due to antibiotics and cytostatics and other drugs, hyperacusis, vertigo improvement in hearing, regrowth of sensory hair cells, etc., as described herein.

### Diseases and Disorders

Diseases and disorders treated by the methods of the present disclosure are associated with a reduced level of sensory hair cells, such as hearing disorders or balance disorders. For example such diseases and disorders include reduction or loss of hearing, sensorineural hearing loss, tinnitus, hyperacusis, vertigo and Meniere's disease. A reduced level of sensory hair cells may arise from a reduction in the number of hair cells in the cochlea or vestibular organs, or hair cells having impaired ability to transduce vibrational stimuli. A reduced level of sensory hair cells can therefore be associated with a reduction of loss of hearing.

In particular embodiments, the disease or disorder treated by the present invention is associated with a reduction or loss of hearing in the subject. In one embodiment, the method reduces the perception of hearing loss by the afflicted individual following the administrations. In another embodiment, the method reduces the degree of hearing loss following the administrations. In another embodiment, the method reduces the frequency of hearing loss episodes following the administrations. In still another embodiment, the method reduces the duration of hearing loss episodes following the administrations.

In many embodiments, the disease or disorder treated by the present invention is selected from hyperacusis, tinnitus, vertigo, and Meniere's disease.

In many embodiments, the disease or disorder is an inner ear hearing or balance disorder. In many embodiments, the disease or disorder is sensorineural hearing loss.

In some embodiments, the disease or disorder treated by the present invention is hyperacusis. In one embodiment, the method reduces the sensitivities to certain sound frequencies that are experienced by the afflicted individual following the administrations. In one embodiment, the method reduces the duration of sensitivities to certain sound frequencies that are experienced by the afflicted individual following the administrations. In one embodiment, the method reduces the frequency of sensitivities to certain sound frequencies that are experienced by the afflicted individual following the administrations. In one embodiment, the method reduces the sensitivities to certain volume ranges that are experienced by the afflicted individual following the administrations. In one embodiment, the method reduces the duration of sensitivities to certain volume ranges that are experienced by the afflicted individual following the administrations. In one embodiment, the method reduces the frequency of sensitivities to certain volume ranges that are experienced by the afflicted individual following the administrations. The administrations may include any mode of administration of a valproic acid and/or Wnt agonist described herein.

In some embodiments, the disease or disorder treated by the present invention is tinnitus. In one embodiment, the method reduces the perception of tinnitus by the afflicted individual following the administrations. In another embodiment, the method reduces the loudness of tinnitus following the administrations. In another embodiment, the method reduces the frequency of tinnitus episodes following the administrations. In still another embodiment, the method reduces the duration of tinnitus episodes following the administrations. The administrations may include any mode of administration of a valproic acid and/or Wnt agonist described herein.

In some embodiments, the disease or disorder treated by the present invention is vertigo. In one embodiment, the method reduces the perception of vertigo by the afflicted individual following the administrations. For example, the individual may experience a change in the sensation or feeling of spinning and/or swaying. For example, the individual may experience a change in symptoms of nausea. In another embodiment, the method reduces the intensity of vertigo following the administrations. In another embodiment, the method reduces the frequency of vertigo episodes following the administrations. In still another embodiment, the method reduces the duration of vertigo episodes following the administrations. The administrations may include any mode of administration of a valproic acid and/or Wnt agonist described herein.

In some embodiments, the disease or disorder treated by the present invention is Meniere's disease. In one embodiment, the method reduces the perception of hearing loss by the afflicted individual following the administrations. In another embodiment, the method reduces the degree of hearing loss following the administrations. In another embodiment, the method reduces the frequency of hearing loss episodes following the administrations. In still another embodiment, the method reduces the duration of hearing loss episodes following the administrations. In one embodiment, the method reduces the perception of tinnitus by the afflicted individual following the administrations. In another embodiment, the method reduces the loudness of tinnitus following the administrations. In another embodiment, the method reduces the frequency of tinnitus episodes following the administrations. In still another embodiment, the method reduces the duration of tinnitus episodes following the administrations. In one embodiment, the method reduces the perception of vertigo by the afflicted individual following the administrations. For example, the individual may experience a change in the sensation or feeling of spinning and/or swaying. For example, the individual may experience a change in symptoms of nausea. In another embodiment, the method reduces the intensity of vertigo following the administrations. In another embodiment, the method reduces the frequency of vertigo episodes following the administrations. In still another embodiment, the method reduces the duration of vertigo episodes following the administrations. The administrations may include any administration of a valproic acid and/or Wnt agonist described herein. In one embodiment, the method reduces the perception of aural fullness by the afflicted individual following the administrations. In another embodiment, the method reduces the intensity of aural fullness following the administrations. In another embodiment, the method reduces the frequency of aural fullness episodes following the administrations. In still another embodiment, the method reduces the duration of aural fullness episodes following the administrations. In many embodiments, the method reduces intensity, degree, frequency, and/or duration of one or more symptoms associated with Meniere's disease. The administrations may include any administration of a valproic acid and/or Wnt agonist described herein.

In some embodiments, the disease or disorder of the ear includes sensory hair cell disorders. In some embodiments, the disease or disorder is associated with a reduced level or number of sensory hair cells in the subject. The disease or disorder is associated with an absence of sensory hair cells in the subject. In some embodiments, the disease or disorder is associated with damaged sensory hair cells in the subject.

In many embodiments, the method treats a disease or disorder responsive to regrowth or regeneration of sensory hair cells in the cochlea. In some embodiments, the method increases the population of stem cells of the sensory hair cells by at least about 1.25-fold following the administrations. In some embodiments, the method increases the population of stem cells of the sensory hair cells by about 1.25-fold to about 20-fold following the administrations. For example, the method increases the population of stem cells of the sensory hair cells by about 1.25-fold, about 1.5-fold, about 1.75-fold, about 2-fold, about 2.25-fold, about 2.5-fold, about 2.75-fold, about 3-fold, about 3.25-fold, about 3.5-fold, about 3.75-fold, about 4-fold, about 4.25-fold, about 4.5-fold, about 4.75-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 11-fold, about 12-fold, about 13-fold, about 14-fold, about 15-fold, about 16-fold, about 17-fold, about 18-fold, about 19-fold, or about 20-fold, inclusive of all ranges defined by any values herein.

In many embodiments, the method treats a disease or disorder wherein treatment results in improved auditory function when assessed by behavioral audiometry, auditory brainstem response (ABR) testing, sound intelligibility assessments (for example, a word recognition test), pure tone audiometry, Distortion product otoacoustic emissions (DPOAEs), extended high frequency (EHF) audiometry.

In many embodiments, methods of the present disclosure are used to treat human beings. In other embodiments, methods of the present disclosure are used to treat animals other than human beings.

Absent any systemic administration of a valproic acid compound, the effective daily dose of locally administered valproic acid compound that is required would be an amount equivalent to at least about 18 mg of valproic acid, for example an amount equivalent to about 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg of valproic acid, or a range between any of the preceding values, e.g. between about 18 and about 50, between about 18 and about 25, between about 22 and about 26, or the like.

Absent any systemic administration of a valproic acid compound, the effective concentration of locally administered valproic acid compound that is required would be an amount equivalent to at least about 88 mg/mL of valproic acid, for example an amount equivalent to about 88, 89, 90, 91, 92, 93, 94, 95, 96, 97, 98, 99, or 100 mg/mL of valproic acid, or a range between any of the preceding values. e.g. between about 88 and about 95, between about 88 and about 92, between about 90 and about 99, or the like.

However in the presence of systemically administered valproic acid compound (e.g. at daily doses as provided herein), the effective daily dose of locally administered valproic acid compound may range from an amount equivalent to about 1 mg to less than about 18 mg of valproic acid, for example an amount equivalent to about 1, 2, 3, 4, 5, 6, 7, 8, 9, 10, 11, 12, 13, 14, 15, 16, 17, or less than 18 mg of valproic acid or a range between any of the preceding values, e.g. between about 5 and about 10, between about 12 and about 16, between about 7 and about 14, or the like. Alternatively the effective amount of a locally administered valproic acid compound is any one of the preceding values, or falls within a range between any of the preceding values, wherein the amount is expressed as a concentration in mg/mL. For example an effective concentration of valproic acid compound can be a concentration (expressed as the equivalent amount of valproic acid) of about 5, 10, 20, 30, 40, 50, or 60 mg/mL of valproic acid.

In various embodiments, the systemic delivery of the valproic acid compound is once or twice daily. In some embodiments, the systemic delivery of the valproic acid compound begins prior to the local administration of the Wnt agonist, for example about 1 to about 14 days prior, including 1, 2, 3, 4, 5, 6, 7, 8, 9, 10, 11, 12, 13, or 14 days prior, inclusive of all ranges there-between. In various embodiments, the local administration of the Wnt agonist is at least once or twice daily. In various embodiments, the local administration of the Wnt agonist is once or twice daily, and the systemic delivery of the valproic acid compound begins prior to the local administration of the Wnt agonist, for example about 1 to about 14 days prior, including 1, 2, 3, 4, 5, 6, 7, 8, 9, 10, 11, 12, 13, or 14 days prior. In various embodiments, the local administration of the Wnt agonist is at least once or twice weekly. In various embodiments, the local administration of the Wnt agonist is at least once or twice weekly, and the systemic delivery of the valproic acid compound begins prior to the local administration of the Wnt agonist, for example about 1 to about 14 days prior, including 1, 2, 3, 4, 5, 6, 7, 8, 9, 10, 11, 12, 13, or 14 days prior. In some embodiments, the systemic delivery of the valproic acid compound occurs at least daily until 1-8 doses of the locally administered Wnt agonist are administered (e.g. using a once or twice daily or once or twice weekly dosing schedule).

Without wishing to be bound by theory, the systemic administration of a valproic acid compound may increase the concentration of valproic acid in the region of the body to be treated (e.g. cochlea) via diffusion from the serum to the perilymph. This increase in concentration in the cochlea may reduce the effective amount or concentration of a locally-administered valproic acid compound that would be required to achieve a similar therapeutic effect upon local administration only. Alternatively, by combining systemic and local administration of valproic acid compound, the amount of systemically administered valproic acid compound can be reduced while still achieving a therapeutic effect similar to that produced by systemic administration only. Furthermore, systemic administration may prevent efflux of the valproic acid compound since the valproic acid compound is actively transported by a number of different tissues, e.g. central nervous system and gastrointestinal tract, thereby extending the half-life in the perilymph. When a valproic acid compound is administered only systemically, the half-life of valproic acid in the perilymph is lower than when administered only locally. When a valproic acid compound is only administered locally, the half-life of valproic acid in the perilymph is longer than if administered only systemically. When a valproic acid compound is administered both systemically and locally, the effective half-life of valproic acid in the perilymph is longer than if administered only locally (or systemically). In various embodiments, systemically administering a valproic acid compound and locally administering a valproic acid compound produce a valproic acid half-life in the perilymph of the subject that is about 1.1-fold, 2-fold, 3-fold, 4-fold, 5-fold, 7-fol, 7-fold, 8-fold, 9-fold or 10-fold longer than a half-life achieved upon locally administering a valproic acid compound alone. In some embodiments, the half-life is about 2-fold longer than the half-life upon local administration alone.

Without wishing to be bound by theory, administering the valproic acid systemically prior to the administration of a Wnt agonist may advantageously prime the inner ear sensory epithelium to respond to the Wnt agonist when it is administered locally, thereby providing an effective therapy for treating a subject who has, or is at risk of, developing an inner ear hearing or balance disorder.

In various embodiments, the locally administered valproic acid compound and the systemically administered valproic acid compound is independently selected from any valproic acid compound described herein. The locally administered valproic acid is the same or different from the systemically administered valproic acid compound.

In embodiments where both Wnt agonist and valproic acid compound are locally administered, the Wnt agonist and valproic acid compound is co-formulated to provide both drugs together. In other such embodiments, the Wnt agonist and valproic acid compound are formulated separately for local administration. When formulated separately, the Wnt agonist and valproic acid compound are co-administered or administered sequentially.

In another aspect, the present disclosure provides a method of treating a disease or disorder of the ear in a subject in need thereof comprising systemically administering an effective amount of a valproic acid compound to the subject and systemically administering a Wnt agonist to the subject.

In certain embodiments, the Wnt agonist may include a valproic acid compound in any of the methods described herein. In other embodiments, the Wnt agonist does not include a valproic acid compound in any of the methods described herein. The locally administered valproic acid compound and the systemically administered valproic acid compound, in any of the methods described herein, may independently be selected from any valproic acid compounds described herein.

Measurement of Sensorineural Hearing Loss

Hearing loss can be assessed by several different tests. Such tests may determine the audibility of a sound to a patient and/or the intelligibility of the sound to a patient prior to or after treatment. The audibility of a sound is a measure of a patient's ability to detect the sound (i.e. whether the patient can determine the presence or absence of a sound). The intelligibility of a sound is a measure of a patient's ability to correctly identify the sound. For instance, hearing may be assessed according to whether a patient can correctly identify a word or not. A patient with hearing loss may therefore neither be able to detect a sound nor correctly identify it (i.e. the sound is inaudible and unintelligible). However, audibility is not necessarily associated with intelligibility, and a patient may, for example, be able detect a sound, but not correctly identify it (i.e. the sound is audible but unintelligible).

Pure Tone Audiometry

Assessment of a patient's audibility function is typically carried out by an audiologist using an audiometer in a hearing test known as pure tone audiometry. Pure tone audiometry is a standard test used to assess the audibility of a sounds and is described in detail elsewhere (see, for example, Katz, J., Medwetsky, L., Burkard, R., & Hood, L. (2009) Handbook of Clinical Audiology. Philadelphia, Pa.: Lippincott Williams and Wilkins). Pure tone audiometry is typically carried out in a sound-treated booth, which reduces ambient noise levels that may interfere with the detection of low-level sound stimuli.

In pure tone audiometry, a patient is exposed to pure tone stimuli at specific frequencies to determine the patient's hearing threshold at each frequency. Standard audiometry measures a patient's pure tone hearing threshold at each of the following frequencies 0.25 kHz. 0.5 kHz, 1 kHz, 2 kHz. 3 kHz, 4 kHz, 6 kHz and 8 kHz. However, a patient's hearing threshold does not need to be determined at all of these frequencies to ascertain whether or not the patient has sensorineural hearing loss. For instance, a subset frequencies, or a single frequency may be tested to identify a patient with sensorineural hearing loss.

To determine the hearing threshold, the volume of the pure tone is altered to determine the lowest level of stimuli that the patient is able to detect. The lowest level of stimuli (corresponding to the quietest sound) is the pure tone hearing threshold at a given frequency. The pure tone threshold is typically measured in a patient using according decibels in hearing level (dB HL) on an audiometer. However, hearing thresholds may also be determined using other methods known to the person skilled in the art. For example, hearing function may be measured by Auditory Brainstem Response (ABR) testing or Auditory Steady State Response (ASSR) testing. Other tests can also be used to determine hearing function in a patient. For instance, Distortion product otoacoustic emissions (DPOAEs) can be used to measure outer hair cell function and loss and may be used in differential diagnosis of hearing loss arising from hair cell loss from hearing loss associated with higher level processing (e.g. auditory neuropathy).

Pure tone thresholds may be plotted on a graph to produce an audiogram for the patient.

Pure tone thresholds measured across different frequencies may also be averaged to provide a pure tone average. For instance, a patient that has pure tone hearing thresholds of 50 dB HL at 0.5 Hz, 60 dB HL at 1 kHz, 65 dB HL at 2 kHz and 70 dB at 4 kHz would have a pure tone average of 61.25 dB HL, when measured across 0.5 kHz. 1 kHz, 2 kHz and 4 kHz.

Pure tone averages may be calculated across different frequencies. Pure tone thresholds at any subset of frequencies may be used to calculate pure tone averages. In some embodiments, the average of the patient hearing threshold is measured across 0.5 kHz, 1 kHz, 2 kHz and 4 kHz. In some embodiments, pure tone average is measured across 4 kHz, 6 kHz and 8 kHz. Measurement of pure tone average across 4 kHz, 6 kHz and 8 kHz is useful when seeking to assess the patient's hearing function at the higher frequencies within the standard audiometric frequencies.

Sensorineural hearing loss can be categorized according to its severity. The severity of hearing loss is determined by the hearing levels at which a threshold level is obtained in a patient by pure tone audiometry. Severity of hearing loss is classified according to hearing thresholds using the following definitions:
Normal: 25 dB HL or less
Mild: at least 25 dB HL and no more than 40 dB HL
Moderate: at least 40 dB HL and no more than 55 dB HL
Moderately Severe: at least 55 dB HL and no more than 70 dB HL
Severe: at least 70 dB HL and no more than 90 dB HL
Profound: at least 90 dB HL or more
   These measures of severity are standard measures in the field (see Goodman, A. (1965). Reference zero levels for pure tone audiometer. ASHA, 7, 262-263). In some embodiments, the severity of hearing loss is classified according to a patient's hearing threshold at a single frequency (for example, 0.25 kHz, 0.5 kHz, 1 kHz, 2 kHz, 3 kHz, 4 kHz, 6 kHz or 8 kHz). For instance, a patient may have mild hearing loss at 8 kHz, and normal hearing at the other standard audiometric frequencies. In some embodiments, the severity of hearing loss is classified according to pure tone average, when measured across a subset of frequencies. In certain such embodiments, the severity of hearing loss is classified according to the pure tone average across 0.5 kHz, 1 kHz. 2 kHz and 4 kHz. For example, a patient may have moderate hearing loss according to their pure tone average across 0.5 kHz, 1 kHz, 2 kHz and 4 kHz, but have moderately severe hearing loss at a single frequency (e.g. 8 kHz). In other embodiments, the severity of hearing loss is classified according to the pure tone average across 4 kHz, 6 kHz and 8 kHz.

A patient that has hearing threshold of 25 dB HL or less at standard audiometric frequencies (i.e. 0.25 kHz, 0.5 kHz, 1 kHz, 2 kHz, 3 kHz, 4 kHz, 6 kHz and 8 kHz) has normal hearing. The patient's audiogram is also a normal audiogram.

### Word Recognition Tests

Alternatively, or in addition to pure tone audiometry, hearing loss may be assessed using a word recognition test. A word recognition test measures the patient's ability to correctly identify a word, thereby providing a measure of sound intelligibility (in particular, speech intelligibility) that may not be provided by pure tone audiometry. In some embodiments, a word recognition score is used to determine the patient's ability to correctly identify words prior to treatment.

A standard word recognition in quiet test, also referred to herein as a standard word recognition test, is a test administered by an audiologist that measures a patient's speech intelligibility in recognizing words in a quiet environment. A quiet environment is an environment with little to no background noise.

A standard word recognition test may be used to determine a person's ability to recognize words selected from a word list and presented to the patient at a given decibel (dB) level. In some embodiments, the standard word recognition test is used to determine a patient's ability to recognize words at more than one decibel level.

In some embodiments, the standard word recognition test assesses the patient's ability to identify 50 words. However, the number of words presented to the patient may be more or less than 50. For example, in some embodiments, the standard word recognition test is for 25 words. In other embodiments, the standard word recognition test is for 10 words.

A standard word recognition test may be used to generate a standard word recognition (%) score which is calculated using the formula:

$$standard word recogntion score ( \% ) = 100 \times ( \frac{words recognised in standard word recognition test}{total words} )$$

In some embodiments, the standard word recognition score is expressed as the number of words that are correctly recognized in the test.

In some embodiments, a list of words is administered to each ear, and a standard word recognition score is calculated for each ear. Herein the results of the standard word recognition score refer to the ear that has been/will be treated.

A standard word recognition test may be carried out using any list of words. However, standard word lists are typically used in a standard word recognition test. In some embodiments, each test word is embedded in a carrier phrase. Example of carrier phrases are: “Say the word ______ again”, “You will say ______” or “Say the word ______”.

In some embodiments, the standard word recognition test is the Maryland consonant-vowel nucleus-consonant (CNC) word test. The Maryland CNC word test has been described, for example, in Mendel, L. L., Mustain, W. D., & Magro, J. (2014). Normative data for the Maryland CNC Test. Journal of the American Academy of Audiology, 25, 775-781.

The Maryland CNC word test is a standard word recognition test that uses phonemically balanced word lists comprising words that are consonant-nucleus-consonant (CNC) monosyllables. These CNC lists are balanced so that each initial consonant, each vowel, and each final consonant appears with the same frequency within each list. The Maryland CNC test has 10 lists of 50 words.

In some embodiments, the Maryland CNC Test uses words from Lehiste and Peterson's phonemically balanced word lists, all of which were CNC monosyllables, for example as described in Lehiste I, Peterson G E. (1959) Linguistic considerations in the study of speech intelligibility. Journal of the Acoustical Society of America 31(3): 280-286.

In some embodiments, the Maryland CNC Test uses words from revised CNC lists that eliminate rare literary words and proper names, for example as described in Peterson G E, Lehiste I. (1962) Revised CNC lists for auditory tests. Journal of Speech and Hearing Disorders 27:62-70.

In some embodiments, the Maryland CNC Test uses words from modified CNC word lists that take into consideration the effects of coarticulation, where the acoustic properties of phonemes are influenced by those phonemes that immediately precede and follow them, for example as described in Causey G D, Hood L J, Hermanson C L, Bowling L S. (1984) The Maryland CNC Test: normative studies. Audiology 23(6): 552-568. The words of the Maryland CNC test are spoken within the carrier phrase: ‘Say the ______ again,’

In some embodiments, the standard word recognition test is the C.I.D Auditory Test W-22 (CID W-22) test. The CID W-22 test has been described, for example, in Hirsh, I. J., Davis, H. Silverman, S. R., Reynolds. E. G., Eldert, E., & Benson, R. W. (1952). Development of Materials for Speech Audiometry. Journal of Speech, Language, and Hearing Research, 17(3), 321-337.

The CID W-22 test uses 200 monosyllabic words which are divided into four lists of 50 words each. Each list is phonetically balanced. The speech sounds within the list occur with the same relative frequency as they do in a representative sample of English speech. There are three criteria for the vocabulary in the phonetically balanced word lists. First, all the words must be one-syllable words with no repetition of words in the different lists. Second, any word chosen should be a familiar word. This second criterion is to minimize the effect of differences in the educational background of subjects. Third, the phonetic composition of each word list should correspond to that of English as a whole as closely as possible. The words of the CID W-22 test are spoken with the carrier phrase: “You will say ______”.

In some embodiments the standard word recognition test is the NU No. 6 test. The NU No. 6 has been described, for example, in Tillman, T. W., & Carhart, R. (1966). An expanded test for speech discrimination utilizing CNC monosyllabic words: Northwestern University Auditory Test No. 6. Northwestern Univ Evanston II Auditory Research Lab.

In some embodiments, the NU No. 6 test uses 4 lists of 50 words, for example, as described in Table 28-2 of Tillman, T. W., & Carhart, R. (1966). The words of the NU No. 6 test are spoken with the carrier phrase: “Say the word ______”.

In some embodiments the standard word recognition test is the Maryland CNC test, using the words list and carrier phrases as defined in Causey G D, Hood L J, Hermanson C L, Bowling L S. (1984) The Maryland CNC Test: normative studies. Audiology 23(6): 552-568. In certain such embodiments, the word signal is provided to the patient at 40 dB above speech perception level.

### Words-In-Noise (WIN) Test

A “Words-in-Noise (WIN) Test” is a test administered by an audiologist to measure a patient's speech intelligibility in recognizing words in the presence of background noise.

The WIN test consists of administering words to an ear at a varying signal-to-noise ratio (SNR) level. The signal-to-noise ratio is the ratio of the strength of the signal carrying information (e.g. the test word signal), relative to the signal of interference (e.g. noise), and is typically expressed in decibels. In some embodiments, the background noise is multi-talker babble at a fixed decibel level.

In some embodiments the multi-talker babble is comprised of six talkers (three female, three male) at a fixed level, for example, as described in Wilson, R. H., Abrams, H. B., & Pillion, A. L. (2003). A word-recognition task in multi-talker babble using a descending presentation mode from 24 dB to 0 dB signal to babble. Journal of Rehabilitation Research and Development. 40(4), 321-328.

In some embodiments, the background noise is maintained at a fixed decibel level, and the variation in the SNR decibel level is achieved by varying the decibel level of the test word signal. The SNR decibel level is therefore the SNR above the background noise. For example if the level of multi-talker babble is fixed at 70 dB SPL, and the level of the test word signal varied from 70 dB SPL to 94 dB SPL, this would give a SNR decibel level variation of 0 dB to 24 dB.

In some embodiments, the test words that are used may be from any list described herein for the word recognition tests. In some embodiments, the word-in-noise test is for 70 words. In other embodiments, the words-in-noise test is for 35 words.

In some embodiments, the test consists of administering 35 or 70 monosyllabic words from the NU No. 6 word lists. The test words may be spoken with the carrier phrase: “Say the word ______”.

In some embodiments, the WIN test is administered in a descending-level SNR paradigm. In these embodiments, the test words at the high SNR decibel level are presented first, followed by test words at gradually lower SNR decibel levels, with words at the lowest SNR decibel level administered last. The high SNR decibel level is the easiest setting for the patient to identify the signal words. The low SNR decibel levels is the most difficult setting for the patient to identify the signal words. In other embodiments, the WIN test is administered in a randomized-level SNR paradigm. In these embodiments, the test words are presented at different SNR decibel levels in a randomized order.

In some embodiments the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR).

In some embodiments the WIN test consists of administering 70 monosyllabic words from the NU No. 6 word lists, where the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR). In this embodiment, the level of multi-talker babble is fixed at 70 dB SPL, and the level of the test word signal varies from 70 dB SPL to 94 dB SPL.

The ‘words-in-noise’ test may be used to generate a words-in-noise score.

In some embodiments the words-in-noise score is given as a percentage of the total correct words recognized by the patient in the test and calculated using the formula:

$$words n noise score ( \% ) = 100 \times ( \frac{words recognised in standard words in noise test}{total words} )$$

### Systemic Administration

Systemic administration of a valproic acid compound is delivered in a therapeutically effective amount (with daily doses as described herein) to a subject in need of treatment via any suitable route of administration. For example, the valproic acid compound is administered via ingestion (e.g. an oral tablet, capsule, suspension, etc.), or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations. Such administration is as a single or multiple oral doses, a bolus injection, multiple injections, or as a short- or long-duration infusion. Implantable devices (e.g. implantable infusion pumps) may also be employed for the periodic parenteral delivery over time of equivalent or varying dosages of the particular formulation. For such parenteral administration, compositions are formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

In various embodiments, the valproic acid compound is an oral dosage form containing an amount of valproic acid compound (e.g. valproic acid and/or sodium valproate) equivalent to about 50 mg, about 100 mg, about 125 mg, about 250 mg, about 500 mg, about 750 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid. In some embodiments, the valproic acid compound is an oral dosage form containing an amount of valproic acid compound (e.g. valproic acid and/or sodium valproate) equivalent to about 250 mg, about 500 mg, about 750 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid.

In various embodiments, the valproic acid compound is an intravenous form and is administered per day at about: 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 mg/kg; or a value or range between any of the preceding values, for example between about 10 mg/kg and about 60 mg/kg, between about 10 mg/kg and about 300 mg/kg, between about 10 mg/kg and about 150 mg/kg, or the like. In some embodiments, the valproic acid compound is administered per day at about 60 mg/kg/day.

As demonstrated herein (see Examples), systemic delivery of a valproic acid compound can improve drug delivery. It can also be advantageous because it is simpler than local administration to the inner ear. Systemic administration may allow subjects to self-administer part or all of a treatment in a way that is not necessarily possible with local administration. It can also be more straightforward to achieve co-administration when one agent is administered systemically and the other locally. Systemic delivery can also allow for flexibility in extending the duration of dose, i.e. extending the duration of a maintained therapeutic plasma level.

The invention is based on the finding that a systemically administered valproic acid compound is capable of reaching the necessary tissues in sufficient quantities in order to provide an effective therapy for diseases or disorders of the ear, when the systemic admiration occurs in conjunction with administration of a Wnt agonist as disclosed herein.

Systemic administration of a Wnt compound is delivered in a therapeutically effective amount (with daily doses as described herein) to a subject in need of treatment via any suitable route of administration. For example, the Wnt agonist is administered via ingestion (e.g. an oral tablet, capsule, suspension, etc.), or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations. Such administration is as a single or multiple oral doses, a bolus injection, multiple injections, or as a short- or long-duration infusion. Implantable devices (e.g. implantable infusion pumps) may also be employed for the periodic parenteral delivery over time of equivalent or varying dosages of the particular formulation. For such parenteral administration, compositions are formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives. In various embodiments, the Wnt agonist is an oral dosage form containing an amount of Wnt agonist of about 1 to about 500 mg, including about 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 mg, inclusive of all value and ranges between any of these values. In some embodiments, the Wnt agonist is an oral dosage form containing an amount of Wnt agonist of about 0.01 μg to about 5000 μg, including about 0.01, 0.05, 0.1, 0.5, 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 55, 60, 65, 70, 75, 80, 85, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, 1000, 1500, 2000, 2500, 3000, 3500, 4000, 4500, or about 5000 μg, inclusive of all value and ranges between any of these values. In other embodiments, the Wnt agonist is an oral dosage form containing an amount of Wnt agonist of about 0.1 μg to about 500 mg.

In some embodiments, a valproic acid compound and a Wnt compound is co-formulated for systemic administration.

### Local Administration

### Middle Ear Administration

Access to the inner ear is achieved through a variety of middle-inner interface tissue structures, such as the round window membrane, the oval window/stapes footplate, the annual ligament, or the endolymphatic sac/endolymphatic duct. The membrane of the round or oval window is the biological barrier to the inner ear space and represents the major obstacle for the local treatment of hearing impairment. The administered active agent must overcome this membrane to reach the inner ear space. The active agent can injected intra-tympanically or surgically placed in the middle ear and can then penetrate through the round window membrane. Substances (e.g. active agents) that penetrate the round window typically distribute in the perilymph and thus reach the hair cells and supporting cells.

Local administration of a Wnt agonist and/or valproic acid compound to the inner ear via the middle ear is accomplished by various delivery techniques. These include, for example, the use of devices to transport and/or deliver the Wnt agonist and/or valproic acid compound in a targeted fashion to the membranes of the round or oval window, where it diffuses into the inner ear or is actively infused. Examples include otowicks (see e.g. U.S. Pat. No. 6,120,484, which is hereby incorporated by reference), round window catheters (see e.g. U.S. Pat. Nos. 5,421,818; 5,474,529; 5,476,446; 6,045,528; 6,377,849; and U.S. Pat. Pub. No. 2002/0082554, each of which is hereby incorporated by reference in its entirely for all purposes), or microimplants (see e.g. WO 2004/064912, which is hereby incorporated by reference in its entirely for all purposes). Other techniques include transtympanic injection (also referred to as “intratympanic injection”), wherein the Wnt agonist and/or valproic acid compound is injected through the tympanic membrane into the middle ear for diffusion across the round window membrane (see e.g. Light, J.; Silverstein, H., Curr. Opin. Otolaryngol. Head Neck Surg. 2004, 12, 378-383, which is hereby incorporated by reference in its entirely for all purposes). For repeated injections, a middle ear ventilation tube is inserted into the tympanic membrane, through which the Wnt agonist and/or valproic acid compound is administered to the middle ear space. Some groups have applied drugs in a sustained manner using microcatheters and microwicks, while the majority have applied them as single or as repeated IT injections (up to 8 injections over periods of up to 2 weeks). Furthermore, drug carriers that are too viscous to be injected are deposited across a small opening in the tympanic membrane with the aid of a surgical instrument.

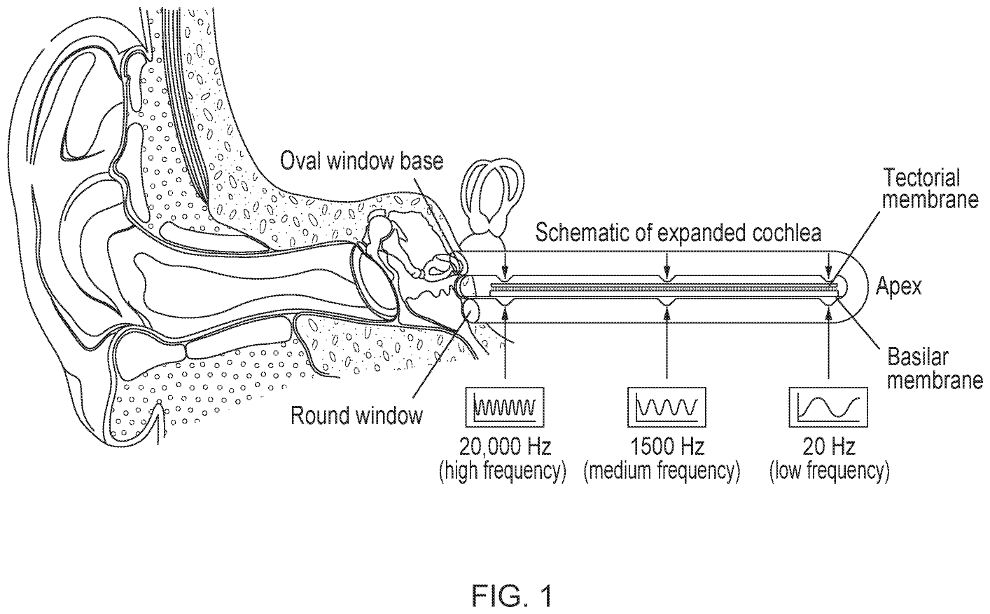

Intratympanically-applied active agents are thought to enter the fluids of the inner ear primarily by crossing the round window (RW) and oval window (OW) membranes. Calculations show that a major factor controlling both the amount of drug entering the ear and the distribution of drug throughout the ear is the duration the drug remains in the middle ear space. Single, ‘one-shot’ applications or applications of aqueous solutions for few hours' duration result in steep drug gradients for the applied substance. Because inner ear fluids are connected, a drug delivered to the inner ear will contact the vestibular organs and cochlea (FIG. 1). The vestibular organs reside in close proximity to the oval window.

Other injection approaches include by osmotic pump, or, by combination with implanted biomaterial, or by injection or infusion. Biomaterials that can aid in controlling release kinetics and distribution of drug include hydrogel materials, degradable materials. One class of materials that are used includes in situ gelling materials. Other materials include collagen or other natural materials including fibrin, gelatin, and decellularized tissues. Other additives or excipients may include, for example, Gelfoam®, hyaluronic gel, Seprapack™, Poloxamer 407, chitosan glycosylated derivatives, chitosan glycerophosphate hydrogel, lipid nanocapsules, silica nanoparticles, PLGA nanoparticles, superparamagnetic iron oxide nanoparticles encapsulated PLGA nanoparticles, lipid core nanocapsules poly-L-lysine (HBPL) nanoparticles, superparamagnetic iron oxide nanoparticles, superparamagnetic iron oxide nanoparticles encapsulated pluronic F127 copolymer, polymersome, thiol-modified hyaluronic acid, glutaraldehyde cross-linking of porcine type collagen, or the like (Acta Pharmaeutica Sinica B 2013, 3(2), 86-96, which is incorporated by reference herein in its entirety for all purposes). Delivery may also be enhanced via alternate means including but not limited to agents added to the delivered composition such as penetration enhancers, or could be through devices via ultrasound, electroporation, or high speed jet.

The diffusion of pharmaceutical compounds across middle-inner ear interface tissue structures, in particular the round window membrane, depends on a variety of factors, such as molecular weight, concentration, liposolubility, electrical charge, and thickness of the membrane (Goycoolea, M.; Lundman, L., Microsc. Res. Techniq. 1997, 36, 201-211, which is incorporated by reference herein in its entirety for all purposes). Furthermore, techniques have been developed for increasing middle-inner ear diffusion. For example, it was shown that hyaluronic acid increases the permeability of the round window membrane, whereby allowing lidocaine to more rapidly diffuse into the inner ear and promote a larger effect (Selivanova, et al. Laryngo. Rhino. Otol. 2003, 82, 235-239, which is incorporated by reference herein in its entirety for all purposes). Further, for example, it was shown that transtympanic injection with histamine allowed for higher concentrations of dexamethasone in the perilymph of the inner ear than when administered without histamine (Chandrasekhar, S., Otol. Neurotol. 2001, 22, 18-23, which is incorporated by reference herein in its entirety for all purposes).

In some embodiments, the Wnt agonist distributes through the cochlea at a rate of about 0.1 mM/(mm*min). In other embodiments, the Wnt agonist is distributed as a concentration gradient through the cochlea with an average concentration for specific Wnt agonists disclosed herein as provided in various disclosed embodiments.

### Inner Ear Administration

Other techniques include the use of devices which are inserted into the cochlear duct or any other part of the cochlea (see e.g. U.S. Pat. No. 6,309,410, which is incorporated by reference herein in its entirety for all purposes). Direct injection into the cochlea through the round window membrane with a micro syringe and narrow-gauge needle is also an available technique. Reciprocating perfusion systems and osmotic mini-pumps directly deliver agents via cannula into the scala tympani (J. Control. Release 2011, 152, 270-277, which is incorporated herein by reference in its entirety for all purposes). Further, an implantable microfluidics-based intracochlear drug delivery device is capable of time-sequence release of multiple agents to the inner ear (Audiol. Neurootol. 2009, 14, 411422, which is incorporated herein by reference in its entirety for all purposes).

### Valproic Acid Compound

Valproic acid (2-propylpentanoic acid or 2-propylvaleric acid) is currently available as a medication in injectable and oral forms used to treat epilepsy and bipolar disorder, and to prevent migraine headaches. Valproic acid is marketed under the brand names Depakote® (valproic acid, sodium valproate mixture) and Epilim® (sodium valproate), among others. As used herein, “valproic acid compound” includes valproic acid, esters thereof, amides thereof, pharmaceutically acceptable salts thereof, and any combination thereof. In some embodiments, the valproic acid compound is valproic acid (VPA). In other embodiments, the valproic acid compound is a pharmaceutically acceptable alkali metal salt of valproic acid. For example, the valproic acid compound is lithium valproate, sodium valproate, potassium valproate, or cesium valproate. In other embodiments, the valproic acid compound is a pharmaceutically acceptable alkaline earth metal salt of valproic acid. For example, the valproic acid compound is magnesium divalproate or calcium divalproate. In other embodiments the valproic acid compound is a valproic acid ester. For example, the valproic acid compound is C₁-C₇ ester, e.g. methyl valproate, ethyl valproate, propyl valproate, isopropyl valproate, butyl valproate, iso-butyl valproate, sec-butyl valproate, pentyl valproate, iso-pentyl valproate, neo-pentyl valproate, phenyl valproate, benzyl valproate, polymeric valproate esters (e.g. 2-hydroxyethyl methacrylate polymers or copolymers) or the like. In some embodiments, the valproic acid compound is methyl valproate. In other embodiments, the valproic acid compound is a valproamide. For example, the valproic acid compound may have an amide nitrogen —C(O)N(R)(R), wherein each R is independently H or C₁-C₄, e.g. —C(O)NH₂, —C(O)NH(CH₃), —C(O)NH(CH₂CH₃), —C(O)NH(CH₂CH₂CH₃), —C(O)NH(CH(CH₃)₂), —C(O)NH(CH(CH₃)CH₂CH₃), —C(O)NH(CH₂CH(CH₃)₂), —C(O)NH(CH₂CH₂CH₂CH₃), —C(O)NH(C(CH₃)₃), —C(O)N(CH₃)₂, —C(O)N(CH₂CH₃)₂, —C(O)N(CH₂CH₂CH₃)₂, —C(O)N(CH(CH₃)₂)₂. —C(O)N(CH(CH₃)CH₂CH)₂, —C(O)N(CH₂CH(CH₃)₂)₂, —C(O)N(CH₂CH₂CH₂CH₃)₂. —C(O)N(C(CH₃)₃)₂, —C(O)N(CH₃)(CH₂CH₃), or the like. In some embodiments, the valproamide is N,N-dimethyl valproamide. In other embodiments, the valproic acid compound may include amino acid valproates such as phenylalanine valproate.

### Wnt Agonists

The Wnt signaling pathways are a group of signal transduction pathways made of proteins that pass signals from outside of a cell through cell surface receptors to the inside of the cell. Three Wnt signaling pathways have been characterized: the canonical Wnt pathway, the noncanonical planar cell polarity pathway, and the noncanonical Wnt/calcium pathway. All three Wnt signaling pathways are activated by the binding of a Wnt-protein ligand to a Frizzled family receptor, which passes the biological signal to the protein Disheveled inside the cell. The canonical Wnt pathway leads to regulation of gene transcription, the noncanonical planar cell polarity pathway regulates the cytoskeleton that is responsible for the shape of the cell, and the noncanonical Wnt/calcium pathway regulates calcium inside the cell. Each pathway plays an important role in the regulation of cell proliferation and differentiation and aberrant activation of the Wnt signaling pathway is known to promote uncontrolled cell growth and survival.

The Wingless/Integrated (Wnt) signaling pathways are a group of signal transduction pathways that pass signals from outside of a cell through cell surface receptors to the inside of the cell. Three major Wnt signaling pathways have been characterized to date: the canonical Wnt pathway, sometimes referred to as the Wnt/β-catenin pathway, and the non-canonical or β-catenin independent Wnt pathways, which can be divided into the Planar Cell Polarity pathway and the non-canonical Wnt/calcium pathway. Wnt signaling regulates a wide array of biological processes including, but not limited to, cell fate determination, body axis formation and organogenesis during development, as well as cell motility, cell polarity, stem cell renewal, synapse formation and inflammation.

A Wnt agonist refers to an agent that increases the expression, levels, and/or activity of a Wnt gene, protein, or signaling pathway (e.g. TCF/LEF, Frizzled receptor family, Wifl, Lefl, Axin2, β-catenin) in a cell, for example, a cochlear cell. A Wnt agonist includes a GSK3 inhibitor, such as a GSK-3α or a GSK-3β inhibitor.

The TCF/LEF family is a group of transcription factors that bind to DNA through a high mobility group domain, and which are involved in the Wnt signaling pathway where they recruit the coactivator beta-catenin to enhancer elements of targeted genes. Frizzled is a family of G protein-coupled receptor proteins that serves as receptors in the Wnt signaling pathway. Frizzled receptors inhibit intracellular beta-catenin degradation and activate TCF/LEF-mediated transcription.

In some embodiments, a Wnt agonist increases Wnt signaling in a cochlear cell by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, a Wnt agonist increases TCF/LEF-mediated transcription in a cochlear cell, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, a Wnt agonist binds and activates a Frizzled receptor family member, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, a Wnt agonist inhibits GSK-3 for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more or more relative to a control, for example relative to a baseline level of activity.

In some embodiments, the Wnt agonist preferentially upregulates Jag-1, Deltex-1 or Hif-1 more that the Wnt agonist upregulates Hes or Hey. In some embodiments, the Wnt agonist increases the expression of Jag-1, Deltex-1 and/or Hif-1 10%, 25%, 50%, 75%, 100%, 125%, 150%, 175%, 200%, 250% or more than it increases the expression or activity of Hes and Hey.

In some embodiments, an agent of having activity as a Wnt agonist is a GSK-3 inhibitor such as AZD1080, LY2090314, GSK3 inhibitor XXII or CHIR99021.

In other embodiments, Wnt agonist and/or GSK-3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione. (Formula A.)

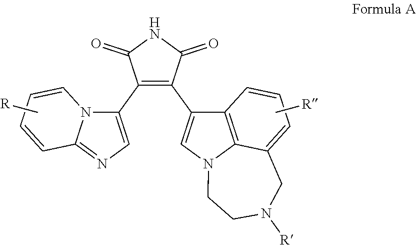

The Wnt agonist can be any selected from WO 2018/125746, which is hereby incorporated by reference. In some embodiments, the Wnt agonist can be the compound as defined in claim 1 of WO 2018/125746. In some embodiments, the Wnt agonist can be the compound as defined in claim 12 of WO 2018/125746.”

Exemplary, substituted 3-Imidazo[1,2-a]pyridin-3-yl-4-(1,2,3,4-tetrahydro-[1,4]diazepino-[6,7,1-hi]indol-7-yl)pyrrole-2,5-dione include: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione: 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile: 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione: 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide: 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile: 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione (LY20900314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is: 3-(imidazo[1,2-a]pyridin-3-yl)-4-(2-(piperidine-1-carbonyl)-9-(trifluormethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione: 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione: 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione: 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione: 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

Exemplary Wnt agonists are disclosed in Tables 1-4. Table 1 includes a selection of exemplary agents known to inhibit GSKβ. Table 2 includes a selection of exemplary agents known to inhibit GSKα. Table 3 includes a selection of Wnt agonists. Table 4 includes a selection GSK inhibitors.

In certain embodiments of the methods disclosed herein, a Wnt agonist may include a GSK inhibitor. In some embodiments, the GSK inhibitor is a GSK-3β inhibitor. In some embodiments, a Wnt agonist may include, but are not limited to, an agent selected from the agents described in Table 1, any agent disclosed in any of the references cited in Table 1, any derivatives thereof, or pharmaceutically acceptable salts thereof. A Wnt agonist is selected from any grouping of agents described or disclosed in Table 1, or any derivatives and pharmaceutically acceptable salts thereof. For example, a Wnt agonist is selected from valproic acid, CHIR99021, and GSK-3β Inhibitor XVIII; CHIR99021, GSK-3β Inhibitor XVIII, GSK-3 Inhibitor IX, and GSK-3 Inhibitor X; GSK-3 inhibitor 1 and GSK-3β Inhibitor XXVI; or the like. Classes of GSK-3β inhibitors for use in various embodiments of the methods disclosed herein include, but are not limited to, those listed in Table 1.

It is to be understood that the compounds of the present disclosure is depicted in neutral or charged form. Neutral form involves all atoms of a compound having a valency of zero. Charged form, i.e. salt form, involves one or more atoms of a compound having a non-zero valency. For example, a nitrogen atom with three bonds has a valency of zero and an oxygen atom having two bonds has a valency of zero. Further, for example, a nitrogen atom have four bonds has a valency of +1 and an oxygen atom having one bond has a valency of −1. Most commonly, a salt form involves the protonation (the addition of hydrogen) or deprotonation (the removal of hydrogen). A charged form may include a nitrogen atom having a valency of +1 due to quaternization, i.e. the addition of a moiety other than hydrogen, for example, an alkyl group, e.g. —CH₃. It is further to be understood that the salt forms includes a counterion for each atom having a non-zero valency. The counterion is any counterion that is pharmaceutically acceptable in the art. Anionic counterions, having a valency <0, may include, for example, acetate, benzoate, besylate, bitartrate, bromide, carbonate, chloride, citrate, edetate, edislate, estolate, fumarate, gluceptate, gluconate, iodide, lactate, lactobionate, malate, maleate, mandelate, mesylate, sulfate, mucate, napsulate nitrate, pamoate, phosphate, diphosphate, salicylate, disalicylate, stearate, succinate, sulfate, tartrate, tosylate, triethiodide, trifluoroacetate, ditrifluoroacetate, valerate, or the like. Cationic counterions, having a valency >0, may include, for example, aluminum, benzathine, calcium, ethylene diamine, lysine, magnesium, meglumine, potassium, procaine, sodium, tromethamine, zinc, or the like.

It is further to be understood that the compounds of the present disclosure is depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

It is further to be understood that the compounds of the present disclosure is depicted as deuterated analogs. Deuterated analogs include one or more hydrogen atoms replaced with deuterium atoms.

It is further to be understood that the compounds of the present disclosure are depicted as stereoisomers, including enantiomers and diastereomers, for example. All stereoisomers are intended to be included by the present disclosure.

TABLE I
GSK-3β Inhibitors

| Agent | | |
|---|---|---|
| CAS No. | Neutral Structure | Example Salt Forms |
| Valproic Acid 99-66-1 | 2-propyl pentanoic acid 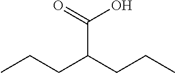 | |
| Sodium Valproate 1069-66-5 | | sodium 2-propylpentanoate 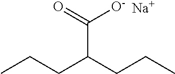 |
| Bikinin | 4-((5-bromo-2-pyridinyl)amino)- | |
| 188011-69-0 | 4-oxobutanoic acid | |
| | 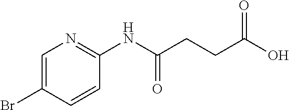 [SMILES: O=C(O)CCC(=O)Nc1ccc(Br)cn1] | |
| Hymenialdisine 82005-12-7 | (Z)-4-(2-amino-5-oxo-3,5- dihydro-4H-imidazol-4-ylidene)- 2-bromo-4,5,6,7- tetrahydropyrrolo[2,3-c]azepin- 8(1H)-one 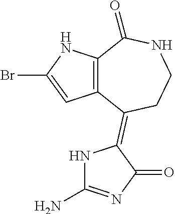 [SMILES: NC1=NC(=O)/C(=C2\CCNC(=O)c3[nH]c(Br)cc32)N1] | (Z)-4-(2-bromo-8-oxo-5,6,7,8- tetrahydropyrrolo[2,3- c]azepin-4(1H)-ylidene)-5- oxoimidazolidin-2-iminium salt 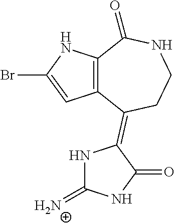 [SMILES: [NH2+]=C1NC(=O)/C(=C2\CCNC(=O)c3[nH]c(Br)cc32)N1] |
| Aloisine A | 4-(7-butyl-5H-pyrrolo[2,3- | |
| 496864-16-5 | b]pyrazin-6-yl)phenol | |
| | 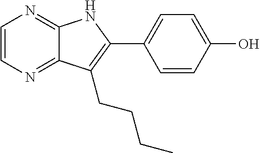 [SMILES: CCCCc1c(-c2ccc(O)cc2)[nH]c2nccnc12] | |
| Aloisine B | 6-(4-chlorophenyl)-7-isopropyl- | |
| 496864-14-3 | 5H-pyrrolo[2,3-b]pyrazine | |
| | 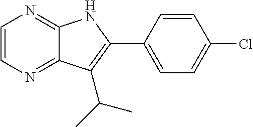 [SMILES: CC(C)c1c(-c2ccc(Cl)cc2)[nH]c2nccnc12] | |
| TWS119 | 3-[[6-(3-aminophenyl)-7H- | 3-[[6-(3-aminophenyl)-7H- |
| 1507095-58-0 | pyrrolo[2,3-d]pyrimidin-4- | pyrrolo[2,3-d]pyrimidin-4- |
| | yl]oxyphenol | yl]oxyphenol ditrifluoroacetate |
| | 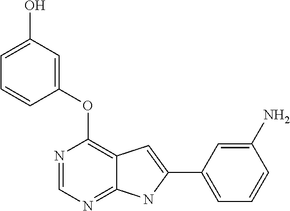 [SMILES: Nc1cccc(-c2cc3c(Oc4cccc(O)c4)ncnc3[nH]2)c1] | 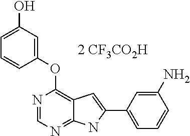 [SMILES: Nc1cccc(-c2cc3c(Oc4cccc(O)c4)ncnc3[nH]2)c1] |
| CT20026 | 1-[2-[[2-[(6-amino-5-nitro-2- | |
| 403808-63-9 | pyridinyl)amino]ethyl]amino]-4- | |
| | (2,4-dichlorophenyl)-5- | |
| | pyrimidinyl]-4-2-methyl- | |
| | piperazinone | |
| | 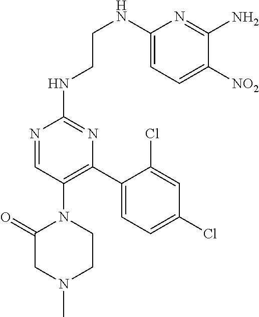 [SMILES: CN1CCN(c2cnc(NCCNc3ccc([N+](=O)[O-])c(N)n3)nc2-c2ccc(Cl)cc2Cl)C(=O)C1] | |
| CHIR99021 | 6-[[2-[[4-(2,4-dichlorophenyl)-5- | |
| (CT99021) | (5-methyl-1H-imidazol-2-yl)-2- | |
| 252917-06-9 | pyrimidinyl]amino]ethyl]amino]- | |
| | 3-pyridinecarbonitrile | |
| | 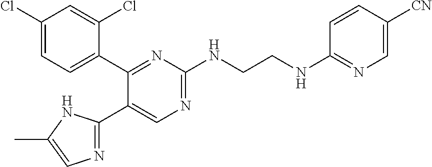 [SMILES: Cc1cnc(-c2cnc(NCCNc3ccc(C#N)cn3)nc2-c2ccc(Cl)cc2Cl)[nH]1] | |
| CHIR98014 | N-6-[2-[[4-(2,4-dichlorophenyl)-5- | |
| (CT98014) | (1H-imidazol-1-yl)-2- | |
| 252935-94-7 | pyrimidinyl]amino]ethyl]-3-nitro- | |
| | 2,6-pyridinediamine | |
| | 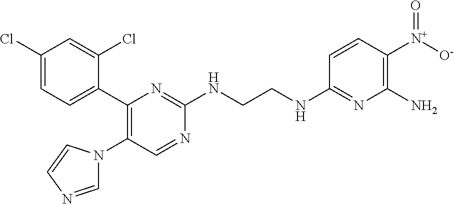 [SMILES: Nc1nc(NCCNc2ncc(-n3ccnc3)c(-c3ccc(Cl)cc3Cl)n2)ccc1[N+](=O)[O-]] | |
| CHIR98023 | N¹-[4-(2,4-dichlorophenyl)-5-(1H- | |
| (CT98023) | imidazol-1-yl)-2-pyrimidinyl]-N²- | |
| 252904-84-0 | (5-nitro-2-pyridinyl)-1,2- | |
| | Ethanediamine | |
| | 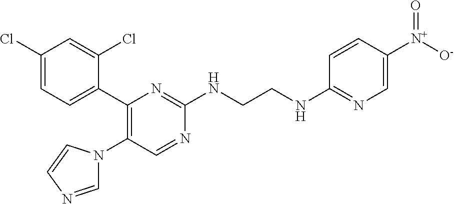 [SMILES: O=[N+]([O-])c1ccc(NCCNc2ncc(-n3ccnc3)c(-c3ccc(Cl)cc3Cl)n2)nc1] | |
| CHIR98024 | N²-(2-((4-(2,4-dichlorophenyl)-5- | |
| (CT98024) | (1H-imidazol-2-yl)pyrimidin-2- | |
| 556813-39-9 | yl)amino)ethyl)-5-nitropyridine- | |
| | 2,6-diamine | |
| | 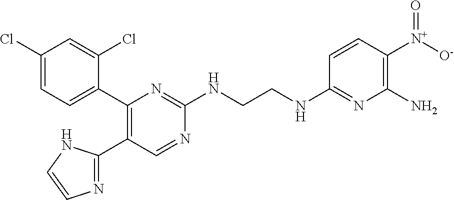 [SMILES: Nc1nc(NCCNc2ncc(-c3ncc[nH]3)c(-c3ccc(Cl)cc3Cl)n2)ccc1[N+](=O)[O-]] | |
| GSK-3β Inhibitor | (2-chloro-4-((4-(thiophen-2- | |
| XVIII | yl)pyrimidin-2- | |
| 1139875-74-3 | yl)amino)phenyl)(4- | |
| | methylpiperazin-1-yl)methanone | |
| | 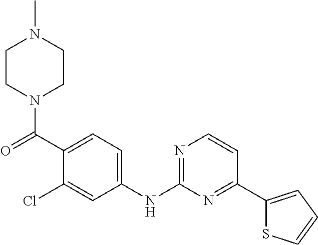 [SMILES: CN1CCN(C(=O)c2ccc(Nc3nccc(-c4cccs4)n3)cc2Cl)CC1] | |
| CGP60474 | 3-[[4-[2-[(3-chlorophenyl)amino]- | |
| 164658-13-3 | 4-pyrimidinyl]-2- | |
| | pyridinyl]amino]-1-propanol | |
| | 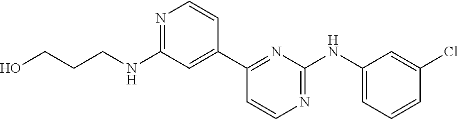 [SMILES: OCCCNc1cc(-c2ccnc(Nc3cccc(Cl)c3)n2)ccn1] | |
| AZD2858 (AR28) | 3-amino-6-[4-[(4-methyl-1- | |
| 486424-20-8 | piperazinyl)sulfonyl]phenyl]-N-3- | |
| | pyridinyl-2-pyrazinecarboxamide | |
| | 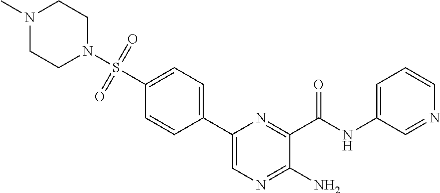 [SMILES: CN1CCN(S(=O)(=O)c2ccc(-c3cnc(N)c(C(=O)Nc4cccnc4)n3)cc2)CC1] | |
| CID 755673 | 7-hydroxy-2,3,4,5-tetrahydro-1H- | |
| 521937-07-5 | [1]benzafuro[2,3-c]azepin-1-one | |
| | 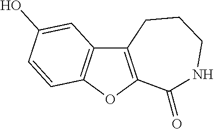 [SMILES: O=C1NCCCc2c1oc1ccc(O)cc21] | |
| TCS 2002 | 2-methyl-5-(3-(4- | |
| 1005201-24-0 | (methylsulfinyl)phenyl)benzofuran- | |
| | 5-yl)-1,3,4-oxadiazole | |
| | 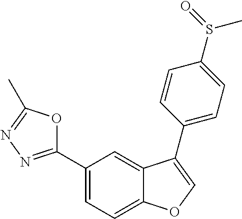 [SMILES: Cc1nnc(-c2ccc3occ(-c4ccc(S(C)=O)cc4)c3c2)o1] | |
| Dibromo- cantharelline 101481-34-9 | (3aR, 11bS)-2-amino-10,11- dibromo-5,6,9,11b-tetrahydro-4H- imidazo[4,5-h]pyrrolo[2,3- f]indolizin-8(3H)-one 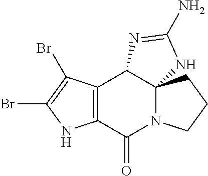 [SMILES: NC1=N[C@H]2c3c([nH]c(Br)c3Br)C(=O)N3CCC[C@]23N1] | (3aR,11bS)-10,11-dibromo-8- oxo-1,5,6,8,9,11b-hexahydro- 4H-imidazo[4,5-h]pyrrolo[2,3- f]indolizin-2(3H)-iminium chloride 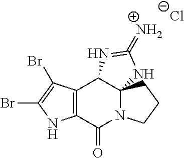 |
| ML320 | 4-(2-methoxyphenyl)-7,7- | |
| 1597438-84-0 | dimethyl-3-(trifluoromethyl)- | |
| | 2,4,6,7,8,5-hexahydro-5H- | |
| | pyrazolo[3,4-b]quinolin-5-one | |
| | 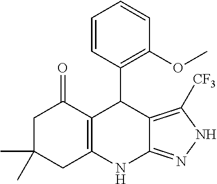 | |
| Flavopiridol | 2-(2-chlorophenyl)-5,7-dihydroxy- | |
| 146426-40-6 | 8-[(3S,4R)-3-hydroxy-1-methyl-4- | |
| | piperidinyl]-4-chromenone | |
| | 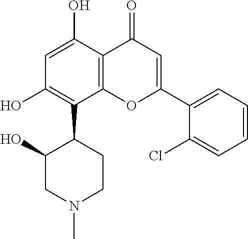 [SMILES: CN1CC[C@H](c2c(O)cc(O)c3c(=O)cc(-c4ccccc4Cl)oc23)[C@H](O)C1] | |
| Compound 100 | N-(6-phenylfuro[2,3-d]pyrimidin- | |
| 744255-19-4 | 4-yl)cyclopentanecarboxamide | |
| | 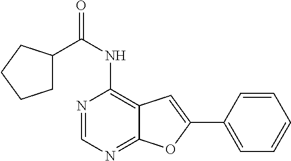 [SMILES: O=C(Nc1ncnc2oc(-c3ccccc3)cc12)C1CCCC1] | |
| Hymenidin | N-[(E)-3-(2-amino-1H-imidazol-5- | |
| 107019-95-4 | yl)prop-2-enyl]-4-bromo-1H- | |
| | pyrrole-2-carboxamide | |
| | 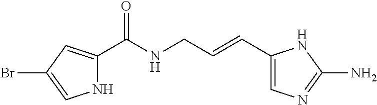 [SMILES: Nc1ncc(/C=C/CNC(=O)c2cc(Br)c[nH]2)[nH]1] | |
| 6-Bromoindirubin- | (2′Z,3′E)-6-bromoindirubin-3′- | |
| 3′-acetoxime | acetoxime, (3Z)-3-[(3E)-3- | |
| 667463-85-6 | [(acetyloxy)imino]-1,3-dihydro- | |
| | 2H-indol-2-ylidene]-6-bromo-1,3- | |
| | dihydro-2H-indol-2-one | |
| | 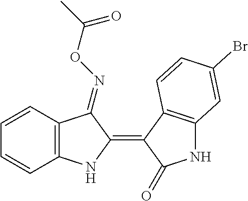 [SMILES: CC(=O)O/N=C1/C(=C2/C(=O)Nc3cc(Br)ccc32)Nc2ccccc21] | |
| GSK-3 Inhibitor IX (6-BIO) 667463-62-9 | (2Z,3E)-6′-chromo-3- (hydroxyimino)-[2,3′- biindolinylidene]-2′-one 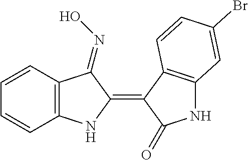 [SMILES: O=C1Nc2cc(Br)ccc2/C1=C1/Nc2ccccc2/C1=N\O] | |
| Indirubin-3′- monoxime 160807-49-8 | (2Z,3E)-3-(hydroxyimino)-[2,3′- biindolinylidene]-2′-one 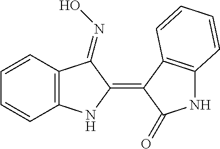 [SMILES: O=C1Nc2ccccc2/C1=C1/Nc2ccccc2/C1=N\O] | |
| 5-Iodo- indirubin-3′- monoxime 331467-03-9 | (2Z,3E)-3-(hydroxyimino)-5′-iodo- [2,3′-biindolinylidene]-2′-one 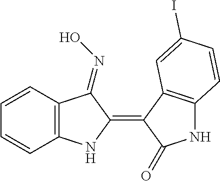 [SMILES: O=C1Nc2ccc(I)cc2/C1=C1/Nc2ccccc2/C1=N\O] | |
| Indirubin-5- sulfonic acid 331467-05-1 342418-97-7 | (2Z,3E)-3-(hydroxyimino)-2′-oxo- [2,3′-biindolinylidene]-5′-sulfonic acid 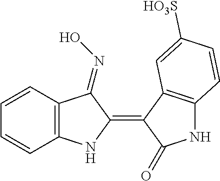 [SMILES: O=C1Nc2ccc(S(=O)(=O)O)cc2/C1=C1/Nc2ccccc2/C1=N\O] | (2Z,3E)-3-(hydroxyimino)-2′- oxo-[2,3′-biindolinylidene]-5′- sulfonic acid sodium salt 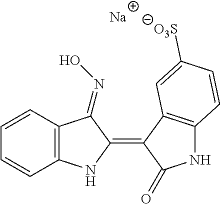 |
| Indirubin | (Z)-[2,3′-biindolinylide]-2′,3- | |
| 479-41-4 | dione | |
| | 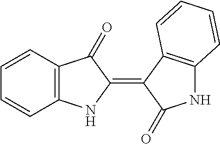 [SMILES: O=C1Nc2ccccc2/C1=C1/Nc2ccccc2C1=O] | |
| GSK-3 | (2Z,3Z)-3-(acetoxyimino)-6′- | |
| Inhibitor X | bromo-[2,3′-biindolinylidene]-2'- | |
| 740841-15-0 | one | |
| | 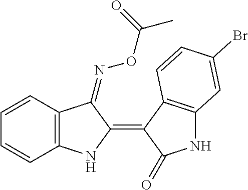 [SMILES: CC(=O)O/N=C1\C(=C2\C(=O)Nc3cc(Br)ccc32)Nc2ccccc21] | |
| Lithium Chloride | | |
| Beryllium salt | | BeSO₄ |
| Zinc salt | | |
| Tungstate salt | | |
| Mercury salt | | |
| Copper salt | | |
| Compound 39 | 2-(cyclopropanecarboxamido)-N- | |
| 1772824-10-8 | (4-(2,4-difluorophenyl)pyridin-3- | |
| | yl)isonicotinamide | |
| | 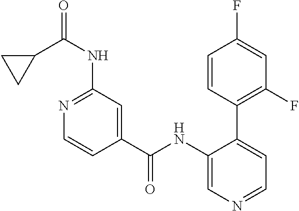 [SMILES: O=C(Nc1cnccc1-c1ccc(F)cc1F)c1ccnc(NC(=O)C2CC2)c1] | |
| Compound 29 | 2-(cyclopropanecarboxamido)-N- | |
| 1772823-37-6 | (4-(4-fluorophenyl)pyridin-3- | |
| | yl)isonicotinamide | |
| | 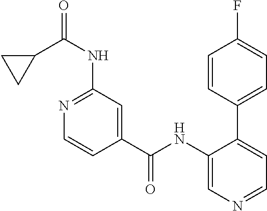 [SMILES: O=C(Nc1cnccc1-c1ccc(F)cc1)c1ccnc(NC(=O)C2CC2)c1] | |
| Compound 33 | N-(4-(4-chlorophenyl)pyridin-3- | |
| 1772823-64-9 | yl)-2- | |
| | (cyclopropanecarboxamido)isonico- | |
| | tinamide | |
| | 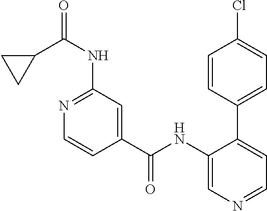 [SMILES: O=C(Nc1cnccc1-c1ccc(Cl)cc1)c1ccnc(NC(=O)C2CC2)c1] | |
| Compound 29′ | (1²E,3²E)-2²,2⁵-dihydro- | |
| 436866-61-4 | 1¹H,2¹H,3¹H-6,9,12,15-tetraoxa- | |
| | 1,3(3,1)-diindola-2(3,4)- | |
| | pyrrolacycloheptadecaphane-2²,2⁵- | |
| | dione | |
| | 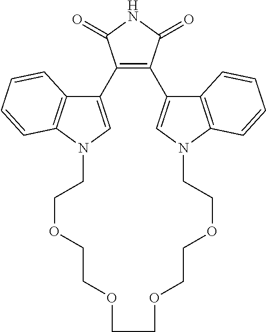 [SMILES: O=C1NC(=O)C2=C1c1cn(c3ccccc13)CCOCCOCCOCCOCCn1cc2c2ccccc21] | |
| Compound 46 | (1²E,3²E)-2²,2⁵-dihydro- | |
| 682807-74-5 | 1¹H,2¹H,3¹H-1,3(3,1)- | |
| | dipyrrolo[2,3-b]pyridina-2(3,4)- | |
| | pyrrola-7(2,5)- | |
| | thiophenacyclodecaphane-2²,2⁵- | |
| | dione | |
| | 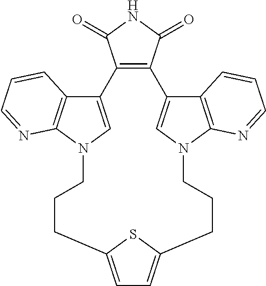 [SMILES: O=C1NC(=O)C2=C1c1cn(c3ncccc13)CCCC1=CC=C(CCCn3cc2c2cccnc23)S1] | |
| Compound 5a | (1²E,3²E)-9-ethyl-2²,2⁵-dihydro- | |
| 436866-54-5 | 1¹H,2¹H,3¹H-6,12-dioxa-9-aza- | |
| | 1,3(3,1)-diindola-2(3,4)- | |
| | pyrrolacyclotetradecaphane-2²,2⁵- | |
| | dione | |
| | 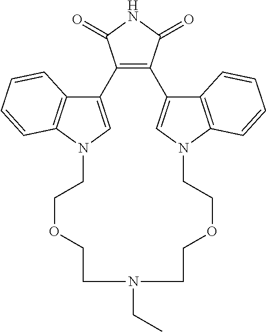 [SMILES: CCN1CCOCCn2cc(c3ccccc32)C2=C(C(=O)NC2=O)c2cn(c3ccccc23)CCOCC1] | |
| GF109203x 176504-36-2 | 3-(1-(3-(dimethylamino)propyl)- 1H-indol-3-yl)-4-(1H-indol-3-yl)- 1H-pyrrole-2,5-dione 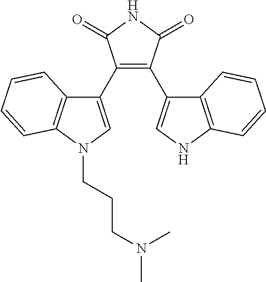 [SMILES: CN(C)CCCn1cc(C2=C(c3c[nH]c4ccccc34)C(=O)NC2=O)c2ccccc21] | 3-(1-(3- (dimethylamino)propyl)-1H- indol-3-yl)-4-(1H-indol-3-yl)- 1H-pyrrole-2,5-dione hydrochloride 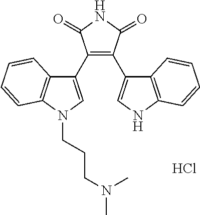 [SMILES: CN(C)CCCn1cc(C2=C(c3c[nH]c4ccccc34)C(=O)NC2=O)c2ccccc21 ; Cl] |
| RO31-8220 125314-64-9 | 3-(3-(4-(1-methyl-1H-indol-3-yl)- 2,5-dioxo-2,5-dihydro-1H-pyrrol- 3-yl)-1H-indol-1-yl)propyl carbamimidothioate 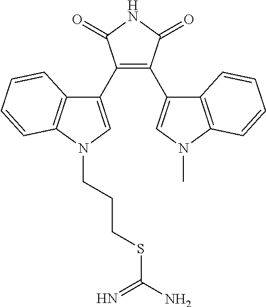 [SMILES: Cn1cc(C2=C(c3cn(CCCSC(=N)N)c4ccccc34)C(=O)NC2=O)c2ccccc21] | 3-(3-(4-(1-methyl-1H-indol-3- yl)-2,5-dioxo-2,5-dihydro-1H- pyrrol-3-yl)-1H-indol-1- yl)propyl carbamimidothioate methanesulfonate 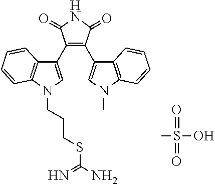 |
| Bisindolyl- maleimide X HCl 131848-97-0 | 3-(8-(aminomethyl)-6,7,8,9- tetrahydropyrido[1,2-a]indol-10- yl)-4-(1-methyl-1H-indol-3-yl)- 1H-pyrrole-2,5-dione 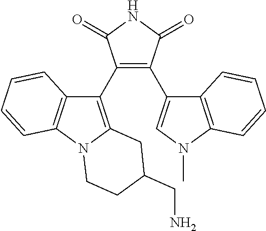 [SMILES: Cn1cc(C2=C(c3c4n(c5ccccc35)CCC(CN)C4)C(=O)NC2=O)c2ccccc21] | 3-(8-(aminomethyl)-6,7,8,9- tetrahydropyrido[1,2-a]indol- 10-yl)-4-(1-methyl-1H-indol-3- yl)-1H-pyrrole-2,5-dione hydrochloride 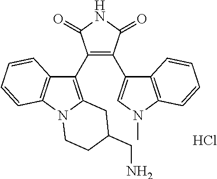 [SMILES: Cn1cc(C2=C(c3c4n(c5ccccc35)CCC(CN)C4)C(=O)NC2=O)c2ccccc21 ; Cl] |
| Enzastaurin | 3-(1-methyl-1H-indol-3-yl)-4-(1- | |
| (LY317615) | (1-(pyridin-2-ylmethyl)piperidin- | |
| 170364-57-5 | 4-yl)-1H-indol-3-yl)-1H-pyrrole- | |
| | 2,5-dione | |
| | 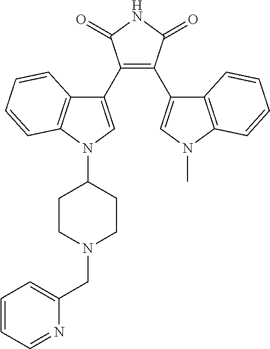 [SMILES: Cn1cc(C2=C(c3cn(C4CCN(Cc5ccccn5)CC4)c4ccccc34)C(=O)NC2=O)c2ccccc21] | |
| I5 | 2-chloro-5-((4-(3-chlorophenyl)- | |
| 264217-24-5 | 2,5-dioxo-2,5-dihydro-1H-pyrrol- | |
| | 3-yl)amino)benzoic acid | |
| | 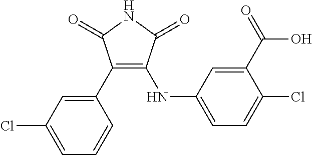 [SMILES: O=C1NC(=O)C(c2cccc(Cl)c2)=C1Nc1ccc(Cl)c(C(=O)O)c1] | |
| SB-216763 | 3-(2,4-dichlorophenyl)-4-(1- | |
| 280744-09-4 | methyl-1H-indol-3-yl)-1H- | |
| | pyrrole-2,5-dione | |
| | 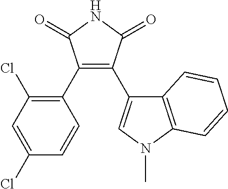 [SMILES: Cn1cc(C2=C(c3ccc(Cl)cc3Cl)C(=O)NC2=O)c2ccccc21] | |
| SB-415286 | 3-((3-chloro-4- | |
| 264218-23-7 | hydroxyphenyl)amino)-4-(2- | |
| | nitrophenyl)-1H-pyrrole-2,5-dione | |
| | 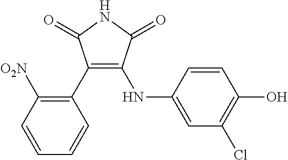 [SMILES: O=C1NC(=O)C(c2ccccc2[N+](=O)[O-])=C1Nc1ccc(O)c(Cl)c1] | |
| 3F8 | 5-ethyl-7,8-dimethoxy-1H- | |
| 159109-11-2 | pyrrolo[3,4-c]isoquinoline- | |
| | 1,3(2H)-dione | |
| | 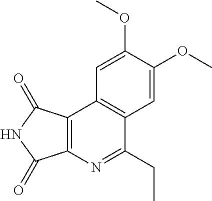 [SMILES: CCc1nc2c(c3cc(OC)c(OC)cc13)C(=O)NC2=O] | |
| TCS 21311 | 3-(5-(4-(2-hydroxy-2- | |
| 1260181-14-3 | methylpropanoyl)piperazin-1-yl)- | |
| | 2-(trifluoromethyl)phenyl)-4-(1H- | |
| | indol-3-yl)-1H-pyrrole-2,5-dione | |
| | 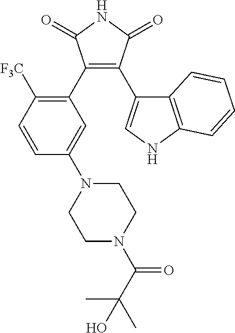 [SMILES: CC(C)(O)C(=O)N1CCN(c2ccc(C(F)(F)F)c(C3=C(c4c[nH]c5ccccc45)C(=O)NC3=O)c2)CC1] | |
| GSK-3 Inhibitor 1 603272-51-1 | 3-(imidazo[1,2-a]pyridin-3-yl)-4- (1,2,3,4-tetrahydro- [1,4]diazepino[6,7,1-hi]indol-7- yl)-1H-pyrrole-2,5-dione 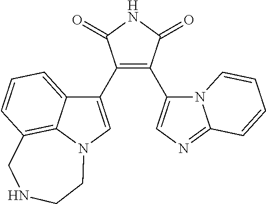 [SMILES: O=C1NC(=O)C(c2cnc3ccccn23)=C1c1cn2c3c(cccc13)CNCC2] | 3-(imidazo[1,2-a]pyridin-3-yl)- 4-(1,2,3,4-tetrahydro- [1,4]diazepino[6,7,1-hi]indol- 7-yl)-1H-pyrrole-2,5-dione hydrochloride 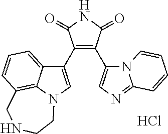 [SMILES: O=C1NC(=O)C(c2cnc3ccccn23)=C1c1cn2c3c(cccc13)CNCC2 ; Cl] |
| LY 2090314 | 3-(9-fluoro-2-(piperidine-1- | |
| 603288-22-8 | carbonyl)-1,2,3,4-tetrahydro- | |
| | [1,4]diazepino[6,7,1-hi]indol-7- | |
| | yl)-4-(imidazo[1,2-a]pyridin-3-yl)- | |
| | 1H-pyrrole-2,5-dione | |
| | 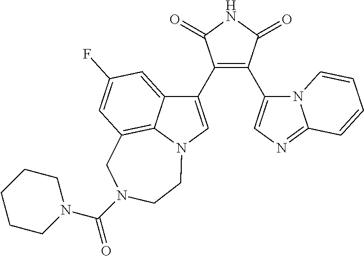 [SMILES: O=C1NC(=O)C(c2cnc3ccccn23)=C1c1cn2c3c(cc(F)cc13)CN(C(=O)N1CCCCC1)CC2] | |
| GSK-3 Inhibitor I | 3-(imidazo[1,2-a]pyridin-3-yl)-4- | |
| 603281-31-8 | (2-(morpholine-4-carbonyl)- | |
| | 1,2,3,4-tetrahydro- | |
| | [1,4]diazepino[6,7,1-hi]indol-7- | |
| | yl)-1H-pyrrole-2,5-dione | |
| | 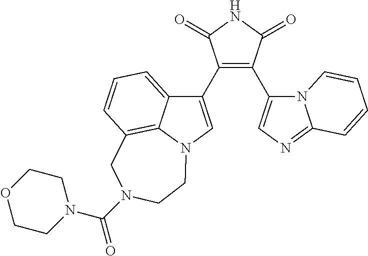 [SMILES: O=C1NC(=O)C(c2cnc3ccccn23)=C1c1cn2c3c(cccc13)CN(C(=O)N1CCOCC1)CC2] | |
| IM-12 | 3-((3-(4- | |
| 1129669-05-1 | fluorophenyl)propyl)amino)-1- | |
| | methyl-4-(2-methyl-1H-indol-3- | |
| | yl)-1H-pyrrole-2,5-dione | |
| | 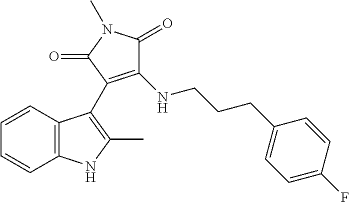 [SMILES: Cc1[nH]c2ccccc2c1C1=C(NCCCc2ccc(F)cc2)C(=O)N(C)C1=O] | |
| Compound 34 | 3-[3-(2,3-dihydroxy- | |
| 396091-16-0 | propylamino)-phenyl]-4-(5-fluoro- | |
| | 1-methyl-1H-indol-3-yl)-pyrrole- | |
| | 2,5-dione | |
| | 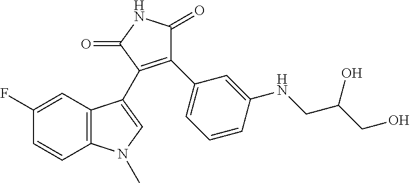 [SMILES: Cn1cc(C2=C(c3cccc(NCC(O)CO)c3)C(=O)NC2=O)c2cc(F)ccc21] | |
| KT 5720 | hexyl (5R,7R,8S)-7-hydroxy-8- | |
| 108068-98-0 | methyl-15-oxo-5,6,7,8,14,15- | |
| | hexahydro-13H-16-oxa-4b,8a,14- | |
| | triaza-5,8- | |
| | methanodibenzo[b,h]cycloocta[jkl] | |
| | cyclopenta[e]-as-indacene-7- | |
| | carboxylate | |
| | 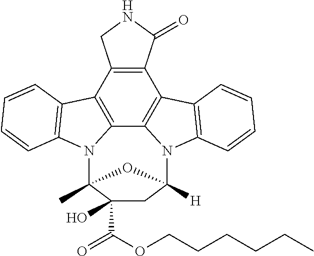 [SMILES: CCCCCCOC(=O)[C@@]1(O)C[C@H]2O[C@]1(C)n1c3ccccc3c3c4c(c5c6ccccc6n2c5c31)C(=O)NC4] | |
| Isogranulatimide | imidazo[1′,5′:1,6]pyrrolo[3′,4′:4,5] | |
| 244148-46-7 | pyrido[2,3-b]indole-1,3(2H,8H)- | |
| | dione | |
| | 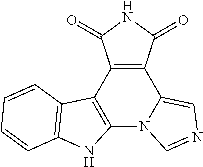 [SMILES: O=C1NC(=O)c2c1c1c3ccccc3[nH]c1n1cncc21] | |
| GSK-3β Inhibitor | 3-(1-(3-hydroxypropyl)-1H- | |
| XI | pyrrolo[2,3-b]pyridin-3-yl)-4- | |
| 626604-39-5 | (pyrazin-2-yl)-1H-pyrrole-2,5- | |
| | dione | |
| | 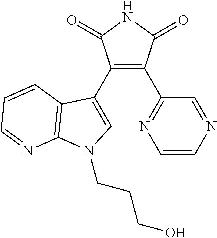 [SMILES: O=C1NC(=O)C(c2cn(CCCO)c3ncccc23)=C1c1cnccn1] | |
| BIP-135 | 3-(benzofuran-3-yl)-4-(5-bromo-1- | |
| 941575-71-9 | methyl-1H-indol-3-yl)-1H- | |
| | pyrrole-2,5-dione | |
| | 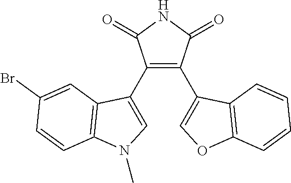 [SMILES: Cn1cc(C2=C(c3coc4ccccc34)C(=O)NC2=O)c2cc(Br)ccc21] | |
| CP21R7 | 3-(3-aminophenyl)-4-(5-bromo-1- | |
| 125314-13-8 | methyl-1H-indol-3-yl)-1H- | |
| | pyrrole-2,5-dione | |
| | 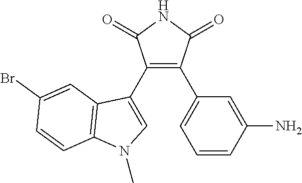 [SMILES: Cn1cc(C2=C(c3cccc(N)c3)C(=O)NC2=O)c2cc(Br)ccc21] | |
| Tivantinib | (3R,4R)-3-(5,6-dihydro-4H- | |
| 905854-02-6 | pyrrolo[3,2,1-ij]quinolin-1-yl)-4- | |
| | (1H-indol-3-yl)pyrrolidine-2,5- | |
| | dione | |
| | 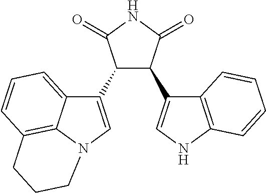 [SMILES: O=C1NC(=O)[C@@H](c2cn3c4c(cccc24)CCC3)[C@@H]1c1c[nH]c2ccccc12] | |
| Compound | (3-aminopyrido[2,3-a]pyrrolo[3,4- | |
| lambda- | c]carbazole-5,7(1H,6H)-dionato- | |
| OS1 | κN¹,κN¹²)carbonylchloro(2- | |
| 1291104-51-2 | pyridinemethanamine-κN¹,κN²)-, | |
| 1292843-11-8 | (OC-6-24-A)-Ruthenium | |
| | 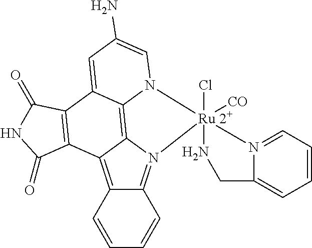 | |
| HB12 | carbonyl(η⁵-2,4-cyclopentadien-1- | |
| 800384-87-6 | yl)(pyrido[2,3-a]pyrrolo[3,4-c] | |
| | carbazole-5,7(1H,6H)-dionato- | |
| | κN¹,κN¹²)-Ruthenium | |
| | 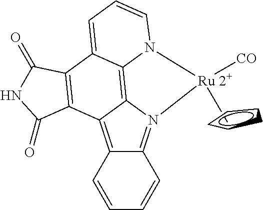 | |
| DW12 | carbonyl(η⁵-2,4-cyclopentadien-1- | |
| 861251-33-4 | yl)(9-hydroxypyrido[2,3-a] | |
| | pyrrolo[3,4-c]carbazole-5,7(1H, | |
| | 6H)-dionato-κN¹,κN¹²)-Ruthenium | |
| | 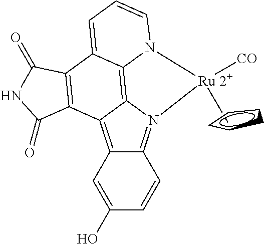 | |
| NP309 | carbonyl(η⁵-2,4-cyclopentadien-1- | |
| 937810-13-4 | yl)(3-fluoro-9-hydroxypyrido[2,3- | |
| | a]pyrrolo[3,4-c]carbazole-5,7(1H, | |
| | 6H)-dionato-κN¹,κN¹²)-Ruthenium | |
| | 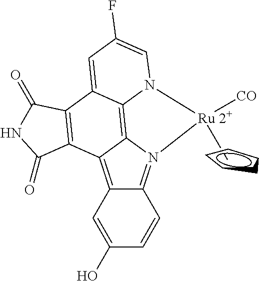 | |
| (RRu)-HB1229 | 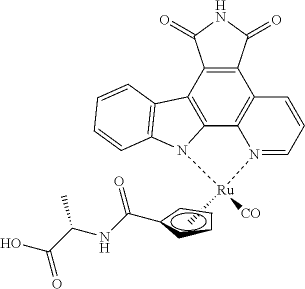 | |
| (RRu)-NP549 | 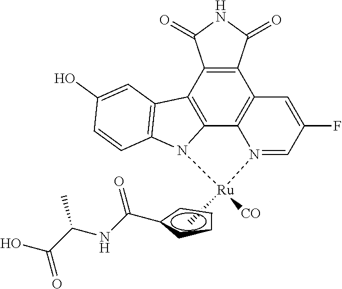 | |
| Compound 3 | 3-(3-aminophenyl)-4-(1-methyl- | |
| 1498285-39-4 | 1H-indol-3-yl)-1H-pyrrole-2,5- | |
| 1498285-48-5 | dione | |
| | 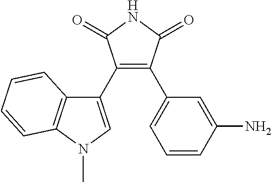 [SMILES: Cn1cc(C2=C(c3cccc(N)c3)C(=O)NC2=O)c2ccccc21] | |
| Compound (R)- | carbonyl(η⁵-2,4-cyclopentadien-1- | |
| DW12 | yl)(9-hydroxypyrido[2,3-a] | |
| 1047684-07-0 | pyrrolo[3,4-c]carbazole-5,7(1H, | |
| | 6H)-dionato-κN¹,κN¹²)-Ruthenium | |
| | 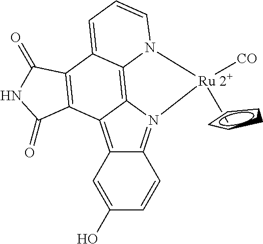 | |
| Staurosporine | (5S,7S,8S,9R)-8-methoxy-9- | |
| 62996-74-1 | methyl-7-(methylamino)- | |
| | 6,7,8,9,15,16-hexahydro-5H,14H- | |
| | 17-oxa-4b,9a,15-triaza-5,9- | |
| | methanodibenzo[b,h]cyclonona[jkl] | |
| | cyclopenta[e]-as-indacen-14-one | |
| | 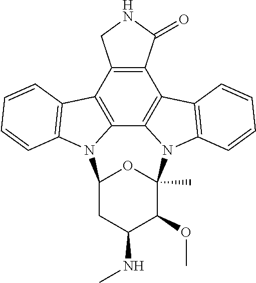 [SMILES: CN[C@H]1C[C@@H]2O[C@](C)([C@H]1OC)n1c3ccccc3c3c4c(c5c6ccccc6n2c5c31)CNC4=O] | |
| GSK-3β Inhibitor XXVI 871843-09-3 | 4,5-bis(1-methyl-1H-indol-3-yl)- 1,2-dihydropyrazol-3-one 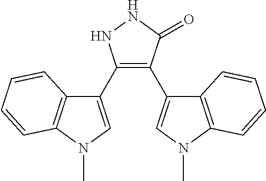 [SMILES: Cn1cc(-c2[nH][nH]c(=O)c2-c2cn(C)c3ccccc23)c2ccccc21] | |
| Manzamine A | (4aR,7S,7aR,13Z,14aR,15aR,18Z)- | |
| 104196-68-1 | 5-(9H-beta-carbolin-1-yl)- | |
| | 4,4a,9,10,11,12,14a,15-octahydro- | |
| | 3H-7,2- | |
| | oct[3]enoazocino[1′,2′:1,5]pyrrolo | |
| | [2,3-i]isoquinolin-7(1H,7aH)-ol | |
| | 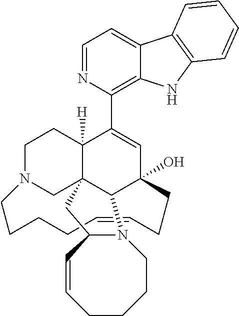 [SMILES: O[C@]12C=C(c3nccc4c3[nH]c3ccccc34)[C@@H]3CCN(CCCC/C=C\CC1)C[C@@]31C[C@@H]3/C=C\CCCCN3[C@H]12] | |
| TC-G 24 | N-(3-chloro-4-methylphenyl)-5-(4- | |
| 1257256-44-2 | nitrophenyl)-1,3,4-oxadiazol-2- | |
| | amine | |
| | 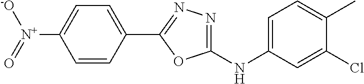 | |
| Compound 14d | 4′-(((5-(benzo[d][1,3]dioxol-5-yl)- | |
| 1374671-64-3 | 1,3,4-oxadiazol-2-yl)thio)methyl)- | |
| | [1,1′-biphenyl]-4-carbonitrile | |
| | 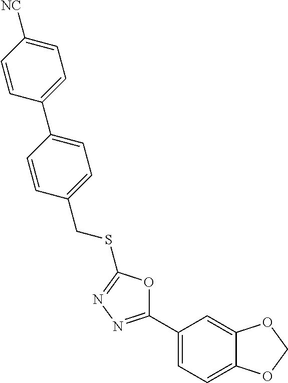 [SMILES: N#Cc1ccc(-c2ccc(CSc3nnc(-c4ccc5c(c4)OCO5)o3)cc2)cc1] | |
| Compound 15b | 4′-(((5-(2,3- | |
| 1374671-66-5 | dihydrobenzo[b][1,4]dioxin-6-yl)- | |
| | 1,3,4-oxadiazol-2-yl)thio)methyl)- | |
| | 4-fluoro-[1,1′-biphenyl]-2- | |
| | carbonitrile | |
| | 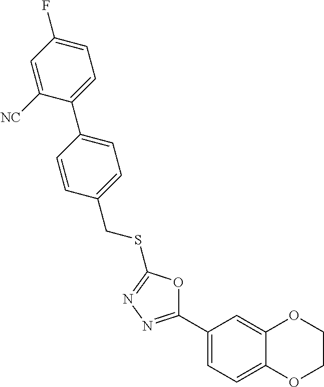 [SMILES: N#Cc1cc(F)ccc1-c1ccc(CSc2nnc(-c3ccc4c(c3)OCCO4)o2)cc1] | |
| Compound 20x | 3-(((5-(1-(4-methoxyphenyl)-1H- | |
| 1005201-80-8 | benzo[d]imidazol-6-yl)-1,3,4- | |
| | oxadiazol-2- | |
| | yl)thio)methyl)benzonitrile | |
| | 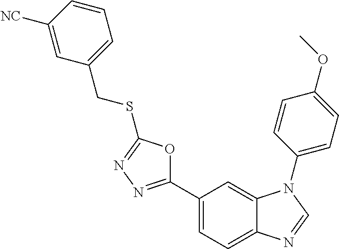 [SMILES: COc1ccc(-n2cnc3ccc(-c4nnc(SCc5cccc(C#N)c5)o4)cc32)cc1] | |
| GSK-3 Inhibitor II | 2-thio(3-iodobenzyl)-5-(1- | |
| 478482-75-6 | pyridyl)-[1,3,4]-oxadiazole | |
| | 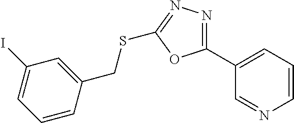 [SMILES: Ic1cccc(CSc2nnc(-c3cccnc3)o2)c1] | |
| GSK-3 Inhibitor 2 | N-(2-(4-amino-1,2,5-oxadiazol-3- | |
| 1377154-01-2 | yl)-1-ethyl-1H-imidazo[4,5- | |
| | c]pyridin-6-yl)-4-(2- | |
| | morpholinoethoxy)benzamide | |
| | 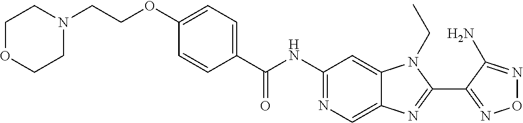 [SMILES: CCn1c(-c2nonc2N)nc2cnc(NC(=O)c3ccc(OCCN4CCOCC4)cc3)cc21] | |
| SU9516 | (Z)-1,3-dihydro-3-(1H-imidazol-4- | |
| 377090-84-1 | ylmethylene)-5-methoxy-2H- | |
| | indol-2-one | |
| | 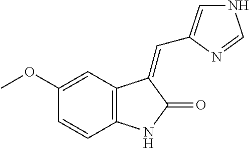 [SMILES: COc1ccc2c(c1)/C(=C/c1c[nH]cn1)C(=O)N2] | |
| AZD 1080 | (Z)-3-(5- | |
| 612487-72-6 | (morpholinomethyl)pyridin-2(1H)- | |
| | ylidene)-2-oxoindoline-5- | |
| | carbonitrile | |
| | 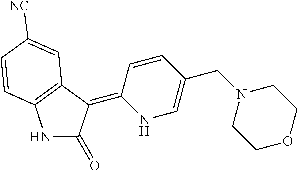 [SMILES: N#Cc1ccc2c(c1)/C(=C1\C=CC(CN3CCOCC3)=CN1)C(=O)N2] | |
| Kenpaullone | 9-bromo-7,12-dihydroindolo[3,2- | |
| 142273-20-9 | d][1]benzazepin-6(5H)-one | |
| | 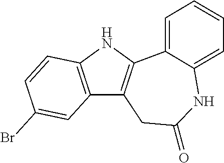 [SMILES: O=C1Cc2c([nH]c3ccc(Br)cc23)-c2ccccc2N1] | |
| Compound 17b | 2-(oxiran-2-yl)-9- | |
| 408532-42-3 | (trifluoromethyl)-7,12- | |
| | dihydrobenzo[2,3]azepino[4,5- | |
| | b]indol-6(5H)-one | |
| | 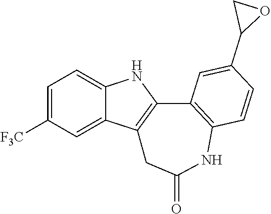 [SMILES: O=C1Cc2c([nH]c3ccc(C(F)(F)F)cc23)-c2cc(C3CO3)ccc2N1] | |
| Azakenpaullone | 9-bromo-7,1.2-dihydro- | |
| 676596-65-9 | pyrido[3′,2′:2,3]azepino[4,5- | |
| | b]indol-6(5H)-one | |
| | 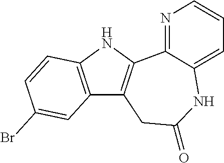 [SMILES: O=C1Cc2c([nH]c3ccc(Br)cc23)-c2ncccc2N1] | |
| Alsterpaullone | 9-nitro-7,12-dihydroindolo-[3,2- | |
| 237430-03-4 | d][1]benzazepin-6(5)-one | |
| | 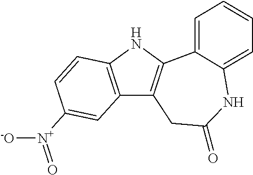 | |
| Alsterpaullone | 2-3-(6-oxo-9-nitro-5,6,7,12- | |
| cyanoethyl | tetrahydroindolo[3,2- | |
| 852529-97-0 | d][1]benzazepin-2-yl)propionitrile | |
| | 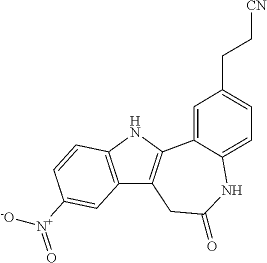 | |
| Cazpaullone | 6-oxo-5,6,7,12- | |
| 914088-64-5 | tetrahydropyrido(3′,2′- | |
| | 2,3)azepino(4,5-b)indole-9- | |
| | carbonitrile | |
| | 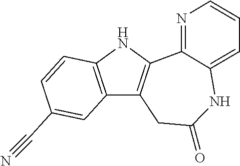 [SMILES: N#Cc1ccc2[nH]c3c(c2c1)CC(=O)Nc1cccnc1-3] | |
| FRATtide | L-seryl-L-glutaminyl-L-prolyl-L-a-glutamyl- | |
| | L-threonyl-L-arginyl-L-threonylglycyl-L-a- | |
| | aspartyl-L-a-aspartyl-L-a-aspartyl-L-prolyl- | |
| | L-histidyl-L-arginyl-L-leucyl-L-leucy-L- | |
| | glutaminyl-L-glutaminyl-L-leucyl-L-valyl-L- | |
| | leucyl-L-serylglycyl-L-asparaginyl-L-leucyl- | |
| | L-isoleucyl-L-lysyl-L-a-glutamyl-L-alanyl-L- | |
| | valyl-L-arginyl-L-arginyl-L-leucyl-L-histidyl- | |
| | L-seryl-L-arginyl-L-arginyl-L-leucyl-L- | |
| | Glutamine, | |
| | (SEQ ID NO: 1) | |
| L803 | KEAPPAPPQS(PO₃)P | |
| 348089-28-1 | (SEQ ID NO: 2) | |
| L803-mts | Myr-N-GKEAPPAPPQS(PO₃H)P—NH₂ | Trifluoroacetate salt |
| 1043881-55-5 | (SEQ ID NO: 3) | |
| | 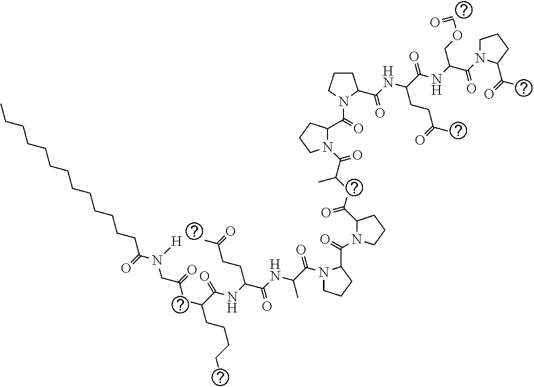 [SMILES: CCCCCCCCCCCCCC(=O)NCC(=O)CC(CCCCC)C(=O)NC(CCC(C)=O)C(=O)NC(C)C(=O)N1CCCC1C(=O)N1CCCC1C(=O)CC(C)C(=O)N1CCCC1C(=O)N1CCCC1C(=O)NC(CCC(C)=O)C(=O)NC(COC=O)C(=O)N1CCCC1C(C)=O] | |
| GSK-3 Inhibitor | 6-methyl-N-[3-[[3-(1- | |
| XXII | methylethoxy)propyl]carbamoyl]- | |
| 1195901-31-5 | 1H-pyrazol-4-yl]pyridine-3- | |
| | carboxamide | |
| | 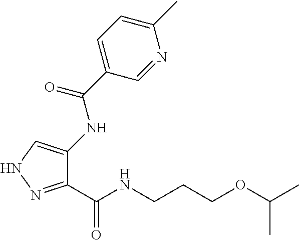 [SMILES: Cc1ccc(C(=O)Nc2c[nH]nc2C(=O)NCCCOC(C)C)cn1] | |
| Compound 4a | 4-(4,4-difluorocyclohexane-1- | |
| 1627557-91-8 | carboxamido)-N-((1r,4r)-4- | |
| | metboxycyclohexyl)-1H-pyrazole- | |
| | 3-carboxamide | |
| | 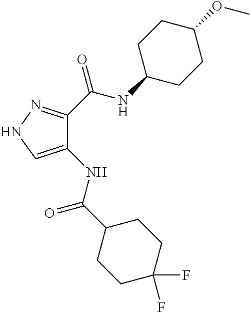 [SMILES: CO[C@H]1CC[C@H](NC(=O)c2n[nH]cc2NC(=O)C2CCC(F)(F)CC2)CC1] | |
| Compound 4t | N-((1r,4r)-4-methoxycyclohexyl)- | |
| 1627558-10-4 | 4-(4-(4-methylpiperazin-1- | |
| | yl)benzamido)-1H-pyrazole-3- | |
| | carboxamide | |
| | 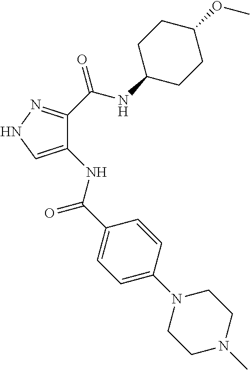 [SMILES: CO[C@H]1CC[C@H](NC(=O)c2n[nH]cc2NC(=O)c2ccc(N3CCN(C)CC3)cc2)CC1] | |
| Compound 4z | N-(3-(((1r,4r)-4- | |
| 1627558-16-0 | methoxycyclohexyl)carbamoyl)- | |
| | 1H-pyrazol-4-yl)-5- | |
| | phenyloxazole-4-carboxamide | |
| | 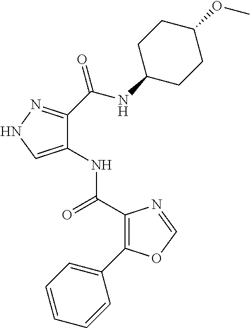 [SMILES: CO[C@H]1CC[C@H](NC(=O)c2n[nH]cc2NC(=O)c2ncoc2-c2ccccc2)CC1] | |
| AT 7519 | 4-(2,6-dichlorobenzamido)-N- | |
| 844442-38-2 | (piperidin-4-yl)-1H-pyrazole-3- | |
| | carboxamide | |
| | 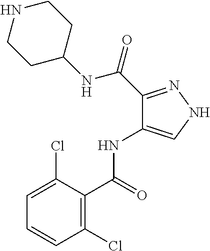 [SMILES: O=C(NC1CCNCC1)c1n[nH]cc1NC(=O)c1c(Cl)cccc1Cl] | |
| Pyrazolopyridine | 3-(dimethylamino)-N-(5-phenyl- | |
| 9 | 1H-pyrazolo[3,4-c]pyridazin-3- | |
| 923029-74-7 | yl)propanamide | |
| | 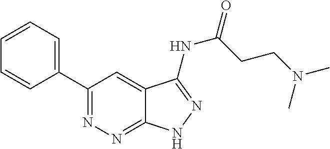 [SMILES: CN(C)CCC(=O)Nc1n[nH]c2nnc(-c3ccccc3)cc12] | |
| Pyrazolopyridine | N-(5-(2-fluorophenyl)-1H- | |
| 18 | pyrazolo[3,4-b]pyridin-3- | |
| 405221-39-8 | yl)butyramide | |
| | 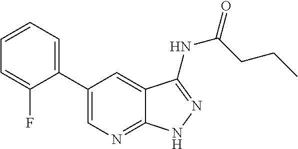 [SMILES: CCCC(=O)Nc1n[nH]c2ncc(-c3ccccc3F)cc12] | |
| Pyrazolopyridine | N-(5-bromo-6-(furan-2-yl)-1H- | |
| 34 | pyrazolo[3,4-b]pyridin-3- | |
| 583039-27-4 | yl)cyclopropanecarboxamide | |
| | 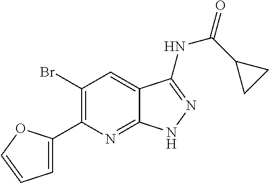 [SMILES: O=C(Nc1n[nH]c2nc(-c3ccco3)c(Br)cc12)C1CC1] | |
| Compound 14 | N-(5-chloro-6-(4-hydroxyphenyl)- | |
| 583038-63-5 | 1H-pyrazolo[3,4-b]pyridin-3- | |
| | yl)cyclopropanecarboxamide | |
| | 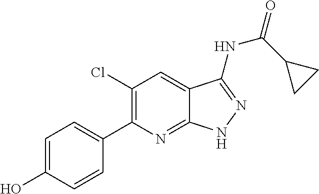 [SMILES: O=C(Nc1n[nH]c2nc(-c3ccc(O)cc3)c(Cl)cc12)C1CC1] | |
| Compound 23 | N-(5-bromo-6-(4-hydroxyphenyl)- | |
| 583038-76-0 | 1H-pyrazolo[3,4-b]pyridin-3-yl)- | |
| | 1-methylpiperidine-4-carboxamide | |
| | 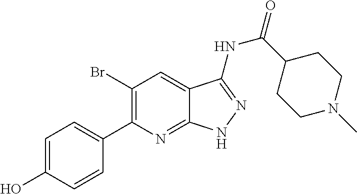 [SMILES: CN1CCC(C(=O)Nc2n[nH]c3nc(-c4ccc(O)cc4)c(Br)cc23)CC1] | |
| Compound 18 | N-(5-(2,3-difluorophenyl)-1H- | |
| 405223-20-3 | pyrazolo[3,4-c]pyridazin-3-yl)-1- | |
| | ethylpiperidine-4-carboxamide | |
| | 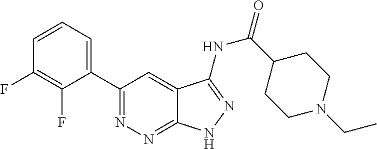 [SMILES: CCN1CCC(C(=O)Nc2n[nH]c3nnc(-c4cccc(F)c4F)cc23)CC1] | |
| Compound 19 | N-(5-(2,3-difluorophenyl)-1H- | |
| 405223-71-4 | pyrazolo[3,4-c]pyridazin-3-yl)-2- | |
| | (1-ethylpiperidin-4-yl)acetamide | |
| | 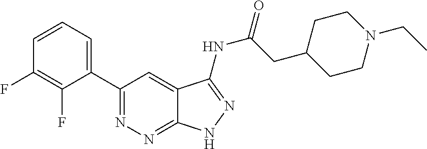 [SMILES: CCN1CCC(CC(=O)Nc2n[nH]c3nnc(-c4cccc(F)c4F)cc23)CC1] | |
| NSC 693868 (Compound 1) 40254-90-8 | 1H-pyrazolo[3,4-b]quinoxalin-3- amine 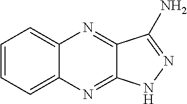 [SMILES: Nc1n[nH]c2nc3ccccc3nc12] | |
| Compound 150 | (S)-6-(2-(2- | |
| 1282042-18-5 | methoxyphenyl)morpholino)-1- | |
| | methyl-4-(pyrimidin-4-yl)pyridin- | |
| | 2(1H)-one | |
| | 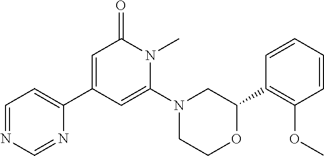 [SMILES: COc1ccccc1[C@H]1CN(c2cc(-c3ccncn3)cc(=O)n2C)CCO1] | |
| GSK-3 Inhibitor XIII 404828-08-6 | (5-methyl-1H-pyrazol-3-yl)-(2- phenylquinazolin-4-yl)amine 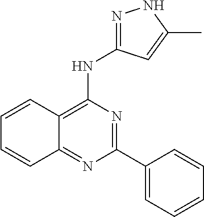 [SMILES: Cc1cc(Nc2nc(-c3ccccc3)nc3ccccc23)n[nH]1] | |
| VP0.7 | N′-decanoyl-1-ethyl-4-hydroxy-2- | |
| 331963-23-6 | oxo-1,2-dihydroquinoline-3- | |
| | carbohydrazide | |
| | 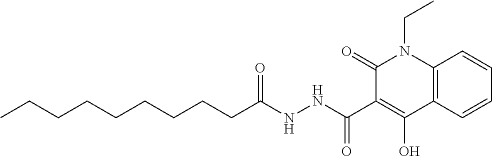 [SMILES: CCCCCCCCCC(=O)NNC(=O)c1c(O)c2ccccc2n(CC)c1=O] | |
| 1132813-46-7 | 9′-amino-10′-fluoro-8′-oxo-11′-((2- | |
| | (pyridin-2-ylamino)ethyl)amino)- | |
| | 2′,3′-dihydro-8′H- | |
| | spiro[cyclopentane-1,4′- | |
| | [1,4]oxazepino[2,3,4-ij]quinoline]- | |
| | 7′-carboxylic acid | |
| | 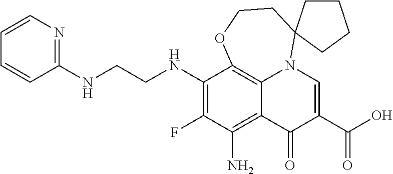 [SMILES: Nc1c(F)c(NCCNc2ccccn2)c2c3c1c(=O)c(C(=O)O)cn3C1(CCCC1)CCO2] | |
| 1132812-98-6 | 8′-amino-9′-fluoro-7′-oxo-10′-((2- | |
| | (pyridin-2-ylamino)ethyl)amino)- | |
| | 2′H,7′H-spiro[cyclopentane-1,3′- | |
| | [1,4]oxazino[2,3,4-ij]quinoline]-6′- | |
| | carboxylic acid | |
| | 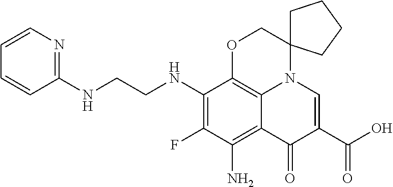 [SMILES: Nc1c(F)c(NCCNc2ccccn2)c2c3c1c(=O)c(C(=O)O)cn3C1(CCCC1)CO2] | |
| 950727-66-9 | (S)-8-amino-9-fluoro-3-tnethyl-7- | |
| | oxo-10-((3-(pyridin-2- | |
| | yl)propyl)amino)-2,3-dihydro-7H- | |
| | [1,4]oxazino[2,3,4-ij]quinoline-6- | |
| | carbonitrile | |
| | 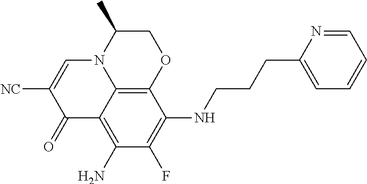 [SMILES: C[C@H]1COc2c(NCCCc3ccccn3)c(F)c(N)c3c(=O)c(C#N)cn1c23] | |
| GSK-3β Inhibitor VII 99-73-0 | 2-bromo-1-(4-bromophenyl)ethan- 1-one 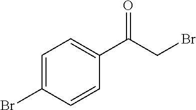 [SMILES: O=C(CBr)c1ccc(Br)cc1] | |
| GSK-3β Inhibitor VI (HMK-32) 62673-69-2 | 2-chloro-1-(4,5-dibromothiophen- 2-yl)ethan-1-one 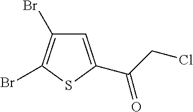 [SMILES: O=C(CCl)c1cc(Br)c(Br)s1] | |
| Palinurin | (R)-5-((S,2E,7E,9E)-13-(furan-3- | |
| 254901-27-4 | yl)-2,6,10-trimethyltrideca-2,7,9- | |
| | trien-1-yl)-4-hydroxy-3- | |
| | methylfuran-2(5H)-one | |
| | 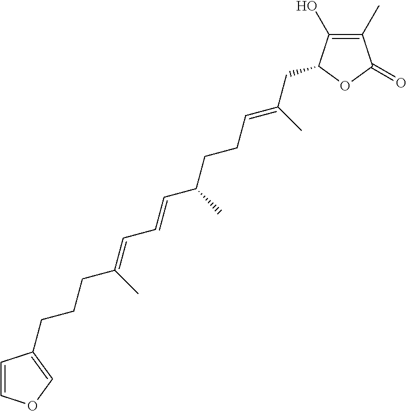 [SMILES: CC1=C(O)[C@@H](C/C(C)=C/CC[C@H](C)/C=C/C=C(\C)CCCc2ccoc2)OC1=O] | |
| Tricantin | 5-((2E,7E,9E)-13-(furan-3-yl)- | |
| 853885-55-9 | 2,6,10-trimethyltrideca-2,7,9-trien- | |
| | 1-yl)furan-2,3,4(5H)-trione | |
| | 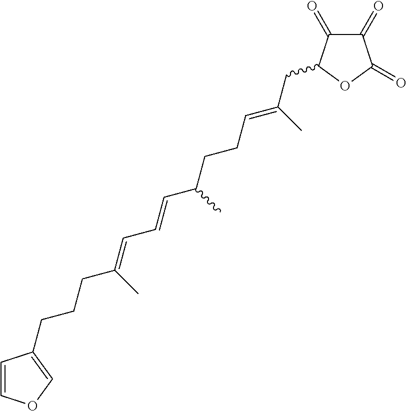 [SMILES: C/C(=C\C=C\C(C)CC/C=C(\C)CC1OC(=O)C(=O)C1=O)CCCc1ccoc1] | |
| GSK-3β Inhibitor I (TDZD-8) 327036-89-5 | 4-benzyl-2-methyl-1,2,4- thiadiazolidine-3,5-dione 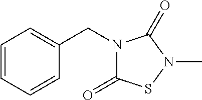 | |
| NP031115 | 2-(benzo[d][1,3]dioxol-4-yl)-4- | |
| 1400575-57-6 | benzyl-1,2,4-thiadiazolidine-3,5- | |
| | dione | |
| | 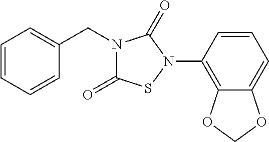 [SMILES: O=c1sn(-c2cccc3c2OCO3)c(=O)n1Cc1ccccc1] | |
| NP031112 (Tideglusib) 865854-05-3 | 4-benzyl-2-(naphthalen-1-yl)- 1,2,4-thiadiazolidine-3,5-dione 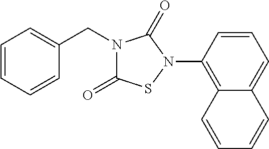 [SMILES: O=c1sn(-c2cccc3ccccc23)c(=O)n1Cc1ccccc1] | |
| Compound 90 | N,3-diphenyl-3H- | |
| 91322-11-1 | [1,2,3]triazolo[4,5-d]pyrimidin-5- | |
| | amine | |
| | 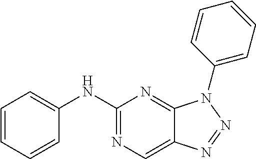 [SMILES: c1ccc(Nc2ncc3nnn(-c4ccccc4)c3n2)cc1] | |
| Compound 92 | 2-(4-((3-phenyl-3H- | |
| 1043429-30-6 | [1,2,3]triazolo[4,5-d]pyrimidin-5- | |
| | yl)amino)phenoxy)acetic acid | |
| | 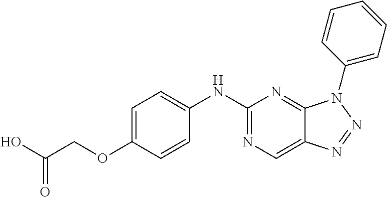 [SMILES: O=C(O)COc1ccc(Nc2ncc3nnn(-c4ccccc4)c3n2)cc1] | |
| GSK-3β Inhibitor VIII (AR-A014418) 487021-52-3 | N-(4-methoxybenzyl)-N′-(5-nitro- 1,3-thiazol-2-yl)urea 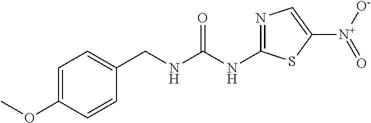 [SMILES: COc1ccc(CNC(=O)Nc2ncc([N+](=O)[O-])s2)cc1] | |
| A-1070722 | 1-(7-methoxyquinolin-4-yl)-3-[6- | |
| 1384424-80-9 | (trifluoromethyl)pyridin-2-yl]urea | |
| | 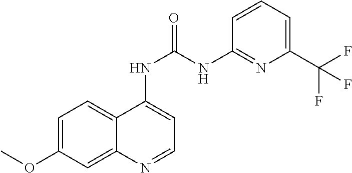 [SMILES: COc1ccc2c(NC(=O)Nc3cccc(C(F)(F)F)n3)ccnc2c1] | |
| Compound 27 | (E)-3-(3-(4-methoxyphenyl)-7H- | |
| 2025388-25-2 | pyrrolo[2,3-b]pyridin-5-yl)-N- | |
| | (pyridin-4-ylmethyl)acrylamide | |
| | 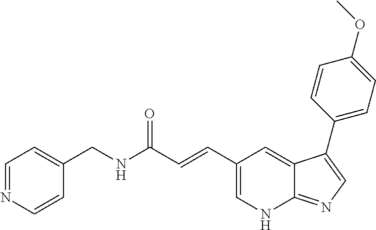 [SMILES: COc1ccc(-c2cnc3[nH]cc(/C=C/C(=O)NCc4ccncc4)cc2-3)cc1] | |
| Compound 12 | (E)-3-(3-(4-acetylphenyl)-7H- | |
| 2025388-10-5 | pyrrolo[2,3-b]pyridin-5-yl)-N-(2- | |
| | (pyridin-2-yl)ethyl)acrylamide | |
| | 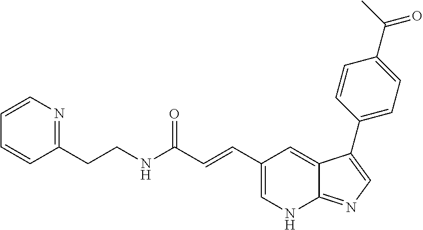 [SMILES: CC(=O)c1ccc(-c2cnc3[nH]cc(/C=C/C(=O)NCCc4ccccn4)cc2-3)cc1] | |
| NP-103 | Kramer 2012 | |
| CG-301338 | | |
| XD-4241 | | |
| CEP-16805 | | |
| AZ13282107 | | |
| SAR 502250 | | |
| (Sanofi) | | |
| 1073653-58-3 | | |
| AR79 | Jiang 2013 | |
| GI179186X | Unpublished structures | |
| CT118637 | | |
| CP-70949 | | |
| GW784752X | | |
| GW784775X | | |
| CT73911 | Dokken 2006 | |
| LY2064827 | Kotiliarova 2008 | |
| 705701 | | |
| 708244 | | |
| 709125 | | |

In some embodiments, the GSK inhibitor is selected from any compound, or a pharmaceutically acceptable salt thereof, disclosed or described in any of the following publications: WO 2008077138, WO 2003037891, U.S. Pat. Nos. 8,207,216, 8,071,591, CN 1319968 C, U.S. Pat. No. 7,514,445, CN 101341138, EP 1961748. WO 2010104205, US 20100292205. WO 2014003098, WO 2011089416, EP 1739087, WO 2001085685, US 20070088080, WO 2006018633, WO 2009017453, WO 2014050779, WO 2006100490, EP 1863904, WO 2014013255, WO2009017455, EP 2765188, WO 2014083132, U.S. Pat. No. 8,771,754, WO 20131244132, U.S. Pat. No. 8,771,754. WO 2013124413, WO 2014059383, WO 2010075551, U.S. Pat. No. 8,686,042, WO 2007102770, Kramer, T., et al., Int. J. Alzheimers Dis. 2012, 381029 (32 pg), doi: 10.1155/2012/381029; Jiang, Y., et al., Mol. Cancer Res. 2013, 11(12), 1597-610; Dokken, B B., et al., Am. J. Physiol. Endocrinol. Metab. 2006, 291(2), E207-E213; Kotiliarova, S., et al., Cancer Res. 2008, 68(16), 6643-6651; and J. Med. Chem. 2016, 59, 9018-9034, each of which is incorporated herein by reference in its entirety.

In some embodiments, the GSK inhibitor is selected from any compound, or a pharmaceutically acceptable salt thereof, disclosed or described in any of the following publications:

ACS Chem. Biol. 2010, 5 (8), 729; ACS Chem. Biol. 2013, 8, 1044; ACS Chem. Biol. 2016, 11, 1952; ACS Chem. Neurosci. 2012, 3, 5; ACS Chem. Neurosci. 2013, 4, 350; ACS Chem. Neurosci. 2014, 5, 194; ACS Infect. Dis. 2016, 2, 518; Acta biochimica et biophysica Sinica (2018), 50(5), 456; Anal. Chem. 2002, 74, 3232; Annual Reports in Medicinal Chemistry (2005), 40, 135; Anti-Cancer Drugs 2018, 29(8), 717; Archiv der Pharmazie (Weinheim, Germany) (2013), 346(5), 349; Biochem. J. (2003) 371, 199; Biochem. J. (2007) 408, 297; Bioorganic & Medicinal Chemistry 12 (2004) 3167; Bioorganic & Medicinal Chemistry 14 (2006) 9; Bioorganic & Medicinal Chemistry 14 (2006) 4987; Bioorganic & Medicinal Chemistry 17 (2009) 2017; Bioorganic & Medicinal Chemistry 17 (2009) 4302; Bioorganic & Medicinal Chemistry 18 (2010) 6785; Bioorganic & Medicinal Chemistry 20 (2012) 1188; Bioorganic & Medicinal Chemistry 23 (2015) 1179; Bioorganic & Medicinal Chemistry 24 (2016) 3116; Bioorganic & Medicinal Chemistry 26 (2018) 4209; Bioorganic & Medicinal Chemistry 26 (2018) 5479, Bioorganic & Medicinal Chemistry Letters 13 2003, 1577; Bioorganic & Medicinal Chemistry Letters 13 2003, 1581; Bioorganic & Medicinal Chemistry Letters 13 2003, 3049; Bioorganic & Medicinal Chemistry Letters 13 2003, 3055; Bioorganic & Medicinal Chemistry Letters 13 2003, 3059: Bioorganic & Medicinal Chemistry Letters 14 2004, 413; Bioorganic & Medicinal Chemistry Letters 14 2004, 3057; Bioorganic & Medicinal Chemistry Letters 14 2004, 3245; Bioorganic & Medicinal Chemistry Letters 14 2004, 3907; Bioorganic & Medicinal Chemistry Letters 15 2005, 899; Bioorganic & Medicinal Chemistry Letters 17 2007 2863; Bioorganic & Medicinal Chemistry Letters 20, 2010, 1693; Bioorganic & Medicinal Chemistry Letters 21 2011, 1429; Bioorganic & Medicinal Chemistry Letters 21 2011, 5948; Bioorganic & Medicinal Chemistry Letters 22 2012, 1005; Bioorganic & Medicinal Chemistry Letters 22, 2012, 1989; Bioorganic & Medicinal Chemistry Letters 23, 2013, 6928; Bioorganic & Medicinal Chemistry Letters 25, 2015, 1856; Bioorganic Chemistry 61 (2015) 21-27; Biophysical Chemistry 128 (2007) 165; British Journal of Pharmacology (2010), 160, 1-19; Cancer Research (2005), 65(16), 7462-9; Chem Biol Drug Des 2006, 68, 154; Chem Biol Drug Des 2011, 77, 199; Chem Biol Drug Des 2015, 86, 746: Chem Bio Chem 2008, 9, 2933; Chem Med Chem 2006, 1, 256; Chem Med Chem 2011, 6(9), 1587; Chem Med Chem 2011, 6(9), 1593; Chem Med Chem 2013, 8, 1127; Chemical Reviews 2001, 101, 2527-2540; Chemical Reviews (2014), 114(14), 7189; Chemical Biology & Drug Design (2017), 89(6), 964; Chemistry Biology (2003), 10. (12), 1255; Current Pharmaceutical Design, 2004, 10, 1105: Current Pharmaceutical Design, 2010, 16, 2666; Current Pharmaceutical Design, 2013, 19, 665: Current Pharmaceutical Design, 2013, 19, 4755; Dalton Trans. 2009, 10882: Diabetes 2002, 51(7), 2190, Diabetes 2002, 51(10), 2903; Diabetes 2003, 52(3), 588; Drug Design, Development and Therapy 2016, 10 1225; Eur. J. Biochem. 2000, 267, 5983; European Journal of Medicinal Chemistry 40, (2005), 655; European Journal of Medicinal Chemistry 42(2007) 1014; European Journal of Medicinal Chemistry 43 (2008) 949; European Journal of Medicinal Chemistry 44 (2009) 2361; European Journal of Medicinal Chemistry 68 (2013) 361; European Journal of Medicinal Chemistry 125 (2017) 464; European Journal of Medicinal Chemistry 144 (2018) 843; FEBS Letters 460 (1999) 433; Frontiers in Molecular Neuroscience 2011, 4, 32, 1; International Journal of Alzheimer's Disease 2012, 1; International Journal of Alzheimer's Disease (2012), 381029, 32: J. Am. Chem. Soc. 2004, 126, 13594: J. Am. Chem. Soc. 2007, 129, 8328; J. Am. Chem. Soc. 2011, 133(15), 5976; J. Chem. Inf. Model. 2009, 49, 824; J. Chem. Inf. Model. 2011, 51, 1431; J. Chem. Inf. Model. 2015, 55, 2540; J. Med. Chem. 2002, 45, 1292; J. Med. Chem. 2003, 46, 222; J. Med. Chem. 2003, 46, 3333: J. Med. Chem. 2003, 46, 4021; J. Med. Chem. 2004, 47, 22; J. Med. Chem. 2004, 47, 935; J. Med. Chem. 2004, 47, 3934; J. Med. Chem. 2004, 47, 4716; J. Med. Chem. 2008, 51, 2062: J. Med. Chem. 2008, 51, 2196; J. Med. Chem. 2008, 51, 4986; J. Med. Chem. 2009, 52, 1853; J. Med. Chem. 2009, 52, 6270; J. Med. Chem. 2010, 53, 994; J. Med. Chem. 2010, 53, 8534; J. Med. Chem. 2011, 54, 4042; J. Med. Chem. 2011, 54, 8461; J. Med. Chem. 2012, 55, 1645; J. Med. Chem. 2012, 55, 9107; J. Med. Chem. 2012, 55, 9531; J. Med. Chem. 2012, 55, 9589; J. Med. Chem. 2013, 56, 264; J. Med. Chem. 2013, 56, 5115; J. Med. Chem. 2013, 56, 10066; J. Med. Chem. 2014, 57, 7536; J. Med. Chem. 2015, 58, 8907; J. Med. Chem. 2015, 58, 8920: J. Med. Chem. 2016, 59, 531; J. Med. Chem. 2016, 59, 1041; J. Med. Chem. 2016, 59, 9018; J. Med. Chem. 2017, 60, 8482; J. Med. Chem. 2018, 61(17), 7640; J. Nat. Prod. 2007, 70, 1397; J. Nat. Prod. 2009, 72, 1520; J. Neurochemistry (2004), 89(6), 1313; J. Neurochemistry (2013), 125 (3, 4), 446; Journal of Cellular Biochemistry (2018), 119(4), 3510; Letters in Drug Design & Discovery, 2012, 9, 780-788: Medicinal Research Reviews, 2002, 22, 4, 373; Medicinal Research Reviews, 2008, 28, 5, 773; Mol Biosyst. 2009, 5(11) 1356-1360; Mol Cancer Res. 2013, 11(12), 1-14: Molecular and Cellular Endocrinology 409 (2015) 1-10; Molecules 2013, 18, 5498; Nature Reviews Drug Discovery, 2018, 17(5), 314; Oncogene (2010) 29, 2325-2336: Oncotarget (2014). 5(10), 2881; PloS one (2015), 10, (4), e0125028: Sci. Signal. 2016, 9(454), 110; Theranostics (2016), 6(4), 571-59; TRENDS in Pharmacological Sciences 2004, 9, 25, CN 1319968, CN 101341138, EP 1739087, EP 1863904, EP 1961748, EP 2765188, JP2005145928A, JP2005298437A, U.S. Pat. Nos. 5,721,245, 6,057,117, 6,153,618. U.S. Pat. Nos. 6,417,185, 6,489,344, 6,608,063, 6,800,632, 6,828,327, 6,849,643, 6,949,547, 6,989,382, 7,037,918, 7,045,519. U.S. Pat. Nos. 7,053,097, 7,250,443, 7,384,942, 7,425,557, 7,470,709, 7,470,709, 7,491,716, 7,514,445, 7,671,049, 7,951,820, 8,008,294, 8,022,065, 8,058,425, 8,071,591, 8,207,216, 8,318,713, 8,389,514, U.S. Pat. Nos. 8,492,378, 8,648,063, 8,686,042, 8,686,150 8,742,132, 8,771,754, 8,802,668, 9,044,487, 9,096,594, US 20020156087 US 2003130289, US 20040192718, US 2005090483, US 2005288321, US 20060052396 US 20070088080, US 20070281949, US 20090203690, US 20090291982, US 20090325293 US 2009105229, US 2009156582, US 20100004308, US 20100292205, US 20110160237 US 2011207721, US 2011294792, US 20120077840, US 20120219593, US 20130039996 US 20130085128, US 20140107141, US 20140235621, US 20150313890, US 20160375006, WO 0038675, WO 0144206, WO 0144246, WO 0156567, WO 0174771, WO 02062795. WO 0210141, WO 0220495, WO 0222604, WO 0224694, WO 0224893, WO 02328% WO 0246183, WO 0246197, WO 03004472, WO 03037869, WO 03037877, WO 03037891 WO 03045949, WO 03049739, WO 03051847, WO 03068773, WO 03068932, WO 03076398, WO 03076442, WO 03082853, WO 03095452, WO 03103663. WO 03104222, WO 2002046183, WO 2002046197, WO 2003037891, WO 2003066629, WO 2003/076442, WO 2004016597, WO 2004018419, WO 2004037791, WO 2004072062, WO 2005000836, WO 2005012304, WO 2005012307, WO 2005027823, WO 2005061519, WO 2005123672, WO 2006070198, WO 2006075023, WO 2006085685, WO 2006091737, WO 2006117212, WO 2006117221. WO 2007008514, WO 2007075911, WO 2007089191. WO 2007089192, WO 2007089193, WO 2007100282, WO 2007102770, WO 2007106537, WO 2007120102, WO 2008074752, WO 2008077138, WO 2008116881, WO 2008130312, WO 2008154241, WO 2009002806, WO 2009006043, WO 2009010542, WO 2009017453, WO 2009017454, WO 2009017455, WO 2009035634, WO 2009035684, WO 2009074259, WO 2009130317. WO 2009138176, WO 2010075551, WO 2001085685, WO 2010104205, WO 2010109005. WO 2011073092, WO 2011089416, WO 2012084683, WO 2012089828, WO 2013124413, WO 2014003098, WO 2014013255, WO 2014050779, WO 2014059383, WO 2014083132, WO 2015069593, WO 2015069594, WO 2018098411, WO 2018163216, WO 9741854, WO 9816528, and WO 9965897, each of which is incorporated herein by reference in its entirety.

In some embodiments, the GSK inhibitor is a GSK3a inhibitor. In some embodiments, a Wnt agonist may include, but not limited to, an agent selected from the agents described in Table 2, any agent disclosed in any of the references cited in Table 2, any derivatives thereof, or pharmaceutically acceptable salts thereof. A Wnt agonist is selected from any grouping of agents described or disclosed in Table 2, or any derivatives and pharmaceutically acceptable salts thereof. For example, a Wnt agonist is selected from GSK-3b XXII, AZD1080, and BRD1652; Compound 33, AT 7519. Pyrazolopyridine 34, and Compound 39; Tivantinib and 15; or the like. Classes of GSK3a inhibitors for use in various embodiments of methods disclosed herein include, but are not limited to, those listed in Table 2. The classes define a structural core moiety of the GSK3α inhibitor. For example, the Wnt agonist of the present disclosure may include an aminopyrimidine moiety.

TABLE 2
GSKα inhibitors

| | | Potency | Potency | Ratio |
|---|---|---|---|---|
| | | (nM) | (nM) | of |
| Agent | | GSK3- | GSK3- | alpha |
| CAS Number | Neutral Structure | alpha | beta | to beta |
| GSK-3β XXII | See Table 1. | 2.3 | 2.0 | 0.87 |
| 1195901-31-5 | | | | |
| AT 7519 | See Table 1. | | 89 | |
| 844442-38-2 | | | | |
| Compound 4a | See Table 1. | | 8 | |
| 1627557-91-8 | | | | |
| Compound 4t | See Table 1. | | <5 | |
| 1627558-10-4 | | | | |
| Compound 4z | See Table 1. | | 5 | |
| 1627558-16-0 | | | | |
| Compound 14 | See Table 1. | 1 | | |
| 583038-63-5 | | | | |
| Compound 23 | See Table 1. | 1 | | |
| 583038-76-0 | | | | |
| Pyrazolopyridine 34 | See Table 1. | 7 | | |
| 583039-27-4 | | | | |
| Compound 18 | See Table 1. | 0.95 | | |
| 405223-20-3 | | | | |
| Compound 19 | See Table 1. | 0.19 | | |
| 405223-71-4 | | | | |
| Compound 15b | See Table 1. | 2 (230) | 185 | 92 |
| 1374671-66-5 | | | (>1K) | (>4.3) |
| Compound 14d | See Table 1. | 6 | 316 | 52 |
| 1374671-64-3 | | | | |
| Compound 27 | See Table 1. | 42 | 140 | 3.3 |
| 1820758-44-8 | | | | |
| AZD1080 | See Table 1. | 6.9 | 31 | 4.5 |
| 612487-72-6 | | | | |
| Compound 39 | See Table 1. | 0.34 | 1.9 | 5.6 |
| 1772824-10-8 | | | | |
| Compound 29 | See Table 1. | 1.7 | 5.2 | 3.0 |
| 1772823-37-6 | | | | |
| Compound 33 | See Table 1. | 2 | 5.9 | 2.9 |
| 1772823-64-9 | | | | |
| Tivantinib | See Table 1. | 659 | 1865 | 2.8 |
| 905854-02-6 | | | | |
| I5 | See Table 1. | 76 | 160 | 2.1 |
| 264217-24-5 | | | | |
| Compound 90 | See Table 1. | 330 | 628 | 1.9 |
| 91322-11-1 | | | | |
| Compound 92 | See Table 1. | 9 | 13 | 1.4 |
| 1043429-30-6 | | | | |
| Compound lambda- | See Table 1. | 0.9 | 6 | 6.8 |
| OS1 | | | | |
| 1292843-11-8 | | | | |
| Compound 3 | See Table 1. | 3 | 10 | 3.3 |
| 1498285-39-4 | | | | |
| 1498285-48-5 | | | | |
| Compound (R)- | See Table 1. | 0.5 | 1 | 2 |
| DW12 | | | | |
| 1047684-07-0 | | | | |
| (R)-BRD4003 | (R)-3,7,7-trimethyl-4-phenyl- | 4800 | 10,200 | 2.1 |
| 1597439-60-5 | 2,4,6,7,8,9-hexahydro-5H- | | | |
| | pyrazolo[3,4-b]quinolin-5-one | | | |
| | 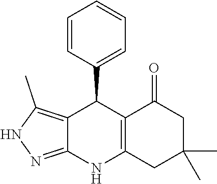 [SMILES: Cc1[nH]nc2c1[C@@H](c1ccccc1)C1=C(CC(C)(C)CC1=O)N2] | | | |
| (S)-BRD4003 | (S)-3,7,7-trimethyl-4-phenyl- | 161 | 232 | 1.4 |
| 1597439-59-2 | 2,4,6,7,8,9-hexahydro-5H- | | | |
| | pyrazolo[3,4-b]quinolin-5-one | | | |
| | 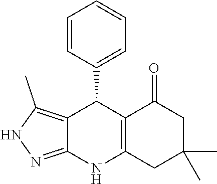 [SMILES: Cc1[nH]nc2c1[C@H](c1ccccc1)C1=C(CC(C)(C)CC1=O)N2] | | | |
| Compound 8 | 4-(2-fluorophenyl)-3,7,7-trimethyl- | 18 | 87 | 4.8 |
| 1597439-01-4 | 2,4,6,7,8,9-hexahydro-5H | | | |
| | pyrazolo[3,4-b]quinolin-5-one | | | |
| | 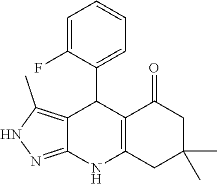 [SMILES: Cc1[nH]nc2c1C(c1ccccc1F)C1=C(CC(C)(C)CC1=O)N2] | | | |
| Compound 9 | 4-(3-fluorophenyl)-3,7,7-trimethyl- | 62 | 156 | 2.5 |
| 1597439-02-5 | 2,4,6,7,8,9-hexahydro-5H- | | | |
| 2056261-29-9 | pyrazolo[3,4-b]quinolin-5-one | | | |
| | 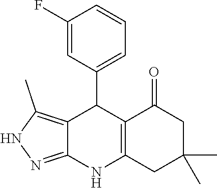 [SMILES: Cc1[nH]nc2c1C(c1cccc(F)c1)C1=C(CC(C)(C)CC1=O)N2] | | | |
| Compound 11 | 3,7,7-trimethyl-4-(o-tolyl)- | 32 | 102 | 3.2 |
| 1597439-12-7 | 2,4,6,7,8,9-hexahydro-5H- | | | |
| | pyrazolo[3,4-b]quinolin-5-one | | | |
| | 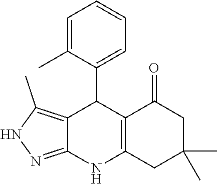 [SMILES: Cc1ccccc1C1C2=C(CC(C)(C)CC2=O)Nc2n[nH]c(C)c21] | | | |
| BRD1172 | (R)-4-(2-methoxyphenyl)-7,7- | 3 | 10 | 3.3 |
| 1597438-86-2 | dimethyl-3-(trifluoromethyl)- | | | |
| | 1,2,4,6,7,8-hexahydro-5H- | | | |
| | pyrazolo[3,4-b]quinolin-5-one | | | |
| | 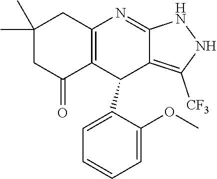 [SMILES: COc1ccccc1[C@@H]1C2=C(CC(C)(C)CC2=O)N=C2NNC(C(F)(F)F)=C21] | | | |
| Compound 16 | (S)-3,4,7,7-tetramethyl-4-phenyl- | 8 | 26 | 3.2 |
| 1597440-17-9 | 2,4,6,7,8,9-hexahydro-5H- | | | |
| | pyrazolo[3,4-b]quinolin-5-one | | | |
| | 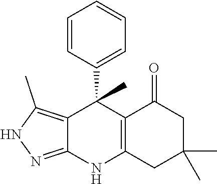 [SMILES: Cc1[nH]nc2c1[C@](C)(c1ccccc1)C1=C(CC(C)(C)CC1=O)N2] | | | |
| BRD1652 | N-4,7,7-trimethyl-4-phenyl-3- | 0.4 | 4 | 10 |
| 1597438-92-0 | (trifluoromethyl)-2,4,6,7,8,9- | | | |
| | hexahydro-5H-pyrazolo[3,4- | | | |
| | b]quinolin-5-one | | | |
| | 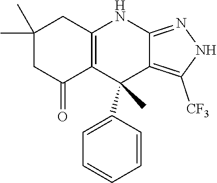 [SMILES: CC1(C)CC(=O)C2=C(C1)Nc1n[nH]c(C(F)(F)F)c1[C@]2(C)c1ccccc1] | | | |
| AR-A014418 | See Table 1. | 28 | 116 | 4.1 |
| 487021-52-3 | | | | |
| CREB knockdown | | | | |

In some embodiments, the GSK inhibitor is selected from any compound, or a pharmaceutically acceptable salt thereof, disclosed or described in any of the following publications: ACS Chem. Biol. 2016, 11, 1952-1963 and PLoS One 2016, 11(4), e0153075, each of which is incorporated herein by reference in its entirety for all purposes.

In some embodiments, the Wnt agonist is selected from agents of Table 3.

TABLE 3
Wnt Agonists

| Class | | |
|---|---|---|
| WNT | Agent | CAS |
| ARFGAP1 | QS 11 | 944328-88-5 |
| ARFGAP1 | WASP-1, ZINC00087877 | 352328-82-6 |
| Axin | Cpd1 | 1357473-75-6 |
| Axin | Cpd2 | 1228659-47-9 |
| Axin | HLY78 | 854847-61-3 |
| Axin | SKL2001 | 909089-13-0 |
| beta-catenin | DCA | 56-47-3 |
| Disrupts the | Compound 2 | 1360540-82-4 |
| Axin Complex | | |
| Disrupts the | Compound 71 | 1622429-71-3 |
| Axin Complex | | |
| Disrupts the | ISX 9 | 832115-62-5 |
| Axin Complex | | |
| DKK1 | WAY-262611 | 1123231-07-1 |
| inhibitor | | |
| MEK | Radicicol | 12772-57-5 |
| MEK | Selumetinib (AZD6244) | 606143-52-6 |
| PP2A | IQ 1 | 331001-62-8 |
| s-FRP-1 | (Dimethylamino)propyl)-2- | 915754-88-0 |
| inhibitor | ethyl-5-(phenylsulfonyl) | |
| | benzenesulfonamide | |
| sFRP-1 | Cyclosporine A (CsA) | 59865-13-3 |
| inhibitor | | |
| sFRP-1 | Cyclosporine analogs | |
| inhibitor | | |
| sFRP-1 | PSC833(Valspodar) | 121584-18-7 |
| inhibitor | | |
| sFRP-1 | WAY 316606 | 915759-45-4 |
| inhibitor | | |
| Target | Diketones | WO 2016029021 A1; |
| Undetermined | | WO 2012024404 A1 |
| Target | Diketones | 1622429-56-4 |
| Undetermined | | |
| Target | Diketones | 1360540-88-0 |
| Undetermined | | |
| Target | Diketones | 1360540-89-1 |
| Undetermined | | |
| Target | Diketones | 1622429-79-1 |
| Undetermined | | |
| Target | Diketones | 1622429-75-7 |
| Undetermined | | |
| Target | Diketones | 1622429-74-6 |
| Undetermined | | |
| Target | Diketones | 1622430-76-5 |
| Undetermined | | |
| Target | Diketones | 1622430-31-2 |
| Undetermined | | |
| Target | Diketones | 1622430-52-7 |
| Undetermined | | |
| Target | Diketones | 1622429-67-7 |
| Undetermined | | |
| Target | Diketones | 1622429-65-5 |
| Undetermined | | |
| Target | Diketones | 1622429-69-9 |
| Undetermined | | |
| van-Gogh- | Compound 109 | 1314885-81-8 |
| like receptor | | |
| proteins | | |
| (Vang1) | | |
| Wnt Ligand | Wnt-1 | Protein |
| Wnt Ligand | Wnt-10a | Protein |
| Wnt Ligand | Wnt-10b/12 | Protein |
| Wnt Ligand | Wnt-11 | Protein |
| Wnt Ligand | Wnt-16 | Protein |
| Wnt Ligand | Wnt-2/Irp (Int-I- | Protein |
| | related protein) | |
| Wnt Ligand | Wnt-2b/13 | Protein |
| Wnt Ligand | Wnt-3/Int-4 | Protein |
| Wnt Ligand | Wnt-3a | Protein |
| Wnt Ligand | Wnt-4 | Protein |
| Wnt Ligand | Wnt-5a | Protein |
| Wnt Ligand | Wnt-5b | Protein |
| Wnt Ligand | Wnt-6 | Protein |
| Wnt Ligand | Wnt-7a | Protein |
| Wnt Ligand | Wnt-7b | Protein |
| Wnt Ligand | Wnt-8a/8d | Protein |
| Wnt Ligand | Wnt-8b | Protein |
| Wnt Ligand | Wnt-9a/14 | Protein |
| Wnt Ligand | Wnt-9b/14b/15 | Protein |
| Wnt Related | Norrin | Protein |
| Protein | | |
| Wnt Related | R-Spondin 1/2/3/4 | Protein |
| Protein | | |
| Wnt-3a/Dkk-1 | BML-284 | 853220-52-7 |
| Wirt-3a/Dkk-1 | Compound 1 | 1084833-94-2 |
| Wnt-3a/Dkk-1 | Compound 25 | 1084834-05-8 |

Non-limiting examples of GSK inhibitors for use in the methods of the present disclosure are presented in Table 4.

TABLE 4
Substituted 3-imidazo[1,2-a]pyridin-3-yl-4-(1,2,3,4-tetrahydro-[1,4]diazepino-
[6,7,1-hi]indol-7-yl)pyrrole-2,5-dione compounds

| | | |
|---|---|---|
| Compound I-1 | 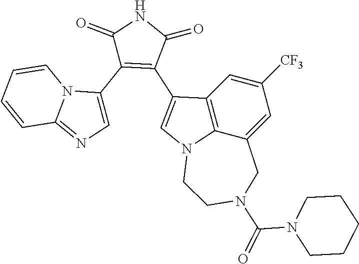 [SMILES: O=C1NC(=O)C(c2cnc3ccccn23)=C1c1cn2c3c(cc(C(F)(F)F)cc13)CN(C(=O)N1CCCCC1)CC2] | 3-(imidazo[1,2-a]pyridin-3-yl)-4-(2- (piperidine-1-carbonyl)-9-(trifluoromethyl)- 1,2,3,4-tetrahydro-[1,4]diazepino[6,7,1- hi]indol-7-yl)-1H-pyrrole-2,5-dione |
| Compound I-2 | 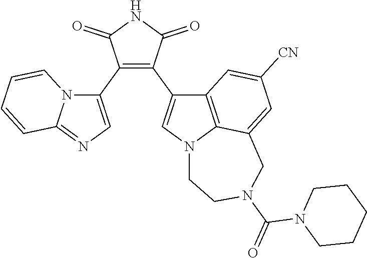 [SMILES: N#Cc1cc2c3c(c1)c(C1=C(c4cnc5ccccn45)C(=O)NC1=O)cn3CCN(C(=O)N1CCCCC1)C2] | 7-(4-(imidazo[1,2-a]pyridin-3-yl)-2,5-dioxo- 2,5-dihydro-1H-pyrrol-3-yl)-2-(piperidine-1- carbonyl)-1,2,3,4-tetrahydro- [1,4]diazepino[6,7,1-hi]indole-9-carbonitrile |
| Compound I-3 | 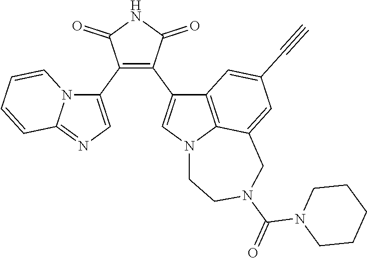 [SMILES: C#Cc1cc2c3c(c1)c(C1=C(c4cnc5ccccn45)C(=O)NC1=O)cn3CCN(C(=O)N1CCCCC1)C2] | 3-(9-ethynyl-2-(piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-4 | 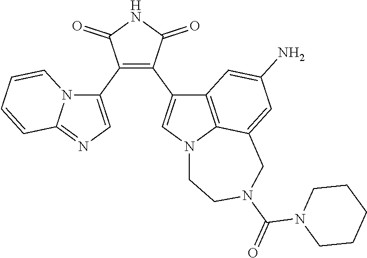 [SMILES: Nc1cc2c3c(c1)c(C1=C(c4cnc5ccccn45)C(=O)NC1=O)cn3CCN(C(=O)N1CCCCC1)C2] | 3-(9-amino-2-(piperidine-1-carbonyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-5 | 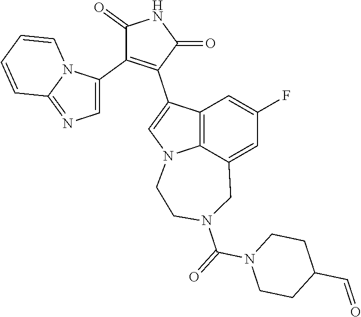 [SMILES: O=CC1CCN(C(=O)N2CCn3cc(C4=C(c5cnc6ccccn56)C(=O)NC4=O)c4cc(F)cc(c43)C2)CC1] | 1-(9-fluoro-7-(4-(imidazo[1,2-a]pyridin-3-yl)- 2,5-dioxo-2,5-dihydro-1H-pyrrol-3-yl)-1,2,3,4- tetrahydro-[1,4]diazepino[6,7,1-hi]indole-2- carbonyl)piperidine-4-carbaldehyde |
| Compound I-6 | 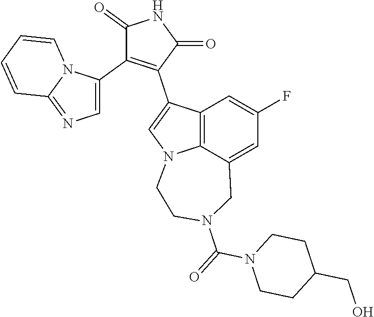 [SMILES: O=C1NC(=O)C(c2cnc3ccccn23)=C1c1cn2c3c(cc(F)cc13)CN(C(=O)N1CCC(CO)CC1)CC2] | 3-(9-fluoro-2-(4-(hydroxymethyl)piperidine- 1-carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-7 | 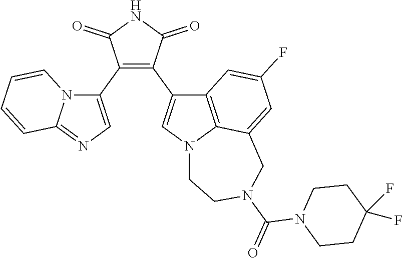 [SMILES: O=C1NC(=O)C(c2cnc3ccccn23)=C1c1cn2c3c(cc(F)cc13)CN(C(=O)N1CCC(F)(F)CC1)CC2] | 3-(2-(4,4-difluoropiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-8 | 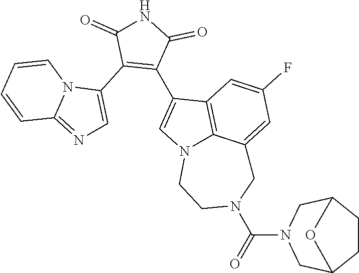 [SMILES: O=C1NC(=O)C(c2cnc3ccccn23)=C1c1cn2c3c(cc(F)cc13)CN(C(=O)N1CC3CCC(C1)O3)CC2] | 3-(2-(8-oxa-3-azabicyclo[3.2.1]octane-3- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-9 | 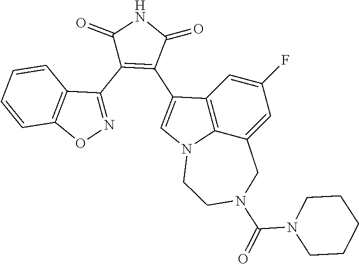 [SMILES: O=C1NC(=O)C(c2cn3c4c(cc(F)cc24)CN(C(=O)N2CCCCC2)CC3)=C1c1noc2ccccc12] | 3-(benzo[d]isoxazol-3-yl)-4-(9-fluoro-2- (piperidine-1-carbonyl)-1,2,3,4-tetrahydro- [1,4]diazepino[6,7,1-hi]indol-7-yl)-1H- pyrrole-2,5-dione |
| Compound I-10 | 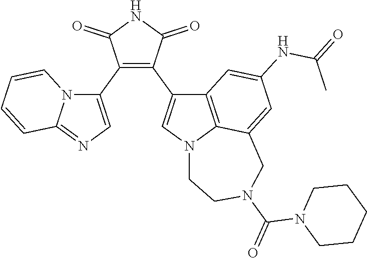 [SMILES: CC(=O)Nc1cc2c3c(c1)c(C1=C(c4cnc5ccccn45)C(=O)NC1=O)cn3CCN(C(=O)N1CCCCC1)C2] | N-(7-(4-(imidazo[1,2-a]pyridin-3-yl)-2,5- dioxo-2,5-dihydro-1H-pyrrol-3-yl)-2- (piperidine-1-carbonyl)-1,2,3,4-tetrahydro- [1,4]diazepino[6,7,1-hi]indol-9-yl)acetamide |
| Compound I-11 | 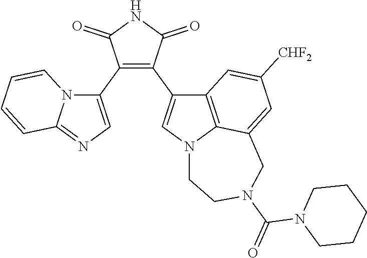 [SMILES: O=C1NC(=O)C(c2cnc3ccccn23)=C1c1cn2c3c(cc(C(F)F)cc13)CN(C(=O)N1CCCCC1)CC2] | 3-(9-(difluoromethyl)-2-(piperidine-1- carbonyl)-1,23,4-tetrahydro- [1,4]diazepino[6,7,1-hi]indol-7-yl)-4- (imidazo[1,2-a]pyridin-3-yl)-1H-pyrrole-2,5- dione |
| Compound I-12 | 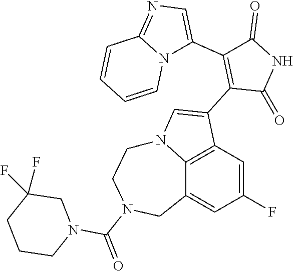 [SMILES: O=C1NC(=O)C(c2cnc3ccccn23)=C1c1cn2c3c(cc(F)cc13)CN(C(=O)N1CCCC(F)(F)C1)CC2] | 3-(2-(3,3-difluoropiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-13 | 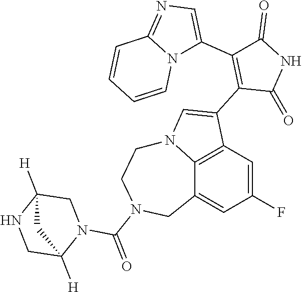 [SMILES: O=C1NC(=O)C(c2cnc3ccccn23)=C1c1cn2c3c(cc(F)cc13)CN(C(=O)N1C[C@H]3C[C@@H]1CN3)CC2] | 3-(2-((1R,4R)-2,5-diazabicyclo[2.2.1]heptane- 2-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-14 | 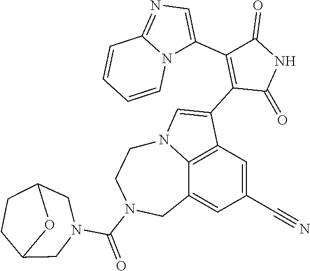 [SMILES: N#Cc1cc2c3c(c1)c(C1=C(c4cnc5ccccn45)C(=O)NC1=O)cn3CCN(C(=O)N1CC3CCC(C1)O3)C2] | 2-(8-oxa-3-azabicyclo[3.2.1]octane-3- carbonyl)-7-(4-(imidazo[1,2-a]pyridin-3-yl)- 2,5-dioxo-2,5-dihydro-1H-pyrrol-3-yl)-1,2,3,4- tetrahydro-[1,4]diazepino[6,7,1-hi]indole-9- carbonitrile |
| Compound I-15 | 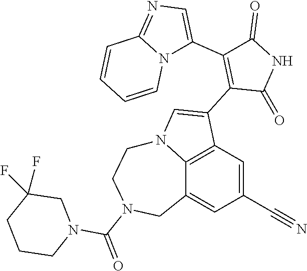 [SMILES: N#Cc1cc2c3c(c1)c(C1=C(c4cnc5ccccn45)C(=O)NC1=O)cn3CCN(C(=O)N1CCCC(F)(F)C1)C2] | 2-(3,3-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-16 | 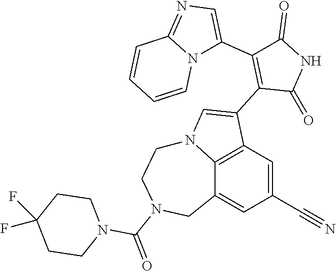 [SMILES: N#Cc1cc2c3c(c1)c(C1=C(c4cnc5ccccn45)C(=O)NC1=O)cn3CCN(C(=O)N1CCC(F)(F)CC1)C2] | 2-(4,4-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-17 | 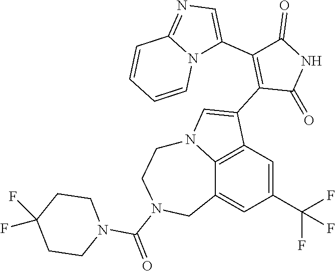 [SMILES: O=C1NC(=O)C(c2cnc3ccccn23)=C1c1cn2c3c(cc(C(F)(F)F)cc13)CN(C(=O)N1CCC(F)(F)CC1)CC2] | 3-(2-(4,4-difluoropiperidine-1-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-18 | 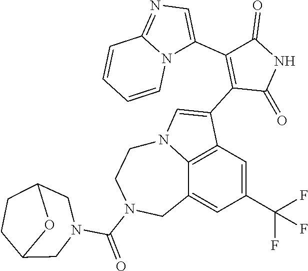 [SMILES: O=C1NC(=O)C(c2cnc3ccccn23)=C1c1cn2c3c(cc(C(F)(F)F)cc13)CN(C(=O)N1CC3CCC(C1)O3)CC2] | 3-(2-(8-oxa-3-azabicyclo[3.2.1]octane-3- carbonyl)-9-(trifluoromethyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-19 | 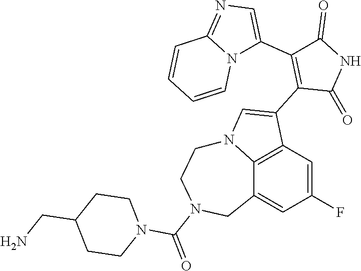 [SMILES: NCC1CCN(C(=O)N2CCn3cc(C4=C(c5cnc6ccccn56)C(=O)NC4=O)c4cc(F)cc(c43)C2)CC1] | 3-(2-(4-(aminomethyl)piperidine-1-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-20 | 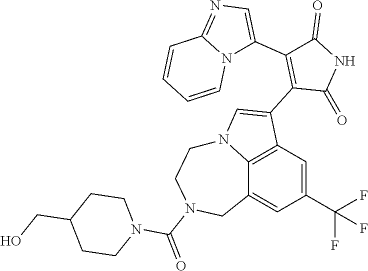 [SMILES: O=C1NC(=O)C(c2cnc3ccccn23)=C1c1cn2c3c(cc(C(F)(F)F)cc13)CN(C(=O)N1CCC(CO)CC1)CC2] | 3-(2-(4-(hydroxymethyl)piperidine-1- carbonyl)-9-(trifluoromethyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-21 | 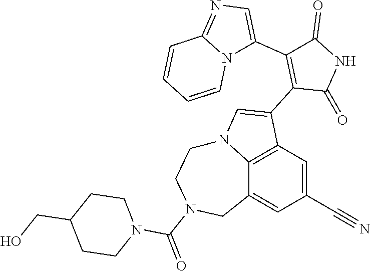 [SMILES: N#Cc1cc2c3c(c1)c(C1=C(c4cnc5ccccn45)C(=O)NC1=O)cn3CCN(C(=O)N1CCC(CO)CC1)C2] | 2-(4-(hydroxymethyl)piperidine-1-carbonyl)- 7-(4-(imidazo[1,2-a]pyridin-3-yl)-2,5-dioxo- 2,5-dihydro-1H-pyrrol-3-yl)-1,2,3,4- tetrahydro-[1,4]diazepino[6,7,1-hi]indole-9- carbonitrile |
| Compound I-22 | 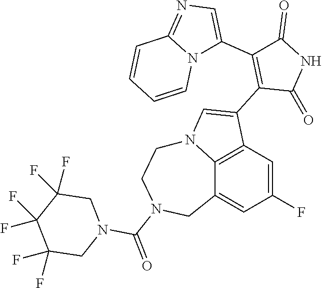 [SMILES: O=C1NC(=O)C(c2cnc3ccccn23)=C1c1cn2c3c(cc(F)cc13)CN(C(=O)N1CC(F)(F)C(F)(F)C(F)(F)C1)CC2] | 3-(9-fluoro-2-(3,3,4,4,5,5- hexafluoropiperidine-1-carbonyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-23 | 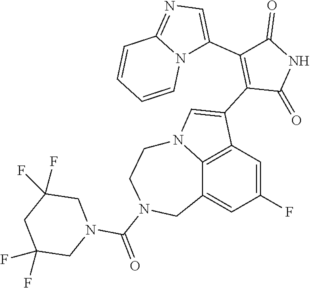 [SMILES: O=C1NC(=O)C(c2cnc3ccccn23)=C1c1cn2c3c(cc(F)cc13)CN(C(=O)N1CC(F)(F)CC(F)(F)C1)CC2] | 3-(9-fluoro-2-(3,3,5,5-tetrafluoropiperidine- 1-carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-24 | 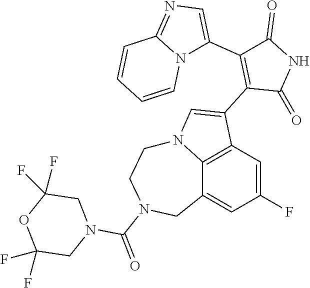 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(9-fluoro-2-(2,2,6,6-tetrafluoromorpholine- 4-carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-25 | 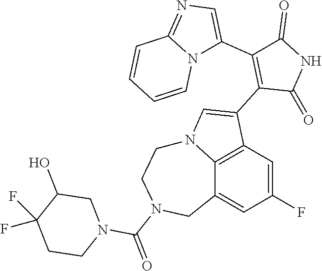 [SMILES: O=C1NC(=O)C(c2cnc3ccccn23)=C1c1cn2c3c(cc(F)cc13)CN(C(=O)N1CCC(F)(F)C(O)C1)CC2] | 3-(2-(4,4-difluoro-3-hydroxypiperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-26 | 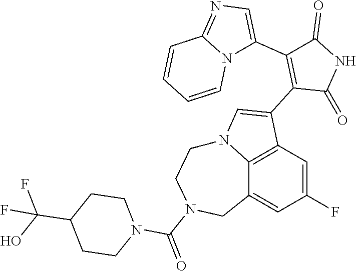 [SMILES: O=C1NC(=O)C(c2cnc3ccccn23)=C1c1cn2c3c(cc(F)cc13)CN(C(=O)N1CCC(C(O)(F)F)CC1)CC2] | 3-(2-(4-(difluoro(hydroxy)methyl)piperidine- 1-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-27 | 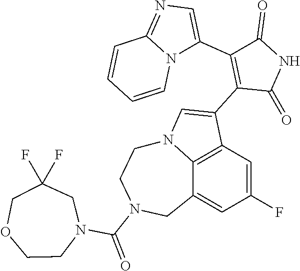 [SMILES: O=C1NC(=O)C(c2cnc3ccccn23)=C1c1cn2c3c(cc(F)cc13)CN(C(=O)N1CCOCC(F)(F)C1)CC2] | 3-(2-(6,6-difluoro-1,4-oxazepane-4-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-28 | 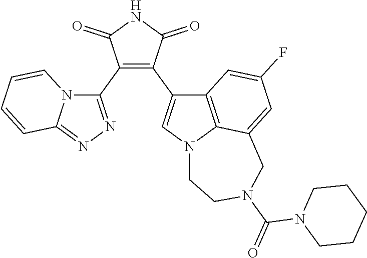 [SMILES: O=C1NC(=O)C(c2nnc3ccccn23)=C1c1cn2c3c(cc(F)cc13)CN(C(=O)N1CCCCC1)CC2] | 3-([1,2,4]triazolo[4,3-a]pyridin-3-yl)-4-(9- fluoro-2-(piperidine-1-carbonyl)-1,2,3,4- tetrahydro-[1,4]diazepino[6,7,1-hi]indol-7- yl)-1H-pyrrole-2,5-dione |
| Compound I-29 | 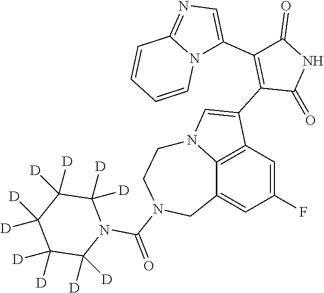 [SMILES: [2H]C1([2H])N(C(=O)N2CCn3cc(C4=C(c5cnc6ccccn56)C(=O)NC4=O)c4cc(F)cc(c43)C2)C([2H])([2H])C([2H])([2H])C([2H])([2H])C1([2H])[2H]] | 3-(9-fluoro-2-(piperidine-1-carbonyl)-d10)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-30 | 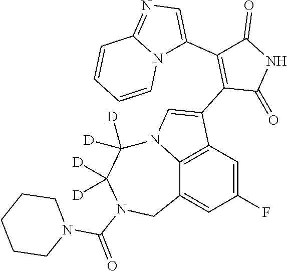 [SMILES: [2H]C1([2H])N(C(=O)N2CCCCC2)Cc2cc(F)cc3c(C4=C(c5cnc6ccccn56)C(=O)NC4=O)cn(c23)C1([2H])[2H]] | 3-(9-fluoro-2-(piperidine-1-carbonyl)-1,2,3,4- tetrahydro-[1,4]diazepino[6,7,1-hi]indol-7-yl)- 3,3,4,4-d4)-4-(imidazo[1,2-a]pyridin-3-yl)-1H- pyrrole-2,5-dione |
| Compound I-31 | 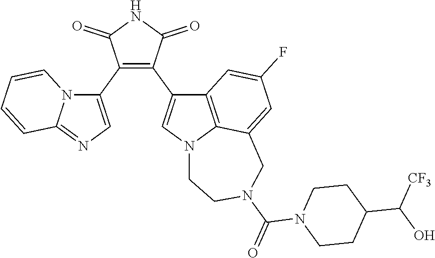 [SMILES: O=C1NC(=O)C(c2cnc3ccccn23)=C1c1cn2c3c(cc(F)cc13)CN(C(=O)N1CCC(C(O)C(F)(F)F)CC1)CC2] | 3-(9-fluoro-2-(4-(2,2,2-trifluoro-1- hydroxyethyl)piperidine-1-carbonyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-32 | 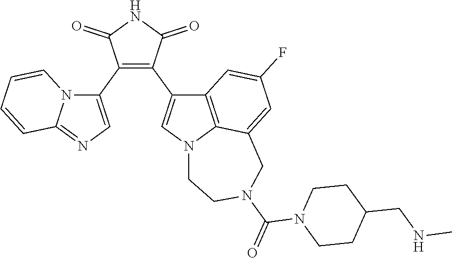 [SMILES: CNCC1CCN(C(=O)N2CCn3cc(C4=C(c5cnc6ccccn56)C(=O)NC4=O)c4cc(F)cc(c43)C2)CC1] | 3-(9-fluoro-2-(4- ((methylamino)methyl)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-33 | 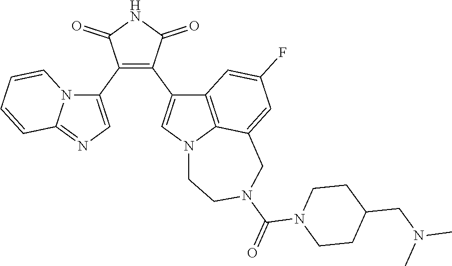 [SMILES: CN(C)CC1CCN(C(=O)N2CCn3cc(C4=C(c5cnc6ccccn56)C(=O)NC4=O)c4cc(F)cc(c43)C2)CC1] | 3-(2-(4-((dimethylamino)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-34 | 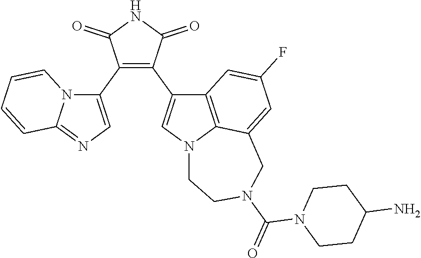 [SMILES: NC1CCN(C(=O)N2CCn3cc(C4=C(c5cnc6ccccn56)C(=O)NC4=O)c4cc(F)cc(c43)C2)CC1] | 3-(2-(4-aminopiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-35 | 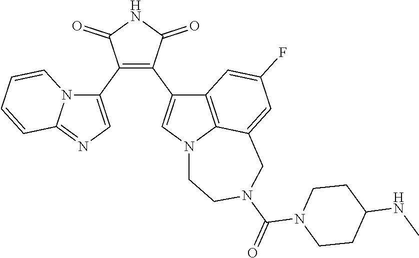 [SMILES: CNC1CCN(C(=O)N2CCn3cc(C4=C(c5cnc6ccccn56)C(=O)NC4=O)c4cc(F)cc(c43)C2)CC1] | 3-(9-fluoro-2-(4-(methylamino)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-36 | 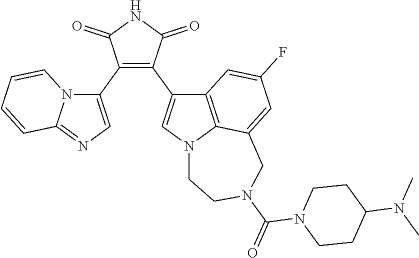 [SMILES: CN(C)C1CCN(C(=O)N2CCn3cc(C4=C(c5cnc6ccccn56)C(=O)NC4=O)c4cc(F)cc(c43)C2)CC1] | 3-(2-(4-(dimethylamino)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-37 | 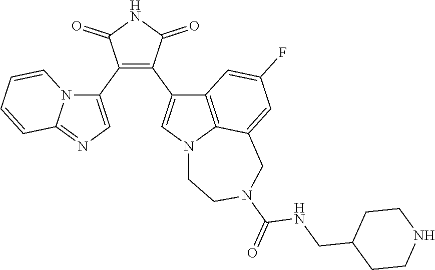 [SMILES: O=C1NC(=O)C(c2cnc3ccccn23)=C1c1cn2c3c(cc(F)cc13)CN(C(=O)NCC1CCNCC1)CC2] | 9-fluoro-7-(4-(imidazo[1,2-a]pyridin-3-yl)-2,5- dioxo-2,5-dihydro-1H-pyrrol-3-yl)-N- (piperidin-4-ylmethyl)-3,4-dihydro- [1,4]diazepino[6,7,1-hi]indole-2(1H)- carboxamide |
| Compound I-38 | 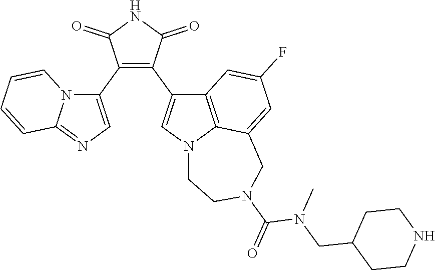 [SMILES: CN(CC1CCNCC1)C(=O)N1CCn2cc(C3=C(c4cnc5ccccn45)C(=O)NC3=O)c3cc(F)cc(c32)C1] | 9-fluoro-7-(4-(imidazo[1,2-a]pyridin-3-yl)-2,5- dioxo-2,5-dihydro-1H-pyrrol-3-yl)-N-methyl- N-(piperidin-4-ylmethyl)-3,4-dihydro- [1,4]diazepino[6,7,1-hi]indole-2(1H)- carboxamide |
| Compound I-39 | 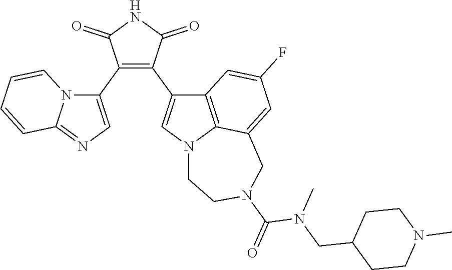 [SMILES: CN1CCC(CN(C)C(=O)N2CCn3cc(C4=C(c5cnc6ccccn56)C(=O)NC4=O)c4cc(F)cc(c43)C2)CC1] | 9-fluoro-7-(4-(imidazo[1,2-a]pyridin-3-yl)-2,5- dioxo-2,5-dihydro-1H-pyrrol-3-yl)-N-methyl- N-((1-methylpiperidin-4-yl)methyl)-3,4- dihydro-[1,4]diazepino[6,7,1-hi]indole-2(1H)- carboxamide |
| Compound I-40 | 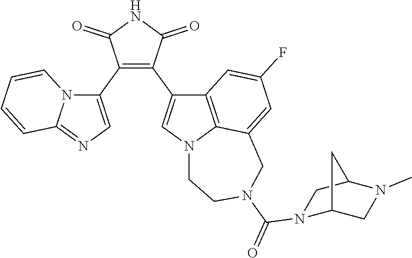 [SMILES: CN1CC2CC1CN2C(=O)N1CCn2cc(C3=C(c4cnc5ccccn45)C(=O)NC3=O)c3cc(F)cc(c32)C1] | 3-(9-fluoro-2-((1R,4R)-5-methyl-2,5- diazabicyclo[2.2.1]heptane-2-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-41 | 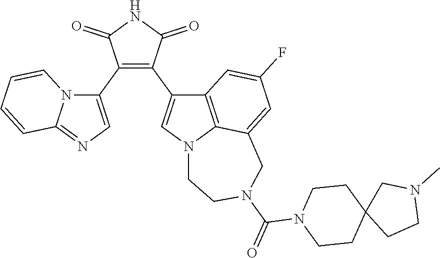 [SMILES: CN1CCC2(CCN(C(=O)N3CCn4cc(C5=C(c6cnc7ccccn67)C(=O)NC5=O)c5cc(F)cc(c54)C3)CC2)C1] | 3-(9-fluoro-2-(2-methyl-2,8- diazaspiro[4.5]decane-8-carbonyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-42 | 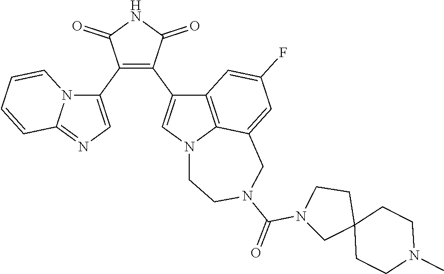 [SMILES: CN1CCC2(CC1)CCN(C(=O)N1CCn3cc(C4=C(c5cnc6ccccn56)C(=O)NC4=O)c4cc(F)cc(c43)C1)C2] | 3-(9-fluoro-2-(8-methyl-2,8- diazaspiro[4.5]decane-2-carbonyl)-1,2,3,4- tetrahydro-[1,4]diazepino[6,7,1-hi]indol-7- yl)-4-(imidazo[1,2-a]pyridin-3-yl)-1H-pyrrole- 2,5-dione |
| Compound I-43 | 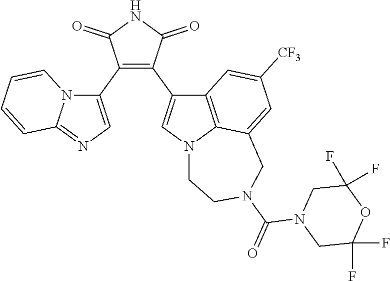 [SMILES: O=C1NC(=O)C(c2cnc3ccccn23)=C1c1cn2c3c(cc(C(F)(F)F)cc13)CN(C(=O)N1CC(F)(F)OC(F)(F)C1)CC2] | 3-(imidazo[1,2-a]pyridin-3-yl)-4-(2-(2,2,6,6- tetrafluoromorpholine-4-carbonyl)-9- (trifluoromethyl)-1,2,3,4-tetrahydro- [1,4]diazepino[6,7,1-hi]indol-7-yl)-1H- pyrrole-2,5-dione |
| Compound I-44 | 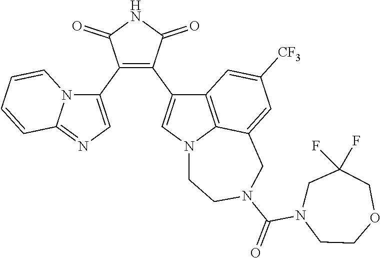 [SMILES: O=C1NC(=O)C(c2cnc3ccccn23)=C1c1cn2c3c(cc(C(F)(F)F)cc13)CN(C(=O)N1CCOCC(F)(F)C1)CC2] | 3-(2-(6,6-difluoro-1,4-oxazepane-4-carbonyl)- 9-(trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-45 | 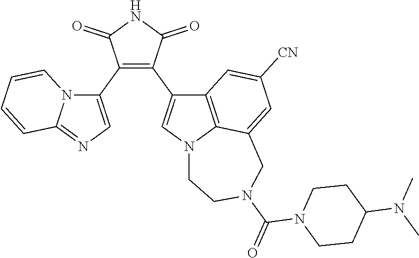 [SMILES: CN(C)C1CCN(C(=O)N2CCn3cc(C4=C(c5cnc6ccccn56)C(=O)NC4=O)c4cc(C#N)cc(c43)C2)CC1] | 2-(4-(dimethylamino)piperidine-1-carbonyl)- 7-(4-(imidazo[1,2-a]pyridin-3-yl)-2,5-dioxo- 2,5-dihydro-1H-pyrrol-3-yl)-1,2,3,4- tetrahydro-[1,4]diazepino[6,7,1-hi]indole-9- carbonitrile |
| Compound I-46 | 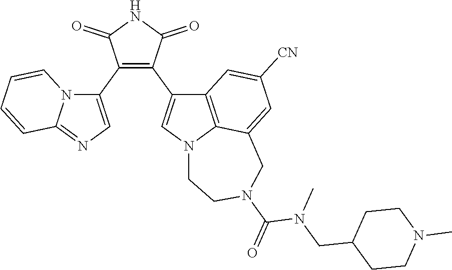 [SMILES: CN1CCC(CN(C)C(=O)N2CCn3cc(C4=C(c5cnc6ccccn56)C(=O)NC4=O)c4cc(C#N)cc(c43)C2)CC1] | 9-cyano-7-(4-(imidazo[1,2-a]pyridin-3-yl)-2,5- dioxo-2,5-dihydro-1H-pyrrol-3-yl)-N-methyl- N-((1-methylpiperidin-4-yl)methyl)-3,4- dihydro-[1,4]diazepino[6,7,1-hi]indole-2(1H)- carboxamide |
| Compound I-47 | 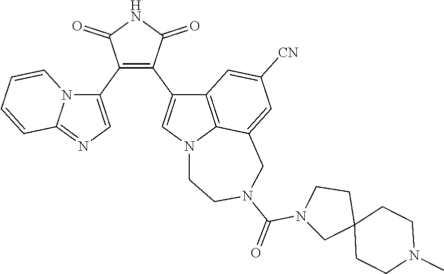 [SMILES: CN1CCC2(CC1)CCN(C(=O)N1CCn3cc(C4=C(c5cnc6ccccn56)C(=O)NC4=O)c4cc(C#N)cc(c43)C1)C2] | 7-(4-(imidazo[1,2-a]pyridin-3-yl)-2,5-dioxo- 2,5-dihydro-1H-pyrrol-3-yl)-2-(8-methyl-2,8- diazaspiro[4.5]decane-2-carbonyl)-1,2,3,4- tetrahydro-[1,4]diazepino[6,7,1-hi]indole-9- carbonitrile |
| Compound I-48 | 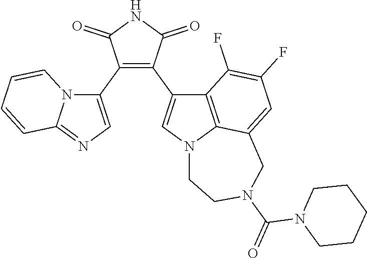 [SMILES: O=C1NC(=O)C(c2cnc3ccccn23)=C1c1cn2c3c(cc(F)c(F)c13)CN(C(=O)N1CCCCC1)CC2] | 3-(8,9-difluoro-2-(piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |

Unless otherwise stated, structures depicted herein are also meant to include compounds which differ only in the presence of one or more isotopically enriched atoms. For example, compounds having the present structure except for the replacement of a hydrogen atom by deuterium or tritium, or the replacement of a carbon atom by 13C or 14C, or the replacement of a nitrogen atom by 15N, or the replacement of an oxygen atom with 17O or 18O are within the scope of the present disclosure. Such isotopically labeled compounds are useful as research or diagnostic tools. In certain embodiments, deuteration can be used to slow metabolism and thus potentially improve the compound half-life. Any or all hydrogens in the compound can be replaced with deuterium.

In certain embodiments, the GSK inhibitor is Compound I-7 [3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione].

In some embodiments of the methods disclosed herein, the Wnt agonist is selected from CHIR99021, LY2090314, AZD1080, GSK3 inhibitor XXII, Compound I-6, Compound I-7, and Compound I-12.

In one embodiment, the Wnt agonist is CHIR99021 or a pharmaceutically acceptable salt thereof.

In one embodiment, the Wnt agonist is GSK-3 Inhibitor XXII or a pharmaceutically acceptable salt thereof.

In one embodiment, the Wnt agonist is a compound of Formula I or a pharmaceutically acceptable salt thereof.

In one embodiment, the Wnt agonist is LY2090314 or a pharmaceutically acceptable salt thereof.

In one embodiment, the Wnt agonist is AZD1080 or a pharmaceutically acceptable salt thereof.

In another embodiment, the Wnt agonist is selected from CHIR99021, GSK-3 Inhibitor XXII, Formula I, LY2090314, AZD1080, and pharmaceutically acceptable salts thereof.

Methods of determining whether or not a compound, protein or nucleic acid is a Wnt agonist will be apparent to one of ordinary skill in the art. For example, Wnt reporters such as the TOP-Flash reporter are known in the art. Wnt reporters comprise a Wnt target promoter (e.g. a promoter containing TCF/LEF binding sites) operably linked to a reporter gene such as luciferase. The person of ordinary skill can transduce or transfect cells with a Wnt reporter, administer a potential Wnt agonist to the cells, and measure the levels of Wnt/β-catenin signaling by measuring luciferase activity from the reporter. Wnt agonists will increase the levels of the luciferase. Alternatively, the person or ordinary skill can measure the levels of Axin2 and β-catenin. Alternatively, or in addition, the person of ordinary skill can measure the effect of the Wnt agonist on phosphorylation of Wnt pathway proteins, for example Dsh or LRP5/6. Methods of detecting and measuring gene expression and/or phosphorylation are known in the art. Levels of protein can be measured, for example, by antibody detection and Western Blot or immunohistochemistry. Phosphorylation of Dsh or LRP5/6 can be detected by use of an antibody to the phosphorylated forms of the proteins and Western Blot or immunohistochemistry.

### Formulations

The methods of the present disclosure can employ various formulations for systemic administration to subjects, e.g. humans and animals, in unit dosage forms, such as tablets, capsules, pills, powders, granules, sterile parenteral solutions or suspensions, oral solutions or suspensions, and oil-water emulsions containing suitable quantities of a valproic acid compound.

Locally administered compositions (e.g. of the Wnt agonist and/or locally administered valproic acid compound) as described herein can be administered by a number of methods sufficient to deliver the composition to the inner ear. Delivering a composition to the inner ear includes administering the composition to the middle ear, such that the composition may diffuse across the round window to the inner ear and administering a composition to the inner ear by direct injection through the round window membrane. Such methods include, but are not limited to auricular administration, by transtympanic wicks or catheters, or parenteral administration, for example, by intraauricular, transtympanic, or intravestibular injection.

In particular embodiments, the valproic acid compounds of the disclosure, and formulations thereof, are systemically administered, and meaning that they are not administered locally. In particular embodiments, the Wnt agonist of the disclosure, and formulations thereof, are locally administered, meaning that they are not administered systemically. In certain embodiments, a valproic acid compound of the disclosure, and formulations thereof, are further also administered locally, meaning a valproic acid compound is administered locally and systemically.

Systemically administered oral pharmaceutical dosage forms (of a valproic acid compound) can be either solid or liquid. The solid dosage forms can be tablets, capsules, granules, and bulk powders. Types of oral tablets include compressed, chewable lozenges and tablets which can be enteric-coated, sugar-coated or film-coated. Capsules can be hard or soft gelatin capsules, while granules and powders can be provided in non-effervescent or effervescent form with the combination of other ingredients known to those skilled in the art. In other embodiments, the oral dosage form is an osmotic-controlled release oral delivery system (OROS). In other embodiments, the oral dosage form may include matrix-embedded dosage forms or related devices. In some embodiments, the present oral dosage forms may include orally-disintegrating tablets.

Pharmaceutically acceptable carriers utilized in tablets include binders, lubricants, diluents, disintegrating agents, coloring agents, flavoring agents, and wetting agents.

Liquid oral dosage forms (of the valproic acid compound) include aqueous solutions, emulsions, suspensions, solutions and/or suspensions reconstituted from non-effervescent granules and effervescent preparations reconstituted from effervescent granules.

Aqueous solutions of the valproic acid compound) include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

Parenteral administration of the systemically administered valproic acid compound formulations of the present disclosure includes intravenous, subcutaneous and intramuscular administrations of immediate, sustained (e.g. depot), extended, and/or modified release formulations (e.g. as described herein). Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions can be either aqueous or non-aqueous. Pharmaceutically acceptable carriers used in parenteral preparations include aqueous vehicles, non-aqueous vehicles, antimicrobial agents, isotonic agents, buffers, antioxidants, local anesthetics, suspending and dispersing agents, emulsifying agents, sequestering or chelating agents and other pharmaceutically acceptable substances.

In some embodiments, locally administered Wnt agonist and optionally valproic acid compound formulations may also contain a round window membrane penetration enhancer, such as anesthetics, endotoxins, exotoxins, histamine, osmotic disturbance agents, and benzyl alcohol.

The concentration of the systemically administered valproic acid compound can be adjusted so that an injection thereof provides an effective amount (as described herein) to produce the desired pharmacological effect. The exact dose depends on the age, weight and condition of the patient or animal, as is known in the art. The unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing a valproic acid compound and/or Wnt agonist is an effective mode of administration.

Systemic dosage forms of the valproic acid compound for rectal administration can be rectal suppositories, capsules and tablets for systemic effect. Rectal suppositories as used herein mean solid bodies for insertion into the rectum which melt or soften at body temperature releasing the pharmacologically and/or therapeutically active ingredients contained in the composition of this disclosure. Pharmaceutically acceptable substances utilized in rectal suppositories are bases or vehicles and agents to raise the melting point. Examples of bases include cocoa butter (theobroma oil), glycerin-gelatin, carbowax, polyoxyethylene glycol and mixtures of mono-, di- and triglycerides of fatty acids. Combinations of the various bases can be used. Agents to raise the melting point of suppositories include spermaceti and wax. Rectal suppositories can be prepared either by the compressed method or by molding. The typical weight of a rectal suppository is about 2 to 3 gm. Tablets and capsules for rectal administration can be manufactured using the same pharmaceutically acceptable substance and by the same methods as for formulations for oral administration.

Formulations for systemic or local administration can provide the Wnt agonist or valproic acid compound as suspended in micronized or other suitable form or can be derivatized to produce a more soluble active product. The form of the resulting composition depends upon a number of factors, including the intended mode of administration and the solubility of the valproic acid compound and/or Wnt agonist in the selected carrier or vehicle. The effective concentration is sufficient for treating or alleviating any of the diseases, disorders, and symptoms thereof as described herein, and can be empirically determined.

The resulting mixture can be a solution, suspension, emulsion or the like, and can be formulated as a gel, emulsion, solution, elixir, suspension, paste, foam, aerosol, irrigation, spray, suppository, transdermal patch, or any other formulation suitable for systemic or local administration.

Pharmaceutical carriers or vehicles suitable for administration of the compositions include any such carriers known to those skilled in the art to be suitable for the particular mode of administration. The carriers are in amounts sufficient to exert a therapeutically useful effect without serious toxic effects on the treated individual. To formulate these compositions, a weight fraction of a valproic acid compound or Wnt agonist is dissolved, suspended, dispersed or otherwise mixed in a selected vehicle at an effective concentration such that the disease or disorder is relieved or ameliorated.

Pharmaceutical compositions for any route of administration of this disclosure contain a therapeutically effective amount of a valproic acid compound and/or Wnt agonist, and, as necessary, inorganic or organic, solid or liquid pharmaceutically acceptable carriers or excipients. Pharmaceutical compositions suited for local administration to the inner ear include aqueous solutions or suspensions, which, e.g. in the case of lyophilized formulations that contain the active ingredient alone or together with a carrier, are prepared prior to use. They further include gels or hydrogels, which are biodegradable such as those derived from silk fibroin, or non-biodegradable, aqueous or non-aqueous, or may include microparticles, microspheres, films, or coatings. Examples of gel-forming biocompatible polymers include, but are not limited to, silk fibroin, hyaluronic acid, hyaluronates, lecithin gels, (poly)alanine derivatives, pluronics, poly(ethyleneglycol), poloxamers, chitosans, xyloglucans, collagens, fibrins, milk proteins, elastins, aloe vera, gelatin, albumin. polyesters, poly(lactides), poly(glycolide) or their co-polymers PLGA, sucrose acetate isobutyrate, and glycerol monooleate. In particular, silk fibroin systems for sustained delivery allow for non-inflammatory biodegradation and drug stabilization: they offer processing options without chemical cross-linkers, are compatible with common sterilization methods, and are purified under aqueous and ambient conditions. Further, silk fibroin-based platforms enhance cell attachment, improve drug-release kinetics, and show superior blood compatibility. In some embodiments, gels can be easily administered into the middle ear, release the active agent over an extended period of time, and allow for a high percentage of the active agent to be delivered into the inner ear.

In some aspects, the present disclosure the pharmaceutical compositions are lyophilized comprising one or more agents described herein and a gelling agent.

In some embodiments, the lyophilized pharmaceutical composition is in the form of a lyophilized cake.

In some embodiments, the lyophilized pharmaceutical composition has a higher stability to oxygen and/or light as compared to a comparable pharmaceutical composition comprising one or more solvents.

In some embodiments, the present disclosure provides a reconstituted solution of the lyophilized pharmaceutical compositions.

As used herein, the term “gelling agent” refers to an agent capable of imparting a gel-like or thickening quality to the pharmaceutical composition or reconstituted solution of the present disclosure upon being subjected to a gelling condition (e.g. a particular temperature or temperature range, the presence of an ion, a pH value or range, or a concentration of gelling agent that causes the gelling agent to undergoing a change or transition from low viscosity to high viscosity, or the reverse). In some embodiments, the gelling condition is a particular temperature (e.g. about 26° C., about 27° C., about 28° C., about 29° C., about 30° C., about 31° C., about 32° C., about 33° C., about 34° C., about 35° C., about 36° C., about 37° C., about 38° C. about 39° C., or about 40° C.). In some embodiments, the gelling condition is a particular temperature range (e.g. about 26° C. or higher, about 27° C. or higher, about 28° C. or higher, about 29° C. or higher, about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, or about 40° C. or higher). In some embodiments, the gelling agent provides a viscosity of between about 1,000 and 10,000,000 centipoise, between about 5,000 and 5,000,000 centipoise, or between about 100,000 and 4,000,000 centipoise, to the pharmaceutical composition or reconstituted solution of the present disclosure. In some embodiments, the gelling agent provides a viscosity of between about 50,000 and 2,000,000 centipoise to the pharmaceutical composition or reconstituted solution of the present disclosure.

In some embodiments, prior to gelling (e.g. at ambient temperature (e.g. between about 20° C. and about 26° C.), the gelling agent provides a viscosity of less than about 100,000 centipoise, less than about 50,000 centipoise, 20,000 centipoise, less than about 10,000 centipoise, less than about 8,000 centipoise, less than about 7,000 centipoise, less than about 6,000 centipoise, less than about 5,000 centipoise, less than about 4,000 centipoise, less than about 3,000 centipoise, less than about 2,000 centipoise, or less than about 1,000 centipoise to the pharmaceutical composition or reconstituted solution of the present disclosure.

In some embodiments, upon gelling (e.g. at the temperature of a human body (e.g. between about 35° C. to about 39° C., between about 36° C. to about 38° C., or at about 37° C.), the gelling agent provides a viscosity of greater than about 1,000 centipoise, greater than about 5,000 centipoise, greater than about 10,000 centipoise, greater than about 20,000 centipoise, greater than about 50,000 centipoise, greater than about 60,000 centipoise, greater than about 70,000 centipoise, greater than about 80,000 centipoise, greater than about 90,000 centipoise, or greater than about 100,000 centipoise.

In some embodiments, upon gelling (e.g. at the temperature of a human body (e.g. between about 36° C. to about 39° C., or at about 37° C.), the viscosity of the pharmaceutical composition or reconstituted solution of the present disclosure, as measured in units of centipoise, being about 2 fold or greater, about 5 fold or greater, about 10 fold or greater, about 20 fold or greater, about 50 fold or greater, about 60 fold or greater, about 7 fold or greater, about 80 fold or greater, about 90 fold or greater, about 100 fold or greater as compared to the viscosity of the pharmaceutical composition or reconstituted solution prior to gelling (e.g. at ambient temperature (e.g. at about 25° C.)).

It is understood that the gelling condition (e.g. gelling temperature) of the pharmaceutical composition or reconstituted solution of the present disclosure is measured with a variety of techniques in the art. In some embodiment, the gelling temperature is determined using a commercially available rheometer having a parallel plate geometry (e.g. with plate distance ranging from 0.5 mm to 1.0 mm). In some embodiments, the analysis is performed over a continuous temperature range (e.g. 15° C. to 40° C.) at a constant rate (e.g. 2 to 3° C./min) and a deformation frequency of 0.74 Hz to 1 Hz. The gelation temperature is determined at the temperature whereby the shear storage modulus (G′) and the shear loss modulus (G″) are equal.

In some embodiments, the gelling agent comprises acacia, alginic acid, bentonite, poly(acrylic acid) (Carbomer), carboxymethyl cellulose, ethylcellulose, gelatin, hydroxyethyl cellulose, hydroxypropyl cellulose, magnesium aluminum silicate (Veegum), methylcellulose, poloxamer, hyaluronic acid sodium, polylacticglycolic acid sodium, chitosan, polyvinyl alcohol, sodium alginate, tragacanth, xanthan gum, or any combination thereof. In some embodiment, the gelling agent comprises poloxamer.

In some embodiments, the gelling agent is a thermoreversible gelling agent.

As used herein, the term “thermoreversible” refers to a capability of being reversible by the application of heat. The “thermoreversible gelling agent” refers to an agent capable of reversibly imparting a gel-like or thickening quality to the pharmaceutical composition or reconstituted solution of the present disclosure upon application of heat.

In some embodiments, the thermoreversible gelling agent comprises a poloxamer.

It is understood that the gelling agent (e.g. the thermoreversible gelling agent) may also be a bulking agent of the pharmaceutical composition or reconstituted solution of the present disclosure. In some embodiments, a poloxamer (e.g. poloxamer 407) is the gelling agent and/or the bulking agent of the pharmaceutical composition or reconstituted solution of the present disclosure. Poloxomers are a general class of commercially available and pharmaceutically acceptable triblock copolymers of polyethylene oxide-polypropylene oxide-polyethylene oxide which exhibit relatively low viscosity at low temperatures (e.g. room temperature or below) but much high viscosities at elevated temperatures (e.g. body temperatures of approximately 37° C.) whereby compositions containing such thermoreversible gelling agents effectively solidify in place. Other thermoreversible gelling agents such as polyethylene oxide-polylactic acid-polyethylene oxide polymers are also suitable in various embodiments of the present invention.

In some embodiments, the poloxamer (e.g. poloxamer 407) is the gelling agent and the bulking agent of the pharmaceutical composition or reconstituted solution of the present disclosure. In some embodiments, the presence of the poloxamer (e.g. poloxamer 407) in the pharmaceutical composition (e.g. the lyophilized pharmaceutical composition) alleviates the need for any other excipient (e.g. additional bulking agent). Such alleviation may provide one or more advantages to the pharmaceutical composition (e.g. enhanced stability and/or reduced reconstitution time).

In some embodiments, the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407.

In some embodiments, the poloxamer is Poloxamer 188 or Poloxamer 407.

In some embodiments, the poloxamer is Poloxamer 407.

In some embodiments, the poloxamer is a purified poloxamer (e.g. purified Poloxamer 407).

In some embodiments, the purified poloxamer (e.g. purified Poloxamer 407) has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.

In some embodiments, the purified poloxamer (e.g. purified Poloxamer 407) has a reduced level of polymer chains with molecular weight below 9 kDa as compared to the unpurified poloxamer (e.g. unpurified Poloxamer 407).

In some embodiments, the purified poloxamer (e.g. purified Poloxamer 407) has about 99% or less, about 98% or less, about 95% or less, about 900% or less, about 80% or less, about 70% or less, about 60% or less, about 50% or less, about 40% or less, about 30% or less, about 20% or less, or about 10% or less of polymer chains with molecular weight below 9 kDa as compared to the unpurified poloxamer (e.g. unpurified Poloxamer 407).

In some embodiments, the purified poloxamer (e.g. purified Poloxamer 407) is prepared by liquid-liquid extraction or size exclusion chromatography.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the poloxamer (e.g. Poloxamer 407) during the purification.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more diblock copolymers (e.g. PEO-PPO), single block polymers (e.g. PEO), and/or aldehydes are removed from the poloxamer (e.g. Poloxamer 407) during the purification.

In some embodiments, the pharmaceutical composition, pharmaceutical composition, the lyophilized pharmaceutical composition or reconstituted solution of the present disclosure comprises a buffering agent. The buffer controls the pH of the reconstituted solution to a range of from about 4 to about 13, from about 5 to about 12, from about 6 to about 11, from about 6.5 to about 10.5, or from about 7 to about 10.

Examples of the buffering agent include, but are not limited to, citrate buffering agents, acetate buffering agents, phosphate buffering agents, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents are selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the buffering agent comprises phosphate buffered saline, TRIS, tris acetate, tris HCl-65, sodium citrate, histidine, arginine, sodium phosphate, tris base-65, hydroxyethyl starch, or any combination thereof.

In some embodiments, the pharmaceutical composition, pharmaceutical composition, the lyophilized pharmaceutical composition or reconstituted solution of the present disclosure comprises a bulking agent.

In some embodiments, the bulking agent comprises poloxamer (e.g. poloxamer 407), mannitol, sucrose, maltose, trehalose, dextrose, sorbitol, glucose, raffinose, glycine, histidine, polyvinylpyrrolidone (e.g. polyvinylpyrrolidone K12 or polyvinylpyrrolidone K17), lactose, or any combination thereof.

In some embodiments, the pharmaceutical composition, pharmaceutical composition, the lyophilized pharmaceutical composition or reconstituted solution of the present disclosure comprises a stabilizing agent.

In some embodiments, the stabilizing agent comprises a cryoprotectant. In some embodiments, the cryoprotectant is a polyol (e.g. a diol or a triol such as propylene glycol (i.e. 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g. NDSB-201 (3-(1-pyridino)-1-propane sulfonate), an osmolyte (e.g. L-proline or trimethylamine N-oxide dihydrate), a polymer (e.g. polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g. polyvinylpyrrolidone K 15), pentaerythritol propoxylate, or polypropylene glycol P 400), an organic solvent (e.g. dimethyl sulfoxide (DMSO) or ethanol), a sugar (e.g. D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g. lithium acetate, lithium chloride, lithium formate, lithium nitrate, lithium sulfate, magnesium acetate, sodium chloride, sodium formate, sodium malonate, sodium nitrate, sodium sulfate, or any hydrate thereof) or any combination thereof.

In some embodiments, the stabilizing agent comprises a salt. In some embodiment, the salt is selected from the group consisting of lithium salts (e.g. lithium acetate, lithium chloride, lithium formate, lithium nitrate, lithium sulfate, or any hydrate thereof), magnesium salts (e.g. magnesium acetate or a hydrate thereof), and sodium salts (e.g. sodium chloride, sodium formate, sodium malonate, sodium nitrate, sodium sulfate, or any hydrate thereof). For another example, the formulation comprises one or more sodium salts. For yet another example, the formulation comprises sodium chloride.

In some embodiment, the stabilizing agent comprises a surfactant. In some embodiments, the surfactant comprises one or more anionic surfactants (e.g. 2-acrylamido-2-methylpropane sulfonic acid, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, or sulfolipid), one or more cationic surfactants (e.g. behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, or thonzonium bromide), one or more zwitterionic surfactants (e.g. cocamidopropyl betaine, cocamidopropyl hydroxysultaine, dipalmitoylphosphatidylcholine, egg lecithin, hydroxysultaine, lecithin, myristamine oxide, peptitergents, or sodium lauroamphoacetate), and/or one or more non-ionic surfactants (e.g. alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, α-tocopheryl polyethylene glycol succinate (TPGS), poloxamer (e.g. poloxamer 407), polyethoxylated tallow amine, polyglycerol polyricinoleate, polysorbate (e.g. polysorbate 20, polysorbate 40, polysorbate 60, or polysorbate 80), sorbitan, sorbitan monolaurate, sorbitan monostearate, sorbitan tristearate, stearyl alcohol, surfactin, triton x-100).

In some embodiments, the pharmaceutical composition, pharmaceutical composition, the lyophilized pharmaceutical composition or reconstituted solution of the present disclosure comprises a tonicity-adjusting agent.

In some embodiments, the tonicity-adjusting agent comprises NaCl, dextrose, dextran, ficoll, gelatin, mannitol, sucrose, glycine, glycerol, or any combination thereof.

In some embodiments, the pharmaceutical composition or reconstituted solution of the present disclosure comprises a soothing agent. In some embodiments, the soothing agent comprises lidocaine

In addition to these components, the pharmaceutical composition, pharmaceutical composition, the lyophilized pharmaceutical composition or reconstituted solution of the present disclosure includes any substance useful in pharmaceutical compositions.

In some embodiments, the pharmaceutical composition, pharmaceutical composition, the lyophilized pharmaceutical composition or reconstituted solution of the present disclosure includes one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents are selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof.

A binding agent is starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan): alginates; polyethylene oxide; polyethylene glycol; inorganic calcium salts; silicic acid: polymethacrylates; waxes: water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®.

Examples of oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, an/or silicone oil.

### Dose

### Systemic Administration Valproic Acid Compound

A therapeutically effective amount or dose is defined as an amount or dose effective to treat the disease or disorder and/or suppress or reduce symptoms of the disease or disorder in a treated individual. A therapeutically effective amount or dose is also the amount effective to treat the disease or disorder and/or suppress or reduce symptoms of the disease or disorder in the afflicted individual.

In many embodiments, a daily dose of a systemically-administered valproic acid compound is equivalent to about 0.5-25000 mg valproic acid. A valproic acid compound is systemically administered at a daily dose equivalent to an amount in mg of valproic acid of about: 0.5, 5, 10, 25, 50, 75, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, 25000; or a value within a range between any of the preceding values, for example, between about 100 and about 500, between about 650 and about 700, between about 400 and about 4000, between about 5000 and about 20000, or the like. The valproic acid compound may include any valproic acid compound described herein. In some embodiments, the valproic acid compound is administered orally.

In other embodiments, a valproic acid compound is orally administered at a dose in mg of valproic acid of about: 50, 100, 125, 250, 500, 1000, 2000, 3000, 4000, 5000, 6000, 7000, 8000, 9000, 10000, 15000, 20000, or 25000; or a value within a range between any of the preceding values, for example, between about 50 and about 250, between about 100 and about 500, between about 500 and about 2000, or the like. In some embodiments the valproic acid compound is valproic acid, sodium valproate, or a combination thereof. In some embodiments the valproic acid compound is sodium valproate.

In some embodiments, a systemically-administered valproic acid compound is administered as a formulation including about 50-200 mg of valproic acid per mL. A valproic acid compound is systemically administered as a formulation including valproic acid in mg/mL of about: 50, 55, 60, 65, 70, 75, 80, 85, 90, 95, 100, 105, 110, 120, 125, 130, 135, 140, 145, 150, 155, 160, 165, 170, 175, 180, 185, 190, 195, or about 200; or a value between any of the preceding values, for example, between about 95-105 mg/mL, or the like. In some embodiments, a valproic acid compound is formulated for systemic administrations at about 95 mg/mL. In some embodiments, a valproic acid compound is formulated for systemic administrations at about 100 mg/mL. The valproic acid compound may include any valproic acid compound described herein, such as sodium valproate, or a mixture of sodium valproate and valproic acid.

In some embodiments, a valproic acid compound is systemically administered about 1, 2, 3, or 4 times per day. In some embodiments, a valproic acid compound is administered once or twice daily. In some embodiments a valproic acid compound is administered once daily. In other embodiments, a valproic acid compound is administered twice daily. In some embodiments, the systemically administered valproic acid compound is administered beginning about 1-14 days prior to initiating local administration of a Wnt agonist.

In some embodiments, systemic administration may begin about: 1, 2, 3, 4, 5, 6, 7, 8, 9, 10, 11, 12, 13, or 14 days prior to local administration of the Wnt agonist, or a value within a range between any of the preceding values, for example, between about 2 and about 4, between about 7 and about 14, between about 5 and about 12, or the like. In some embodiments, systemic administration may begin 1, 2, 3, 4, 7, or 14 days prior to local administration.

In some embodiments, the systemically administered valproic acid compound is administered once or twice per day until about 1-8 doses of the Wnt agonist have been locally administered (as described herein). In some embodiments, a valproic acid compound is systemically administered once per day until about 1, 2, 3, 4, 5, 6, 7, or 8 dose(s) of Wnt agonist has/have been administered, or a value within a range of any of the preceding values, for example, between about 2 and about 4, between about 2 and about 6, or the like. In some embodiments, the dosing regimen for a systemically-delivered valproic acid compound may change, upon administration of 1-8 doses of Wnt agonist, from once per day to 1-4 times weekly or monthly.

### Local Administration Wnt Agonist

In some embodiments, a locally-administered Wnt agonist is administered at a formulation concentration of about 1 nM to about 1000 mM. A Wnt agonist is locally administered at a formulation concentration of about: 1 nM, 10 nM, 50 nM, 100 nM, 250 nM, 500 nM, 750 nM, 1 μM, 10 μM, 50 μM, 100 μM 250 μM, 500 μM, 750 μM, 1 mM, 10 mM, 50 nM, 100 mM, 250 mM, 500 mM, 750 mM, or 1,000 mM, or a value between any of the preceding values, for example, between about 1 nM and about 10 nM, between about 100 nM to about 250 μM, between about 1 μM and about 1,000 nM, or the like. The Wnt agonist is any Wnt agonist described herein. In some embodiments, the Wnt agonist is administered intratympanically. In some embodiments, the locally-administered Wnt agonist forms a concentration gradient upon diffusing into the cochlea, with an average perilymph concentration of about 100 nM to about 20 μM (depending on the Wnt agonist), for example average perilymph concentrations of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM or 20 μM, inclusive of any range defined by the preceding values.

In several embodiments, the locally administering of a Wnt agonist may provide a systemic plasma concentration of 0.01-10 μg/mL. The locally administering of a Wnt agonist may provide a systemic plasma concentration in μg/mL of about: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, or 10; or a value between any of the preceding values, for example, between about 1-10 μg/mL, or the like.

In some embodiments, the locally administering of a Wnt agonist may occur at a time when the subject has a systemic plasma concentration of valproic acid of about 5 μg/mL to about 5000 μg/mL, e.g. about, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 1000, 1500, 2000, 2500, 3000, 3500, 4000, 4500, or 5000 μg/mL; or a value or range between any of the preceding values. In some embodiments, the Wnt agonist is locally administered when the subject has a systemic valproic acid plasma concentration of about 5 μg/mL to about 500 μg/mL. In further embodiments, the systemic valproic acid plasma concentration is maintained at about 5 μg/mL to about 5000 μg/mL for at least about 1-10 hours prior to the locally administering. In further embodiments, the systemic valproic acid plasma concentration is maintained at about 5 μg/mL to about 5000 μg/mL for at least about 1-10 hours after the locally administering. In some embodiments, the systemic valproic acid plasma concentration is maintained at about 5 μg/mL to about 500 μg/mL for at least about 1-10 hours prior to the locally administering. In some embodiments, the systemic valproic acid plasma concentration is maintained at about 5 μg/mL to about 500 μg/mL for at least about 1-10 hours after the locally administering. In some embodiments, the systemic valproic acid plasma concentration is maintained at about 500 μg/mL to about 5000 μg/mL for at least about 1-10 hours prior to the locally administering. In some embodiments, the systemic valproic acid plasma concentration is maintained at about 500 μg/mL to about 5000 μg/mL for at least about 1-10 hours after the locally administering. The maintaining of a systemic valproic acid plasma concentration may include systemically administering a valproic acid compound and measuring and monitoring the valproic acid plasma concentration and adjusting the frequency and amount of valproic acid compound administered.

In some embodiments, the locally-administered Wnt agonist is CHIR99021, or a pharmaceutically acceptable salt thereof, and is administered at a formulation concentration of about 1 mM to about 10 mM, including about: 1 mM, 2 mM, 3 mM, 4 mM, 5 mM, 6 mM, 7 mM, 8 mM, 9 mM, or 10 mM; or a value between any of the preceding values. In some embodiments. In some embodiments, CHIR99021, or a pharmaceutically acceptable salt thereof, is administered intratympanically, for example at a concentration of about 8 mM, and a volume of about 50-200 μL.

In some embodiments, the locally-administered Wnt agonist is CHIR99021, or a pharmaceutically acceptable salt thereof, and is administered in an amount between about 50 μg to about 1000 μg; e.g. 50, 60, 70, 80, 90, 100, 120, 140, 160, 180, 190, 200, 225, 250, 275, 300, 325, 250, 275, 400, 425, 450, 475, 500, 525, 550, 575, 600, 625, 650, 675, 700, 750, 800, 850, 900, 950, or 1000; or a value or range between any of the preceding values. In some embodiments, CHIR99021, or a pharmaceutically acceptable salt thereof, is administered in an amount between about 125 μg to about 650 μg. In some embodiments, CHIR99021 is administered in an amount between about 157 μg to about 628 μg.

In some embodiments, the Wnt agonist is CHIR99021, or a pharmaceutically acceptable salt thereof, and is administered, for example, to a cochlear cell in an amount sufficient to achieve a concentration of about 0.001 mM to 10 mM, about 0.01 mM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM, in the perilymph fluid in the inner ear.

In some embodiments, CHIR99021, or a pharmaceutically acceptable salt thereof, is administered in an amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear.

In some embodiments, the Wnt agonist is CHIR99021, or a pharmaceutically acceptable salt thereof, and is administered to a subject, for example, to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, CHIR99021, or a pharmaceutically acceptable salt thereof, is administered to a subject, for example, to the middle ear at a concentration of about 1 mM, 2 mM, 3 mM, 4 mM, 5 mM, 6 mM, 7 mM, 8 mM, 9 mM, or 10 mM. In some embodiments, CHIR99021, or a pharmaceutically acceptable salt thereof, is administered to a subject, for example, to the middle ear at a concentration of 6.7 mM.

In some embodiments, the locally-administered Wnt agonist is LY2090314, or a pharmaceutically acceptable salt thereof, and is administered at a formulation concentration of about 1 μM to about 50 μM, e.g. 1 μM, 5 μM, 10 μM, 15 μM, 20 μM, 30 μM, 50 μM; or a value between any of the preceding values. In some embodiments, LY2090314, or a pharmaceutically acceptable salt thereof, is administered intratympanically, for example at a concentration of about 10 AM, and a volume of about 50-200 μL.

In some embodiments, the locally-administered Wnt agonist is LY2090314, or a pharmaceutically acceptable salt thereof, and is administered in an amount between about 0.1 μg to about 5 μg; e.g. 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.2, 2.4, 2.6, 2.8, 3.0, 3.5, 4.0, 4.5, or 5.0; or a value or range between any of the preceding values. In some embodiments, LY2090314, or a pharmaceutically acceptable salt thereof, is administered in an amount between about 0.39 μg to about 1.57 μg.

In some embodiments, the Wnt agonist is LY2090314, or a pharmaceutically acceptable salt thereof, and is administered, for example, to a cochlear cell in an amount sufficient to achieve a concentration of about 0.001 nM to 10 mM, about 0.01 nM to 1 μM, about 0.1 nM to 100 nM, about 0.001 nM to 0.0l nM, about 0.01 nM to 0.1 nM, about 0.1 nM to 1 nM, about 1 nM to 10 nM, about 10 nM to 100 nM, about 100 nM to 1 μM, or about 1 μM to 10 μM, in the perilymph fluid in the inner ear.

In some embodiments, LY2090314, or a pharmaceutically acceptable salt thereof, is administered in an amount sufficient to achieve a concentration of about 1 nM, 5 nM, 10 nM, 15 nM, 20 nM, or 40 nM, in the perilymph fluid in the inner ear.

In some embodiments, the Wnt agonist is LY2090314, or a pharmaceutically acceptable salt thereof, and is administered to a subject, for example, to the middle ear at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, LY2090314, or a pharmaceutically acceptable salt thereof, is administered to a subject, for example, to the middle ear at a concentration of about 1 μM, 5 μM, 10 μM, 15 μM, 20 μM, or 40 μM.

In some embodiments, the locally-administered Wnt agonist is GSK-3 Inhibitor XXII, or a pharmaceutically acceptable salt thereof, and is administered at a formulation concentration of about 100 μM to about 5000 μM, e.g. about 100 μM, about 250 μM, about 500 μM, about 750 μM, about 1000 μM, about 2000 μM, about 3000 μM, about 4000 μM, about 5000 μM; or a value between any of the preceding values. For example, GSK-3 Inhibitor XXII, or a pharmaceutically acceptable salt thereof, is administered at a formulation concentration of about 100 μM to about 750 μM. In some embodiments, GSK-3 Inhibitor XXII, or a pharmaceutically acceptable salt thereof, is administered intratympanically, for example at a concentration of about 500 μM, and a volume of about 50-200 μL.

In some embodiments, the locally-administered Wnt agonist is GSK-3 Inhibitor XXII, or a pharmaceutically acceptable salt thereof, and is administered in an amount between about 1 μg to about 100 μg. e.g. 1, 5, 10, 15, 20, 25, 30, 35, 40, 45, 50, 55, 60, 65, 70, 75, 80, 85, 90, 95 or 100; or a value or range between any of the preceding values. In some embodiments, GSK-3 Inhibitor XXII, or a pharmaceutically acceptable salt thereof, is administered in an amount between about 15 μg to about 85 μg. In some embodiments, GSK-3 Inhibitor XXII, or a pharmaceutically acceptable salt thereof, is administered in an amount between about 19.6 μg to about 78.5 μg.

In some embodiments, the Wnt agonist is GSK-3 Inhibitor XXII, or a pharmaceutically acceptable salt thereof, and is administered, for example, to a cochlear cell in an amount sufficient to achieve a concentration of about 0.1 nM to 1 mM, about 1 nM to 100 μM, about 10 nM to 10 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM, in the perilymph fluid in the inner ear.

In some embodiments, GSK-3 Inhibitor XXII is administered in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear.

In some embodiments, the Wnt agonist is GSK-3 Inhibitor XXII, or a pharmaceutically acceptable salt thereof, and is administered to a subject, for example, to the middle ear at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, GSK-3 Inhibitor XXII, or a pharmaceutically acceptable salt thereof, is administered to a subject, for example, to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the locally-administered Wnt agonist is AZD1080, or a pharmaceutically acceptable salt thereof, and is administered at a formulation concentration of about 100 μM to about 5000 μM, e.g. 100 μM, 250 μM, 500 μM, 750 μM, 1000 μM, 2000 μM, 3000 μM, 4000 μM, 5000 μM; or a value between any of the preceding values. For example, AZD1080, or a pharmaceutically acceptable salt thereof, is administered at a formulation concentration of about 100 μM to about 750 μM. In some embodiments, AZD1080, or a pharmaceutically acceptable salt thereof, is administered intratympanically, for example at a concentration of about 3000 μM, and a volume of about 50-200 μL.

In some embodiments, the locally-administered Wnt agonist is AZD1080, or a pharmaceutically acceptable salt thereof, and is administered in an amount between about 50 μg to about 600 μg: e.g. 50, 60, 70, 80, 90, 100, 110, 120, 130, 140, 150, 200, 250, 300, 350, 400, 450, 500, 550, or 600; or a value or range between any of the preceding values. In some embodiments. AZD1080, or a pharmaceutically acceptable salt thereof, is administered in an amount between about 115 μg to about 475 μg. In some embodiments, AZD1080, or a pharmaceutically acceptable salt thereof, is administered in an amount between about 118 μg to about 471 μg.

In some embodiments, the Wnt agonist is AZD1080, or a pharmaceutically acceptable salt thereof, and is administered, for example, to a cochlear cell in an amount sufficient to achieve a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM in the perilymph fluid in the inner ear.

In some embodiments, AZD1080, or a pharmaceutically acceptable salt thereof, is administered in an amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear.

In some embodiments, Wnt agonist is AZD1080, or a pharmaceutically acceptable salt thereof, and is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, AZD1080, or a pharmaceutically acceptable salt thereof, is administered to a subject, for example to the middle ear at a concentration of about 1 mM, 2 mM, 3 mM, 4 mM, 5 mM, 6 mM, 7 mM, 8 mM, 9 mM, or 10 mM.

Exemplary agents having activity as a Wnt agonists, including pharmaceutically acceptable salts thereof, are provided in Table 5 below.

TABLE 5

| | GSK-3 | | GSK-3 | | Lgr5 + | | Perilymph | | Formul. Conc. | |
|---|---|---|---|---|---|---|---|---|---|---|
| Agent | alpha | | alpha | | Assay | | Conc. | | Intraymp | |
| CHIR99021 | 4.4 | nM | 6.6 | nM | 2-6 | uM | 2-6 | uM | −8 | mM |
| AZD 1080 | 6.9 | nM | 31 | nM | 1-5 | uM | 1-5 | uM | 1-5 | mM |
| GSK-3 | 2.3 | nM | 2.0 | nM | 0.2-1 | uM | 0.2-1 | uM | 0.2-1 | mM |
| Inhibitor XXII | | | | | | | | | | |
| LY2090314 | 2.1 | nM | 0.9 | nM | 5-20 | nM | 5-20 | nM | 5-20 | uM |

In some embodiments, a Wnt agonist is locally administered at least once daily. In some embodiments, the Wnt agonist is locally administered about 1, 2, 3, or 4 times per day. In some embodiments, the Wnt agonist is administered once or twice daily. In some embodiments, the Wnt agonist is administered once daily. In other embodiments, the Wnt agonist is administered twice daily.

In some embodiments, a Wnt agonist is locally administered at least once weekly. In some embodiments, the Wnt agonist is administered about 1, 2, 3, or 4 times per week. In some embodiments, the Wnt agonist is administered once weekly. In some embodiments, the Wnt agonist is administered twice weekly. In other embodiments, the Wnt agonist is locally administered about 1, 2, 3, or 4 times per month.

### Local Administration Valproic Acid Compound

In some embodiments, a valproic acid compound is locally administered in a formulation concentration of about 5 mg/mL to about 110 mg/mL. A valproic acid compound is locally administered at a formulation concentration of about: 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, or 110 mg/mL, or a value between any of the preceding values, for example, between about 20 mg/mL and about 30 mg/mL, between about 10 mg/mL to about 40 mg/mL, between about 5 mg/mL and about 60 mg/mL, between about 88 and about 90, or the like. In some embodiments, a valproic acid compound is locally administered at a formulation concentration selected from 5 mg/mL, about 10 mg/mL, about 20 mg/mL, about 30 mg/mL, about 40 mg/mL, about 50 mg/mL, 60 mg/mL, 70 mg/mL, 80 mg/mL, 90 mg/mL, and about 100 mg/mL. In some embodiments, a valproic acid compound is locally administered at a formulation concentration of about 88.6 mg/mL. The valproic acid compound is, or includes, any valproic acid compound described herein. In some embodiments, the valproic acid compound is administered intratympanically.

In some embodiments, the locally-administered valproic acid compound forms a concentration gradient upon diffusing into the cochlea, with an average perilymph concentration of about 100 μM to about 20 mM (depending on the valproic acid compound), for example average perilymph concentrations of about 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM or 20 mM, inclusive of any range defined by the preceding values.

In other embodiments, a valproic acid compound is locally administered in an amount of about 1 mg to about less than 18 mg. A valproic acid compound is locally administered in an amount of about: 1, 2, 3, 4, 5, 6, 7, 8, 9, 10, 11, 12, 13, 14, 15, 16, 17 or about 18 mg, or a value between any of the preceding values, for example, between about 1 mg and about 12 mg, between about 4 mg and about 10 mg, between about 8 mg and about 13 mg, or the like. In some embodiments, a valproic acid compound is locally administered in an amount of about 17.7 mg.

In some embodiments, a valproic acid compound is administered locally, for example, to a cochlear cell in amount sufficient to achieve a concentration of about 10 μM to 4 mM in the perilymph fluid in the inner ear.

In some embodiments, a valproic acid compound is administered to a subject, for example, to the middle ear at a concentration about 100 mM to 4,000 mM.

In some embodiments, locally administering a valproic acid compound in addition to systemically administering a valproic acid compound, as described in Example 4, may provide a valproic acid blood plasma concentration in a mammal of greater than about 90 μg/mL at about 3 h post administration.

### Systemic Administration of Wnt Agonist

In some embodiments, a Wnt agonist is administered systemically. The Wnt agonist is, or includes, any Wnt agonist described herein. For example the Wnt agonist may include CHIR99021. For example the Wnt agonist may include CHIR99021. For example, the Wnt agonist may include GSK-3 Inhibitor XXII. For example, the Wnt agonist may include LY2090314. For example the Wnt agonist may include AZD1080. For example, the Wnt agonist may include a substituted 3-imidazo[1,2-a]pyridin-3-yl-4-(1,2,3,4-tetrahydro-[1,4]diazepino-[6,7,1-hi]indol-7-yl)pyrrole-2,5-dione.

### Dosing Regimen

### Systemic Valproic Acid Compound and Local Wnt Agonist

In some embodiments, the valproic acid compound is administered once or twice daily. In some embodiments, the valproic acid compound is administered once daily. In some embodiments, the valproic acid compound is administered twice daily.

In some embodiments, the Wnt agonist is administered at least once daily. In other embodiments, the Wnt agonist is administered once daily. In some embodiments, the Wnt agonist is administered at least twice daily. In other embodiments, the Wnt agonist is administered twice daily. In other embodiments, the Wnt agonist is administered once per week. In other embodiments, the Wnt agonist is administered twice per week.

In some embodiments, the valproic acid compound and the Wnt agonist is administered simultaneously. In some embodiments, the valproic acid compound and the Wnt agonist is administered at about the same time. For example, the valproic acid compound and the Wnt agonist is administered sequentially, e.g. within minutes or about an hour of each other.

In some embodiments, the valproic acid compound is administered one or more times prior to the local administration of the Wnt agonist. For example, the valproic acid compound is administered once per day for a period of 1-14 days prior to the administration of the Wnt agonist.

In many embodiments, the valproic acid compound may include sodium valproate administered once daily in an amount of about 0.5 to about 25000 mg, and the Wnt agonist may include CHIR99021 administered at about 125 μg to about 650 μg per day. In some embodiments, CHIR99021 is administered once daily. In some embodiments, CHIR99021 is administered twice daily.

In many embodiments, the valproic acid compound may include sodium valproate administered once daily in an amount of about 0.5 to about 25000 mg, and the Wnt agonist may include LY 2090314 administered at about 0.1 μg to about 2.5 μg per day. In some embodiments LY 2090314, is administered once daily. In some embodiments LY 2090314, is administered twice daily.

In many embodiments, the valproic acid compound may include sodium valproate administered once daily in an amount of about 0.5 to about 25000 mg, and the Wnt agonist may include GSK-3 Inhibitor XXII administered at about 15 μg to about 85 μg per day. In some embodiments, GSK-3 Inhibitor XXII is administered once daily. In some embodiments, GSK-3 Inhibitor XXII is administered twice daily.

In many embodiments, the valproic acid compound may include sodium valproate administered once daily in an amount of about 0.5 to about 25000 mg, and the Wnt agonist may include AZD 1080 administered at about 115 μg to about 475 μg per day. In some embodiments, AZD 1080 is administered once daily. In some embodiments, AZD 1080 is administered twice daily.

In many embodiments, the valproic acid compound may include sodium valproate administered once daily in an amount of about 0.5 to about 25000 mg, and the Wnt agonist may include a substituted 3-imidazo[1,2-a]pyridin-3-yl-4-(1,2,3,4-tetrahydro-[1,4]diazepino-[6,7,1-hi]indol-7-yl)pyrrole-2,5-dione administered at about 1 μg to about 1000 μg per day. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered once daily. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered twice daily.

### Systemic Valproic Acid Compound, Local Wnt Agonist, and Local Valproic Acid Compound

In some embodiments, the systemically administered valproic acid compound is administered once or twice daily. In some embodiments, the systemically administered valproic acid compound is administered once daily. In some embodiments, the systemically administered valproic acid compound is administered twice daily.

In some embodiments, the Wnt agonist is administered at least once daily. In other embodiments, the Wnt agonist is administered once daily. In some embodiments, the Wnt agonist is administered at least twice daily. In other embodiments, the Wnt agonist is administered twice daily. In other embodiments, the Wnt agonist is administered once per week. In other embodiments, the Wnt agonist is administered twice per week.

In some embodiments, the systemically administered valproic acid compound and the Wnt agonist is administered simultaneously. In some embodiments, the systemically administered valproic acid compound and the Wnt agonist is administered at about the same time. For example, the systemically administered valproic acid compound and the Wnt agonist is administered sequentially, e.g. within minutes or about an hour of each other.

In some embodiments, the systemically administered valproic acid compound is administered one or more times prior to the local administration of the Wnt agonist. For example, the systemically administered valproic acid compound is administered once per day for a period of 1-14 days prior to the administration of the Wnt agonist.

In some embodiments, the locally administered valproic acid compound is administered at least once daily. In other embodiments, the locally administered valproic acid compound is administered once daily. In some embodiments, the locally administered valproic acid compound is administered at least twice daily. In other embodiments, the locally administered valproic acid compound is administered twice daily. In other embodiments, the locally administered valproic acid compound is administered once per week. In other embodiments, the locally administered valproic acid compound is administered twice per week.

In some embodiments, the locally administered valproic acid compound is administered one or more times prior to the local administration of the Wnt agonist. For example, the locally administered valproic acid compound is administered once per day for a period of 1-14 days prior to the administration of the Wnt agonist.

In many embodiments, the systemically administered valproic acid compound may include sodium valproate administered once daily in an amount of about 0.5 to about 25000 mg, the Wnt agonist may include CHIR99021 administered at about 125 μg to about 650 μg per day, and the locally administered valproic acid may include sodium valproate administered at about 1 mg to about 12 mg per day. In some embodiments, CHIR99021 is administered once daily. In some embodiments, CHIR99021 is administered twice daily.

In many embodiments, the systemically administered valproic acid compound may include sodium valproate administered once daily in an amount of about 0.5 to about 25000 mg, the Wnt agonist may include LY 2090314 administered at about 0.1 μg to about 2.5 μg per day, and the locally administered valproic acid may include sodium valproate administered at about 1 mg to about 12 mg per day. In some embodiments LY 2090314, is administered once daily. In some embodiments LY 2090314, is administered twice daily.

In many embodiments, the systemically administered valproic acid compound may include sodium valproate administered once daily in an amount of about 0.5 to about 25000 mg, the Wnt agonist may include GSK-3 Inhibitor XXII administered at about 15 μg to about 85 μg per day, and the locally administered valproic acid may include sodium valproate administered at about 1 mg to about 12 mg per day. In some embodiments, GSK-3 Inhibitor XXII is administered once daily. In some embodiments, GSK-3 Inhibitor XXII is administered twice daily.

In many embodiments, the systemically administered valproic acid compound may include sodium valproate administered once daily in an amount of about 0.5 to about 25000 mg, the Wnt agonist may include AZD 1080 administered at about 115 μg to about 475 μg per day, and the locally administered valproic acid may include sodium valproate administered at about 1 mg to about 12 mg per day. In some embodiments, AZD 1080 is administered once daily. In some embodiments, AZD 1080 is administered twice daily.

In many embodiments, the systemically administered valproic acid compound may include sodium valproate administered once daily in an amount of about 0.5 to about 25000 mg, the Wnt agonist may include a substituted 3-imidazo[1,2-a]pyridin-3-yl-4-(1,2,3,4-tetrahydro-[1,4]diazepino-[6,7,1-hi]indol-7-yl)pyrrole-2,5-dione administered at about 1 μg to about 1000 μg per day, and the locally administered valproic acid may include sodium valproate administered at about 1 mg to about 12 mg per day. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered once daily. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered twice daily.

In some embodiments, the systemic valproic acid compound and Wnt agonist is administered simultaneously. In other embodiments, the systemic valproic acid compound and Wnt agonist is administered sequentially.

In some embodiments, the local valproic acid compound and the Wnt agonist is administered simultaneously. In some embodiments, the local valproic acid compound and a Wnt agonist are co-formulated in a fixed-dose combination, and thus are administered simultaneously. In other embodiments, the local valproic acid compound and the Wnt agonist is administered sequentially in separate formulations, e.g. within minutes or about an hour of each other.

### Systemic Valproic Acid Compound and Systemic Wnt Agonist

In many embodiments, the valproic acid compound and the Wnt agonist is administered simultaneously. In some embodiments, the valproic acid compound and a Wnt agonist are co-formulated in a fixed-dose combination, and thus are administered simultaneously. In some embodiments, the valproic acid compound and the Wnt agonist is administered sequentially in separate formulations, e.g. within minutes or about an hour of each other.

In many embodiments, the valproic acid compound may include sodium valproate administered once daily in an amount of about 0.5 to about 25000 mg, and the Wnt agonist may include CHIR99021. In some embodiments, CHIR99021 is administered once daily. In some embodiments, CHIR99021 is administered twice daily.

In many embodiments, the valproic acid compound may include sodium valproate administered once daily in an amount of about 0.5 to about 25000 mg, and the Wnt agonist may include LY 2090314. In some embodiments LY 2090314, is administered once daily. In some embodiments LY 2090314, is administered twice daily.

In many embodiments, the valproic acid compound may include sodium valproate administered once daily in an amount of about 0.5 to about 25000 mg, and the Wnt agonist may include GSK-3 Inhibitor XXII. In some embodiments, GSK-3 Inhibitor XXII is administered once daily. In some embodiments, GSK-3 Inhibitor XXII is administered twice daily.

In many embodiments, the valproic acid compound may include sodium valproate administered once daily in an amount of about 0.5 to about 25000 mg, and the Wnt agonist may include AZD 1080. In some embodiments, AZD 1080 is administered once daily. In some embodiments, AZD 1080 is administered twice daily.

In many embodiments, the valproic acid compound may include sodium valproate administered once daily in an amount of about 0.5 to about 25000 mg, and the Wnt agonist may include a substituted 3-imidazo[1,2-a]pyridin-3-yl-4-(1,2,3,4-tetrahydro-[1,4]diazepino-[6,7,1-hi]indol-7-yl)pyrrole-2,5-dione. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered once daily. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered twice daily.

### Combinations

In many embodiments, any valproic acid compound described herein (for systemic administration) is combined with any Wnt agonist described herein (for local administration).

In many embodiments, any valproic acid compound described herein (for systemic administration) is combined with any Wnt agonist described herein (for local administration), and may further be combined with any valproic acid compound described herein (for local administration).

In many embodiments, any valproic acid compound described herein (for local administration) is combined with any Wnt agonist described herein (for local administration).

In many embodiments, any valproic acid compound described herein (for systemic administration) is combined with any Wnt agonist described herein (for systemic administration).

The following combinations may relate to any of the methods described herein. The following combinations may relate to any dose or dosing regimen described herein. The following combinations may relate to any kit described herein.

### Combinations: Systemic IPA Compound and Local Wnt Agonist

In some embodiments, the systemic valproic acid compound may include sodium valproate and the local Wnt agonist is bikinin, or a pharmaceutically acceptable salt thereof. In some embodiments, the systemic valproic acid compound may further include valproic acid.

In some embodiments, the systemic valproic acid compound may include sodium valproate and the local Wnt agonist is hymenialdisine, or a pharmaceutically acceptable salt thereof. In some embodiments, the systemic valproic acid compound may further include valproic acid.

In some embodiments, the systemic valproic acid compound may include sodium valproate and the local Wnt agonist is aloisine A, or a pharmaceutically acceptable salt thereof. In some embodiments, the systemic valproic acid compound may further include valproic acid.

In some embodiments, the systemic valproic acid compound may include sodium valproate and the local Wnt agonist is aloisine B, or a pharmaceutically acceptable salt thereof. In some embodiments, the systemic valproic acid compound may further include valproic acid.

In some embodiments, the systemic valproic acid compound may include sodium valproate and the local Wnt agonist is TWS119, or a pharmaceutically acceptable salt thereof. In some embodiments, the systemic valproic acid compound may further include valproic acid.

In some embodiments, the systemic valproic acid compound may include sodium valproate and the local Wnt agonist is CT20026, or a pharmaceutically acceptable salt thereof. In some embodiments, the systemic valproic acid compound may further include valproic acid.

In some embodiments, the systemic valproic acid compound may include sodium valproate and the local Wnt agonist is CHIR98014, or a pharmaceutically acceptable salt thereof. In some embodiments, the systemic valproic acid compound may further include valproic acid.

In some embodiments, the systemic valproic acid compound may include sodium valproate and the local Wnt agonist is CHIR98023, or a pharmaceutically acceptable salt thereof. In some embodiments, the systemic valproic acid compound may further include valproic acid.

In some embodiments, the systemic valproic acid compound may include sodium valproate and the local Wnt agonist is CHIR98024, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3β Inhibitor XVIII, or a pharmaceutically acceptable salt thereof. In some embodiments, the systemic valproic acid compound may further include valproic acid.

In some embodiments, the systemic valproic acid compound may include sodium valproate and the local Wnt agonist is CGP60474, or a pharmaceutically acceptable salt thereof. In some embodiments, the systemic valproic acid compound may further include valproic acid.

In some embodiments, the systemic valproic acid compound may include sodium valproate and the local Wnt agonist is AZD2858, or a pharmaceutically acceptable salt thereof. In some embodiments, the systemic valproic acid compound may further include valproic acid.

In some embodiments, the systemic valproic acid compound may include sodium valproate and the local Wnt agonist is CID 755673, or a pharmaceutically acceptable salt thereof.

In some embodiments, the systemic valproic acid compound may include sodium valproate and the local Wnt agonist is TCS 2002, or a pharmaceutically acceptable salt thereof. In some embodiments, the systemic valproic acid compound may further include valproic acid.

In some embodiments, the systemic valproic acid compound may include sodium valproate and the local Wnt agonist is dibromocantharelline, or a pharmaceutically acceptable salt thereof. In some embodiments, the systemic valproic acid compound may further include valproic acid.

In some embodiments, the systemic valproic acid compound may include sodium valproate and the local Wnt agonist is ML320, or a pharmaceutically acceptable salt thereof. In some embodiments, the systemic valproic acid compound may further include valproic acid.

In some embodiments, the systemic valproic acid compound may include sodium valproate and the local Wnt agonist is flavopiridol, or a pharmaceutically acceptable salt thereof. In some embodiments, the systemic valproic acid compound may further include valproic acid.

In some embodiments, the systemic valproic acid compound may include sodium valproate and the local Wnt agonist is compound 100, or a pharmaceutically acceptable salt thereof. In some embodiments, the systemic valproic acid compound may further include valproic acid.

In some embodiments, the systemic valproic acid compound may include sodium valproate and the local Wnt agonist is hymenidin, or a pharmaceutically acceptable salt thereof. In some embodiments, the systemic valproic acid compound may further include valproic acid.

In some embodiments, the systemic valproic acid compound may include sodium valproate and the local Wnt agonist is 6-bromoindirubin-3′-acetoxime, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor IX, or a pharmaceutically acceptable salt thereof. In some embodiments, the systemic valproic acid compound may further include valproic acid.

In some embodiments, the systemic valproic acid compound may include sodium valproate and the local Wnt agonist is indirubin-3′-monoxime, or a pharmaceutically acceptable salt thereof. In some embodiments, the systemic valproic acid compound may further include valproic acid.

In some embodiments, the systemic valproic acid compound may include sodium valproate and the local Wnt agonist is 5-iodo-indirubin-3′-monoxime, or a pharmaceutically acceptable salt thereof. In some embodiments, the systemic valproic acid compound may further include valproic acid.

In some embodiments, the systemic valproic acid compound may include sodium valproate and the local Wnt agonist is indirubin-5-sulfonic acid, or a pharmaceutically acceptable salt thereof. In some embodiments, the systemic valproic acid compound may further include valproic acid.

In some embodiments, the systemic valproic acid compound may include sodium valproate and the local Wnt agonist is indirubin, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor X, or a pharmaceutically acceptable salt thereof. In some embodiments, the systemic valproic acid compound may further include valproic acid.

In some embodiments, the systemic valproic acid compound may include sodium valproate and the local Wnt agonist is lithium chloride. In some embodiments, the systemic valproic acid compound may further include valproic acid.

In some embodiments, the systemic valproic acid compound may include sodium valproate and the local Wnt agonist is a beryllium salt, e.g. BeSO4. In some embodiments, the systemic valproic acid compound may further include valproic acid.

In some embodiments, the systemic valproic acid compound may include sodium valproate and the local Wnt agonist is a zinc salt. In some embodiments, the systemic valproic acid compound may further include valproic acid.

In some embodiments, the systemic valproic acid compound may include sodium valproate and the local Wnt agonist is a tungstate salt. In some embodiments, the systemic valproic acid compound may further include valproic acid.

In some embodiments, the systemic valproic acid compound may include sodium valproate and the local Wnt agonist is a mercury salt. In some embodiments, the systemic valproic acid compound may further include valproic acid.

In some embodiments, the systemic valproic acid compound may include sodium valproate and the local Wnt agonist is a copper salt. In some embodiments, the systemic valproic acid compound may further include valproic acid.

In some embodiments, the systemic valproic acid compound may include sodium valproate and the local Wnt agonist is compound 39, or a pharmaceutically acceptable salt thereof. In some embodiments, the systemic valproic acid compound may further include valproic acid.

In some embodiments, the systemic valproic acid compound may include sodium valproate and the local Wnt agonist is compound 29 (CAS 1772823-37-6), or a pharmaceutically acceptable salt thereof. In some embodiments, the systemic valproic acid compound may further include valproic acid.

In some embodiments, the systemic valproic acid compound may include sodium valproate and the local Wnt agonist is compound 33, or a pharmaceutically acceptable salt thereof. In some embodiments, the systemic valproic acid compound may further include valproic acid.

In some embodiments, the systemic valproic acid compound may include sodium valproate and the local Wnt agonist is compound 29′ (CAS 436866-61-4), or a pharmaceutically acceptable salt thereof. In some embodiments, the systemic valproic acid compound may further include valproic acid.

In some embodiments, the systemic valproic acid compound may include sodium valproate and the local Wnt agonist is compound 46, or a pharmaceutically acceptable salt thereof. In some embodiments, the systemic valproic acid compound may further include valproic acid.

In some embodiments, the systemic valproic acid compound may include sodium valproate and the local Wnt agonist is compound 5a, or a pharmaceutically acceptable salt thereof. In some embodiments, the systemic valproic acid compound may further include valproic acid.

In some embodiments, the systemic valproic acid compound may include sodium valproate and the local Wnt agonist is GF109203x, or a pharmaceutically acceptable salt thereof. In some embodiments, the systemic valproic acid compound may further include valproic acid.

In some embodiments, the systemic valproic acid compound may include sodium valproate and the local Wnt agonist is RO31-8220, or a pharmaceutically acceptable salt thereof. In some embodiments, the systemic valproic acid compound may further include valproic acid.

In some embodiments, the systemic valproic acid compound may include sodium valproate and the local Wnt agonist is bisindolyl maleimide X, or a pharmaceutically acceptable salt thereof. In some embodiments, the systemic valproic acid compound may further include valproic acid.

In some embodiments, the systemic valproic acid compound may include sodium valproate and the local Wnt agonist is enzastaurin (LY317615), or a pharmaceutically acceptable salt thereof. In some embodiments, the systemic valproic acid compound may further include valproic acid.

In some embodiments, the systemic valproic acid compound may include sodium valproate and the local Wnt agonist is I5, or a pharmaceutically acceptable salt thereof. In some embodiments, the systemic valproic acid compound may further include valproic acid.

In some embodiments, the systemic valproic acid compound may include sodium valproate and the local Wnt agonist is SB-216763, or a pharmaceutically acceptable salt thereof. In some embodiments, the systemic valproic acid compound may further include valproic acid.

In some embodiments, the systemic valproic acid compound may include sodium valproate and the local Wnt agonist is SB-415286, or a pharmaceutically acceptable salt thereof. In some embodiments, the systemic valproic acid compound may further include valproic acid.

In some embodiments, the systemic valproic acid compound may include sodium valproate and the local Wnt agonist is 3F8, or a pharmaceutically acceptable salt thereof. In some embodiments, the systemic valproic acid compound may further include valproic acid.

In some embodiments, the systemic valproic acid compound may include sodium valproate and the local Wnt agonist is TCS 21311, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor 1, or a pharmaceutically acceptable salt thereof. In some embodiments, the systemic valproic acid compound may further include valproic acid.

In some embodiments, the systemic valproic acid compound may include sodium valproate and the local Wnt agonist is LY2090314, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor-1, or a pharmaceutically acceptable salt thereof. In some embodiments, the systemic valproic acid compound may further include valproic acid.

In some embodiments, the systemic valproic acid compound may include sodium valproate and the local Wnt agonist is IM-12, or a pharmaceutically acceptable salt thereof. In some embodiments, the systemic valproic acid compound may further include valproic acid.

In some embodiments, the systemic valproic acid compound may include sodium valproate and the local Wnt agonist is compound 34, or a pharmaceutically acceptable salt thereof. In some embodiments, the systemic valproic acid compound may further include valproic acid.

In some embodiments, the systemic valproic acid compound may include sodium valproate and the local Wnt agonist is KT 5720, or a pharmaceutically acceptable salt thereof. In some embodiments, the systemic valproic acid compound may further include valproic acid.

In some embodiments, the systemic valproic acid compound may include sodium valproate and the local Wnt agonist is isogranulatimide, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3β Inhibitor XI, or a pharmaceutically acceptable salt thereof. In some embodiments, the systemic valproic acid compound may further include valproic acid.

In some embodiments, the systemic valproic acid compound may include sodium valproate and the local Wnt agonist is BIP-135, or a pharmaceutically acceptable salt thereof. In some embodiments, the systemic valproic acid compound may further include valproic acid.

In some embodiments, the systemic valproic acid compound may include sodium valproate and the local Wnt agonist is CP21R7, or a pharmaceutically acceptable salt thereof. In some embodiments, the systemic valproic acid compound may further include valproic acid.

In some embodiments, the systemic valproic acid compound may include sodium valproate and the local Wnt agonist is tivantinib, or a pharmaceutically acceptable salt thereof. In some embodiments, the systemic valproic acid compound may further include valproic acid.

In some embodiments, the systemic valproic acid compound may include sodium valproate and the local Wnt agonist is compound lambda-OS1, or a pharmaceutically acceptable salt thereof. In some embodiments, the systemic valproic acid compound may further include valproic acid.

In some embodiments, the systemic valproic acid compound may include sodium valproate and the local Wnt agonist is HB12, or a pharmaceutically acceptable salt thereof. In some embodiments, the systemic valproic acid compound may further include valproic acid.

In some embodiments, the systemic valproic acid compound may include sodium valproate and the local Wnt agonist is DW12, or a pharmaceutically acceptable salt thereof. In some embodiments, the systemic valproic acid compound may further include valproic acid.

In some embodiments, the systemic valproic acid compound may include sodium valproate and the local Wnt agonist is NP309, or a pharmaceutically acceptable salt thereof. In some embodiments, the systemic valproic acid compound may further include valproic acid.

In some embodiments, the systemic valproic acid compound may include sodium valproate and the local Wnt agonist is (RRu)-HB1229, or pharmaceutically acceptable salt thereof. In some embodiments, the systemic valproic acid compound may further include valproic acid.

In some embodiments, the systemic valproic acid compound may include sodium valproate and the local Wnt agonist is (RRu)-NP549, or pharmaceutically acceptable salt thereof. In some embodiments, the systemic valproic acid compound may further include valproic acid.

In some embodiments, the systemic valproic acid compound may include sodium valproate and the local Wnt agonist is compound 3, or a pharmaceutically acceptable salt thereof. In some embodiments, the systemic valproic acid compound may further include valproic acid.

In some embodiments, the systemic valproic acid compound may include sodium valproate and the local Wnt agonist is compound (R)-DW12, or a pharmaceutically acceptable salt thereof. In some embodiments, the systemic valproic acid compound may further include valproic acid.

In some embodiments, the systemic valproic acid compound may include sodium valproate and the local Wnt agonist is staurosporine, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3β Inhibitor XXVI, or a pharmaceutically acceptable salt thereof. In some embodiments, the systemic valproic acid compound may further include valproic acid.

In some embodiments, the systemic valproic acid compound may include sodium valproate and the local Wnt agonist is manzamine A, or a pharmaceutically acceptable salt thereof. In some embodiments, the systemic valproic acid compound may further include valproic acid.

In some embodiments, the systemic valproic acid compound may include sodium valproate and the local Wnt agonist is TC-G 24, or a pharmaceutically acceptable salt thereof. In some embodiments, the systemic valproic acid compound may further include valproic acid.

In some embodiments, the systemic valproic acid compound may include sodium valproate and the local Wnt agonist is compound 14d, or a pharmaceutically acceptable salt thereof. In some embodiments, the systemic valproic acid compound may further include valproic acid.

In some embodiments, the systemic valproic acid compound may include sodium valproate and the local Wnt agonist is compound 15b, or a pharmaceutically acceptable salt thereof. In some embodiments, the systemic valproic acid compound may further include valproic acid.

In some embodiments, the systemic valproic acid compound may include sodium valproate and the local Wnt agonist is compound 20x, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor 2, or a pharmaceutically acceptable salt thereof. In some embodiments, the systemic valproic acid compound may further include valproic acid.

In some embodiments, the systemic valproic acid compound may include sodium valproate and the local Wnt agonist is SU9516, or a pharmaceutically acceptable salt thereof. In some embodiments, the systemic valproic acid compound may further include valproic acid.

In some embodiments, the systemic valproic acid compound may include sodium valproate and the local Wnt agonist is AZD1080, or a pharmaceutically acceptable salt thereof. In some embodiments, the systemic valproic acid compound may further include valproic acid.

In some embodiments, the systemic valproic acid compound may include sodium valproate and the local Wnt agonist is kenpaullone, or a pharmaceutically acceptable salt thereof. In some embodiments, the systemic valproic acid compound may further include valproic acid.

In some embodiments, the systemic valproic acid compound may include sodium valproate and the local Wnt agonist is compound 17b, or a pharmaceutically acceptable salt thereof. In some embodiments, the systemic valproic acid compound may further include valproic acid.

In some embodiments, the systemic valproic acid compound may include sodium valproate and the local Wnt agonist is azakenpaullone, or a pharmaceutically acceptable salt thereof. In some embodiments, the systemic valproic acid compound may further include valproic acid.

In some embodiments, the systemic valproic acid compound may include sodium valproate and the local Wnt agonist is alsterpaullone, or a pharmaceutically acceptable salt thereof. In some embodiments, the systemic valproic acid compound may further include valproic acid.

In some embodiments, the systemic valproic acid compound may include sodium valproate and the local Wnt agonist is alsterpaullone 2-cyanoethyl, or a pharmaceutically acceptable salt thereof. In some embodiments, the systemic valproic acid compound may further include valproic acid.

In some embodiments, the systemic valproic acid compound may include sodium valproate and the local Wnt agonist is cazpaullone, or a pharmaceutically acceptable salt thereof. In some embodiments, the systemic valproic acid compound may further include valproic acid.

In some embodiments, the systemic valproic acid compound may include sodium valproate and the local Wnt agonist is FRATtide (SEQ ID NO: 1), or a pharmaceutically acceptable salt thereof. In some embodiments, the systemic valproic acid compound may further include valproic acid.

In some embodiments, the systemic valproic acid compound may include sodium valproate and the local Wnt agonist is L803 (SEQ ID NO: 2), or a pharmaceutically acceptable salt thereof. In some embodiments, the systemic valproic acid compound may further include valproic acid.

In some embodiments, the systemic valproic acid compound may include sodium valproate and the local Wnt agonist is L803-mts (SEQ ID NO: 3), or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor XXII, or a pharmaceutically acceptable salt thereof. In some embodiments, the systemic valproic acid compound may further include valproic acid.

In some embodiments, the systemic valproic acid compound may include sodium valproate and the local Wnt agonist is compound 4a, or a pharmaceutically acceptable salt thereof. In some embodiments, the systemic valproic acid compound may further include valproic acid.

In some embodiments, the systemic valproic acid compound may include sodium valproate and the local Wnt agonist is compound 4t, or a pharmaceutically acceptable salt thereof. In some embodiments, the systemic valproic acid compound may further include valproic acid.

In some embodiments, the systemic valproic acid compound may include sodium valproate and the local Wnt agonist is compound 4z, or a pharmaceutically acceptable salt thereof. In some embodiments, the systemic valproic acid compound may further include valproic acid.

In some embodiments, the systemic valproic acid compound may include sodium valproate and the local Wnt agonist is AT 7519, or a pharmaceutically acceptable salt thereof. In some embodiments, the systemic valproic acid compound may further include valproic acid.

In some embodiments, the systemic valproic acid compound may include sodium valproate and the local Wnt agonist is pyrazolopyridine 9, or a pharmaceutically acceptable salt thereof. In some embodiments, the systemic valproic acid compound may further include valproic acid.

In some embodiments, the systemic valproic acid compound may include sodium valproate and the local Wnt agonist is pyrazolopyridine 18, or a pharmaceutically acceptable salt thereof. In some embodiments, the systemic valproic acid compound may further include valproic acid.

In some embodiments, the systemic valproic acid compound may include sodium valproate and the local Wnt agonist is pyrazolopyridine 34, or a pharmaceutically acceptable salt thereof. In some embodiments, the systemic valproic acid compound may further include valproic acid.

In some embodiments, the systemic valproic acid compound may include sodium valproate and the local Wnt agonist is compound 14, or a pharmaceutically acceptable salt thereof. In some embodiments, the systemic valproic acid compound may further include valproic acid.

In some embodiments, the systemic valproic acid compound may include sodium valproate and the local Wnt agonist is compound 23, or a pharmaceutically acceptable salt thereof. In some embodiments, the systemic valproic acid compound may further include valproic acid.

In some embodiments, the systemic valproic acid compound may include sodium valproate and the local Wnt agonist is compound 19, or a pharmaceutically acceptable salt thereof. In some embodiments, the systemic valproic acid compound may further include valproic acid.

In some embodiments, the systemic valproic acid compound may include sodium valproate and the local Wnt agonist is NSC 693868, or a pharmaceutically acceptable salt thereof. In some embodiments, the systemic valproic acid compound may further include valproic acid.

In some embodiments, the systemic valproic acid compound may include sodium valproate and the local Wnt agonist is compound 150, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3 Inhibitor XIII, or a pharmaceutically acceptable salt thereof. In some embodiments, the systemic valproic acid compound may further include valproic acid.

In some embodiments, the systemic valproic acid compound may include sodium valproate and the local Wnt agonist is VP0.7, or a pharmaceutically acceptable salt thereof. In some embodiments, the systemic valproic acid compound may further include valproic acid.

In some embodiments, the systemic valproic acid compound may include sodium valproate and the local Wnt agonist is a compound having a CAS number 1132813-46-7, or a pharmaceutically acceptable salt thereof. In some embodiments, the systemic valproic acid compound may further include valproic acid.

In some embodiments, the systemic valproic acid compound may include sodium valproate and the local Wnt agonist is a compound having a CAS number 1132812-98-6, or a pharmaceutically acceptable salt thereof. In some embodiments, the systemic valproic acid compound may further include valproic acid.

In some embodiments, the systemic valproic acid compound may include sodium valproate and the local Wnt agonist is a compound having a CAS number 950727-66-9, or a pharmaceutically acceptable salt thereof. In some embodiments, the systemic valproic acid compound may further include valproic acid.

In some embodiments, the systemic valproic acid compound may include sodium valproate and the local Wnt agonist is GSK-30 Inhibitor VII, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3β Inhibitor VI, or a pharmaceutically acceptable salt thereof. In some embodiments, the systemic valproic acid compound may further include valproic acid.

In some embodiments, the systemic valproic acid compound may include sodium valproate and the local Wnt agonist is palinurin, or a pharmaceutically acceptable salt thereof. In some embodiments, the systemic valproic acid compound may further include valproic acid.

In some embodiments, the systemic valproic acid compound may include sodium valproate and the local Wnt agonist is tricantin, or a pharmaceutically acceptable salt thereof. In some embodiments, the systemic valproic acid compound may further include valproic acid.

In some embodiments, the systemic valproic acid compound may include sodium valproate and the local Wnt agonist is GSK-3β Inhibitor I, or a pharmaceutically acceptable salt thereof. In some embodiments, the systemic valproic acid compound may further include valproic acid.

In some embodiments, the systemic valproic acid compound may include sodium valproate and the local Wnt agonist is NP031115, or a pharmaceutically acceptable salt thereof. In some embodiments, the systemic valproic acid compound may further include valproic acid.

In some embodiments, the systemic valproic acid compound may include sodium valproate and the local Wnt agonist is NP031112, or a pharmaceutically acceptable salt thereof. In some embodiments, the systemic valproic acid compound may further include valproic acid.

In some embodiments, the systemic valproic acid compound may include sodium valproate and the local Wnt agonist is compound 90, or a pharmaceutically acceptable salt thereof. In some embodiments, the systemic valproic acid compound may further include valproic acid.

In some embodiments, the systemic valproic acid compound may include sodium valproate and the local Wnt agonist is compound 92, or a pharmaceutically acceptable salt thereof. In some embodiments, the systemic valproic acid compound may further include valproic acid.

In some embodiments, the systemic valproic acid compound may include sodium valproate and the local Wnt agonist is GSK-30 Inhibitor VIII, or a pharmaceutically acceptable salt thereof. In some embodiments, the systemic valproic acid compound may further include valproic acid.

In some embodiments, the systemic valproic acid compound may include sodium valproate and the local Wnt agonist is A-1070722, or a pharmaceutically acceptable salt thereof. In some embodiments, the systemic valproic acid compound may further include valproic acid.

In some embodiments, the systemic valproic acid compound may include sodium valproate and the local Wnt agonist is compound 27, or a pharmaceutically acceptable salt thereof. In some embodiments, the systemic valproic acid compound may further include valproic acid.

In some embodiments, the systemic valproic acid compound may include sodium valproate and the local Wnt agonist is compound 12, or a pharmaceutically acceptable salt thereof. In some embodiments, the systemic valproic acid compound may further include valproic acid.

In some embodiments, the systemic valproic acid compound may include sodium valproate and the local Wnt agonist is NP-103, or a pharmaceutically acceptable salt thereof. In some embodiments, the systemic valproic acid compound may further include valproic acid.

In some embodiments, the systemic valproic acid compound may include sodium valproate and the local Wnt agonist is CG-301338, or a pharmaceutically acceptable salt thereof. In some embodiments, the systemic valproic acid compound may further include valproic acid.

In some embodiments, the systemic valproic acid compound may include sodium valproate and the local Wnt agonist is XD-4241, or a pharmaceutically acceptable salt thereof. In some embodiments, the systemic valproic acid compound may further include valproic acid.

In some embodiments, the systemic valproic acid compound may include sodium valproate and the local Wnt agonist is CEP-16805, or a pharmaceutically acceptable salt thereof. In some embodiments, the systemic valproic acid compound may further include valproic acid.

In some embodiments, the systemic valproic acid compound may include sodium valproate and the local Wnt agonist is AZD13282107, or a pharmaceutically acceptable salt thereof. In some embodiments, the systemic valproic acid compound may further include valproic acid.

In some embodiments, the systemic valproic acid compound may include sodium valproate and the local Wnt agonist is SAR 502250, or a pharmaceutically acceptable salt thereof. In some embodiments, the systemic valproic acid compound may further include valproic acid.

In some embodiments, the systemic valproic acid compound may include sodium valproate and the local Wnt agonist is AR79, or a pharmaceutically acceptable salt thereof. In some embodiments, the systemic valproic acid compound may further include valproic acid.

In some embodiments, the systemic valproic acid compound may include sodium valproate and the local Wnt agonist is GI179186X, or a pharmaceutically acceptable salt thereof. In some embodiments, the systemic valproic acid compound may further include valproic acid.

In some embodiments, the systemic valproic acid compound may include sodium valproate and the local Wnt agonist is CT118637, or a pharmaceutically acceptable salt thereof. In some embodiments, the systemic valproic acid compound may further include valproic acid.

In some embodiments, the systemic valproic acid compound may include sodium valproate and the local Wnt agonist is CP-70949, or a pharmaceutically acceptable salt thereof. In some embodiments, the systemic valproic acid compound may further include valproic acid.

In some embodiments, the systemic valproic acid compound may include sodium valproate and the local Wnt agonist is GW784752X, or a pharmaceutically acceptable salt thereof. In some embodiments, the systemic valproic acid compound may further include valproic acid.

In some embodiments, the systemic valproic acid compound may include sodium valproate and the local Wnt agonist is GW784775X, or a pharmaceutically acceptable salt thereof. In some embodiments, the systemic valproic acid compound may further include valproic acid.

In some embodiments, the systemic valproic acid compound may include sodium valproate and the local Wnt agonist is CT73911, or a pharmaceutically acceptable salt thereof. In some embodiments, the systemic valproic acid compound may further include valproic acid.

In some embodiments, the systemic valproic acid compound may include sodium valproate and the local Wnt agonist is LY2064827, or a pharmaceutically acceptable salt thereof. In some embodiments, the systemic valproic acid compound may further include valproic acid.

In some embodiments, the systemic valproic acid compound may include sodium valproate and the local Wnt agonist is 705701, or a pharmaceutically acceptable salt thereof. In some embodiments, the systemic valproic acid compound may further include valproic acid.

In some embodiments, the systemic valproic acid compound may include sodium valproate and the local Wnt agonist is 708244, or a pharmaceutically acceptable salt thereof. In some embodiments, the systemic valproic acid compound may further include valproic acid.

In some embodiments, the systemic valproic acid compound may include sodium valproate and the local Wnt agonist is 709125, or a pharmaceutically acceptable salt thereof. In some embodiments, the systemic valproic acid compound may further include valproic acid.

In some embodiments, the systemic valproic acid compound may include sodium valproate and the local Wnt agonist is (R)-BRD4003, or a pharmaceutically acceptable salt thereof. In some embodiments, the systemic valproic acid compound may further include valproic acid.

In some embodiments, the systemic valproic acid compound may include sodium valproate and the local Wnt agonist is (S)-BRD4003, or a pharmaceutically acceptable salt thereof. In some embodiments, the systemic valproic acid compound may further include valproic acid.

In some embodiments, the systemic valproic acid compound may include sodium valproate and the local Wnt agonist is compound 8, or a pharmaceutically acceptable salt thereof. In some embodiments, the systemic valproic acid compound may further include valproic acid.

In some embodiments, the systemic valproic acid compound may include sodium valproate and the local Wnt agonist is compound 9, or a pharmaceutically acceptable salt thereof. In some embodiments, the systemic valproic acid compound may further include valproic acid.

In some embodiments, the systemic valproic acid compound may include sodium valproate and the local Wnt agonist is compound 11, or a pharmaceutically acceptable salt thereof. In some embodiments, the systemic valproic acid compound may further include valproic acid.

In some embodiments, the systemic valproic acid compound may include sodium valproate and the local Wnt agonist is BRD1172, or a pharmaceutically acceptable salt thereof. In some embodiments, the systemic valproic acid compound may further include valproic acid.

In some embodiments, the systemic valproic acid compound may include sodium valproate and the local Wnt agonist is compound 16, or a pharmaceutically acceptable salt thereof. In some embodiments, the systemic valproic acid compound may further include valproic acid.

In some embodiments, the systemic valproic acid compound may include sodium valproate and the local Wnt agonist is BRD1652, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-1, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-2, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-3, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-4, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-5, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-6, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-7, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-8, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-9, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-10, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-11, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-12, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-13, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-14, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-15, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-16, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-17, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-18, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-19, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-20, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-21, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-22, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-23, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-24, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-25, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-26, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-27, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-28, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-29, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-30, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-31, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-32, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-33, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-34, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-35, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-36, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-37, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-38, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-39, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-40, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-41, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-42, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-43, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-44, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-45, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-46, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-47, or a pharmaceutically acceptable salt thereof. In some embodiments, the systemic valproic acid compound may further include valproic acid.

In some embodiments, the systemic valproic acid compound may include sodium valproate and the local Wnt agonist is Compound I-48, or a pharmaceutically acceptable salt thereof. In some embodiments, the systemic valproic acid compound may further include valproic acid.

In some embodiments, the systemic valproic acid compound may include sodium valproate and the local Wnt agonist is selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, the systemic valproic acid compound may further include valproic acid.

### Combinations: Systemic VPA Compound, Local Wnt Agonist, and Local VPA Compound

In some embodiments, the systemic valproic acid compound may include sodium valproate, the local Wnt agonist is bikinin, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is hymenialdisine, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is aloisine A, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is aloisine B, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is TWS119, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CT20026, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CHIR98014, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CHIR98023, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CHIR98024, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor XVIII, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CGP60474, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is AZD2858, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CID 755673, or a pharmaceutically acceptable salt thereof.

In some embodiments, the systemic valproic acid compound may include sodium valproate, the local Wnt agonist is TCS 2002, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is dibromocantharelline, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is ML320, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is flavopiridol, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 100, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is hymenidin, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 6-bromoindirubin-3′-acetoxime, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor IX, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is indirubin-3′-monoxime, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 5-iodo-indirubin-3′-monoxime, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is indirubin-5-sulfonic acid, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is indirubin, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor X, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is lithium chloride. In some embodiments, the systemic valproic acid compound may further include valproic acid.

In some embodiments, the systemic valproic acid compound may include sodium valproate, the local Wnt agonist is a beryllium salt, BeSO4. In some embodiments, the systemic valproic acid compound may further include valproic acid.

In some embodiments, the systemic valproic acid compound may include sodium valproate, the local Wnt agonist is a zinc salt. In some embodiments, the systemic valproic acid compound may further include valproic acid.

In some embodiments, the systemic valproic acid compound may include sodium valproate, the local Wnt agonist is a tungstate salt. In some embodiments, the systemic valproic acid compound may further include valproic acid.

In some embodiments, the systemic valproic acid compound may include sodium valproate and the local Wnt agonist is a mercury salt. In some embodiments, the systemic valproic acid compound may further include valproic acid.

In some embodiments, the systemic valproic acid compound may include sodium valproate and the local Wnt agonist is a copper salt. In some embodiments, the systemic valproic acid compound may further include valproic acid.

In some embodiments, the systemic valproic acid compound may include sodium valproate, the local Wnt agonist is compound 39, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 29 (CAS 1772823-37-6), or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 33, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 29′ (CAS 436866-61-4), or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 46, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 5a, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GF109203x, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is RO31-8220, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is bisindolyl maleimide X, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is enzastaurin (LY317615), or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 15, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is SB-216763, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is SB-415286, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 3F8, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is TCS 21311, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor 1, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is LY2090314, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor-I, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is IM-12, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 34, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is KT 5720, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is isogranulatimide, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor XI, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is BIP-135, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CP21R7, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is tivantinib, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound lambda-OS1, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is HB12, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is DW12, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is NP309, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is (RRu)-HB1229, or a pharmaceutically acceptable salt thereof. In some embodiments, the systemic valproic acid compound may further include valproic acid.

In some embodiments, the systemic valproic acid compound may include sodium valproate, the local Wnt agonist is (RRu)-NP549, or a pharmaceutically acceptable salt thereof. In some embodiments, the systemic valproic acid compound may further include valproic acid.

In some embodiments, the systemic valproic acid compound may include sodium valproate, the local Wnt agonist is compound 3, or a pharmaceutically acceptable salt thereof. In some embodiments, the systemic valproic acid compound may further include valproic acid.

In some embodiments, the systemic valproic acid compound may include sodium valproate, the local Wnt agonist is compound (R)-DW12, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is staurosporine, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor XXVI, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is manzamine A, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is TC-G 24, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 14d, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 15b, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 20x, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor 2, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is SU9516, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is AZD1080, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is kenpaullone, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 17b, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is azakenpaullone, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is alsterpaullone, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is alsterpaullone 2-cyanoethyl, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is cazpaullone, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is FRATtide (SEQ ID NO: 1), or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is L803 (SEQ ID NO: 2), or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is L803-mts (SEQ ID NO: 3), or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor XXII, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 4a, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 4t, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 4z, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is AT 7519, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is pyrazolopyridine 9, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is pyrazolopyridine 18, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is pyrazolopyridine 34, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 14, or a pharmaceutically acceptable salt thereof. In some embodiments, the systemic valproic acid compound may further include valproic acid.

In some embodiments, the systemic valproic acid compound may include sodium valproate, the local Wnt agonist is compound 23, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 19, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is NSC 693868, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 150, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3 Inhibitor XIII, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is VP0.7, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is a compound having a CAS number 1132813-46-7, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is a compound having a CAS number 1132812-98-6, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is a compound having a CAS number 950727-66-9, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor VII, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor VI, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is palinurin, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is tricantin, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor I, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is NP031115, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is NP031112, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 90, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 92, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GSK-3β Inhibitor VIII, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is A-1070722, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 27, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 12, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is NP-103, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CG-301338, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is XD-4241, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CEP-16805, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is AZD13282107, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is SAR 502250, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is AR79, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GI179186X, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CT118637, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CP-70949, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GW784752X, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is GW784775X, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is CT73911, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is LY2064827, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 705701, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 708244, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is 709125, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is (R)-BRD4003, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate and the local Wnt agonist is (S)-BRD4003, or a pharmaceutically acceptable salt thereof. In some embodiments, the systemic valproic acid compound may further include valproic acid.

In some embodiments, the systemic valproic acid compound may include sodium valproate, the local Wnt agonist is compound 8, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 9, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 11, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is BRD1172, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is compound 16, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is BRD1652, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-1, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-2, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-3, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-4, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-5, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-6, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-7, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-8, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-9, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-10, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-11, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-12, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-13, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-14, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-15, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-16, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-17, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-18, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-19, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-20, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-21, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-22, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-23, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-24, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-25, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-26, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-27, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-28, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-29, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-30, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-31, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-32, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-33, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-34, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-35, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-36, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-37, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-38, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-39, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I40, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-41, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-42, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-43, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-44, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-45, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-46, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-47, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is Compound I-48, or a pharmaceutically acceptable salt thereof, and the local valproic acid compound may include sodium valproate. In some embodiments, one or more of the systemic and local valproic acid compound may further include valproic acid.

In some embodiments, the systemic valproic acid compound may include sodium valproate, the local Wnt agonist is selected from Compounds I-1, I-2, I-3, I-6, I-7, I-8, I-9, I-12, I-16, I-18, I-20, I-22, I-23. I-24, I-25, I-26. I-27, I-29, I-30, I-31, I-33, I-36, I-39, I-43, I-44, and I-48, or a pharmaceutically acceptable salt thereof, and the local valproic acid compound may include sodium valproate. In some embodiments, one or more of the systemic and local valproic acid compound may further include valproic acid.

### Combinations: Local Wnt Agonist and Local VPA Compound

In some embodiments, the local Wnt agonist is bikinin, or a pharmaceutically acceptable salt thereof, and the local valproic acid compound may include sodium valproate. In some embodiments, the local valproic acid compound may further include valproic acid.

In some embodiments, the local Wnt agonist is hymenialdisine, or a pharmaceutically acceptable salt thereof, and the local valproic acid compound may include sodium valproate. In some embodiments, the local valproic acid compound may further include valproic acid.

In some embodiments, the local Wnt agonist is aloisine A, or a pharmaceutically acceptable salt thereof, and the local valproic acid compound may include sodium valproate. In some embodiments, the local valproic acid compound may further include valproic acid.

In some embodiments, the local Wnt agonist is aloisine B, or a pharmaceutically acceptable salt thereof, and the local valproic acid compound may include sodium valproate. In some embodiments, the local valproic acid compound may further include valproic acid.

In some embodiments, the local Wnt agonist is TWS119, or a pharmaceutically acceptable salt thereof, and the local valproic acid compound may include sodium valproate. In some embodiments, the local valproic acid compound may further include valproic acid.

In some embodiments, the local Wnt agonist is CT20026, or a pharmaceutically acceptable salt thereof, and the local valproic acid compound may include sodium valproate. In some embodiments, the local valproic acid compound may further include valproic acid.

In some embodiments, the local Wnt agonist is CHIR98014, or a pharmaceutically acceptable salt thereof, and the local valproic acid compound may include sodium valproate. In some embodiments, the local valproic acid compound may further include valproic acid.

In some embodiments, the local Wnt agonist is CHIR98023, or a pharmaceutically acceptable salt thereof, and the local valproic acid compound may include sodium valproate. In some embodiments, the local valproic acid compound may further include valproic acid.

In some embodiments, the local Wnt agonist is CHIR98024, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor XVIII, or a pharmaccutically acceptable salt thereof, and the local valproic acid compound may include sodium valproate. In some embodiments, the local valproic acid compound may further include valproic acid.

In some embodiments, the local Wnt agonist is CGP60474, or a pharmaceutically acceptable salt thereof, and the local valproic acid compound may include sodium valproate. In some embodiments, the local valproic acid compound may further include valproic acid.

In some embodiments, the local Wnt agonist is AZD2858, or a pharmaceutically acceptable salt thereof, and the local valproic acid compound may include sodium valproate. In some embodiments, the local valproic acid compound may further include valproic acid.

In some embodiments, the local Wnt agonist is CID 755673, or a pharmaceutically acceptable salt thereof.

In some embodiments, the local Wnt agonist is TCS 2002, or a pharmaceutically acceptable salt thereof, and the local valproic acid compound may include sodium valproate. In some embodiments, the local valproic acid compound may further include valproic acid.

In some embodiments, the local Wnt agonist is dibromocantharelline, or a pharmaceutically acceptable salt thereof, and the local valproic acid compound may include sodium valproate. In some embodiments, the local valproic acid compound may further include valproic acid.

In some embodiments, the local Wnt agonist is ML320, or a pharmaceutically acceptable salt thereof, and the local valproic acid compound may include sodium valproate. In some embodiments, the local valproic acid compound may further include valproic acid.

In some embodiments, the local Wnt agonist is flavopiridol, or a pharmaceutically acceptable salt thereof, and the local valproic acid compound may include sodium valproate. In some embodiments, the local valproic acid compound may further include valproic acid.

In some embodiments, the local Wnt agonist is compound 100, or a pharmaceutically acceptable salt thereof, and the local valproic acid compound may include sodium valproate. In some embodiments, the local valproic acid compound may further include valproic acid.

In some embodiments, the local Wnt agonist is hymenidin, or a pharmaceutically acceptable salt thereof, and the local valproic acid compound may include sodium valproate. In some embodiments, the local valproic acid compound may further include valproic acid.

In some embodiments, the local Wnt agonist is 6-bromoindirubin-3′-acetoxime, or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor IX, or a pharmaceutically acceptable salt thereof, and the local valproic acid compound may include sodium valproate. In some embodiments, the local valproic acid compound may further include valproic acid.

In some embodiments, the local Wnt agonist is indirubin-3′-monoxime, or a pharmaceutically acceptable salt thereof, and the local valproic acid compound may include sodium valproate. In some embodiments, the local valproic acid compound may further include valproic acid.

In some embodiments, the local Wnt agonist is 5-iodo-indirubin-3′-monoxime, or a pharmaceutically acceptable salt thereof, and the local valproic acid compound may include sodium valproate. In some embodiments, the local valproic acid compound may further include valproic acid.

In some embodiments, the local Wnt agonist is indirubin-5-sulfonic acid, or a pharmaceutically acceptable salt thereof, and the local valproic acid compound may include sodium valproate. In some embodiments, the local valproic acid compound may further include valproic acid.

In some embodiments, the local Wnt agonist is indirubin, or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor X, or a pharmaceutically acceptable salt thereof, and the local valproic acid compound may include sodium valproate. In some embodiments, the local valproic acid compound may further include valproic acid.

In some embodiments, the local Wnt agonist is lithium chloride. In some embodiments, the systemic valproic acid compound may further include valproic acid.

In some embodiments, the local Wnt agonist is a beryllium salt, e.g. BeSO4. In some embodiments, the systemic valproic acid compound may further include valproic acid.

In some embodiments, the local Wnt agonist is a zinc salt. In some embodiments, the systemic valproic acid compound may further include valproic acid.

In some embodiments, the local Wnt agonist is a tungstate salt. In some embodiments, the systemic valproic acid compound may further include valproic acid.

In some embodiments, the local Wnt agonist is a mercury salt. In some embodiments, the systemic valproic acid compound may further include valproic acid.

In some embodiments, the local Wnt agonist is a copper salt. In some embodiments, the systemic valproic acid compound may further include valproic acid.

In some embodiments, the local Wnt agonist is compound 39, or a pharmaceutically acceptable salt thereof, and the local valproic acid compound may include sodium valproate. In some embodiments, the local valproic acid compound may further include valproic acid.

In some embodiments, the local Wnt agonist is compound 29 (CAS 1772823-37-6), or a pharmaceutically acceptable salt thereof, and the local valproic acid compound may include sodium valproate. In some embodiments, the local valproic acid compound may further include valproic acid.

In some embodiments, the local Wnt agonist is compound 33, or a pharmaceutically acceptable salt thereof, and the local valproic acid compound may include sodium valproate. In some embodiments, the local valproic acid compound may further include valproic acid.

In some embodiments, the local Wnt agonist is compound 29′ (CAS 436866-61-4), or a pharmaceutically acceptable salt thereof, and the local valproic acid compound may include sodium valproate. In some embodiments, the local valproic acid compound may further include valproic acid.

In some embodiments, the local Wnt agonist is compound 46, or a pharmaceutically acceptable salt thereof, and the local valproic acid compound may include sodium valproate. In some embodiments, the local valproic acid compound may further include valproic acid.

In some embodiments, the local Wnt agonist is compound 5a, or a pharmaceutically acceptable salt thereof, and the local valproic acid compound may include sodium valproate. In some embodiments, the local valproic acid compound may further include valproic acid.

In some embodiments, the local Wnt agonist is GF109203x, or a pharmaceutically acceptable salt thereof, and the local valproic acid compound may include sodium valproate. In some embodiments, the local valproic acid compound may further include valproic acid.

In some embodiments, the local Wnt agonist is RO31-8220, or a pharmaceutically acceptable salt thereof, and the local valproic acid compound may include sodium valproate. In some embodiments, the local valproic acid compound may further include valproic acid.

In some embodiments, the local Wnt agonist is bisindolyl maleimide X, or a pharmaceutically acceptable salt thereof, and the local valproic acid compound may include sodium valproate. In some embodiments, the local valproic acid compound may further include valproic acid.

In some embodiments, the local Wnt agonist is enzastaurin (LY317615), or a pharmaceutically acceptable salt thereof, and the local valproic acid compound may include sodium valproate. In some embodiments, the local valproic acid compound may further include valproic acid.

In some embodiments, the local Wnt agonist is I5, or a pharmaceutically acceptable salt thereof, and the local valproic acid compound may include sodium valproate. In some embodiments, the local valproic acid compound may further include valproic acid.

In some embodiments, the local Wnt agonist is SB-216763, or a pharmaceutically acceptable salt thereof, and the local valproic acid compound may include sodium valproate. In some embodiments, the local valproic acid compound may further include valproic acid.

In some embodiments, the local Wnt agonist is SB-415286, or a pharmaceutically acceptable salt thereof, and the local valproic acid compound may include sodium valproate. In some embodiments, the local valproic acid compound may further include valproic acid.

In some embodiments, the local Wnt agonist is 3F8, or a pharmaceutically acceptable salt thereof, and the local valproic acid compound may include sodium valproate. In some embodiments, the local valproic acid compound may further include valproic acid.

In some embodiments, the local Wnt agonist is TCS 21311, or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor 1, or a pharmaceutically acceptable salt thereof, and the local valproic acid compound may include sodium valproate. In some embodiments, the local valproic acid compound may further include valproic acid.

In some embodiments, the local Wnt agonist is LY2090314, or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor-I, or a pharmaceutically acceptable salt thereof, and the local valproic acid compound may include sodium valproate. In some embodiments, the local valproic acid compound may further include valproic acid.

In some embodiments, the local Wnt agonist is IM-12, or a pharmaceutically acceptable salt thereof, and the local valproic acid compound may include sodium valproate. In some embodiments, the local valproic acid compound may further include valproic acid.

In some embodiments, the local Wnt agonist is compound 34, or a pharmaceutically acceptable salt thereof, and the local valproic acid compound may include sodium valproate. In some embodiments, the local valproic acid compound may further include valproic acid.

In some embodiments, the local Wnt agonist is KT 5720, or a pharmaceutically acceptable salt thereof, and the local valproic acid compound may include sodium valproate. In some embodiments, the local valproic acid compound may further include valproic acid.

In some embodiments, the local Wnt agonist is isogranulatimide, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor XI, or a pharmaceutically acceptable salt thereof, and the local valproic acid compound may include sodium valproate. In some embodiments, the local valproic acid compound may further include valproic acid.

In some embodiments, the local Wnt agonist is BIP-135, or a pharmaceutically acceptable salt thereof, and the local valproic acid compound may include sodium valproate. In some embodiments, the local valproic acid compound may further include valproic acid.

In some embodiments, the local Wnt agonist is CP21R7, or a pharmaceutically acceptable salt thereof, and the local valproic acid compound may include sodium valproate. In some embodiments, the local valproic acid compound may further include valproic acid.

In some embodiments, the local Wnt agonist is tivantinib, or a pharmaceutically acceptable salt thereof, and the local valproic acid compound may include sodium valproate. In some embodiments, the local valproic acid compound may further include valproic acid.

In some embodiments, the local Wnt agonist is compound lambda-OS1, or a pharmaceutically acceptable salt thereof, and the local valproic acid compound may include sodium valproate. In some embodiments, the local valproic acid compound may further include valproic acid.

In some embodiments, the local Wnt agonist is HB12, or a pharmaceutically acceptable salt thereof, and the local valproic acid compound may include sodium valproate. In some embodiments, the local valproic acid compound may further include valproic acid.

In some embodiments, the local Wnt agonist is DW12, or a pharmaceutically acceptable salt thereof, and the local valproic acid compound may include sodium valproate. In some embodiments, the local valproic acid compound may further include valproic acid.

In some embodiments, the local Wnt agonist is NP309, or a pharmaceutically acceptable salt thereof, and the local valproic acid compound may include sodium valproate. In some embodiments, the local valproic acid compound may further include valproic acid.

In some embodiments, the local Wnt agonist is (RRu)-HB1229, or a pharmaceutically acceptable salt thereof. In some embodiments, the systemic valproic acid compound may further include valproic acid.

In some embodiments, the local Wnt agonist is (RRu)-NP549, or a pharmaceutically acceptable salt thereof. In some embodiments, the systemic valproic acid compound may further include valproic acid.

In some embodiments, the local Wnt agonist is compound 3, or a pharmaceutically acceptable salt thereof. In some embodiments, the systemic valproic acid compound may further include valproic acid.

In some embodiments, the local Wnt agonist is compound (R)-DW12, or a pharmaceutically acceptable salt thereof, and the local valproic acid compound may include sodium valproate. In some embodiments, the local valproic acid compound may further include valproic acid.

In some embodiments, the local Wnt agonist is staurosporine, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor XXVI, or a pharmaceutically acceptable salt thereof, and the local valproic acid compound may include sodium valproate. In some embodiments, the local valproic acid compound may further include valproic acid.

In some embodiments, the local Wnt agonist is manzamine A, or a pharmaceutically acceptable salt thereof, and the local valproic acid compound may include sodium valproate. In some embodiments, the local valproic acid compound may further include valproic acid.

In some embodiments, the local Wnt agonist is TC-G 24, or a pharmaceutically acceptable salt thereof, and the local valproic acid compound may include sodium valproate. In some embodiments, the local valproic acid compound may further include valproic acid.

In some embodiments, the local Wnt agonist is compound 14d, or a pharmaceutically acceptable salt thereof, and the local valproic acid compound may include sodium valproate. In some embodiments, the local valproic acid compound may further include valproic acid.

In some embodiments, the local Wnt agonist is compound 15b, or a pharmaceutically acceptable salt thereof, and the local valproic acid compound may include sodium valproate. In some embodiments, the local valproic acid compound may further include valproic acid.

In some embodiments, the local Wnt agonist is compound 20x, or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor 2, or a pharmaceutically acceptable salt thereof, and the local valproic acid compound may include sodium valproate. In some embodiments, the local valproic acid compound may further include valproic acid.

In some embodiments, the local Wnt agonist is SU9516, or a pharmaceutically acceptable salt thereof, and the local valproic acid compound may include sodium valproate. In some embodiments, the local valproic acid compound may further include valproic acid.

In some embodiments, the local Wnt agonist is AZD1080, or a pharmaceutically acceptable salt thereof, and the local valproic acid compound may include sodium valproate. In some embodiments, the local valproic acid compound may further include valproic acid.

In some embodiments, the local Wnt agonist is kenpaullone, or a pharmaceutically acceptable salt thereof, and the local valproic acid compound may include sodium valproate. In some embodiments, the local valproic acid compound may further include valproic acid.

In some embodiments, the local Wnt agonist is compound 17b, or a pharmaceutically acceptable salt thereof, and the local valproic acid compound may include sodium valproate. In some embodiments, the local valproic acid compound may further include valproic acid.

In some embodiments, the local Wnt agonist is azakenpaullone, or a pharmaceutically acceptable salt thereof, and the local valproic acid compound may include sodium valproate. In some embodiments, the local valproic acid compound may further include valproic acid.

In some embodiments, the local Wnt agonist is alsterpaullone, or a pharmaceutically acceptable salt thereof, and the local valproic acid compound may include sodium valproate. In some embodiments, the local valproic acid compound may further include valproic acid.

In some embodiments, the local Wnt agonist is alsterpaullone 2-cyanoethyl, or a pharmaceutically acceptable salt thereof, and the local valproic acid compound may include sodium valproate. In some embodiments, the local valproic acid compound may further include valproic acid.

In some embodiments, the local Wnt agonist is cazpaullone, or a pharmaceutically acceptable salt thereof, and the local valproic acid compound may include sodium valproate. In some embodiments, the local valproic acid compound may further include valproic acid.

In some embodiments, the local Wnt agonist is FRATtide (SEQ ID NO: 1), or a pharmaceutically acceptable salt thereof, and the local valproic acid compound may include sodium valproate. In some embodiments, the local valproic acid compound may further include valproic acid.

In some embodiments, the local Wnt agonist is L803 (SEQ ID NO: 2), or a pharmaceutically acceptable salt thereof, and the local valproic acid compound may include sodium valproate. In some embodiments, the local valproic acid compound may further include valproic acid.

In some embodiments, the local Wnt agonist is L803-mts (SEQ ID NO: 3), or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor XXII, or a pharmaceutically acceptable salt thereof, and the local valproic acid compound may include sodium valproate. In some embodiments, the local valproic acid compound may further include valproic acid.

In some embodiments, the local Wnt agonist is compound 4a, or a pharmaceutically acceptable salt thereof, and the local valproic acid compound may include sodium valproate. In some embodiments, the local valproic acid compound may further include valproic acid.

In some embodiments, the local Wnt agonist is compound 4t, or a pharmaceutically acceptable salt thereof, and the local valproic acid compound may include sodium valproate. In some embodiments, the local valproic acid compound may further include valproic acid.

In some embodiments, the local Wnt agonist is compound 4z, or a pharmaceutically acceptable salt thereof, and the local valproic acid compound may include sodium valproate. In some embodiments, the local valproic acid compound may further include valproic acid.

In some embodiments, the local Wnt agonist is AT 7519, or a pharmaceutically acceptable salt thereof, and the local valproic acid compound may include sodium valproate. In some embodiments, the local valproic acid compound may further include valproic acid.

In some embodiments, the local Wnt agonist is pyrazolopyridine 9, or a pharmaceutically acceptable salt thereof, and the local valproic acid compound may include sodium valproate. In some embodiments, the local valproic acid compound may further include valproic acid.

In some embodiments, the local Wnt agonist is pyrazolopyridine 18, or a pharmaceutically acceptable salt thereof, and the local valproic acid compound may include sodium valproate. In some embodiments, the local valproic acid compound may further include valproic acid.

In some embodiments, the local Wnt agonist is pyrazolopyridine 34, or a pharmaceutically acceptable salt thereof, and the local valproic acid compound may include sodium valproate. In some embodiments, the local valproic acid compound may further include valproic acid.

In some embodiments, the local Wnt agonist is compound 14, or a pharmaceutically acceptable salt thereof. In some embodiments, the systemic valproic acid compound may further include valproic acid.

In some embodiments, the local Wnt agonist is compound 23, or a pharmaceutically acceptable salt thereof, and the local valproic acid compound may include sodium valproate. In some embodiments, the local valproic acid compound may further include valproic acid.

In some embodiments, the local Wnt agonist is compound 19, or a pharmaceutically acceptable salt thereof, and the local valproic acid compound may include sodium valproate. In some embodiments, the local valproic acid compound may further include valproic acid.

In some embodiments, the local Wnt agonist is NSC 693868, or a pharmaceutically acceptable salt thereof, and the local valproic acid compound may include sodium valproate. In some embodiments, the local valproic acid compound may further include valproic acid.

In some embodiments, the local Wnt agonist is compound 150, or a pharmaceutically acceptable salt thereof, and the local valproic acid compound may include sodium valproate. In some embodiments, the local valproic acid compound may further include valproic acid.

In some embodiments, the local Wnt agonist is GSK-3 Inhibitor XIII, or a pharmaceutically acceptable salt thereof, and the local valproic acid compound may include sodium valproate. In some embodiments, the local valproic acid compound may further include valproic acid.

In some embodiments, the local Wnt agonist is VP0.7, or a pharmaceutically acceptable salt thereof, and the local valproic acid compound may include sodium valproate. In some embodiments, the local valproic acid compound may further include valproic acid.

In some embodiments, the local Wnt agonist is a compound having a CAS number 113281346-7, or a pharmaceutically acceptable salt thereof, and the local valproic acid compound may include sodium valproate. In some embodiments, the local valproic acid compound may further include valproic acid.

In some embodiments, the local Wnt agonist is a compound having a CAS number 1132812-98-6, or a pharmaceutically acceptable salt thereof, and the local valproic acid compound may include sodium valproate. In some embodiments, the local valproic acid compound may further include valproic acid.

In some embodiments, the local Wnt agonist is a compound having a CAS number 950727-66-9, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor VII, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor VI, or a pharmaceutically acceptable salt thereof, and the local valproic acid compound may include sodium valproate. In some embodiments, the local valproic acid compound may further include valproic acid.

In some embodiments, the local Wnt agonist is palinurin, or a pharmaceutically acceptable salt thereof, and the local valproic acid compound may include sodium valproate. In some embodiments, the local valproic acid compound may further include valproic acid.

In some embodiments, the local Wnt agonist is tricantin, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor I, or a pharmaceutically acceptable salt thereof, and the local valproic acid compound may include sodium valproate. In some embodiments, the local valproic acid compound may further include valproic acid.

In some embodiments, the local Wnt agonist is NP031115, or a pharmaceutically acceptable salt thereof, and the local valproic acid compound may include sodium valproate. In some embodiments, the local valproic acid compound may further include valproic acid.

In some embodiments, the local Wnt agonist is NP031112, or a pharmaceutically acceptable salt thereof, and the local valproic acid compound may include sodium valproate. In some embodiments, the local valproic acid compound may further include valproic acid.

In some embodiments, the local Wnt agonist is compound 90, or a pharmaceutically acceptable salt thereof, and the local valproic acid compound may include sodium valproate. In some embodiments, the local valproic acid compound may further include valproic acid.

In some embodiments, the local Wnt agonist is compound 92, or a pharmaceutically acceptable salt thereof, and the local valproic acid compound may include sodium valproate. In some embodiments, the local valproic acid compound may further include valproic acid.

In some embodiments, the local Wnt agonist is GSK-3β Inhibitor VIII, or a pharmaceutically acceptable salt thereof, and the local valproic acid compound may include sodium valproate. In some embodiments, the local valproic acid compound may further include valproic acid.

In some embodiments, the local Wnt agonist is A-1070722, or a pharmaceutically acceptable salt thereof, and the local valproic acid compound may include sodium valproate. In some embodiments, the local valproic acid compound may further include valproic acid.

In some embodiments, the local Wnt agonist is compound 27, or a pharmaceutically acceptable salt thereof, and the local valproic acid compound may include sodium valproate. In some embodiments, the local valproic acid compound may further include valproic acid.

In some embodiments, the local Wnt agonist is compound 12, or a pharmaceutically acceptable salt thereof, and the local valproic acid compound may include sodium valproate. In some embodiments, the local valproic acid compound may further include valproic acid.

In some embodiments, the local Wnt agonist is NP-103, or a pharmaceutically acceptable salt thereof, and the local valproic acid compound may include sodium valproate. In some embodiments, the local valproic acid compound may further include valproic acid.

In some embodiments, the local Wnt agonist is CG-301338, or a pharmaceutically acceptable salt thereof, and the local valproic acid compound may include sodium valproate. In some embodiments, the local valproic acid compound may further include valproic acid.

In some embodiments, the local Wnt agonist is XD-4241, or a pharmaceutically acceptable salt thereof, and the local valproic acid compound may include sodium valproate. In some embodiments, the local valproic acid compound may further include valproic acid.

In some embodiments, the local Wnt agonist is CEP-16805, or a pharmaceutically acceptable salt thereof, and the local valproic acid compound may include sodium valproate. In some embodiments, the local valproic acid compound may further include valproic acid.

In some embodiments, the local Wnt agonist is AZD13282107, or a pharmaceutically acceptable salt thereof, and the local valproic acid compound may include sodium valproate. In some embodiments, the local valproic acid compound may further include valproic acid.

In some embodiments, the local Wnt agonist is SAR 502250, or a pharmaceutically acceptable salt thereof, and the local valproic acid compound may include sodium valproate. In some embodiments, the local valproic acid compound may further include valproic acid.

In some embodiments, the local Wnt agonist is AR79, or a pharmaceutically acceptable salt thereof, and the local valproic acid compound may include sodium valproate. In some embodiments, the local valproic acid compound may further include valproic acid.

In some embodiments, the local Wnt agonist is GI179186X, or a pharmaceutically acceptable salt thereof, and the local valproic acid compound may include sodium valproate. In some embodiments, the local valproic acid compound may further include valproic acid.

In some embodiments, the local Wnt agonist is CT118637, or a pharmaceutically acceptable salt thereof, and the local valproic acid compound may include sodium valproate. In some embodiments, the local valproic acid compound may further include valproic acid.

In some embodiments, the local Wnt agonist is CP-70949, or a pharmaceutically acceptable salt thereof, and the local valproic acid compound may include sodium valproate. In some embodiments, the local valproic acid compound may further include valproic acid.

In some embodiments, the local Wnt agonist is GW784752X, or a pharmaceutically acceptable salt thereof, and the local valproic acid compound may include sodium valproate. In some embodiments, the local valproic acid compound may further include valproic acid.

In some embodiments, the local Wnt agonist is GW784775X, or a pharmaceutically acceptable salt thereof, and the local valproic acid compound may include sodium valproate. In some embodiments, the local valproic acid compound may further include valproic acid.

In some embodiments, the local Wnt agonist is CT73911, or a pharmaceutically acceptable salt thereof, and the local valproic acid compound may include sodium valproate. In some embodiments, the local valproic acid compound may further include valproic acid.

In some embodiments, the local Wnt agonist is LY2064827, or a pharmaceutically acceptable salt thereof, and the local valproic acid compound may include sodium valproate. In some embodiments, the local valproic acid compound may further include valproic acid.

In some embodiments, the local Wnt agonist is 705701, or a pharmaceutically acceptable salt thereof, and the local valproic acid compound may include sodium valproate. In some embodiments, the local valproic acid compound may further include valproic acid.

In some embodiments, the local Wnt agonist is 708244, or a pharmaceutically acceptable salt thereof, and the local valproic acid compound may include sodium valproate. In some embodiments, the local valproic acid compound may further include valproic acid.

In some embodiments, the local Wnt agonist is 709125, or a pharmaceutically acceptable salt thereof, and the local valproic acid compound may include sodium valproate. In some embodiments, the local valproic acid compound may further include valproic acid.

In some embodiments, the local Wnt agonist is (R)-BRD4003, or a pharmaceutically acceptable salt thereof, and the local valproic acid compound may include sodium valproate. In some embodiments, the local valproic acid compound may further include valproic acid.

In some embodiments, the local Wnt agonist is (S)-BRD4003, or a pharmaceutically acceptable salt thereof, and the local valproic acid compound may include sodium valproate. In some embodiments, the local valproic acid compound may further include valproic acid.

In some embodiments, the local Wnt agonist is compound 8, or a pharmaceutically acceptable salt thereof, and the local valproic acid compound may include sodium valproate. In some embodiments, the local valproic acid compound may further include valproic acid.

In some embodiments, the local Wnt agonist is compound 9, or a pharmaceutically acceptable salt thereof, and the local valproic acid compound may include sodium valproate. In some embodiments, the local valproic acid compound may further include valproic acid.

In some embodiments, the local Wnt agonist is compound 11, or a pharmaceutically acceptable salt thereof, and the local valproic acid compound may include sodium valproate. In some embodiments, the local valproic acid compound may further include valproic acid.

In some embodiments, the local Wnt agonist is BRD1172, or a pharmaceutically acceptable salt thereof, and the local valproic acid compound may include sodium valproate. In some embodiments, the local valproic acid compound may further include valproic acid.

In some embodiments, the local Wnt agonist is compound 16, or a pharmaceutically acceptable salt thereof, and the local valproic acid compound may include sodium valproate. In some embodiments, the local valproic acid compound may further include valproic acid.

In some embodiments, the local Wnt agonist is BRD1652, or a pharmaceutically acceptable salt thereof, and the local valproic acid compound may include sodium valproate. In some embodiments, the local valproic acid compound may further include valproic acid.

In some embodiments, the local Wnt agonist is Compound I-1, or a pharmaceutically acceptable salt thereof, and the local valproic acid compound may include sodium valproate. In some embodiments, the local valproic acid compound may further include valproic acid.

In some embodiments, the local Wnt agonist is Compound I-2, or a pharmaceutically acceptable salt thereof, and the local valproic acid compound may include sodium valproate. In some embodiments, the local valproic acid compound may further include valproic acid.

In some embodiments, the local Wnt agonist is Compound I-3, or a pharmaceutically acceptable salt thereof, and the local valproic acid compound may include sodium valproate. In some embodiments, the local valproic acid compound may further include valproic acid.

In some embodiments, the local Wnt agonist is Compound I-4, or a pharmaceutically acceptable salt thereof, and the local valproic acid compound may include sodium valproate. In some embodiments, the local valproic acid compound may further include valproic acid.

In some embodiments, the local Wnt agonist is Compound I-5, or a pharmaceutically acceptable salt thereof, and the local valproic acid compound may include sodium valproate. In some embodiments, the local valproic acid compound may further include valproic acid.

In some embodiments, the local Wnt agonist is Compound I-6, or a pharmaceutically acceptable salt thereof, and the local valproic acid compound may include sodium valproate. In some embodiments, the local valproic acid compound may further include valproic acid.

In some embodiments, the local Wnt agonist is Compound I-7, or a pharmaceutically acceptable salt thereof, and the local valproic acid compound may include sodium valproate. In some embodiments, the local valproic acid compound may further include valproic acid.

In some embodiments, the local Wnt agonist is Compound I-8, or a pharmaceutically acceptable salt thereof, and the local valproic acid compound may include sodium valproate. In some embodiments, the local valproic acid compound may further include valproic acid.

In some embodiments, the local Wnt agonist is Compound I-9, or a pharmaceutically acceptable salt thereof, and the local valproic acid compound may include sodium valproate. In some embodiments, the local valproic acid compound may further include valproic acid.

In some embodiments, the local Wnt agonist is Compound I-10, or a pharmaceutically acceptable salt thereof, and the local valproic acid compound may include sodium valproate. In some embodiments, the local valproic acid compound may further include valproic acid.

In some embodiments, the local Wnt agonist is Compound I-11, or a pharmaceutically acceptable salt thereof, and the local valproic acid compound may include sodium valproate. In some embodiments, the local valproic acid compound may further include valproic acid.

In some embodiments, the local Wnt agonist is Compound I-12, or a pharmaceutically acceptable salt thereof, and the local valproic acid compound may include sodium valproate. In some embodiments, the local valproic acid compound may further include valproic acid.

In some embodiments, the local Wnt agonist is Compound I-13, or a pharmaceutically acceptable salt thereof, and the local valproic acid compound may include sodium valproate. In some embodiments, the local valproic acid compound may further include valproic acid.

In some embodiments, the local Wnt agonist is Compound I-14, or a pharmaceutically acceptable salt thereof, and the local valproic acid compound may include sodium valproate. In some embodiments, the local valproic acid compound may further include valproic acid.

In some embodiments, the local Wnt agonist is Compound I-15, or a pharmaceutically acceptable salt thereof, and the local valproic acid compound may include sodium valproate. In some embodiments, the local valproic acid compound may further include valproic acid.

In some embodiments, the local Wnt agonist is Compound I-16, or a pharmaceutically acceptable salt thereof, and the local valproic acid compound may include sodium valproate. In some embodiments, the local valproic acid compound may further include valproic acid.

In some embodiments, the local Wnt agonist is Compound I-17, or a pharmaceutically acceptable salt thereof, and the local valproic acid compound may include sodium valproate. In some embodiments, the local valproic acid compound may further include valproic acid.

In some embodiments, the local Wnt agonist is Compound I-18, or a pharmaccutically acceptable salt thereof, and the local valproic acid compound may include sodium valproate. In some embodiments, the local valproic acid compound may further include valproic acid.

In some embodiments, the local Wnt agonist is Compound I-19, or a pharmaceutically acceptable salt thereof, and the local valproic acid compound may include sodium valproate. In some embodiments, the local valproic acid compound may further include valproic acid.

In some embodiments, the local Wnt agonist is Compound I-20, or a pharmaceutically acceptable salt thereof, and the local valproic acid compound may include sodium valproate. In some embodiments, the local valproic acid compound may further include valproic acid.

In some embodiments, the local Wnt agonist is Compound I-21, or a pharmaceutically acceptable salt thereof, and the local valproic acid compound may include sodium valproate. In some embodiments, the local valproic acid compound may further include valproic acid.

In some embodiments, the local Wnt agonist is Compound I-22, or a pharmaceutically acceptable salt thereof, and the local valproic acid compound may include sodium valproate. In some embodiments, the local valproic acid compound may further include valproic acid.

In some embodiments, the local Wnt agonist is Compound I-23, or a pharmaceutically acceptable salt thereof, and the local valproic acid compound may include sodium valproate. In some embodiments, the local valproic acid compound may further include valproic acid.

In some embodiments, the local Wnt agonist is Compound I-24, or a pharmaceutically acceptable salt thereof, and the local valproic acid compound may include sodium valproate. In some embodiments, the local valproic acid compound may further include valproic acid.

In some embodiments, the local Wnt agonist is Compound I-25, or a pharmaceutically acceptable salt thereof, and the local valproic acid compound may include sodium valproate. In some embodiments, the local valproic acid compound may further include valproic acid.

In some embodiments, the local Wnt agonist is Compound I-26, or a pharmaceutically acceptable salt thereof, and the local valproic acid compound may include sodium valproate. In some embodiments, the local valproic acid compound may further include valproic acid.

In some embodiments, the local Wnt agonist is Compound I-27, or a pharmaceutically acceptable salt thereof, and the local valproic acid compound may include sodium valproate. In some embodiments, the local valproic acid compound may further include valproic acid.

In some embodiments, the local Wnt agonist is Compound I-28, or a pharmaceutically acceptable salt thereof, and the local valproic acid compound may include sodium valproate. In some embodiments, the local valproic acid compound may further include valproic acid.

In some embodiments, the local Wnt agonist is Compound I-29, or a pharmaceutically acceptable salt thereof, and the local valproic acid compound may include sodium valproate. In some embodiments, the local valproic acid compound may further include valproic acid.

In some embodiments, the local Wnt agonist is Compound I-30, or a pharmaceutically acceptable salt thereof, and the local valproic acid compound may include sodium valproate. In some embodiments, the local valproic acid compound may further include valproic acid.

In some embodiments, the local Wnt agonist is Compound I-31, or a pharmaceutically acceptable salt thereof, and the local valproic acid compound may include sodium valproate. In some embodiments, the local valproic acid compound may further include valproic acid.

In some embodiments, the local Wnt agonist is Compound I-32, or a pharmaceutically acceptable salt thereof, and the local valproic acid compound may include sodium valproate. In some embodiments, the local valproic acid compound may further include valproic acid.

In some embodiments, the local Wnt agonist is Compound I-33, or a pharmaccutically acceptable salt thereof, and the local valproic acid compound may include sodium valproate. In some embodiments, the local valproic acid compound may further include valproic acid.

In some embodiments, the local Wnt agonist is Compound I-34, or a pharmaceutically acceptable salt thereof, and the local valproic acid compound may include sodium valproate. In some embodiments, the local valproic acid compound may further include valproic acid.

In some embodiments, the local Wnt agonist is Compound I-35, or a pharmaceutically acceptable salt thereof, and the local valproic acid compound may include sodium valproate. In some embodiments, the local valproic acid compound may further include valproic acid.

In some embodiments, the local Wnt agonist is Compound I-36, or a pharmaceutically acceptable salt thereof, and the local valproic acid compound may include sodium valproate. In some embodiments, the local valproic acid compound may further include valproic acid.

In some embodiments, the local Wnt agonist is Compound I-37, or a pharmaceutically acceptable salt thereof, and the local valproic acid compound may include sodium valproate. In some embodiments, the local valproic acid compound may further include valproic acid.

In some embodiments, the local Wnt agonist is Compound I-38, or a pharmaceutically acceptable salt thereof, and the local valproic acid compound may include sodium valproate. In some embodiments, the local valproic acid compound may further include valproic acid.

In some embodiments, the local Wnt agonist is Compound I-39, or a pharmaceutically acceptable salt thereof, and the local valproic acid compound may include sodium valproate. In some embodiments, the local valproic acid compound may further include valproic acid.

In some embodiments, the local Wnt agonist is Compound I-40, or a pharmaceutically acceptable salt thereof, and the local valproic acid compound may include sodium valproate. In some embodiments, the local valproic acid compound may further include valproic acid.

In some embodiments, the local Wnt agonist is Compound I-41, or a pharmaceutically acceptable salt thereof, and the local valproic acid compound may include sodium valproate. In some embodiments, the local valproic acid compound may further include valproic acid.

In some embodiments, the local Wnt agonist is Compound I-42, or a pharmaceutically acceptable salt thereof, and the local valproic acid compound may include sodium valproate. In some embodiments, the local valproic acid compound may further include valproic acid.

In some embodiments, the local Wnt agonist is Compound I-43, or a pharmaceutically acceptable salt thereof, and the local valproic acid compound may include sodium valproate. In some embodiments, the local valproic acid compound may further include valproic acid.

In some embodiments, the local Wnt agonist is Compound I-44, or a pharmaceutically acceptable salt thereof, and the local valproic acid compound may include sodium valproate. In some embodiments, the local valproic acid compound may further include valproic acid.

In some embodiments, the local Wnt agonist is Compound 145, or a pharmaceutically acceptable salt thereof, and the local valproic acid compound may include sodium valproate. In some embodiments, the local valproic acid compound may further include valproic acid.

In some embodiments, the local Wnt agonist is Compound I-46, or a pharmaceutically acceptable salt thereof, and the local valproic acid compound may include sodium valproate. In some embodiments, the local valproic acid compound may further include valproic acid.

In some embodiments, the local Wnt agonist is Compound I-47, or a pharmaceutically acceptable salt thereof, and the local valproic acid compound may include sodium valproate. In some embodiments, the local valproic acid compound may further include valproic acid.

In some embodiments, the local Wnt agonist is Compound 148, or a pharmaccutically acceptable salt thereof, and the local valproic acid compound may include sodium valproate. In some embodiments, the local valproic acid compound may further include valproic acid.

In some embodiments, the local Wnt agonist is selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof, and the local valproic acid compound may include sodium valproate. In some embodiments, the local valproic acid compound may further include valproic acid.

### Combinations: Systemic VPA Compound and Systemic Wnt Agonist

In some embodiments, the systemic valproic acid compound may include sodium valproate and the systemic Wnt agonist is bikinin, or a pharmaceutically acceptable salt thereof. In some embodiments, the systemic valproic acid compound may further include valproic acid.

In some embodiments, the systemic valproic acid compound may include sodium valproate and the systemic Wnt agonist is hymenialdisine, or a pharmaceutically acceptable salt thereof. In some embodiments, the systemic valproic acid compound may further include valproic acid.

In some embodiments, the systemic valproic acid compound may include sodium valproate and the systemic Wnt agonist is aloisine A, or a pharmaceutically acceptable salt thereof. In some embodiments, the systemic valproic acid compound may further include valproic acid.

In some embodiments, the systemic valproic acid compound may include sodium valproate and the systemic Wnt agonist is aloisine B, or a pharmaceutically acceptable salt thereof. In some embodiments, the systemic valproic acid compound may further include valproic acid.

In some embodiments, the systemic valproic acid compound may include sodium valproate and the systemic Wnt agonist is TWS119, or a pharmaceutically acceptable salt thereof. In some embodiments, the systemic valproic acid compound may further include valproic acid.

In some embodiments, the systemic valproic acid compound may include sodium valproate and the systemic Wnt agonist is CT20026, or a pharmaceutically acceptable salt thereof. In some embodiments, the systemic valproic acid compound may further include valproic acid.

In some embodiments, the systemic valproic acid compound may include sodium valproate and the systemic Wnt agonist is CHIR98014, or a pharmaceutically acceptable salt thereof. In some embodiments, the systemic valproic acid compound may further include valproic acid.

In some embodiments, the systemic valproic acid compound may include sodium valproate and the systemic Wnt agonist is CHIR98023, or a pharmaceutically acceptable salt thereof. In some embodiments, the systemic valproic acid compound may further include valproic acid.

In some embodiments, the systemic valproic acid compound may include sodium valproate and the systemic Wnt agonist is CHIR98024, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-30 Inhibitor XVIII, or a pharmaceutically acceptable salt thereof. In some embodiments, the systemic valproic acid compound may further include valproic acid.

In some embodiments, the systemic valproic acid compound may include sodium valproate and the systemic Wnt agonist is CGP60474, or a pharmaceutically acceptable salt thereof. In some embodiments, the systemic valproic acid compound may further include valproic acid.

In some embodiments, the systemic valproic acid compound may include sodium valproate and the systemic Wnt agonist is AZD2858, or a pharmaceutically acceptable salt thereof. In some embodiments, the systemic valproic acid compound may further include valproic acid.

In some embodiments, the systemic valproic acid compound may include sodium valproate and the systemic Wnt agonist is CID 755673, or a pharmaceutically acceptable salt thereof.

In some embodiments, the systemic valproic acid compound may include sodium valproate and the systemic Wnt agonist is TCS 2002, or a pharmaceutically acceptable salt thereof. In some embodiments, the systemic valproic acid compound may further include valproic acid.

In some embodiments, the systemic valproic acid compound may include sodium valproate and the systemic Wnt agonist is dibromocantharelline, or a pharmaceutically acceptable salt thereof. In some embodiments, the systemic valproic acid compound may further include valproic acid.

In some embodiments, the systemic valproic acid compound may include sodium valproate and the systemic Wnt agonist is ML320, or a pharmaceutically acceptable salt thereof. In some embodiments, the systemic valproic acid compound may further include valproic acid.

In some embodiments, the systemic valproic acid compound may include sodium valproate and the systemic Wnt agonist is flavopiridol, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 100, or a pharmaceutically acceptable salt thereof. In some embodiments, the systemic valproic acid compound may further include valproic acid.

In some embodiments, the systemic valproic acid compound may include sodium valproate and the systemic Wnt agonist is hymenidin, or a pharmaceutically acceptable salt thereof. In some embodiments, the systemic valproic acid compound may further include valproic acid.

In some embodiments, the systemic valproic acid compound may include sodium valproate and the systemic Wnt agonist is 6-bromoindirubin-3′-acetoxime, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor IX, or a pharmaceutically acceptable salt thereof. In some embodiments, the systemic valproic acid compound may further include valproic acid.

In some embodiments, the systemic valproic acid compound may include sodium valproate and the systemic Wnt agonist is indirubin-3′-monoxime, or a pharmaceutically acceptable salt thereof. In some embodiments, the systemic valproic acid compound may further include valproic acid.

In some embodiments, the systemic valproic acid compound may include sodium valproate and the systemic Wnt agonist is 5-iodo-indirubin-3′-monoxime, or a pharmaceutically acceptable salt thereof. In some embodiments, the systemic valproic acid compound may further include valproic acid.

In some embodiments, the systemic valproic acid compound may include sodium valproate and the systemic Wnt agonist is indirubin-5-sulfonic acid, or a pharmaceutically acceptable salt thereof. In some embodiments, the systemic valproic acid compound may further include valproic acid.

In some embodiments, the systemic valproic acid compound may include sodium valproate and the systemic Wnt agonist is indirubin, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor X, or a pharmaceutically acceptable salt thereof. In some embodiments, the systemic valproic acid compound may further include valproic acid.

In some embodiments, the systemic valproic acid compound may include sodium valproate and the systemic Wnt agonist is lithium chloride. In some embodiments, the systemic valproic acid compound may further include valproic acid.

In some embodiments, the systemic valproic acid compound may include sodium valproate and the systemic Wnt agonist is a beryllium salt, e.g. BeSO4. In some embodiments, the systemic valproic acid compound may further include valproic acid.

In some embodiments, the systemic valproic acid compound may include sodium valproate and the systemic Wnt agonist is a zinc salt. In some embodiments, the systemic valproic acid compound may further include valproic acid.

In some embodiments, the systemic valproic acid compound may include sodium valproate and the systemic Wnt agonist is a tungstate salt. In some embodiments, the systemic valproic acid compound may further include valproic acid.

In some embodiments, the systemic valproic acid compound may include sodium valproate and the systemic Wnt agonist is a mercury salt. In some embodiments, the systemic valproic acid compound may further include valproic acid.

In some embodiments, the systemic valproic acid compound may include sodium valproate and the systemic Wnt agonist is a copper salt. In some embodiments, the systemic valproic acid compound may further include valproic acid.

In some embodiments, the systemic valproic acid compound may include sodium valproate and the systemic Wnt agonist is compound 39, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 29 (CAS 1772823-37-6), or a pharmaceutically acceptable salt thereof. In some embodiments, the systemic valproic acid compound may further include valproic acid.

In some embodiments, the systemic valproic acid compound may include sodium valproate and the systemic Wnt agonist is compound 33, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 29′ (CAS 436866-61-4), or a pharmaceutically acceptable salt thereof. In some embodiments, the systemic valproic acid compound may further include valproic acid.

In some embodiments, the systemic valproic acid compound may include sodium valproate and the systemic Wnt agonist is compound 46, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 5a, or a pharmaceutically acceptable salt thereof. In some embodiments, the systemic valproic acid compound may further include valproic acid.

In some embodiments, the systemic valproic acid compound may include sodium valproate and the systemic Wnt agonist is GF109203x, or a pharmaceutically acceptable salt thereof. In some embodiments, the systemic valproic acid compound may further include valproic acid.

In some embodiments, the systemic valproic acid compound may include sodium valproate and the systemic Wnt agonist is RO31-8220, or a pharmaceutically acceptable salt thereof. In some embodiments, the systemic valproic acid compound may further include valproic acid.

In some embodiments, the systemic valproic acid compound may include sodium valproate and the systemic Wnt agonist is bisindolyl maleimide X, or a pharmaceutically acceptable salt thereof. In some embodiments, the systemic valproic acid compound may further include valproic acid.

In some embodiments, the systemic valproic acid compound may include sodium valproate and the systemic Wnt agonist is enzastaurin (LY317615), or a pharmaceutically acceptable salt thereof. In some embodiments, the systemic valproic acid compound may further include valproic acid.

In some embodiments, the systemic valproic acid compound may include sodium valproate and the systemic Wnt agonist is 15, or a pharmaceutically acceptable salt thereof. In some embodiments, the systemic valproic acid compound may further include valproic acid.

In some embodiments, the systemic valproic acid compound may include sodium valproate and the systemic Wnt agonist is SB-216763, or a pharmaceutically acceptable salt thereof. In some embodiments, the systemic valproic acid compound may further include valproic acid.

In some embodiments, the systemic valproic acid compound may include sodium valproate and the systemic Wnt agonist is SB-415286, or a pharmaceutically acceptable salt thereof. In some embodiments, the systemic valproic acid compound may further include valproic acid.

In some embodiments, the systemic valproic acid compound may include sodium valproate and the systemic Wnt agonist is 3F8, or a pharmaceutically acceptable salt thereof. In some embodiments, the systemic valproic acid compound may further include valproic acid.

In some embodiments, the systemic valproic acid compound may include sodium valproate and the systemic Wnt agonist is TCS 21311, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor 1, or a pharmaceutically acceptable salt thereof. In some embodiments, the systemic valproic acid compound may further include valproic acid.

In some embodiments, the systemic valproic acid compound may include sodium valproate and the systemic Wnt agonist is LY2090314, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor-I, or a pharmaceutically acceptable salt thereof. In some embodiments, the systemic valproic acid compound may further include valproic acid.

In some embodiments, the systemic valproic acid compound may include sodium valproate and the systemic Wnt agonist is IM-12, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 34, or a pharmaceutically acceptable salt thereof. In some embodiments, the systemic valproic acid compound may further include valproic acid.

In some embodiments, the systemic valproic acid compound may include sodium valproate and the systemic Wnt agonist is KT 5720, or a pharmaceutically acceptable salt thereof. In some embodiments, the systemic valproic acid compound may further include valproic acid.

In some embodiments, the systemic valproic acid compound may include sodium valproate and the systemic Wnt agonist is isogranulatimide, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-30 Inhibitor XI, or a pharmaceutically acceptable salt thereof. In some embodiments, the systemic valproic acid compound may further include valproic acid.

In some embodiments, the systemic valproic acid compound may include sodium valproate and the systemic Wnt agonist is BIP-135, or a pharmaceutically acceptable salt thereof. In some embodiments, the systemic valproic acid compound may further include valproic acid.

In some embodiments, the systemic valproic acid compound may include sodium valproate and the systemic Wnt agonist is CP21R7, or a pharmaceutically acceptable salt thereof. In some embodiments, the systemic valproic acid compound may further include valproic acid.

In some embodiments, the systemic valproic acid compound may include sodium valproate and the systemic Wnt agonist is tivantinib, or a pharmaceutically acceptable salt thereof. In some embodiments, the systemic valproic acid compound may further include valproic acid.

In some embodiments, the systemic valproic acid compound may include sodium valproate and the systemic Wnt agonist is compound lambda-OS1, or a pharmaceutically acceptable salt thereof. In some embodiments, the systemic valproic acid compound may further include valproic acid.

In some embodiments, the systemic valproic acid compound may include sodium valproate and the systemic Wnt agonist is HB12, or a pharmaceutically acceptable salt thereof. In some embodiments, the systemic valproic acid compound may further include valproic acid.

In some embodiments, the systemic valproic acid compound may include sodium valproate and the systemic Wnt agonist is DW12, or a pharmaceutically acceptable salt thereof. In some embodiments, the systemic valproic acid compound may further include valproic acid.

In some embodiments, the systemic valproic acid compound may include sodium valproate and the systemic Wnt agonist is NP309, or a pharmaceutically acceptable salt thereof. In some embodiments, the systemic valproic acid compound may further include valproic acid.

In some embodiments, the systemic valproic acid compound may include sodium valproate and the systemic Wnt agonist is (RRu)-HB1229, or a pharmaceutically acceptable salt thereof. In some embodiments, the systemic valproic acid compound may further include valproic acid.

In some embodiments, the systemic valproic acid compound may include sodium valproate and the systemic Wnt agonist is (RRu)-NP549, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 3, or a pharmaceutically acceptable salt thereof. In some embodiments, the systemic valproic acid compound may further include valproic acid.

In some embodiments, the systemic valproic acid compound may include sodium valproate and the systemic Wnt agonist is compound (R)-DW12, or a pharmaceutically acceptable salt thereof. In some embodiments, the systemic valproic acid compound may further include valproic acid.

In some embodiments, the systemic valproic acid compound may include sodium valproate and the systemic Wnt agonist is staurosporine, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3β Inhibitor XXVI, or a pharmaceutically acceptable salt thereof. In some embodiments, the systemic valproic acid compound may further include valproic acid.

In some embodiments, the systemic valproic acid compound may include sodium valproate and the systemic Wnt agonist is manzamine A, or a pharmaceutically acceptable salt thereof. In some embodiments, the systemic valproic acid compound may further include valproic acid.

In some embodiments, the systemic valproic acid compound may include sodium valproate and the systemic Wnt agonist is TC-G 24, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 14d, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 15b, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 20x, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor 2, or a pharmaceutically acceptable salt thereof. In some embodiments, the systemic valproic acid compound may further include valproic acid.

In some embodiments, the systemic valproic acid compound may include sodium valproate and the systemic Wnt agonist is SU9516, or a pharmaceutically acceptable salt thereof. In some embodiments, the systemic valproic acid compound may further include valproic acid.

In some embodiments, the systemic valproic acid compound may include sodium valproate and the systemic Wnt agonist is AZD1080, or a pharmaceutically acceptable salt thereof. In some embodiments, the systemic valproic acid compound may further include valproic acid.

In some embodiments, the systemic valproic acid compound may include sodium valproate and the systemic Wnt agonist is kenpaullone, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 17b, or a pharmaceutically acceptable salt thereof. In some embodiments, the systemic valproic acid compound may further include valproic acid.

In some embodiments, the systemic valproic acid compound may include sodium valproate and the systemic Wnt agonist is azakenpaullone, or a pharmaceutically acceptable salt thereof. In some embodiments, the systemic valproic acid compound may further include valproic acid.

In some embodiments, the systemic valproic acid compound may include sodium valproate and the systemic Wnt agonist is alsterpaullone, or a pharmaceutically acceptable salt thereof. In some embodiments, the systemic valproic acid compound may further include valproic acid.

In some embodiments, the systemic valproic acid compound may include sodium valproate and the systemic Wnt agonist is alsterpaullone 2-cyanoethyl, or a pharmaceutically acceptable salt thereof. In some embodiments, the systemic valproic acid compound may further include valproic acid.

In some embodiments, the systemic valproic acid compound may include sodium valproate and the systemic Wnt agonist is cazpaullone, or a pharmaceutically acceptable salt thereof. In some embodiments, the systemic valproic acid compound may further include valproic acid.

In some embodiments, the systemic valproic acid compound may include sodium valproate and the systemic Wnt agonist is FRATtide (SEQ ID NO: 1), or a pharmaceutically acceptable salt thereof. In some embodiments, the systemic valproic acid compound may further include valproic acid.

In some embodiments, the systemic valproic acid compound may include sodium valproate and the systemic Wnt agonist is L803 (SEQ ID NO: 2), or a pharmaceutically acceptable salt thereof. In some embodiments, the systemic valproic acid compound may further include valproic acid.

In some embodiments, the systemic valproic acid compound may include sodium valproate and the systemic Wnt agonist is L803-mts (SEQ ID NO: 3), or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor XXII, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 4a, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 4t, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 4z, or a pharmaceutically acceptable salt thereof. In some embodiments, the systemic valproic acid compound may further include valproic acid.

In some embodiments, the systemic valproic acid compound may include sodium valproate and the systemic Wnt agonist is AT 7519, or a pharmaceutically acceptable salt thereof. In some embodiments, the systemic valproic acid compound may further include valproic acid.

In some embodiments, the systemic valproic acid compound may include sodium valproate and the systemic Wnt agonist is pyrazolopyridine 9, or a pharmaceutically acceptable salt thereof. In some embodiments, the systemic valproic acid compound may further include valproic acid.

In some embodiments, the systemic valproic acid compound may include sodium valproate and the systemic Wnt agonist is pyrazolopyridine 18, or a pharmaceutically acceptable salt thereof. In some embodiments, the systemic valproic acid compound may further include valproic acid.

In some embodiments, the systemic valproic acid compound may include sodium valproate and the systemic Wnt agonist is pyrazolopyridine 34, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 14, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 23, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 19, or a pharmaceutically acceptable salt thereof. In some embodiments, the systemic valproic acid compound may further include valproic acid.

In some embodiments, the systemic valproic acid compound may include sodium valproate and the systemic Wnt agonist is NSC 693868, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 150, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3 Inhibitor XIII, or a pharmaceutically acceptable salt thereof. In some embodiments, the systemic valproic acid compound may further include valproic acid.

In some embodiments, the systemic valproic acid compound may include sodium valproate and the systemic Wnt agonist is VP0.7, or a pharmaceutically acceptable salt thereof. In some embodiments, the systemic valproic acid compound may further include valproic acid.

In some embodiments, the systemic valproic acid compound may include sodium valproate and the systemic Wnt agonist is a compound having a CAS number 1132813-46-7, or a pharmaceutically acceptable salt thereof. In some embodiments, the systemic valproic acid compound may further include valproic acid.

In some embodiments, the systemic valproic acid compound may include sodium valproate and the systemic Wnt agonist is a compound having a CAS number 1132812-98-6, or a pharmaceutically acceptable salt thereof. In some embodiments, the systemic valproic acid compound may further include valproic acid.

In some embodiments, the systemic valproic acid compound may include sodium valproate and the systemic Wnt agonist is a compound having a CAS number 950727-66-9, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3β Inhibitor VII, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3β Inhibitor VI, or a pharmaceutically acceptable salt thereof. In some embodiments, the systemic valproic acid compound may further include valproic acid.

In some embodiments, the systemic valproic acid compound may include sodium valproate and the systemic Wnt agonist is palinurin, or a pharmaceutically acceptable salt thereof. In some embodiments, the systemic valproic acid compound may further include valproic acid.

In some embodiments, the systemic valproic acid compound may include sodium valproate and the systemic Wnt agonist is tricantin, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3β Inhibitor I, or a pharmaceutically acceptable salt thereof. In some embodiments, the systemic valproic acid compound may further include valproic acid.

In some embodiments, the systemic valproic acid compound may include sodium valproate and the systemic Wnt agonist is NP031115, or a pharmaceutically acceptable salt thereof. In some embodiments, the systemic valproic acid compound may further include valproic acid.

In some embodiments, the systemic valproic acid compound may include sodium valproate and the systemic Wnt agonist is NP031112, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 90, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 92, or a pharmaceutically acceptable salt thereof. In some embodiments, the systemic valproic acid compound may further include valproic acid.

In some embodiments, the systemic valproic acid compound may include sodium valproate and the systemic Wnt agonist is GSK-3β Inhibitor VIII, or a pharmaceutically acceptable salt thereof. In some embodiments, the systemic valproic acid compound may further include valproic acid.

In some embodiments, the systemic valproic acid compound may include sodium valproate and the systemic Wnt agonist is A-1070722, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 27, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 12, or a pharmaceutically acceptable salt thereof. In some embodiments, the systemic valproic acid compound may further include valproic acid.

In some embodiments, the systemic valproic acid compound may include sodium valproate and the systemic Wnt agonist is NP-103, or a pharmaceutically acceptable salt thereof. In some embodiments, the systemic valproic acid compound may further include valproic acid.

In some embodiments, the systemic valproic acid compound may include sodium valproate and the systemic Wnt agonist is CG-301338, or a pharmaceutically acceptable salt thereof. In some embodiments, the systemic valproic acid compound may further include valproic acid.

In some embodiments, the systemic valproic acid compound may include sodium valproate and the systemic Wnt agonist is XD-4241, or a pharmaceutically acceptable salt thereof. In some embodiments, the systemic valproic acid compound may further include valproic acid.

In some embodiments, the systemic valproic acid compound may include sodium valproate and the systemic Wnt agonist is CEP-16805, or a pharmaceutically acceptable salt thereof. In some embodiments, the systemic valproic acid compound may further include valproic acid.

In some embodiments, the systemic valproic acid compound may include sodium valproate and the systemic Wnt agonist is AZD13282107, or a pharmaceutically acceptable salt thereof. In some embodiments, the systemic valproic acid compound may further include valproic acid.

In some embodiments, the systemic valproic acid compound may include sodium valproate and the systemic Wnt agonist is SAR 502250, or a pharmaceutically acceptable salt thereof. In some embodiments, the systemic valproic acid compound may further include valproic acid.

In some embodiments, the systemic valproic acid compound may include sodium valproate and the systemic Wnt agonist is AR79, or a pharmaceutically acceptable salt thereof. In some embodiments, the systemic valproic acid compound may further include valproic acid.

In some embodiments, the systemic valproic acid compound may include sodium valproate and the systemic Wnt agonist is GI179186X, or a pharmaceutically acceptable salt thereof. In some embodiments, the systemic valproic acid compound may further include valproic acid.

In some embodiments, the systemic valproic acid compound may include sodium valproate and the systemic Wnt agonist is CT118637, or a pharmaceutically acceptable salt thereof. In some embodiments, the systemic valproic acid compound may further include valproic acid.

In some embodiments, the systemic valproic acid compound may include sodium valproate and the systemic Wnt agonist is CP-70949, or a pharmaceutically acceptable salt thereof. In some embodiments, the systemic valproic acid compound may further include valproic acid.

In some embodiments, the systemic valproic acid compound may include sodium valproate and the systemic Wnt agonist is GW784752X, or a pharmaceutically acceptable salt thereof. In some embodiments, the systemic valproic acid compound may further include valproic acid.

In some embodiments, the systemic valproic acid compound may include sodium valproate and the systemic Wnt agonist is GW784775X, or a pharmaceutically acceptable salt thereof. In some embodiments, the systemic valproic acid compound may further include valproic acid.

In some embodiments, the systemic valproic acid compound may include sodium valproate and the systemic Wnt agonist is CT73911, or a pharmaceutically acceptable salt thereof. In some embodiments, the systemic valproic acid compound may further include valproic acid.

In some embodiments, the systemic valproic acid compound may include sodium valproate and the systemic Wnt agonist is LY2064827, or a pharmaceutically acceptable salt thereof. In some embodiments, the systemic valproic acid compound may further include valproic acid.

In some embodiments, the systemic valproic acid compound may include sodium valproate and the systemic Wnt agonist is 705701, or a pharmaceutically acceptable salt thereof. In some embodiments, the systemic valproic acid compound may further include valproic acid.

In some embodiments, the systemic valproic acid compound may include sodium valproate and the systemic Wnt agonist is 708244, or a pharmaceutically acceptable salt thereof. In some embodiments, the systemic valproic acid compound may further include valproic acid.

In some embodiments, the systemic valproic acid compound may include sodium valproate and the systemic Wnt agonist is 709125, or a pharmaceutically acceptable salt thereof. In some embodiments, the systemic valproic acid compound may further include valproic acid.

In some embodiments, the systemic valproic acid compound may include sodium valproate and the systemic Wnt agonist is (R)-BRD4003, or a pharmaceutically acceptable salt thereof. In some embodiments, the systemic valproic acid compound may further include valproic acid.

In some embodiments, the systemic valproic acid compound may include sodium valproate and the systemic Wnt agonist is (S)-BRD4003, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 8, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 9, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 11, or a pharmaceutically acceptable salt thereof. In some embodiments, the systemic valproic acid compound may further include valproic acid.

In some embodiments, the systemic valproic acid compound may include sodium valproate and the systemic Wnt agonist is BRD1172, or a pharmaceutically acceptable salt thereof. In some embodiments, the systemic valproic acid compound may further include valproic acid.

In some embodiments, the systemic valproic acid compound may include sodium valproate and the systemic Wnt agonist is compound 16, or a pharmaceutically acceptable salt thereof. In some embodiments, the systemic valproic acid compound may further include valproic acid.

In some embodiments, the systemic valproic acid compound may include sodium valproate and the systemic Wnt agonist is BRD1652, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-1, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-2, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-3, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-4, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-5, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-6, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-7, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-8, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-9, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-10, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-11, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-12, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-13, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-14, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-15, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-16, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-17, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-18, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-19, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-20, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-21, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-22, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-23, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-24, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-25, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-26, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-27, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-28, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-29, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-30, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-31, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-32, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-33, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-34, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-35, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-36, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-37, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-38, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-39, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-40, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-41, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-42, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-43, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-44, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-45, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-46, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-47, or a pharmaceutically acceptable salt thereof. In some embodiments, the systemic valproic acid compound may further include valproic acid.

In some embodiments, the systemic valproic acid compound may include sodium valproate and the systemic Wnt agonist is Compound I-48, or a pharmaceutically acceptable salt thereof. In some embodiments, the systemic valproic acid compound may further include valproic acid.

In some embodiments, the systemic valproic acid compound may include sodium valproate and the systemic Wnt agonist is selected from Compounds I-1, I-2, I-3, I-6, I-7, I-8, I-9, I-12, I-16, I-18. I-20, I-22, I-23, I-24, I-25. I-26, I-27, I-29, I-30, I-31, I-33, I-36, I-39, I-43, I-44, and I-48, or a pharmaceutically acceptable salt thereof. In some embodiments, the systemic valproic acid compound may further include valproic acid.

Combinations with CHIR99021

In many embodiments, a) a valproic acid compound is administered systemically and b) CHIR99021 or a pharmaceutically acceptable salt thereof is administered locally in any of the methods described herein. The a) systemic and b) local administrations may occur in any order or occur simultaneously.

In further embodiments, c) a valproic acid compound is administered locally. The a) systemic, b) local, and c) local administrations may occur in any order or two or more a), b), and c) may occur simultaneously.

In some embodiments, steps a), b) and optionally c) are used to treat one or more of tinnitus, hyperacusis, vertigo, and Meniere's disease.

In some embodiments, steps a), b), and optionally c) are used to treat tinnitus.

In some embodiments, steps a), b), and optionally c) are used to treat hyperacusis.

In some embodiments, steps a), b), and optionally c) are used to treat vertigo.

In some embodiments, steps a), b), and optionally c) are used to treat Meniere's disease.

In many embodiments, a valproic acid compound is administered systemically in an amount equivalent to 0.5-25000 mg valproic acid, and CHIR99021 or a pharmaceutically acceptable salt thereof is administered locally in an amount of about 50 μg to about 1000 μg. For example, sodium valproate is administered systemically in an amount equivalent to about 50 mg to about 25000 mg valproic acid, and CHIR99021 or a pharmaceutically acceptable salt thereof is administered locally in an amount of about 125 μg to about 650 μg.

In many embodiments, a valproic acid compound is administered systemically in an amount equivalent to 0.5-25000 mg valproic acid, and CHIR99021 or a pharmaceutically acceptable salt thereof is administered locally in an amount of about 50 μg to about 1000 μg. For example, sodium valproate is administered systemically in an amount equivalent to about 50 mg to about 25000 mg valproic acid, and CHIR99021 or a pharmaceutically acceptable salt thereof is administered locally in an amount of about 125 μg to about 650 μg. In some embodiments, the administering treats tinnitus.

In many embodiments, a valproic acid compound is administered systemically in an amount equivalent to 0.5-25000 mg valproic acid, and CHIR99021 or a pharmaceutically acceptable salt thereof is administered locally in an amount of about 50 μg to about 1000 μg. For example, sodium valproate is administered systemically in an amount equivalent to about 50 mg to about 25000 mg valproic acid, and CHIR99021 or a pharmaceutically acceptable salt thereof is administered locally in an amount of about 125 μg to about 650 μg. In some embodiments, the administering treats hyperacusis.

In many embodiments, a valproic acid compound is administered systemically in an amount equivalent to 0.5-25000 mg valproic acid, and CHIR99021 or a pharmaceutically acceptable salt thereof is administered locally in an amount of about 50 μg to about 1000 μg. For example, sodium valproate is administered systemically in an amount equivalent to about 50 mg to about 25000 mg valproic acid, and CHIR99021 or a pharmaceutically acceptable salt thereof is administered locally in an amount of about 125 μg to about 650 μg. In some embodiments, the administering treats vertigo.

In many embodiments, a valproic acid compound is administered systemically in an amount equivalent to 0.5-25000 mg valproic acid, and CHIR99021 or a pharmaceutically acceptable salt thereof is administered locally in an amount of about 50 μg to about 1000 μg. For example, sodium valproate is administered systemically in an amount equivalent to about 50 mg to about 25000 mg valproic acid, and CHIR99021 or a pharmaceutically acceptable salt thereof is administered locally in an amount of about 125 μg to about 650 μg. In some embodiments, the administering treats Meniere's disease.

In many embodiments, a valproic acid compound is administered systemically in an amount equivalent to 0.5-25000 mg valproic acid, CHIR99021 or a pharmaceutically acceptable salt thereof is administered locally in an amount of about 50 μg to about 1000 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, CHIR99021 or a pharmaceutically acceptable salt thereof is administered locally in an amount of about 125 μg to about 650 μg, and sodium valproate is administered locally in an amount equivalent to about 1 mg to less than about 18 mg valproic acid.

In many embodiments, a valproic acid compound is administered systemically in an amount equivalent to 0.5-25000 mg valproic acid, CHIR99021 or a pharmaceutically acceptable salt thereof is administered locally in an amount of about 50 μg to about 1000 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, CHIR99021 or a pharmaceutically acceptable salt thereof is administered locally in an amount of about 125 μg to about 650 μg, and sodium valproate is administered locally in an amount equivalent to about 1 mg to less than about 18 mg valproic acid. In some embodiments, the administering treats tinnitus.

In many embodiments, a valproic acid compound is administered systemically in an amount equivalent to 0.5-25000 mg valproic acid, CHIR99021 or a pharmaceutically acceptable salt thereof is administered locally in an amount of about 50 μg to about 1000 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, CHIR99021 or a pharmaceutically acceptable salt thereof is administered locally in an amount of about 125 μg to about 650 μg, and sodium valproate is administered locally in an amount equivalent to about 1 mg to less than about 18 mg valproic acid. In some embodiments, the administering treats hyperacusis.

In many embodiments, a valproic acid compound is administered systemically in an amount equivalent to 0.5-25000 mg valproic acid, CHIR99021 or a pharmaceutically acceptable salt thereof is administered locally in an amount of about 50 μg to about 1000 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, CHIR99021 or a pharmaceutically acceptable salt thereof is administered locally in an amount of about 125 μg to about 650 μg, and sodium valproate is administered locally in an amount equivalent to about 1 mg to less than about 18 mg valproic acid. In some embodiments, the administering treats vertigo.

In many embodiments, a valproic acid compound is administered systemically in an amount equivalent to 0.5-25000 mg valproic acid, CHIR99021 or a pharmaceutically acceptable salt thereof is administered locally in an amount of about 50 μg to about 1000 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, CHIR99021 or a pharmaceutically acceptable salt thereof is administered locally in an amount of about 125 μg to about 650 μg, and sodium valproate is administered locally in an amount equivalent to about 1 mg to less than about 18 mg valproic acid. In some embodiments, the administering treats Meniere's disease.

In many embodiments, a valproic acid compound and CHIR99021 or a pharmaceutically acceptable salt thereof is locally administered simultaneously. In many embodiments, a valproic acid compound and CHIR99021 or a pharmaceutically acceptable salt thereof is locally administered at about the same time. For example, the locally administered valproic acid compound and CHIR99021 or a pharmaceutically acceptable salt thereof is administered sequentially in separate formulations, e.g. within minutes or about an hour of each other. In some embodiments, the valproic acid compound and CHIR99021 or a pharmaceutically acceptable salt thereof are co-formulated in a fixed-dose combination, and thus are administered simultaneously.

Combinations with LY2090314

In many embodiments, a) a valproic acid compound is administered systemically and b) LY2090314 or a pharmaceutically acceptable salt thereof is administered locally in any of the methods described herein. The a) systemic and b) local administrations may occur in any order or occur simultaneously.

In further embodiments, c) a valproic acid compound is administered locally. The a) systemic, b) local, and c) local administrations may occur in any order or two or more a), b), and c) may occur simultaneously.

In some embodiments, steps a), b) and optionally c) are used to treat one or more of tinnitus, hyperacusis, vertigo, and Meniere's disease.

In some embodiments, steps a), b), and optionally c) are used to treat tinnitus.

In some embodiments, steps a), b), and optionally c) are used to treat hyperacusis.

In some embodiments, steps a), b), and optionally c) are used to treat vertigo.

In some embodiments, steps a), b), and optionally c) are used to treat Meniere's disease.

In many embodiments, a valproic acid compound is administered systemically in an amount equivalent to 0.5-25000 mg valproic acid, and LY2090314 or a pharmaceutically acceptable salt thereof is administered locally in an amount of about 0.1 μg to about 5 μg. For example, sodium valproate is administered systemically in an amount equivalent to about 50 mg to about 25000 mg valproic acid, and LY2090314 or a pharmaceutically acceptable salt thereof is administered locally in an amount of about 0.1 μg to about 2.5 μg.

In many embodiments, a valproic acid compound is administered systemically in an amount equivalent to 0.5-25000 mg valproic acid, and LY2090314 or a pharmaceutically acceptable salt thereof is administered locally in an amount of about 0.1 μg to about 5 μg. For example, sodium valproate is administered systemically in an amount equivalent to about 50 mg to about 25000 mg valproic acid, and LY2090314 or a pharmaceutically acceptable salt thereof is administered locally in an amount of about 0.1 μg to about 2.5 μg. In some embodiments, the administering treats tinnitus.

In many embodiments, a valproic acid compound is administered systemically in an amount equivalent to 0.5-25000 mg valproic acid, and LY2090314 or a pharmaceutically acceptable salt thereof is administered locally in an amount of about 0.1 μg to about 5 μg. For example, sodium valproate is administered systemically in an amount equivalent to about 50 mg to about 25000 mg valproic acid, and LY2090314 or a pharmaceutically acceptable salt thereof is administered locally in an amount of about 0.1 μg to about 2.5 μg. In some embodiments, the administering treats hyperacusis.

In many embodiments, a valproic acid compound is administered systemically in an amount equivalent to 0.5-25000 mg valproic acid, and LY2090314 or a pharmaceutically acceptable salt thereof is administered locally in an amount of about 0.1 μg to about 5 μg. For example, sodium valproate is administered systemically in an amount equivalent to about 50 mg to about 25000 mg valproic acid, and LY2090314 or a pharmaceutically acceptable salt thereof is administered locally in an amount of about 0.1 μg to about 2.5 μg. In some embodiments, the administering treats vertigo.

In many embodiments, a valproic acid compound is administered systemically in an amount equivalent to 0.5-25000 mg valproic acid, and LY2090314 or a pharmaceutically acceptable salt thereof is administered locally in an amount of about 0.1 μg to about 5 μg. For example, sodium valproate is administered systemically in an amount equivalent to about 50 mg to about 25000 mg valproic acid, and LY2090314 or a pharmaceutically acceptable salt thereof is administered locally in an amount of about 0.1 μg to about 2.5 μg. In some embodiments, the administering treats Meniere's disease.

In many embodiments, a valproic acid compound is administered systemically in an amount equivalent to 0.5-25000 mg valproic acid, LY2090314 or a pharmaceutically acceptable salt thereof is administered locally in an amount of about 0.1 μg to about 5 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, LY2090314 or a pharmaceutically acceptable salt thereof is administered locally in an amount of about 0.1 μg to about 2.5 μg, and sodium valproate is administered locally in an amount equivalent to about 1 mg to less than about 18 mg valproic acid.

In many embodiments, a valproic acid compound is administered systemically in an amount equivalent to 0.5-25000 mg valproic acid, LY2090314 or a pharmaceutically acceptable salt thereof is administered locally in an amount of about 0.1 μg to about 5 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, LY2090314 or a pharmaceutically acceptable salt thereof is administered locally in an amount of about 0.1 μg to about 2.5 μg, and sodium valproate is administered locally in an amount equivalent to about 1 mg to less than about 18 mg valproic acid. In some embodiments, the administering treats tinnitus.

In many embodiments, a valproic acid compound is administered systemically in an amount equivalent to 0.5-25000 mg valproic acid. LY2090314 or a pharmaceutically acceptable salt thereof is administered locally in an amount of about 0.1 μg to about 5 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, LY2090314 or a pharmaceutically acceptable salt thereof is administered locally in an amount of about 0.1 μg to about 2.5 μg, and sodium valproate is administered locally in an amount equivalent to about 1 mg to less than about 18 mg valproic acid. In some embodiments, the administering treats hyperacusis.

In many embodiments, a valproic acid compound is administered systemically in an amount equivalent to 0.5-25000 mg valproic acid, LY2090314 or a pharmaceutically acceptable salt thereof is administered locally in an amount of about 0.1 μg to about 5 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, LY2090314 or a pharmaceutically acceptable salt thereof is administered locally in an amount of about 0.1 μg to about 2.5 μg, and sodium valproate is administered locally in an amount equivalent to about 1 mg to less than about 18 mg valproic acid. In some embodiments, the administering treats vertigo.

In many embodiments, a valproic acid compound is administered systemically in an amount equivalent to 0.5-25000 mg valproic acid, LY2090314 or a pharmaceutically acceptable salt thereof is administered locally in an amount of about 0.1 μg to about 5 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, LY2090314 or a pharmaceutically acceptable salt thereof is administered locally in an amount of about 0.1 μg to about 2.5 μg, and sodium valproate is administered locally in an amount equivalent to about 1 mg to less than about 18 mg valproic acid. In some embodiments, the administering treats Meniere's disease.

In many embodiments, a valproic acid compound and LY2090314 or a pharmaceutically acceptable salt thereof is locally administered simultaneously. In many embodiments, a valproic acid compound and LY2090314 or a pharmaceutically acceptable salt thereof is locally administered at about the same time. For example, the locally administered valproic acid compound and LY2090314 or a pharmaceutically acceptable salt thereof is administered sequentially in separate formulations, e.g. within minutes or about an hour of each other. In some embodiments, the valproic acid compound and LY2090314 or a pharmaceutically acceptable salt thereof are co-formulated in a fixed-dose combination, and thus are administered simultaneously.

Combinations with GSK-3 Inhibitor XVI

In many embodiments, a) a valproic acid compound is administered systemically and b) GSK-3 Inhibitor XXII or a pharmaceutically acceptable salt thereof is administered locally in any of the methods described herein. The a) systemic and b) local administrations may occur in any order or occur simultaneously.

In further embodiments, c) a valproic acid compound is administered locally. The a) systemic, b) local, and c) local administrations may occur in any order or two or more a), b), and c) may occur simultaneously.

In some embodiments, steps a), b) and optionally c) are used to treat one or more of tinnitus, hyperacusis, vertigo, and Meniere's disease.

In some embodiments, steps a), b), and optionally c) are used to treat tinnitus.

In some embodiments, steps a), b), and optionally c) are used to treat hyperacusis.

In some embodiments, steps a), b), and optionally c) are used to treat vertigo.

In some embodiments, steps a), b), and optionally c) are used to treat Meniere's disease.

In many embodiments, a valproic acid compound is administered systemically in an amount equivalent to 0.5-25000 mg valproic acid, and GSK-3 Inhibitor XXII or a pharmaceutically acceptable salt thereof is administered locally in an amount of about 1 μg to about 100 μg. For example, sodium valproate is administered systemically in an amount equivalent to about 50 mg to about 25000 mg valproic acid, and GSK-3 Inhibitor XXII or a pharmaceutically acceptable salt thereof is administered locally in an amount of about 15 μg to about 85 μg.

In many embodiments, a valproic acid compound is administered systemically in an amount equivalent to 0.5-25000 mg valproic acid, and GSK-3 Inhibitor XXII or a pharmaceutically acceptable salt thereof is administered locally in an amount of about 1 μg to about 100 μg. For example, sodium valproate is administered systemically in an amount equivalent to about 50 mg to about 25000 mg valproic acid, and GSK-3 Inhibitor XXII or a pharmaceutically acceptable salt thereof is administered locally in an amount of about 15 μg to about 85 μg. In some embodiments, the administering treats tinnitus.

In many embodiments, a valproic acid compound is administered systemically in an amount equivalent to 0.5-25000 mg valproic acid, and GSK-3 Inhibitor XXII or a pharmaceutically acceptable salt thereof is administered locally in an amount of about 1 μg to about 100 μg. For example, sodium valproate is administered systemically in an amount equivalent to about 50 mg to about 25000 mg valproic acid, and GSK-3 Inhibitor XXII or a pharmaceutically acceptable salt thereof is administered locally in an amount of about 15 μg to about 85 μg. In some embodiments, the administering treats hyperacusis.

In many embodiments, a valproic acid compound is administered systemically in an amount equivalent to 0.5-25000 mg valproic acid, and GSK-3 Inhibitor XXII or a pharmaceutically acceptable salt thereof is administered locally in an amount of about 1 μg to about 100 μg. For example, sodium valproate is administered systemically in an amount equivalent to about 50 mg to about 25000 mg valproic acid, and GSK-3 Inhibitor XXII or a pharmaceutically acceptable salt thereof is administered locally in an amount of about 15 μg to about 85 μg. In some embodiments, the administering treats vertigo.

In many embodiments, a valproic acid compound is administered systemically in an amount equivalent to 0.5-25000 mg valproic acid, and GSK-3 Inhibitor XXII or a pharmaceutically acceptable salt thereof is administered locally in an amount of about 1 μg to about 100 μg. For example, sodium valproate is administered systemically in an amount equivalent to about 50 mg to about 25000 mg valproic acid, and GSK-3 Inhibitor XXII or a pharmaceutically acceptable salt thereof is administered locally in an amount of about 15 μg to about 85 μg. In some embodiments, the administering treats Meniere's disease.

In many embodiments, a valproic acid compound is administered systemically in an amount equivalent to 0.5-25000 mg valproic acid, GSK-3 Inhibitor XXII or a pharmaceutically acceptable salt thereof is administered locally in an amount of about 1 μg to about 100 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, GSK-3 Inhibitor XXII or a pharmaceutically acceptable salt thereof is administered locally in an amount of about 15 μg to about 85 μg, and sodium valproate is administered locally in an amount equivalent to about 1 mg to less than about 18 mg valproic acid.

In many embodiments, a valproic acid compound is administered systemically in an amount equivalent to 0.5-25000 mg valproic acid, GSK-3 Inhibitor XXII or a pharmaceutically acceptable salt thereof is administered locally in an amount of about 1 μg to about 100 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, GSK-3 Inhibitor XXII or a pharmaceutically acceptable salt thereof is administered locally in an amount of about 15 μg to about 85 μg, and sodium valproate is administered locally in an amount equivalent to about 1 mg to less than about 18 mg valproic acid. In some embodiments, the administering treats tinnitus.

In many embodiments, a valproic acid compound is administered systemically in an amount equivalent to 0.5-25000 mg valproic acid, GSK-3 Inhibitor XXII or a pharmaceutically acceptable salt thereof is administered locally in an amount of about 1 μg to about 100 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, GSK-3 Inhibitor XXII or a pharmaceutically acceptable salt thereof is administered locally in an amount of about 15 μg to about 85 μg, and sodium valproate is administered locally in an amount equivalent to about 1 mg to less than about 18 mg valproic acid. In some embodiments, the administering treats hyperacusis.

In many embodiments, a valproic acid compound is administered systemically in an amount equivalent to 0.5-25000 mg valproic acid, GSK-3 Inhibitor XXII or a pharmaceutically acceptable salt thereof is administered locally in an amount of about 1 μg to about 100 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, GSK-3 Inhibitor XXII or a pharmaceutically acceptable salt thereof is administered locally in an amount of about 15 μg to about 85 μg, and sodium valproate is administered locally in an amount equivalent to about 1 mg to less than about 18 mg valproic acid. In some embodiments, the administering treats vertigo.

In many embodiments, a valproic acid compound is administered systemically in an amount equivalent to 0.5-25000 mg valproic acid, GSK-3 Inhibitor XXII or a pharmaceutically acceptable salt thereof is administered locally in an amount of about 1 μg to about 100 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, GSK-3 Inhibitor XXII or a pharmaceutically acceptable salt thereof is administered locally in an amount of about 15 μg to about 85 μg, and sodium valproate is administered locally in an amount equivalent to about 1 mg to less than about 18 mg valproic acid. In some embodiments, the administering treats Meniere's disease.

In many embodiments, a valproic acid compound and GSK-3 Inhibitor XXII or a pharmaceutically acceptable salt thereof is locally administered simultaneously. In many embodiments, a valproic acid compound and GSK-3 Inhibitor XXII or a pharmaceutically acceptable salt thereof is locally administered at about the same time. For example, the locally administered valproic acid compound and GSK-3 Inhibitor XXII or a pharmaceutically acceptable salt thereof is administered sequentially in separate formulations, e.g. within minutes or about an hour of each other. In some embodiments, the valproic acid compound and GSK-3 Inhibitor XXII or a pharmaceutically acceptable salt thereof are co-formulated in a fixed-dose combination, and thus are administered simultaneously.

Combinations with AZD1080

In many embodiments, a) a valproic acid compound is administered systemically and b) AZD1080 or a pharmaceutically acceptable salt thereof is administered locally in any of the methods described herein. The a) systemic and b) local administrations may occur in any order or occur simultaneously.

In further embodiments, c) a valproic acid compound is administered locally. The a) systemic, b) local, and c) local administrations may occur in any order or two or more a), b), and c) may occur simultaneously.

In some embodiments, steps a), b) and optionally c) are used to treat one or more of tinnitus, hyperacusis, vertigo, and Meniere's disease.

In some embodiments, steps a), b), and optionally c) are used to treat tinnitus.

In some embodiments, steps a), b), and optionally c) are used to treat hyperacusis.

In some embodiments, steps a), b), and optionally c) are used to treat vertigo.

In some embodiments, steps a), b), and optionally c) are used to treat Meniere's disease.

In many embodiments, a valproic acid compound is administered systemically in an amount equivalent to 0.5-25000 mg valproic acid, and AZD1080 or a pharmaceutically acceptable salt thereof is administered locally in an amount of about 50 μg to about 600 μg. For example, sodium valproate is administered systemically in an amount equivalent to about 50 mg to about 25000 mg valproic acid, and AZD1080 or a pharmaceutically acceptable salt thereof is administered locally in an amount of about 115 μg to about 475 μg.

In many embodiments, a valproic acid compound is administered systemically in an amount equivalent to 0.5-25000 mg valproic acid, and AZD1080 or a pharmaceutically acceptable salt thereof is administered locally in an amount of about 50 μg to about 600 μg. For example, sodium valproate is administered systemically in an amount equivalent to about 50 mg to about 25000 mg valproic acid, and AZD1080 or a pharmaceutically acceptable salt thereof is administered locally in an amount of about 115 μg to about 475 μg. In some embodiments, the administering treats tinnitus.

In many embodiments, a valproic acid compound is administered systemically in an amount equivalent to 0.5-25000 mg valproic acid, and AZD1080 or a pharmaceutically acceptable salt thereof is administered locally in an amount of about 50 μg to about 600 μg. For example, sodium valproate is administered systemically in an amount equivalent to about 50 mg to about 25000 mg valproic acid, and AZD1080 or a pharmaceutically acceptable salt thereof is administered locally in an amount of about 115 μg to about 475 μg. In some embodiments, the administering treats hyperacusis.

In many embodiments, a valproic acid compound is administered systemically in an amount equivalent to 0.5-25000 mg valproic acid, and AZD1080 or a pharmaceutically acceptable salt thereof is administered locally in an amount of about 50 μg to about 600 μg. For example, sodium valproate is administered systemically in an amount equivalent to about 50 mg to about 25000 mg valproic acid, and AZD1080 or a pharmaceutically acceptable salt thereof is administered locally in an amount of about 115 μg to about 475 μg. In some embodiments, the administering treats vertigo.

In many embodiments, a valproic acid compound is administered systemically in an amount equivalent to 0.5-25000 mg valproic acid, and AZD1080 or a pharmaceutically acceptable salt thereof is administered locally in an amount of about 50 μg to about 600 μg. For example, sodium valproate is administered systemically in an amount equivalent to about 50 mg to about 25000 mg valproic acid, and AZD1080 or a pharmaceutically acceptable salt thereof is administered locally in an amount of about 115 μg to about 475 μg. In some embodiments, the administering treats Meniere's disease.

In many embodiments, a valproic acid compound is administered systemically in an amount equivalent to 0.5-25000 mg valproic acid, AZD1080 or a pharmaceutically acceptable salt thereof is administered locally in an amount of about 50 μg to about 600 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, AZD1080 or a pharmaceutically acceptable salt thereof is administered locally in an amount of about 115 μg to about 475 μg, and sodium valproate is administered locally in an amount equivalent to about 1 mg to less than about 18 mg valproic acid.

In many embodiments, a valproic acid compound is administered systemically in an amount equivalent to 0.5-25000 mg valproic acid, AZD1080 or a pharmaceutically acceptable salt thereof is administered locally in an amount of about 50 μg to about 600 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, AZD1080 or a pharmaceutically acceptable salt thereof is administered locally in an amount of about 115 μg to about 475 μg, and sodium valproate is administered locally in an amount equivalent to about 1 mg to less than about 18 mg valproic acid. In some embodiments, the administering treats tinnitus.

In many embodiments, a valproic acid compound is administered systemically in an amount equivalent to 0.5-25000 mg valproic acid, AZD1080 or a pharmaceutically acceptable salt thereof is administered locally in an amount of about 50 μg to about 600 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, AZD1080 or a pharmaceutically acceptable salt thereof is administered locally in an amount of about 115 μg to about 475 μg, and sodium valproate is administered locally in an amount equivalent to about 1 mg to less than about 18 mg valproic acid. In some embodiments, the administering treats hyperacusis.

In many embodiments, a valproic acid compound is administered systemically in an amount equivalent to 0.5-25000 mg valproic acid, AZD1080 or a pharmaceutically acceptable salt thereof is administered locally in an amount of about 50 μg to about 600 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, AZD1080 or a pharmaceutically acceptable salt thereof is administered locally in an amount of about 115 μg to about 475 μg, and sodium valproate is administered locally in an amount equivalent to about 1 mg to less than about 18 mg valproic acid. In some embodiments, the administering treats vertigo.

In many embodiments, a valproic acid compound is administered systemically in an amount equivalent to 0.5-25000 mg valproic acid, AZD1080 or a pharmaceutically acceptable salt thereof is administered locally in an amount of about 50 μg to about 600 μg, and a valproic acid compound is administered locally in an amount equivalent to about 1 mg to less than about 18 mg of valproic acid. For example, sodium valproate is administered systemically in an amount equivalent to about 50 mg to about 25000 mg valproic acid, AZD1080 or a pharmaceutically acceptable salt thereof is administered locally in an amount of about 115 μg to about 475 μg, and sodium valproate is administered locally in an amount equivalent to about 1 mg to less than about 18 mg valproic acid. In some embodiments, the administering treats Meniere's disease.

In many embodiments, a valproic acid compound and AZD1080 or a pharmaceutically acceptable salt thereof is locally administered simultaneously. In many embodiments, a valproic acid compound and AZD1080 or a pharmaceutically acceptable salt thereof is locally administered at about the same time. For example, the locally administered valproic acid compound and AZD1080 or a pharmaceutically acceptable salt thereof is administered sequentially in separate formulations, e.g. within minutes or about an hour of each other. In some embodiments, the valproic acid compound and AZD1080 or a pharmaceutically acceptable salt thereof are co-formulated in a fixed-dose combination, and thus are administered simultaneously.

### Combinations with substituted 3-imidazo[1,2-a]pyridin-3-yl-4-(1,2,3,4-tetrahydro-[1,4]diazepino-[6,7,1-hi]indol-7-yl)pyrrole-2,5-diones

In many embodiments, a) a valproic acid compound is administered systemically and b) a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, is administered locally in any of the methods described herein. The a) systemic and b) local administrations may occur in any order or occur simultaneously.

In further embodiments, c) a valproic acid compound is administered locally. The a) systemic, b) local, and c) local administrations may occur in any order or two or more a), b), and c) may occur simultaneously.

In some embodiments, steps a), b) and optionally c) are used to treat one or more of tinnitus, hyperacusis, vertigo, and Meniere's disease.

In some embodiments, steps a), b), and optionally c) are used to treat tinnitus.

In some embodiments, steps a), b), and optionally c) are used to treat hyperacusis.

In some embodiments, steps a), b), and optionally c) are used to treat vertigo.

In some embodiments, steps a), b), and optionally c) are used to treat Meniere's disease.

In many embodiments, a valproic acid compound is administered systemically in an amount equivalent to 0.5-25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, is administered locally in an amount of about 1 μg to about 1000 μg. For example, sodium valproate is administered systemically in an amount equivalent to about 50 mg to about 25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg.

In many embodiments, a valproic acid compound is administered systemically in an amount equivalent to 0.5-25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, is administered locally in an amount of about 1 μg to about 1000 μg. For example, sodium valproate is administered systemically in an amount equivalent to about 50 mg to about 25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg. In some embodiments, the administering treats tinnitus.

In many embodiments, a valproic acid compound is administered systemically in an amount equivalent to 0.5-25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, is administered locally in an amount of about 1 μg to about 1000 μg. For example, sodium valproate is administered systemically in an amount equivalent to about 50 mg to about 25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg. In some embodiments, the administering treats hyperacusis.

In many embodiments, a valproic acid compound is administered systemically in an amount equivalent to 0.5-25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, is administered locally in an amount of about 1 μg to about 1000 μg. For example, sodium valproate is administered systemically in an amount equivalent to about 50 mg to about 25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg. In some embodiments, the administering treats vertigo.

In many embodiments, a valproic acid compound is administered systemically in an amount equivalent to 0.5-25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, is administered locally in an amount of about 1 μg to about 1000 μg. For example, sodium valproate is administered systemically in an amount equivalent to about 50 mg to about 25000 mg valproic acid, and a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg. In some embodiments, the administering treats Meniere's disease.

In many embodiments, a valproic acid compound is administered systemically in an amount equivalent to 0.5-25000 mg valproic acid, and Compound I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, or I-48, or a pharmaceutically acceptable salt thereof, is administered locally in an amount of about 1 μg to about 1000 μg. For example, sodium valproate is administered systemically in an amount equivalent to about 50 mg to about 25000 mg valproic acid, and Compound I-1, I-2, I-3, I-6. I-7, I-8. I-9, I-12, I-16, I-18, I-20, I-22, I-23, I-24, I-25, I-26, I-27, I-29, I-30, I-31, I-33, I-36, I-39, I-43, I-44, or I-48, or a pharmaceutically acceptable salt thereof, is administered locally in an amount of about 1 μg to about 1000 μg. In some embodiments, Compound I-1 is administered. In some embodiments, Compound I-2 is administered. In some embodiments, Compound I-3 is administered. In some embodiments, Compound I-6 is administered. In some embodiments, Compound I-7 is administered. In some embodiments, Compound I-8 is administered. In some embodiments, Compound I-9 is administered. In some embodiments, Compound I-12 is administered. In some embodiments, Compound I-16 is administered. In some embodiments, Compound I-18 is administered. In some embodiments, Compound I-20 is administered. In some embodiments, Compound I-22 is administered. In some embodiments, Compound I-23 is administered. In some embodiments, Compound I-24 is administered. In some embodiments, Compound I-25 is administered. In some embodiments, Compound I-26 is administered. In some embodiments, Compound I-27 is administered. In some embodiments, Compound I-29 is administered. In some embodiments, Compound I-30 is administered. In some embodiments, Compound I-31 is administered. In some embodiments, Compound I-33 is administered. In some embodiments, Compound I-36 is administered. In some embodiments, Compound I-39 is administered. In some embodiments, Compound 143 is administered. In some embodiments, Compound I-44 is administered. In some embodiments, Compound I-48 is administered. In some embodiments, the administering treats tinnitus. In some embodiments, the administering treats hyperacusis. In some embodiments, the administering treats vertigo. In some embodiments, the administering treats Meniere's disease.

In many embodiments, a valproic acid compound is administered systemically in an amount equivalent to 0.5-25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and a valproic acid compound is administered locally in an amount equivalent to about 1 mg to less than about 18 mg of valproic acid. For example, sodium valproate is administered systemically in an amount equivalent to 50 mg to about 25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and sodium valproate is administered locally in an amount equivalent to 1 mg to about 12 mg valproic acid.

In many embodiments, a valproic acid compound is administered systemically in an amount equivalent to 0.5-25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and a valproic acid compound is administered locally in an amount equivalent to about 1 mg to less than about 18 mg of valproic acid. For example, sodium valproate is administered systemically in an amount equivalent to 50 mg to about 25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and sodium valproate is administered locally in an amount equivalent to 1 mg to about 12 mg valproic acid. In some embodiments, the administering treats tinnitus.

In many embodiments, a valproic acid compound is administered systemically in an amount equivalent to 0.5-25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and a valproic acid compound is administered locally in an amount equivalent to about 1 mg to less than about 18 mg of valproic acid. For example, sodium valproate is administered systemically in an amount equivalent to 50 mg to about 25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and sodium valproate is administered locally in an amount equivalent to 1 mg to about 12 mg valproic acid. In some embodiments, the administering treats hyperacusis.

In many embodiments, a valproic acid compound is administered systemically in an amount equivalent to 0.5-25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and a valproic acid compound is administered locally in an amount equivalent to about 1 mg to less than about 18 mg of valproic acid. For example, sodium valproate is administered systemically in an amount equivalent to 50 mg to about 25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and sodium valproate is administered locally in an amount equivalent to 1 mg to about 12 mg valproic acid. In some embodiments, the administering treats vertigo.

In many embodiments, a valproic acid compound is administered systemically in an amount equivalent to 0.5-25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and a valproic acid compound is administered locally in an amount equivalent to about 1 mg to less than about 18 mg of valproic acid. For example, sodium valproate is administered systemically in an amount equivalent to 50 mg to about 25000 mg valproic acid, a substituted 3-imidazo[1,2-a]pyridin-3-yl-4-(1,2,3,4-tetrahydro-[1,4]diazepino-[6,7,1-hi]indol-7-yl)pyrrole-2,5-dione is administered locally in an amount of about 1 μg to about 1000 μg, and sodium valproate is administered locally in an amount equivalent to 1 mg to about 12 mg valproic acid. In some embodiments, the administering treats Meniere's disease.

In many embodiments, a valproic acid compound is administered systemically in an amount equivalent to 0.5-25000 mg valproic acid. Compound I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, or I-48, or a pharmaceutically acceptable salt thereof, is administered locally in an amount of about 1 μg to about 1000 μg, and a valproic acid compound is administered locally in an amount equivalent to about 1 mg to less than about 18 mg of valproic acid. For example, sodium valproate is administered systemically in an amount equivalent to 50 mg to about 25000 mg valproic acid, Compound I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, or I-48, or a pharmaceutically acceptable salt thereof, is administered locally in an amount of about 1 μg to about 1000 μg, and sodium valproate is administered locally in an amount equivalent to 1 mg to about 12 mg valproic acid. In some embodiments, Compound I-1 is administered. In some embodiments, Compound I-2 is administered. In some embodiments, Compound I-3 is administered. In some embodiments, Compound I-6 is administered. In some embodiments, Compound I-7 is administered. In some embodiments, Compound I-8 is administered. In some embodiments, Compound I-9 is administered. In some embodiments, Compound I-12 is administered. In some embodiments, Compound I-16 is administered. In some embodiments, Compound I-18 is administered. In some embodiments, Compound I-20 is administered. In some embodiments, Compound I-22 is administered. In some embodiments, Compound I-23 is administered. In some embodiments, Compound I-24 is administered. In some embodiments, Compound I-25 is administered. In some embodiments, Compound I-26 is administered. In some embodiments, Compound I-27 is administered. In some embodiments, Compound I-29 is administered. In some embodiments, Compound I-30 is administered. In some embodiments, Compound I-31 is administered. In some embodiments, Compound I-33 is administered. In some embodiments, Compound I-36 is administered. In some embodiments, Compound I-39 is administered. In some embodiments, Compound I-43 is administered. In some embodiments, Compound I-44 is administered. In some embodiments, Compound I-48 is administered. In some embodiments, the administering treats tinnitus. In some embodiments, the administering treats hyperacusis. In some embodiments, the administering treats vertigo. In some embodiments, the administering treats Meniere's disease.

In many embodiments, a valproic acid compound and a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-30, is locally administered simultaneously. In many embodiments, a valproic acid compound and Wnt agonist is locally administered at about the same time. For example, the locally administered valproic acid compound and a substituted 3-imidazo[1,2-a]pyridin-3-yl-4-(1,2,3,4-tetrahydro-[1,4]diazepino-[6,7,1-hi]indol-7-yl)pyrrole-2,5-dione is administered sequentially in separate formulations, e.g. within minutes or about an hour of each other. In some embodiments, the valproic acid compound and a substituted 3-imidazo[l1,2-a]pyridin-3-yl-4-(1,2,3,4-tetrahydro-[1,4]diazepino-[6,7,1-hi]indol-7-yl)pyrrole-2,5-dione are co-formulated in a fixed-dose combination, and thus are administered simultaneously.

### Kits

### Multiple Pharmaceutical Compositions

In many embodiments, a kit is provided. The kit may include a first pharmaceutical composition including a valproic acid compound described herein, a second pharmaceutical composition including a Wnt agonist described herein, and a set of instructions. The set of instructions may direct a user to a) systemically administer the first pharmaceutical composition to a subject, and b) locally administer the second pharmaceutical composition to the middle or inner ear of the subject. The set of instructions may direct the user to administer steps a) and b) in any order or administer steps a) and b) simultaneously. Alternatively, the set of instructions may direct a user to a) locally administer the first pharmaceutical composition to a subject, and b) locally administer the second pharmaceutical composition to the middle or inner ear of the subject, in combination with a systemically administered valproic acid compound. The set of instructions may direct the user to administer steps a) and b) in any order or administer steps a) and b) simultaneously.

In many embodiments, the first pharmaceutical composition may include a valproic acid compound in an amount equivalent to 0.5-25000 mg valproic acid.

In some embodiments, the first pharmaceutical composition may include a valproic acid compound in an amount equivalent to about 1 mg to less than about 18 mg valproic acid.

In many embodiments, the second pharmaceutical composition may include a Wnt agonist in about 0.1 μg to about 500 mg.

In some embodiments, the first pharmaceutical composition may include a valproic acid compound in an amount equivalent to 0.5-25000 mg valproic acid, and the second pharmaceutical composition may include a Wnt agonist in about 0.1 μg to about 500 mg.

In some embodiments, the first pharmaceutical composition may include a valproic acid compound in an amount equivalent to about 1 mg to less than about 18 mg valproic acid, and the second pharmaceutical composition may include a Wnt agonist in about 0.1 μg to about 500 mg.

In some embodiments, the kit may include a third pharmaceutical composition including a valproic acid compound described herein, and the set of instructions may direct the user to c) locally administer the third pharmaceutical composition to the middle or inner ear of the subject. The set of instructions may direct the user to administer steps a), b), and c) in any order or administer two or more steps simultaneously.

In some embodiments, the third pharmaceutical composition may include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include a Wnt agonist in about 0.1 μg to about 500 mg, and the third pharmaceutical composition may include a valproic acid compound in an amount equivalent to about 1 mg to less than about 18 mg valproic acid.

In some embodiments, the second pharmaceutical composition may further include a valproic acid compound described herein.

In some embodiments, the second pharmaceutical composition may further include a valproic acid compound in an amount equivalent to about 1 mg to less than about 18 mg valproic acid. In some embodiments, the second pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include a Wnt agonist in about 0.1 μg to about 500 mg, and the second pharmaceutical composition may further include a valproic acid compound in an amount equivalent to about 1 mg to less than about 18 mg valproic acid.

In many embodiments, the set of instructions may further direct a user to administer steps a) and b) according to any dosing regimen described herein.

In many embodiments, the set of instructions may further direct a user to administer steps a), b), and c) according to any dosing regimen described herein.

### Single Pharmaceutical Composition

In many embodiments, a kit is provided. The kit may include a pharmaceutical composition including a valproic acid compound described herein, a Wnt agonist described herein, and a set of instructions. The set of instructions may direct a user to locally administer the pharmaceutical composition to the middle or inner ear of a subject.

In many embodiments, the pharmaceutical composition may include a valproic acid compound in an amount equivalent to about 1 mg to less than about 18 mg valproic acid. In many embodiments, the pharmaceutical composition may include a Wnt agonist in about 0.1 μg to about 500 mg.

In many embodiments, the set of instructions may further direct a user to administer according to any dosing regimen described herein.

### CHIR99021 Kits

In many embodiments, a kit is provided. The kit may include a first pharmaceutical composition including a valproic acid compound described herein, a second pharmaceutical composition including CHIR99021 or a pharmaceutically acceptable salt thereof, and a set of instructions. The set of instructions may direct a user to a) systemically administer the first pharmaceutical composition to a subject, and b) locally administer the second pharmaceutical composition to the middle or inner ear of the subject. The set of instructions may direct the user to administer steps a) and b) in any order or administer steps a) and b) simultaneously. Alternatively, the set of instructions may direct a user to a) locally administer the first pharmaceutical composition to a subject, and b) locally administer the second pharmaceutical composition to the middle or inner ear of the subject, in combination with a systemically administered valproic acid compound. The set of instructions may direct the user to administer steps a) and b) in any order or administer steps a) and b) simultaneously.

In many embodiments, the first pharmaceutical composition may include a valproic acid compound in an amount equivalent to 0.5-25000 mg valproic acid.

In some embodiments, the first pharmaceutical composition may include a valproic acid compound in an amount equivalent to about 1 mg to less than about 18 mg valproic acid. In some embodiments, the first pharmaceutical composition may further include a valproic acid compound in an amount equivalent to about 1 mg to about 12 mg valproic acid.

In many embodiments, the second pharmaceutical composition may include CHIR99021, or a pharmaceutically acceptable salt thereof, in about 50 μg to about 1000 μg. In some embodiments, the second pharmaceutical composition may include CHIR99021, or a pharmaceutically acceptable salt thereof, in about 125 μg to about 650 μg.

In some embodiments, the first pharmaceutical composition may include a valproic acid compound in an amount equivalent to 0.5-25000 mg valproic acid, and the second pharmaceutical composition may include may include CHIR99021, or a pharmaceutically acceptable salt thereof, in about 50 μg to about 1000 μg.

In some embodiments, the first pharmaceutical composition may include a valproic acid compound in an amount equivalent to 0.5-25000 mg valproic acid, and the second pharmaceutical composition may include may include CHIR99021, or a pharmaceutically acceptable salt thereof, in about 125 μg to about 650 μg.

In some embodiments, the first pharmaceutical composition may include a valproic acid compound in an amount equivalent to about 1 mg to less than about 18 mg valproic acid, and the second pharmaceutical composition may include CHIR99021, or a pharmaceutically acceptable salt thereof, in about 50 μg to about 1000 μg.

In some embodiments, the first pharmaceutical composition may include a valproic acid compound in an amount equivalent to about 1 mg to less than about 18 mg valproic acid, and the second pharmaceutical composition may include CHIR99021, or a pharmaceutically acceptable salt thereof, in about 125 μg to about 650 μg.

In some embodiments, the kit may include a third pharmaceutical composition including a valproic acid compound described herein, and the set of instructions may direct the user to c) locally administer the third pharmaceutical composition to the middle or inner ear of the subject. The set of instructions may direct the user to administer steps a), b), and c) in any order or administer two or more steps simultaneously.

In some embodiments, the third pharmaceutical composition may include a valproic acid compound in an amount equivalent to about 1 mg to less than about 18 mg valproic acid. In some embodiments, the third pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include CHIR99021, or a pharmaceutically acceptable salt thereof, in about 50 μg to about 1000 μg, and the third pharmaceutical composition may include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include CHIR99021, or a pharmaceutically acceptable salt thereof, in about 125 μg to about 650 μg, and the third pharmaceutical composition may include a valproic acid compound in an amount equivalent to about 1 mg to less than about 18 mg valproic acid.

In some embodiments, the second pharmaceutical composition may further include a valproic acid compound described herein.

In some embodiments, the second pharmaceutical composition may further include a valproic acid compound in an amount equivalent to about 1 mg to less than about 18 mg valproic acid. In some embodiments, the second pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include CHIR99021, or a pharmaceutically acceptable salt thereof, in about 50 μg to about 1000 μg, and the second pharmaceutical composition may further include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include CHIR99021, or a pharmaceutically acceptable salt thereof, in about 125 μg to about 650 μg, and the second pharmaceutical composition may further include a valproic acid compound in an amount equivalent to about 1 mg to less than about 18 mg valproic acid.

In many embodiments, the set of instructions may further direct a user to administer steps a) and b) according to any dosing regimen described herein.

In many embodiments, the set of instructions may further direct a user to administer steps a), b), and c) according to any dosing regimen described herein.

In many embodiments, a kit is provided. The kit may include a pharmaceutical composition including a valproic acid compound described herein, CHIR99021, or a pharmaceutically acceptable salt thereof, and a set of instructions. The set of instructions may direct a user to locally administer the pharmaceutical composition to the middle or inner ear of a subject.

In many embodiments, the pharmaceutical composition may include a valproic acid compound in an amount equivalent to about 1 mg to less than about 18 mg valproic acid. In many embodiments, the pharmaceutical composition may include CHIR99021, or a pharmaceutically acceptable salt thereof, in an amount of in about 50 μg to about 1000 μg.

In many embodiments, the pharmaceutical composition may include a valproic acid compound in an amount equivalent to about 1 mg to less than about 18 mg valproic acid. In many embodiments, the pharmaceutical composition may include CHIR99021, or a pharmaceutically acceptable salt thereof, in about 125 μg to about 650 μg.

In many embodiments, the set of instructions may further direct a user to administer according to any dosing regimen described herein.

### LY2090314 Kits

In many embodiments, a kit is provided. The kit may include a first pharmaceutical composition including a valproic acid compound described herein, a second pharmaceutical composition including LY2090314 or a pharmaceutically acceptable salt thereof, and a set of instructions. The set of instructions may direct a user to a) systemically administer the first pharmaceutical composition to a subject, and b) locally administer the second pharmaceutical composition to the middle or inner ear of the subject. The set of instructions may direct the user to administer steps a) and b) in any order or administer steps a) and b) simultaneously. Alternatively, the set of instructions may direct a user to a) locally administer the first pharmaceutical composition to a subject, and b) locally administer the second pharmaceutical composition to the middle or inner ear of the subject, in combination with a systemically administered valproic acid compound. The set of instructions may direct the user to administer steps a) and b) in any order or administer steps a) and b) simultaneously.

In many embodiments, the first pharmaceutical composition may include a valproic acid compound in an amount equivalent to 0.5-25000 mg valproic acid.

In some embodiments, the first pharmaceutical composition may include a valproic acid compound in an amount equivalent to about 1 mg to less than about 18 mg valproic acid. In some embodiments, the second pharmaceutical composition may further include a valproic acid compound in an amount equivalent to about 1 mg to about 12 mg valproic acid.

In many embodiments, the second pharmaceutical composition may include a LY2090314, or a pharmaceutically acceptable salt thereof, in an amount of about 0.1 μg to about 5 μg. In some embodiments, the second pharmaceutical composition may include a LY2090314, or a pharmaceutically acceptable salt thereof, in an amount of about 0.1 μg to about 2.5 μg.

In some embodiments, the first pharmaceutical composition may include a valproic acid compound in an amount equivalent to 0.5-25000 mg valproic acid, and the second pharmaceutical composition may include LY2090314, or a pharmaceutically acceptable salt thereof, in an amount of about 0.1 μg to about 5 μg.

In some embodiments, the first pharmaceutical composition may include a valproic acid compound in an amount equivalent to 0.5-25000 mg valproic acid, and the second pharmaceutical composition may include LY2090314, or a pharmaceutically acceptable salt thereof, in an amount of about 0.1 μg to about 2.5 μg.

In some embodiments, the first pharmaceutical composition may include a valproic acid compound in an amount equivalent to about 1 mg to less than about 18 mg valproic acid, and the second pharmaceutical composition may include LY2090314, or a pharmaceutically acceptable salt thereof, in an amount of about 0.1 μg to about 5 μg.

In some embodiments, the first pharmaceutical composition may include a valproic acid compound in an amount equivalent to about 1 mg to less than about 18 mg valproic acid, and the second pharmaceutical composition may include LY2090314, or a pharmaceutically acceptable salt thereof, in an amount of about 0.1 μg to about 2.5 μg.

In some embodiments, the kit may include a third pharmaceutical composition including a valproic acid compound described herein, and the set of instructions may direct the user to c) locally administer the third pharmaceutical composition to the middle or inner ear of the subject. The set of instructions may direct the user to administer steps a), b), and c) in any order or administer two or more steps simultaneously.

In some embodiments, the third pharmaceutical composition may include a valproic acid compound in an amount equivalent to about 1 mg to less than about 18 mg valproic acid. In some embodiments, the third pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include LY2090314, or a pharmaceutically acceptable salt thereof, in an amount of about 0.1 μg to about 5 μg, and the third pharmaceutical composition may include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include LY2090314, or a pharmaceutically acceptable salt thereof, in an amount of about 0.1 μg to about 2.5 μg, and the third pharmaceutical composition may include a valproic acid compound in an amount equivalent to about 1 mg to less than about 18 mg valproic acid.

In some embodiments, the second pharmaceutical composition may further include a valproic acid compound described herein.

In some embodiments, the second pharmaceutical composition may further include a valproic acid compound in an amount equivalent to about 1 mg to less than about 18 mg valproic acid. In some embodiments, the second pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include LY2090314, or a pharmaceutically acceptable salt thereof, in an amount of about 0.1 μg to about 5 μg, and second pharmaceutical composition may further include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include LY2090314, or a pharmaceutically acceptable salt thereof, in an amount of about 0.1 μg to about 2.5 μg, and second pharmaceutical composition may further include a valproic acid compound in an amount equivalent to about 1 to mg less than about 18 mg valproic acid.

In many embodiments, the set of instructions may further direct a user to administer steps a) and b) according to any dosing regimen described herein.

In many embodiments, the set of instructions may further direct a user to administer steps a), b), and c) according to any dosing regimen described herein.

In many embodiments, a kit is provided. The kit may include a pharmaceutical composition including a valproic acid compound described herein, LY2090314, or a pharmaceutically acceptable salt thereof, and a set of instructions. The set of instructions may direct a user to locally administer the pharmaceutical composition to the middle or inner ear of a subject.

In many embodiments, the pharmaceutical composition may include a valproic acid compound in an amount equivalent to about 1 mg to less than about 18 mg valproic acid. In many embodiments, the pharmaceutical composition may include LY2090314, or a pharmaceutically acceptable salt thereof, in an amount of about 0.1 μg to about 5 μg.

In many embodiments, the pharmaceutical composition may include a valproic acid compound in an amount equivalent to about 1 mg to less than about 18 mg valproic acid. In many embodiments, the pharmaceutical composition may include LY2090314, or a pharmaceutically acceptable salt thereof, in an amount of about 0.1 μg to about 2.5 μg.

In many embodiments, the set of instructions may further direct a user to administer according to any dosing regimen described herein.

### GSK-3 Inhibitor XXII Kits

In many embodiments, a kit is provided. The kit may include a first pharmaceutical composition including a valproic acid compound described herein, a second pharmaceutical composition including GSK-3 Inhibitor XXII or a pharmaceutically acceptable salt thereof, and a set of instructions. The set of instructions may direct a user to a) systemically administer the first pharmaceutical composition to a subject, and b) locally administer the second pharmaceutical composition to the middle or inner ear of the subject. The set of instructions may direct the user to administer steps a) and b) in any order or administer steps a) and b) simultaneously. Alternatively, the set of instructions may direct a user to a) locally administer the first pharmaceutical composition to a subject, and b) locally administer the second pharmaceutical composition to the middle or inner ear of the subject, in combination with a systemically administered valproic acid compound. The set of instructions may direct the user to administer steps a) and b) in any order or administer steps a) and b) simultaneously.

In many embodiments, the first pharmaceutical composition may include a valproic acid compound in an amount equivalent to 0.5-25000 mg valproic acid.

In some embodiments, the first pharmaceutical composition may include a valproic acid compound in an amount equivalent to about 1 mg to less than about 18 mg valproic acid. In some embodiments, the first pharmaceutical composition may further include a valproic acid compound in an amount equivalent to about 1 mg to about 12 mg valproic acid.

In many embodiments, the second pharmaceutical composition may include GSK-3 Inhibitor XXII, or a pharmaceutically acceptable salt thereof, in an amount of about 1 μg to about 100 μg. In some embodiments, the second pharmaceutical composition may include GSK-3 Inhibitor XXII, or a pharmaceutically acceptable salt thereof, in an amount of about 15 μg to about 85 μg.

In some embodiments, the first pharmaceutical composition may include a valproic acid compound in an amount equivalent to 0.5-25000 mg valproic acid, and the second pharmaceutical composition may include GSK-3 Inhibitor XXII, or a pharmaceutically acceptable salt thereof, in an amount of about 1 μg to about 100 μg.

In some embodiments, the first pharmaceutical composition may include a valproic acid compound in an amount equivalent to 0.5-25000 mg valproic acid, and the second pharmaceutical composition may include GSK-3 Inhibitor XXII, or a pharmaceutically acceptable salt thereof, in about 15 μg to about 85 μg.

In some embodiments, the first pharmaceutical composition may include a valproic acid compound in an amount equivalent to about 1 mg to less than about 18 mg valproic acid, and the second pharmaceutical composition may include GSK-3 Inhibitor XXII, or a pharmaceutically acceptable salt thereof, in an amount of about 1 μg to about 100 μg.

In some embodiments, the first pharmaceutical composition may include a valproic acid compound in an amount equivalent to about 1 mg to less than about 18 mg valproic acid, and the second pharmaceutical composition may include GSK-3 Inhibitor XXII, or a pharmaceutically acceptable salt thereof, in about 15 μg to about 85 μg.

In some embodiments, the kit may include a third pharmaceutical composition including a valproic acid compound described herein, and the set of instructions may direct the user to c) locally administer the third pharmaceutical composition to the middle or inner ear of the subject. The set of instructions may direct the user to administer steps a), b), and c) in any order or administer two or more steps simultaneously.

In some embodiments, the third pharmaceutical composition may include a valproic acid compound in an amount equivalent to about 1 mg to less than about 18 mg valproic acid. In some embodiments, the third pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include GSK-3 Inhibitor XXII, or a pharmaceutically acceptable salt thereof, in an amount of about 1 μg to about 100 μg, and the third pharmaceutical composition may include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include GSK-3 Inhibitor XXII, or a pharmaceutically acceptable salt thereof, in an amount of about 15 μg to about 85 μg, and the third pharmaceutical composition may include a valproic acid compound in an amount equivalent to about 1 mg to less than about 18 mg valproic acid.

In some embodiments, the second pharmaceutical composition may further include a valproic acid compound described herein.

In some embodiments, the second pharmaceutical composition may further include a valproic acid compound in an amount equivalent to about 1 mg to less than about 18 mg valproic acid. In some embodiments, the second pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include GSK-3 Inhibitor XXII, or a pharmaceutically acceptable salt thereof, in an amount of about 1 μg to about 100 μg, and the second pharmaceutical composition may further include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include GSK-3 Inhibitor XXII, or a pharmaceutically acceptable salt thereof, in an amount of about 15 μg to about 85 μg, and the second pharmaceutical composition may further include a valproic acid compound in an amount equivalent to about 1 mg to less than about 18 mg valproic acid.

In many embodiments, the set of instructions may further direct a user to administer steps a) and b) according to any dosing regimen described herein.

In many embodiments, the set of instructions may further direct a user to administer steps a), b), and c) according to any dosing regimen described herein.

In many embodiments, a kit is provided. The kit may include a pharmaceutical composition including a valproic acid compound described herein, GSK-3 Inhibitor XXII, or a pharmaceutically acceptable salt thereof, and a set of instructions. The set of instructions may direct a user to locally administer the pharmaceutical composition to the middle or inner ear of a subject.

In many embodiments, the pharmaceutical composition may include a valproic acid compound in an amount equivalent to about 1 mg to less than about 18 mg valproic acid. In many embodiments, the pharmaceutical composition may include GSK-3 Inhibitor XXII, or a pharmaceutically acceptable salt thereof, in an amount of about 1 μg to about 100 μg.

In many embodiments, the pharmaceutical composition may include a valproic acid compound in an amount equivalent to about 1 mg to less than about 18 mg valproic acid. In many embodiments, the pharmaceutical composition may include GSK-3 Inhibitor XXII, or a pharmaceutically acceptable salt thereof, in an amount of about 15 μg to about 85 μg.

In many embodiments, the set of instructions may further direct a user to administer according to any dosing regimen described herein.

### AZD1080 Kits

In many embodiments, a kit is provided. The kit may include a first pharmaceutical composition including a valproic acid compound described herein, a second pharmaceutical composition including AZD1080 or a pharmaceutically acceptable salt thereof, and a set of instructions. The set of instructions may direct a user to a) systemically administer the first pharmaceutical composition to a subject, and b) locally administer the second pharmaceutical composition to the middle or inner ear of the subject. The set of instructions may direct the user to administer steps a) and b) in any order or administer steps a) and b) simultaneously. Alternatively, the set of instructions may direct a user to a) locally administer the first pharmaceutical composition to a subject, and b) locally administer the second pharmaceutical composition to the middle or inner ear of the subject, in combination with a systemically administered valproic acid compound. The set of instructions may direct the user to administer steps a) and b) in any order or administer steps a) and b) simultaneously.

In many embodiments, the first pharmaceutical composition may include a valproic acid compound in an amount equivalent to 0.5-25000 mg valproic acid.

In some embodiments, the first pharmaceutical composition may include a valproic acid compound in an amount equivalent to about 1 mg to less than about 18 mg valproic acid. In some embodiments, the first pharmaceutical composition may further include a valproic acid compound in an amount equivalent to about 1 mg to about 12 mg valproic acid.

In many embodiments, the second pharmaceutical composition may include AZD1080, or a pharmaceutically acceptable salt thereof, in an amount of about 50 μg to about 600 μg. In some embodiments, the second pharmaceutical composition may include AZD1080, or a pharmaceutically acceptable salt thereof, in an amount of about 115 μg to about 475 μg.

In some embodiments, the first pharmaceutical composition may include a valproic acid compound in an amount equivalent to 0.5-25000 mg valproic acid, and the second pharmaceutical composition may include AZD1080, or a pharmaceutically acceptable salt thereof, in an amount of about 50 μg to about 600 μg.

In some embodiments, the first pharmaceutical composition may include a valproic acid compound in an amount equivalent to 0.5-25000 mg valproic acid, and the second pharmaceutical composition may include AZD1080, or a pharmaceutically acceptable salt thereof, in an amount of about 115 μg to about 475 μg.

In some embodiments, the first pharmaceutical composition may include a valproic acid compound in an amount equivalent to about 1 mg to less than about 18 mg valproic acid, and the second pharmaceutical composition may include AZD1080, or a pharmaceutically acceptable salt thereof, in an amount of about 50 μg to about 600 μg.

In some embodiments, the first pharmaceutical composition may include a valproic acid compound in an amount equivalent to about 1 mg to less than about 18 mg valproic acid, and the second pharmaceutical composition may include AZD1080, or a pharmaceutically acceptable salt thereof, in an amount of about 115 μg to about 475 μg.

In some embodiments, the kit may include a third pharmaceutical composition including a valproic acid compound described herein, and the set of instructions may direct the user to c) locally administer the third pharmaceutical composition to the middle or inner ear of the subject. The set of instructions may direct the user to administer steps a), b), and c) in any order or administer two or more steps simultaneously.

In some embodiments, the third pharmaceutical composition may include a valproic acid compound in an amount equivalent to about 1 mg to less than about 18 mg valproic acid. In some embodiments, the third pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include AZD1080, or a pharmaceutically acceptable salt thereof, in an amount of about 50 μg to about 600 μg, and the third pharmaceutical composition may include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include AZD1080, or a pharmaceutically acceptable salt thereof, in an amount of about 115 μg to about 475 μg, and the third pharmaceutical composition may include a valproic acid compound in an amount equivalent to about 1 mg to less than about 18 mg valproic acid.

In some embodiments, the second pharmaceutical composition may further include a valproic acid compound described herein.

In some embodiments, the second pharmaceutical composition may further include a valproic acid compound in an amount equivalent to about 1 mg to less than about 18 mg valproic acid. In some embodiments, the second pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include AZD1080, or a pharmaceutically acceptable salt thereof, in an amount of about 50 μg to about 600 μg, and the second pharmaceutical composition may further include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include AZD1080, or a pharmaceutically acceptable salt thereof, in an amount of about 115 μg to about 475 μg, and the second pharmaceutical composition may further include a valproic acid compound in an amount equivalent to about 1 mg to less than about 18 mg valproic acid.

In many embodiments, the set of instructions may further direct a user to administer steps a) and b) according to any dosing regimen described herein.

In many embodiments, the set of instructions may further direct a user to administer steps a), b), and c) according to any dosing regimen described herein

In many embodiments, a kit is provided. The kit may include a pharmaceutical composition including a valproic acid compound described herein, GSK-3 Inhibitor XXII, or a pharmaceutically acceptable salt thereof, and a set of instructions. The set of instructions may direct a user to locally administer the pharmaceutical composition to the middle or inner ear of a subject.

In many embodiments, the pharmaceutical composition may include a valproic acid compound in an amount equivalent to about 1 mg to less than about 18 mg valproic acid. In many embodiments, the pharmaceutical composition may include GSK-3 Inhibitor XXII, or a pharmaceutically acceptable salt thereof, in an amount of about 1 μg to about 100 μg.

In many embodiments, the pharmaceutical composition may include a valproic acid compound in an amount equivalent to about 1 mg to less than about 18 mg valproic acid. In many embodiments, the pharmaceutical composition may include GSK-3 Inhibitor XXII, or a pharmaceutically acceptable salt thereof, in an amount of about 15 μg to about 85 μg.

In many embodiments, the set of instructions may further direct a user to administer according to any dosing regimen described herein.

### Substituted 3-imidazo[1,2-a]pyridin-3-yl-4-(1,2,3,4-tetrahydro-[1,4]diazepino-[6,7,1-hi]indol-7-yl)pyrrole-2,5-dione Kits

In many embodiments, a kit is provided. The kit may include a first pharmaceutical composition including a valproic acid compound described herein, a second pharmaceutical composition including a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, and a set of instructions. The set of instructions may direct a user to a) systemically administer the first pharmaceutical composition to a subject, and b) locally administer the second pharmaceutical composition to the middle or inner ear of the subject. The set of instructions may direct the user to administer steps a) and b) in any order or administer steps a) and b) simultaneously. Alternatively, the set of instructions may direct a user to a) locally administer the first pharmaceutical composition to a subject, and b) locally administer the second pharmaceutical composition to the middle or inner ear of the subject, in combination with a systemically administered valproic acid compound. The set of instructions may direct the user to administer steps a) and b) in any order or administer steps a) and b) simultaneously. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In many embodiments, the first pharmaceutical composition may include a valproic acid compound in an amount equivalent to 0.5-25000 mg valproic acid.

In some embodiments, the first pharmaceutical composition may include a valproic acid compound in an amount equivalent to about 1 mg to less than about 18 mg valproic acid. In some embodiments, the first pharmaceutical composition may further include a valproic acid compound in an amount equivalent to about 1 mg to about 12 mg valproic acid.

In many embodiments, the second pharmaceutical composition may include a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, in an amount of about 1 μg to about 1000 μg. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In some embodiments, the first pharmaceutical composition may include a valproic acid compound m an amount equivalent to 0.5-25000 mg valproic acid, and the second pharmaceutical composition may include a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, in an amount of about 1 μg to about 1000 pg. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I44, and I-48, or a pharmaceutically acceptable salt thereof.

In some embodiments, the first pharmaceutical composition may include a valproic acid compound in an amount equivalent to about 1 mg to less than about 18 mg valproic acid, and the second pharmaceutical composition may include a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, in an amount of about 1 μg to about 1000 μg. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In some embodiments, the kit may include a third pharmaceutical composition including a valproic acid compound described herein, and the set of instructions may direct the user to c) locally administer the third pharmaceutical composition to the middle or inner ear of the subject. The set of instructions may direct the user to administer steps a), b), and c) in any order or administer two or more steps simultaneously.

In some embodiments, the third pharmaceutical composition may include a valproic acid compound in an amount equivalent to about 1 mg to less than about 18 mg valproic acid. In some embodiments, the third pharmaceutical composition may further include a valproic acid compound in an amount equivalent to about 1 mg to about 12 mg valproic acid.

In some embodiments, the first pharmaceutical composition may include a valproic acid compound in an amount equivalent to 0.5-25000 mg valproic acid, and the second pharmaceutical composition may include a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, in an amount of about 1 μg to about 1000 pg, and the third pharmaceutical composition may include a valproic acid compound in an amount equivalent to about 1 mg to less than about 18 mg valproic acid. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, 143, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In some embodiments, the second pharmaceutical composition may further include a valproic acid compound described herein.

In some embodiments, the second pharmaceutical composition may further include a valproic acid compound in an amount equivalent to about 1 mg to less than about 18 mg valproic acid.

In some embodiments, the first pharmaceutical composition may include a valproic acid compound in an amount equivalent to 0.5-25000 mg valproic acid, and the second pharmaceutical composition may include a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, in an amount of about 1 μg to about 1000 pg, and the second pharmaceutical composition may further include a valproic acid compound in an amount equivalent to about 1 mg to less than about 18 mg valproic acid. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In many embodiments, the set of instructions may further direct a user to administer steps a) and b) according to any dosing regimen described herein.

In many embodiments, the set of instructions may further direct a user to administer steps a), b), and c) according to any dosing regimen described herein.

In many embodiments, a kit is provided. The kit may include a pharmaceutical composition including a valproic acid compound described herein, a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, and a set of instructions. The set of instructions may direct a user to locally administer the pharmaceutical composition to the middle or inner ear of a subject. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In many embodiments, the pharmaceutical composition may include a valproic acid compound in an amount equivalent to about 1 mg to less than about 18 mg valproic acid. In many embodiments, the pharmaceutical composition may include a substituted 3-imidazo[1,2-a]pyridin-3-yl-4-(1,2,3,4-tetrahydro-[1,4]diazepino-[6,7,1-hi]indol-7-yl)pyrrole-2,5-dione, e.g. Compounds I-1-I-48, or a pharmaceutically acceptable salt thereof, in an amount of about 1 μg to about 1000 μg. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-1. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-2. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-3. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-6. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-7. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-8. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-9. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-12. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-16. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-18. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-20. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-22. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-23. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-24. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-25. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-26. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-27. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-29. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-30. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-31. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-33. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-36. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-39. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-43. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I44. In some embodiments, a substituted 3-imidazo[1,2-a]pyridin-3-yl-4-(1,2,3,4-tetrahydro-[1,4]diazepino-[6,7,1-hi]indol-7-yl)pyrrole-2,5-dione is Compound I-48.

In many embodiments, the set of instructions may further direct a user to administer according to any dosing regimen described herein

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

In this application, the use of “or” includes “and/or” unless stated otherwise. As used in this application, the term “comprise” and variations of the term, such as “comprising” and “comprises,” are not intended to exclude other additives, components, integers or steps. By “consisting of” is meant including, and limited to, whatever follows the phrase “consisting of.” Thus, the phrase “consisting of” indicates that the listed elements are required or mandatory, and that no other elements is present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements.

The term “about” when immediately preceding a numerical value means a range of plus or minus 10% of that value, e.g. “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g. more than 49.5 to less than 52.5. Furthermore, the phrases “less than about” a value or “greater than about” a value should be understood in view of the definition of the term “about” provided herein.

The terms “about” and “approximately” are used as equivalents. Any numerals used in this disclosure with or without about/approximately are meant to cover any normal fluctuations appreciated by one of ordinary skill in the relevant art. In certain embodiments, the term “approximately” or “about” refers to a range of values that fall within 25%. 20%, 19%, 18%, 17%, 16%, 15%, 14%, 13%, 12%, 11%, 10%, 9%, 8%, 7%, 6%, 5%, 4%, 3%. 2%, 1%, or less in either direction (greater than or less than) of the stated reference value unless otherwise stated or otherwise evident from the context (except where such number would exceed 100%, of a possible value).

“Any reference to a compound is also a reference to a pharmaceutically acceptable salt of that compound (regardless of whether or not pharmaceutically acceptable salts are explicitly mentioned). Any compound can be provided for use in the invention in any pharmaceutically acceptable solid form, e.g. salt, solvate, hydrate, polymorph, amorphous material form etc. Any references to a compound also include references to artificially deuterated forms of that compound

The terms “administer,” “administering” or “administration” as used herein refer to either directly administering a compound or pharmaceutically acceptable salt of the compound or a composition to a subject. “Administration” refers to introducing a substance into a subject. In some embodiments, administration is auricular, intraauricular, intracochlear, intravestibular, or transtympanically, e.g. by injection. In some embodiments, administration is directly to the inner ear, e.g. injection through the round window, optic capsule, or vestibular canals. In some embodiments, administration is directly into the inner ear via a cochlear implant delivery system. In some embodiments, the substance is injected transtympanically to the middle ear. In certain embodiments the substance is administered systemically (e.g. orally or parenterally). In certain embodiments “causing to be administered” refers to administration of a second component after a first component has already been administered (e.g. at a different time and/or by a different actor).

“Agonist” refers to an agent that causes an increase in the expression, levels, and/or activity of a target gene, protein, and/or pathway. In some instances, an agonist directly binds to and activates a target protein. In some instances, an agonist increases the activity of a pathway by binding to and modulating the activity of one or more pathway components, for example, by inhibiting the activity of negative regulator(s) of the pathway, or by activating upstream or downstream regulator(s) of the pathway.

“Auricular administration” refers to a method of using a catheter or wick device to administer a composition across the tympanic membrane to the inner ear of the subject. To facilitate insertion of the wick or catheter, the tympanic membrane is pierced using a suitably sized syringe or pipette. The devices could also be inserted using any other methods known to those of skill in the art, e.g. surgical implantation of the device. In particular embodiments, the wick or catheter device is a stand-alone device, meaning that it is inserted into the ear of the subject and then the composition is controllably released to the inner ear. In other particular embodiments, the wick or catheter device is attached or coupled to a pump or other device that allows for the administration of additional compositions. The pump is automatically programmed to deliver dosage units or is controlled by the subject or medical professional.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

“Cochlear Concentration” as used herein will be the concentration of a given agent as measured through sampling cochlear fluid or tissue. Unless otherwise noted, the sample should contain a substantial enough portion of the cochlear fluid or tissue so that it is approximately representative of the average concentration of the agent in the cochlea. For example, samples is drawn from a vestibular canal, and a series of fluid samples drawn in series such that individual samples are comprised of cochlear fluid in specified portions of the cochlea

The phrase “daily dose” is used in the disclosure to mean the total dose of a particular active agent (e.g. valproic acid, a valproic acid compound, a Wnt agonist, etc.) administered in a 24 hour period. A daily dose may include a single dose of an active agent if administered once-daily, or is the sum total of two or more doses for active agents that are administered in two or more individual doses during a 24 hour period (e.g. measured from midnight to midnight of the following day).

The term “disease” is used in the disclosure to mean a result from a pathophysiological response to external or internal factors, and is used interchangeably with the term “disorder.”

The term “disorder” is used in this disclosure to mean a disruption to the normal or regular functions in the body or a part of the body, and is used interchangeably with, the terms condition, syndrome, or illness, unless otherwise indicated.

The terms “effective amount” and “therapeutically effective amount” are used interchangeably in this disclosure and refer to an amount of an active agent that, when administered to a subject, is capable of producing a clinically relevant reduction in one or more symptoms of a disease or disorder in a subject. The actual amount which comprises the “effective amount” or “therapeutically effective amount” may vary depending on a number of factors including, but not limited to, the severity of the disease or disorder, the size and health status of the patient, the route of administration of the active agent, and so forth. A skilled medical practitioner can readily determine the appropriate amount using methods known in the medical arts.

“intraauricular administration” refers to administration of a composition to the middle or inner ear of a subject by directly injecting the composition.

“Intracochlear” administration refers to direct injection of a composition across the tympanic membrane and across the round window membrane into the cochlea.

“Intravestibular” administration refers to direct injection of a composition across the tympanic membrane and across the round window or oval window membrane into the vestibular organs.

The phrase “local administration” or “locally administering” as used herein refers to administration of an active agent to a specific location or region of the patient's body, thereby providing a relatively localized concentration of the active agent, and typically providing a localized physiological effect. “Relatively localized” means that the active agent is distributed at the site of administration, or may diffuse or migrate from the site of administration, but is not systemically distributed. In most embodiments, the locally administered active agent, as provided herein, is deposited in the body at or near the site where the active agent provides the desired physiological effect. For example, when the active agent (e.g. a valproic acid compound or Wnt agonist as described herein) is administered locally to the inner ear of a patient, the concentration of the active agent is higher in the inner or middle ear than systemically. Alternatively, the active agent can be locally administered to the middle, whereby a therapeutically effective amount of the active agent can diffuse to the inner ear to provide the desired physiological effect.

The term “maintained” or “maintaining” a systemic plasma concentration refers to providing a systemic plasma level of a specified active agent or agents within a defined concentration range and defined time period as noted herein. For example. “maintaining” a systemic plasma level (e.g. of valproic acid) within a range of e.g. 5 μg/mL to about 500 μg/mL refers to providing a dose of a valproic acid compound to a patient whereby the systemic plasma level of valproic acid in the patient falls within 5 μg/mL to about 500 μg/mL for a clinically relevant period of time (e.g. 1, 2, 3, 4, 5, 6, 7, 8, 9, 10, 11, 12, 13, 14, 15, 16, 17, 18, 19, 20, 21, 22, 23, or 24 hours). In some embodiments, maintaining a systemic plasma level within a defined range refers to a dosing regimen whereby the patient' systemic plasma levels of the active agent fall within the defined range continuously over the course of treatment, for example over a period of days or weeks as described herein

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable salt” as used herein refers to the salt form of a basic or acidic active agent, formed by reacting the active agent with, respectively a pharmaceutically acceptable acid or base, respectively. For example, a pharmaceutically acceptable salt of valproic acid includes sodium valproate (including hemi-sodium salts), tris-valproate (the tromethamine or tris(hydroxymethyl)aminomethane salt of valproic acid). A pharmaceutically acceptable salt of a weakly basic Wnt agonist such as CHIR99021 includes acid addition salts such as the hydrochloride, hydrobromide, phosphate, sulfate, bicarbonate, citrate, acetate, maleate, malate, succinate, pamoate, oleate, etc.

“Transtympanic” administration refers to direct injection of a composition across the tympanic membrane into the middle ear.

“Treating” as used herein in connection with a cell population means delivering a substance to the population to affect an outcome. In the case of in vitro populations, the substance is directly (or even indirectly) delivered to the population. In the case of in vivo populations, the substance is delivered by administration to the host subject.

It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that is afflicted with or predisposed to the state, disorder or condition but does not yet experience or display clinical or subclinical symptoms of the state, disorder or condition, (2) inhibiting the state, disorder or condition, i.e. arresting, reducing or delaying the development of the disease or a relapse thereof (in case of maintenance treatment) or at least one clinical or subclinical symptom thereof, or (3) relieving or attenuating the disease, i.e. causing regression of the state, disorder or condition or at least one of its clinical or subclinical symptoms.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic. 3-hydroxybutyric, galactaric and galacturonic acid.

The term “subject” includes, without limitation, a human or an animal. Exemplary animals include, but are not limited to, mammals such as mouse, rat, guinea pig, dog, cat, sheep, horse, cow, pig, monkey, chimpanzee, baboon, or rhesus monkey. In certain embodiments, a mammal may include a sheep. In certain embodiments, a mammal may include a pig. In certain embodiments, a mammal is a monkey. In certain embodiments, a mammal is a baboon. In certain embodiments, a mammal is a chimpanzee. In certain embodiments, a mammal is a baboon. In certain embodiments, a mammal is a rhesus monkey. In certain embodiments, a mammal is a baboon. In certain embodiments, a mammal is a cat. In certain embodiments, a mammal is a guinea pig.

The phrase “systemic administration” or “systemically administering” as used herein refers to a mode of administration intended to deliver the active agent throughout the body. For example, most orally administered active agents ar absorbed in the gastrointestinal tract and distributed widely throughout the body (systemically) via the circulatory system. Alternatively, the active agent is administered intravenously and be distributed widely throughout the body (systemically) via the circulatory system. Systemic administration can also be achieved by depositing the active agent topically, e.g. by means of a transdermal or sublingual patch, sublingual tablet, etc., such that the active agent is transported through the skin or mucosa and into the bloodstream of the patient, whereby the active agent is distributed throughout the body.

The phrase “substantially similar therapeutic effect” refers to therapeutic effects obtained under different conditions of dosing that provide nearly the same clinical result—e.g. that differ by no more than 10%, 20%4, or 30% when measured by a relevant numerical scale, or which a clinician would classify similarly using subjective classifications known in the relevant therapeutic area.

The term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. For example, keto-enol tautomerism is a simultaneous shift of electrons and a hydrogen atom.

The phrase “therapeutic effect” as used herein refers to a clinically relevant reduction in symptoms of a disease or disorder, or a clinically relevant change in the physiological state of the patient. In most embodiments, the therapeutic effect is the effect intended by the physician to treat the disease or disorder.

The term “valproic acid compound” refers to valproic acid, salts of valproic acid (as described herein), and prodrugs of valproic acid, including esters, amides, anhydrides, and other pharmaceutically acceptable derivatives which upon administration release valproic acid (or physiological salts thereof) in the body. It is understood in the art that depending on the pH, valproic acid is present in various amounts or concentrations in its protonated form and/or as a salt thereof. Unless indicated otherwise, levels of valproic acid measured in vivo or in vitro (e.g. amounts or concentrations) are expressed as the equivalent amount of valproic acid.

The term “Wnt agonist” refers to an agent that increases the expression, levels, and/or activity of a Wnt gene, protein, or signaling pathway (e.g. TCF/LEF, Frizzled receptor family, Wifl, Lefl, Axin2, β-catenin) in a cell, for example, a cochlear cell. A Wnt agonist includes a GSK3 inhibitor, such as a GSK3-α or a GSK3-β inhibitor. Gene expression is measured using methods known in the art such as by PCR, Nanostring, immunostaining, RNAseq, RNA hybridization, or Western blot analysis.

### Enumerated Embodiments

In further embodiments, enumerated as embodiments 1-509 below, the present disclosure includes:
Embodiment 1. The method of treating or preventing a disease or disorder of the ear in a subject in need thereof, comprising:
   a) systemically administering a valproic acid compound to the subject; and
   b) locally administering a Wnt agonist to the middle or inner ear of the subject;
   wherein steps a) and b) can occur in any order or simultaneously.
Embodiment 2. The method of treating or preventing a disease or disorder of the ear in a subject in need thereof comprising a) systemically administering a valproic acid compound to the middle or inner ear of the subject, wherein the subject has been, or will be, b) administered a Wnt agonist locally to the middle or inner ear of the subject.
Embodiment 3. A method of treating or preventing a disease or disorder of the ear in a subject in need thereof comprising b) locally administering a Wnt agonist to the middle or inner ear of the subject, wherein the subject has been, or will be, a) administered a valproic acid compound systemically.
Embodiment 4. The method of any of the above enumerated embodiments wherein the a valproic acid compound is:

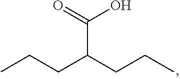

or pharmaceutically acceptable salts thereof.
Embodiment 5. The method of any of embodiments 1-3, wherein the valproic acid compound is valproic acid.
Embodiment 6. The method of any of embodiments 1-3 wherein the valproic acid compound is a valproic acid ester.
Embodiment 7. The method of any of embodiments 1-3 wherein the valproic acid compound is a valproic acid amide.
Embodiment 8. The method of any of the above enumerated embodiments wherein the valproic acid compound is administered systemically.
Embodiment 9. The method of any of the above enumerated embodiments wherein the valproic acid compound is administered systemically to the middle ear.
Embodiment 10. The method of any of the above enumerated embodiments wherein the valproic acid compound is administered systemically to the inner ear.
Embodiment 11. The method of any of the above enumerated embodiments wherein the valproic acid compound is administered orally.
Embodiment 12. The method of any of the above enumerated embodiment wherein the Wnt agonist is administered locally to middle ear of the subject.
Embodiment 13. The method of any of the above enumerated embodiment wherein the Wnt agonist is administered locally to inner ear of the subject.
Embodiment 14. The method of any of the above enumerated embodiments wherein the valproic acid compound is administered before the Wnt agonist.
Embodiment 15. The method of any of the above enumerated embodiments wherein the valproic acid compound is administered after the Wnt agonist.
Embodiment 16. The method of any of the above enumerated embodiments wherein the valproic acid compound is administered simultaneously with the Wnt agonist.
Embodiment 17. The method of any of the above enumerated embodiments wherein the valproic acid compound is administered locally to the middle ear of the subject.
Embodiment 18. The method of any of the above enumerated embodiments wherein the valproic acid compound is administered locally to the inner ear of the subject.
Embodiment 19. The method of any of the above enumerated embodiments wherein the valproic acid compound administered systemically is different to the valproic acid compound administered locally.
Embodiment 20. The method of any of the above enumerated embodiments wherein the subject has an inner ear hearing disorder.
Embodiment 21. The method of any of the above enumerated embodiments wherein the disorder is a balance disorder.
Embodiment 22. The method of any of the above enumerated embodiments wherein the disease or disorder of the ear is sensorineural hearing loss.
Embodiment 23. The method of any of the above enumerated embodiments wherein the treatment results in improved auditory function when assessed by behavioural audiometry, auditory brainstem response (ABR) testing, sound intelligibility assessments, pure tone audiometry, Distortion product otoacoustic emissions (DPOAEs), or extended high frequency (EHF) audiometry.
Embodiment 24. The method of any of the above enumerated embodiments wherein the method is for treating a subject who has hearing loss.
Embodiment 25. The method of any of the above enumerated embodiments wherein the method is for treating a subject who has reduced auditory function.
Embodiment 26. The method of any of the above enumerated embodiments wherein the method is for treating a subject who is at risk of developing hearing loss.
Embodiment 27. The method of any of the above enumerated embodiments wherein the method is for treating a subject who is at risk of developing reduced auditory function.
Embodiment 28. The method of any of the above enumerated embodiments wherein the method is for treating acute ear disease and hearing loss.
Embodiment 29. The method of any of the above enumerated embodiments wherein the method is for treating chronic ear disease and hearing loss.
Embodiment 30. The method of any of the above enumerated embodiments wherein the method is for treating dizziness and balance problems especially of sudden hearing loss.
Embodiment 31. The method of any of the above enumerated embodiments wherein the method is for treating acoustic trauma.
Embodiment 32. The method of any of the above enumerated embodiments wherein the method is for treating hearing loss due to chronic noise exposure.
Embodiment 33. The method of any of the above enumerated embodiments wherein the method is for treating presbycusis.
Embodiment 34. The method of any of the above enumerated embodiments wherein the method is for treating trauma during implantation of the inner ear prosthesis (insertion trauma).
Embodiment 35. The method of any of the above enumerated embodiments wherein the method is for treating dizziness due to diseases of the inner ear area.
Embodiment 36. The method of any of the above enumerated embodiments wherein the method is for treating dizziness related and/or as a symptom of Meniere's disease.
Embodiment 37. The method of any of the above enumerated embodiments wherein the method is for treating vertigo related and/or as a symptom of Meniere's disease.
Embodiment 38. The method of any of the above enumerated embodiments wherein the method is for treating tinnitus.
Embodiment 39. The method of any of the above enumerated embodiments wherein the method is for treating hearing loss due to antibiotics.
Embodiment 40. The method of any of the above enumerated embodiments wherein the method is for treating hearing loss due to cytostatics.
Embodiment 41. The method of any of the above enumerated embodiments wherein the method is for treating hearing loss due to other drugs.
Embodiment 42. The method of any of the above enumerated embodiments wherein the method is used to prevent, reduce or treat the incidence of inner ear disorders involving inner ear hair cells and their progenitors.
Embodiment 43. The method of any of the above enumerated embodiments wherein the method is used to prevent, reduce or treat the severity of inner ear disorders involving inner ear hair cells and their progenitors.
Embodiment 44. The method of any of the above enumerated embodiments wherein the method is used to prevent, reduce or treat the incidence of hearing impairments involving inner ear hair cells and their progenitors.
Embodiment 45. The method of any of the above enumerated embodiments wherein the method is used to prevent, reduce or treat the severity of hearing impairments involving inner ear hair cells and their progenitors.
Embodiment 46. The method of any of the above enumerated embodiments wherein the condition being treated is arising as an unwanted side-effect of ototoxic therapeutic drugs including cisplatin and its analogs.
Embodiment 47. The method of any of the above enumerated embodiments wherein the condition being treated is arising as an unwanted side-effect of ototoxic therapeutic drugs including aminoglycoside antibiotics.
Embodiment 48. The method of any of the above enumerated embodiments wherein the condition being treated is arising as an unwanted side-effect of ototoxic therapeutic drugs including salicylate and its analogs.
Embodiment 49. The method of any of the above enumerated embodiments wherein the condition being treated is arising as an unwanted side-effect of ototoxic therapeutic drugs including loop diuretics.
Embodiment 50. The method of any of the above enumerated embodiments wherein the subject experiences an improvement in hearing as measured by behavior audiometry.
Embodiment 51. The method of any of the above enumerated embodiments wherein the subject experiences an improvement in hearing as measured by auditory brainstem response (ABR) testing.
Embodiment 52. The method of any of the above enumerated embodiments comprising a pharmaceutically-acceptable salt.
Embodiment 53. The method of any of the above enumerated embodiments wherein the valproic acid compound is a pharmaceutically acceptable alkali metal salt of valproic acid.
Embodiment 54. The method of any of the above enumerated embodiments wherein the pharmaceutically acceptable salt of valproic acid is sodium valproate (including hemi-sodium salts).
Embodiment 55. The method of any of the above enumerated embodiments wherein the pharmaceutically acceptable salt of valproic acid is the tromethamine salt.
Embodiment 56. The method of any of the above enumerated embodiments wherein the pharmaceutically acceptable salt of valproic acid is the tris(hydroxymethyl)aminomethane salt.
Embodiment 57. The method of any of the above enumerated embodiments wherein the effective daily dose of locally administered valproic acid compound ranges from an amount equivalent to about 1 mg to less than about 18 mg of valproic acid.
Embodiment 58. The method of any of the above enumerated embodiments wherein the effective daily dose of locally administered valproic acid compound is an amount equivalent to about 1 mg of valproic acid.
Embodiment 59. The method of any of the above enumerated embodiments wherein the effective daily dose of locally administered valproic acid compound is an amount equivalent to about 2 mg of valproic acid.
Embodiment 60. The method of any of the above enumerated embodiments wherein the effective daily dose of locally administered valproic acid compound is an amount equivalent to about 3 mg of valproic acid.
Embodiment 61. The method of any of the above enumerated embodiments wherein the effective daily dose of locally administered valproic acid compound is an amount equivalent to about 4 mg of valproic acid.
Embodiment 62. The method of any of the above enumerated embodiments wherein the effective daily dose of locally administered valproic acid compound is an amount equivalent to about 5 mg of valproic acid.
Embodiment 63. The method of any of the above enumerated embodiments wherein the effective daily dose of locally administered valproic acid compound is an amount equivalent to about 6 mg of valproic acid.
Embodiment 64. The method of any of the above enumerated embodiments wherein the effective daily dose of locally administered valproic acid compound is an amount equivalent to about 7 mg of valproic acid.
Embodiment 65. The method of any of the above enumerated embodiments wherein the effective daily dose of locally administered valproic acid compound is an amount equivalent to about 8 mg of valproic acid.
Embodiment 66. The method of any of the above enumerated embodiments wherein the effective daily dose of locally administered valproic acid compound is an amount equivalent to about 9 mg of valproic acid.
Embodiment 67. The method of any of the above enumerated embodiments wherein the effective daily dose of locally administered valproic acid compound is an amount equivalent to about 10 mg of valproic acid.
Embodiment 68. The method of any of the above enumerated embodiments wherein the effective daily dose of locally administered valproic acid compound is an amount equivalent to about 11 mg of valproic acid.
Embodiment 69. The method of any of the above enumerated embodiments wherein the effective daily dose of locally administered valproic acid compound is an amount equivalent to about 12 mg of valproic acid.
Embodiment 70. The method of any of the above enumerated embodiments wherein the effective daily dose of locally administered valproic acid compound is an amount equivalent to about 13 mg of valproic acid.
Embodiment 71. The method of any of the above enumerated embodiments wherein the effective daily dose of locally administered valproic acid compound is an amount equivalent to about 14 mg of valproic acid.
Embodiment 72. The method of any of the above enumerated embodiments wherein the effective daily dose of locally administered valproic acid compound is an amount equivalent to about 15 mg of valproic acid.
Embodiment 73. The method of any of the above enumerated embodiments wherein the effective daily dose of locally administered valproic acid compound is an amount equivalent to about 16 mg of valproic acid.
Embodiment 74. The method of any of the above enumerated embodiments wherein the effective daily dose of locally administered valproic acid compound is an amount equivalent to about 17 mg of valproic acid.
Embodiment 75. The method of any of the above enumerated embodiments wherein the effective daily dose of locally administered valproic acid compound is an amount equivalent to less than 17.7 mg of valproic acid.
Embodiment 76. The method of any of the above enumerated embodiments wherein the effective daily dose of locally administered valproic acid compound is an amount equivalent to less than 18 mg of valproic acid.
Embodiment 77. The method of any of the above enumerated embodiments wherein the effective daily dose of locally administered valproic acid compound ranges from an amount equivalent to about 5 mg to about 10 mg of valproic acid.
Embodiment 78. The method of any of the above enumerated embodiments wherein the effective daily dose of locally administered valproic acid compound ranges from an amount equivalent to about 12 mg to about 16 mg of valproic acid.
Embodiment 79. The method of any of the above enumerated embodiments wherein the effective daily dose of locally administered valproic acid compound ranges from an amount equivalent to about 7 mg to about 14 mg of valproic acid.
Embodiment 80. The method of any of the above enumerated embodiments wherein the effective concentration of valproic acid compound (expressed as the equivalent amount of valproic acid) is about 5 mg/mL of valproic acid.
Embodiment 81. The method of any of the above enumerated embodiments wherein the effective concentration of valproic acid compound (expressed as the equivalent amount of valproic acid) is about 10 mg/mL of valproic acid.
Embodiment 82. The method of any of the above enumerated embodiments wherein the effective concentration of valproic acid compound (expressed as the equivalent amount of valproic acid) is about 15 mg/mL of valproic acid.
Embodiment 83. The method of any of the above enumerated embodiments wherein the effective concentration of valproic acid compound (expressed as the equivalent amount of valproic acid) is about 20 mg/mL of valproic acid.
Embodiment 84. The method of any of the above enumerated embodiments wherein the effective concentration of valproic acid compound (expressed as the equivalent amount of valproic acid) is about 30 mg/mL of valproic acid.
Embodiment 85. The method of any of the above enumerated embodiments wherein the effective concentration of valproic acid compound (expressed as the equivalent amount of valproic acid) is about 40 mg/mL of valproic acid.
Embodiment 86. The method of any of the above enumerated embodiments wherein the effective concentration of valproic acid compound (expressed as the equivalent amount of valproic acid) is about 50 mg/mL of valproic acid.
Embodiment 87. The method of any of the above enumerated embodiments wherein the effective concentration of valproic acid compound (expressed as the equivalent amount of valproic acid) is about 60 mg/mL of valproic acid.
Embodiment 88. The method of any of the above enumerated embodiments wherein the effective concentration of valproic acid compound (expressed as the equivalent amount of valproic acid) is about 70 mg/mL of valproic acid.
Embodiment 89. The method of any of the above enumerated embodiments wherein the effective concentration of valproic acid compound (expressed as the equivalent amount of valproic acid) is about 80 mg/mL of valproic acid.
Embodiment 90. The method of any of the above enumerated embodiments wherein the effective concentration of valproic acid compound (expressed as the equivalent amount of valproic acid) is about 90 mg/mL of valproic acid.
Embodiment 91. The method of any of the above enumerated embodiments wherein the effective concentration of valproic acid compound (expressed as the equivalent amount of valproic acid) is about 100 mg/mL of valproic acid.
Embodiment 92. The method of any of the above enumerated embodiments wherein the effective concentration of valproic acid compound (expressed as the equivalent amount of valproic acid) is about 88.6 mg/mL of valproic acid.
Embodiment 93. The method of any of the above enumerated embodiments wherein the valproic acid compound is administered systemically at a dosage containing an equivalent amount to about 50 mg of valproic acid.
Embodiment 94. The method of any of the above enumerated embodiments wherein the valproic acid compound is administered systemically at a dosage containing an equivalent amount to about 100 mg of valproic acid.
Embodiment 95. The method of any of the above enumerated embodiments wherein the valproic acid compound is administered systemically at a dosage containing an equivalent amount to about 125 mg of valproic acid.
Embodiment 96. The method of any of the above enumerated embodiments wherein the valproic acid compound is administered systemically at a dosage containing an equivalent amount to about 250 mg of valproic acid.
Embodiment 97. The method of any of the above enumerated embodiments wherein the valproic acid compound is administered systemically at a dosage containing an equivalent amount to about 500 mg of valproic acid.
Embodiment 98. The method of any of the above enumerated embodiments wherein the valproic acid compound is administered systemically at a dosage containing an equivalent amount to about 750 mg of valproic acid.
Embodiment 99. The method of any of the above enumerated embodiments wherein the valproic acid compound is administered systemically at a dosage containing an equivalent amount to about 1000 mg of valproic acid.
Embodiment 100. The method of any of the above enumerated embodiments wherein the valproic acid compound is administered systemically at a dosage containing an equivalent amount to about 2000 mg of valproic acid.
Embodiment 101. The method of any of the above enumerated embodiments wherein the valproic acid compound is administered systemically at a dosage containing an equivalent amount to about 3000 mg of valproic acid.
Embodiment 102. The method of any of the above enumerated embodiments wherein the valproic acid compound is administered systemically at a dosage containing an equivalent amount to about 4000 mg of valproic acid.
Embodiment 103. The method of any of the above enumerated embodiments wherein the valproic acid compound is administered systemically at a dosage containing an equivalent amount to about 5000 mg of valproic acid.
Embodiment 104. The method of any of the above enumerated embodiments wherein the valproic acid compound is administered systemically at a dosage containing an equivalent amount to about 10000 mg of valproic acid.
Embodiment 105. The method of any of the above enumerated embodiments wherein the valproic acid compound is administered systemically at a dosage containing an equivalent amount to about 15000 mg of valproic acid.
Embodiment 106. The method of any of the above enumerated embodiments wherein the valproic acid compound is administered systemically at a dosage containing an equivalent amount to about 20000 mg of valproic acid.
Embodiment 107. The method of any of the above enumerated embodiments wherein the valproic acid compound is administered systemically at a dosage containing an equivalent amount to about 25000 mg of valproic acid.
Embodiment 108. The method of any of the above enumerated embodiments wherein the Valproic acid compound is administered 1 day or more before the Wnt agonist.
Embodiment 109. The method of any of the above enumerated embodiments wherein the Valproic acid compound is administered 2 days or more before the Wnt agonist.
Embodiment 110. The method of any of the above enumerated embodiments wherein the Valproic acid compound is administered 3 days or more before the Wnt agonist.
Embodiment 111. The method of any of the above enumerated embodiments wherein the Valproic acid compound is administered 4 days or more before the Wnt agonist.
Embodiment 112. The method of any of the above enumerated embodiments wherein the Valproic acid compound is administered 5 days or more before the Wnt agonist.
Embodiment 113. The method of any of the above enumerated embodiments wherein the Valproic acid compound is administered 6 days or more before the Wnt agonist.
Embodiment 114. The method of any of the above enumerated embodiments wherein the Valproic acid compound is administered 7 days or more before the Wnt agonist.
Embodiment 115. The method of any of the above enumerated embodiments wherein the Valproic acid compound is administered 8 days or more before the Wnt agonist.
Embodiment 116. The method of any of the above enumerated embodiments wherein the Valproic acid compound is administered 9 days or more before the Wnt agonist.
Embodiment 117. The method of any of the above enumerated embodiments wherein the Valproic acid compound is administered 10 days or more before the Wnt agonist.
Embodiment 118. The method of any of the above enumerated embodiments wherein the Valproic acid compound is administered 11 days or more before the Wnt agonist.
Embodiment 119. The method of any of the above enumerated embodiments wherein the Valproic acid compound is administered 12 days or more before the Wnt agonist.
Embodiment 120. The method of any of the above enumerated embodiments wherein the Valproic acid compound is administered 13 days or more before the Wnt agonist.
Embodiment 121. The method of any of the above enumerated embodiments wherein the Valproic acid compound is administered 14 days or more before the Wnt agonist.
Embodiment 122. The method of any of the above enumerated embodiments wherein the local administration of the Wnt agonist is at least once or twice daily.
Embodiment 123. The method of any of the above enumerated embodiments wherein the local administration of the Wnt agonist is at least once or twice weekly.
Embodiment 124. The method of any of the above enumerated embodiments wherein the systemic delivery of the valproic acid compound occurs at least daily until 1-8 doses of the locally administered Wnt agonist are administered.
Embodiment 125. The method of any of the above enumerated embodiments wherein the Wnt agonist is administered at a concentration of about 0.01 uM to 1000 mM.
Embodiment 126. The method of any of the above enumerated embodiments wherein the Wnt agonist is administered at a concentration of about 0.1 μM to 1000 mM.
Embodiment 127. The method of any of the above enumerated embodiments wherein the Wnt agonist is administered at a concentration of about 1 μM to 100 mM.
Embodiment 128. The method of any of the above enumerated embodiments wherein the Wnt agonist is administered at a concentration of about 10 μM to 10 mM.
Embodiment 129. The method of any of the above enumerated embodiments wherein the Wnt agonist is administered at a concentration of about 1 μM to 10 μM.
Embodiment 130. The method of any of the above enumerated embodiments wherein the Wnt agonist is administered at a concentration of about 10 μM to 100 μM.
Embodiment 131. The method of any of the above enumerated embodiments wherein the Wnt agonist is administered at a concentration of about 100 μM to 1000 μM.
Embodiment 132. The method of any of the above enumerated embodiments wherein the Wnt agonist is administered at a concentration of about 1 mM to 10 mM.
Embodiment 133. The method of any of the above enumerated embodiments wherein the Wnt agonist is administered at a concentration of about 10 mM to 100 mM.
Embodiment 134. The method of any of the above enumerated embodiments wherein the Wnt agonist is administered at a concentration of about 0.1 μM.
Embodiment 135. The method of any of the above enumerated embodiments wherein the Wnt agonist is administered at a concentration of about 0.2 μM.
Embodiment 136. The method of any of the above enumerated embodiments wherein the Wnt agonist is administered at a concentration of about 0.3 μM.
Embodiment 137. The method of any of the above enumerated embodiments wherein the Wnt agonist is administered at a concentration of about 0.4 μM.
Embodiment 138. The method of any of the above enumerated embodiments wherein the Wnt agonist is administered at a concentration of about 0.5 μM.
Embodiment 139. The method of any of the above enumerated embodiments wherein the Wnt agonist is administered at a concentration of about 0.6 μM.
Embodiment 140. The method of any of the above enumerated embodiments wherein the Wnt agonist is administered at a concentration of about 0.7 μM.
Embodiment 141. The method of any of the above enumerated embodiments wherein the Wnt agonist is administered at a concentration of about 0.8 μM.
Embodiment 142. The method of any of the above enumerated embodiments wherein the Wnt agonist is administered at a concentration of about 0.9 μM.
Embodiment 143. The method of any of the above enumerated embodiments wherein the Wnt agonist is administered at a concentration of about 1 μM.
Embodiment 144. The method of any of the above enumerated embodiments wherein the Wnt agonist is administered at a concentration of about 2 μM.
Embodiment 145. The method of any of the above enumerated embodiments wherein the Wnt agonist is administered at a concentration of about 3 μM.
Embodiment 146. The method of any of the above enumerated embodiments wherein the Wnt agonist is administered at a concentration of about 4 μM.
Embodiment 147. The method of any of the above enumerated embodiments wherein the Wnt agonist is administered at a concentration of about 5 μM
Embodiment 148. The method of any of the above enumerated embodiments wherein the Wnt agonist is administered at a concentration of about 6 μM.
Embodiment 149. The method of any of the above enumerated embodiments wherein the Wnt agonist is administered at a concentration of about 7 μM.
Embodiment 150. The method of any of the above enumerated embodiments wherein the Wnt agonist is administered at a concentration of about 8 μM
Embodiment 151. The method of any of the above enumerated embodiments wherein the Wnt agonist is administered at a concentration of about 9 μM.
Embodiment 152. The method of any of the above enumerated embodiments wherein the Wnt agonist is administered at a concentration of about 10 μM.
Embodiment 153. The method of any of the above enumerated embodiments wherein the Wnt agonist is administered at a concentration of about 15 μM.
Embodiment 154. The method of any of the above enumerated embodiments wherein the Wnt agonist is administered at a concentration of about 20 μM.
Embodiment 155. The method of any of the above enumerated embodiments wherein the Wnt agonist is administered at a concentration of about 30 μM.
Embodiment 156. The method of any of the above enumerated embodiments wherein the Wnt agonist is administered at a concentration of about 50 μM.
Embodiment 157. The method of any of the above enumerated embodiments wherein the Wnt agonist is administered at a concentration of about 100 μM.
Embodiment 158. The method of any of the above enumerated embodiments wherein the Wnt agonist is administered at a concentration of about 250 μM.
Embodiment 159. The method of any of the above enumerated embodiments wherein the Wnt agonist is administered at a concentration of about 500 μM.
Embodiment 160. The method of any of the above enumerated embodiments wherein the Wnt agonist is administered at a concentration of about 100 μM.
Embodiment 161. The method of any of the above enumerated embodiments wherein the Wnt agonist is administered at a concentration of about 1500 μM.
Embodiment 162. The method of any of the above enumerated embodiments wherein the Wnt agonist is administered at a concentration of about 10 μM to 1,000,000 mM.
Embodiment 163. The method of any of the above enumerated embodiments wherein the Wnt agonist is administered at a concentration of about 1000 μM to 100.000 mM.
Embodiment 164. The method of any of the above enumerated embodiments wherein the Wnt agonist is administered at a concentration of about 10,000 μM to 10,000 mM.
Embodiment 165. The method of any of the above enumerated embodiments wherein the Wnt agonist is administered at a concentration of about 1000 μM to 10,000 μM.
Embodiment 166. The method of any of the above enumerated embodiments wherein the Wnt agonist is administered at a concentration of about 10,000 μM to 100,000 μM.
Embodiment 167. The method of any of the above enumerated embodiments wherein the Wnt agonist is administered at a concentration of about 100,000 μM to 1,000,000 μM.
Embodiment 168. The method of any of the above enumerated embodiments wherein the Wnt agonist is administered at a concentration of about 1,000 mM to 10,000 mM.
Embodiment 169. The method of any of the above enumerated embodiments wherein the Wnt agonist is administered at a concentration of about 10,000 mM to 100,000 mM.
Embodiment 170. The method of any of the above enumerated embodiments wherein the Wnt agonist is administered at a concentration of about 0.1 mM.
Embodiment 171. The method of any of the above enumerated embodiments wherein the Wnt agonist is administered at a concentration of about 0.2 mM.
Embodiment 172. The method of any of the above enumerated embodiments wherein the Wnt agonist is administered at a concentration of about 0.3 mM.
Embodiment 173. The method of any of the above enumerated embodiments wherein the Wnt agonist is administered at a concentration of about 0.4 mM.
Embodiment 174. The method of any of the above enumerated embodiments wherein the Wnt agonist is administered at a concentration of about 0.5 mM.
Embodiment 175. The method of any of the above enumerated embodiments wherein the Wnt agonist is administered at a concentration of about 0.6 mM.
Embodiment 176. The method of any of the above enumerated embodiments wherein the Wnt agonist is administered at a concentration of about 0.7 mM.
Embodiment 177. The method of any of the above enumerated embodiments wherein the Wnt agonist is administered at a concentration of about 0.8 mM.
Embodiment 178. The method of any of the above enumerated embodiments wherein the Wnt agonist is administered at a concentration of about 0.9 mM.
Embodiment 179. The method of any of the above enumerated embodiments wherein the Wnt agonist is administered at a concentration of about 1 mM.
Embodiment 180. The method of any of the above enumerated embodiments wherein the Wnt agonist is administered at a concentration of about 2 mM.
Embodiment 181. The method of any of the above enumerated embodiments wherein the Wnt agonist is administered at a concentration of about 3 mM.
Embodiment 182. The method of any of the above enumerated embodiments wherein the Wnt agonist is administered at a concentration of about 4 mM.
Embodiment 183. The method of any of the above enumerated embodiments wherein the Wnt agonist is administered at a concentration of about 5 mM.
Embodiment 184. The method of any of the above enumerated embodiments wherein the Wnt agonist is administered at a concentration of about 6 mM.
Embodiment 185. The method of any of the above enumerated embodiments wherein the Wnt agonist is administered at a concentration of about 7 mM.
Embodiment 186. The method of any of the above enumerated embodiments wherein the Wnt agonist is administered at a concentration of about 8 mM.
Embodiment 187. The method of any of the above enumerated embodiments wherein the Wnt agonist is administered at a concentration of about 9 mM.
Embodiment 188. The method of any of the above enumerated embodiments wherein the Wnt agonist is administered at a concentration of about 10 mM.
Embodiment 189. The method of any of the above enumerated embodiments wherein the Wnt agonist is administered at a concentration of about 15 nM.
Embodiment 190. The method of any of the above enumerated embodiments wherein the Wnt agonist is administered at a concentration of about 20 nM.
Embodiment 191. The method of any of the above enumerated embodiments wherein the Wnt agonist is administered at a concentration of about 0.01 μM to 1000 mM.
Embodiment 192. The method of any of the above enumerated embodiments wherein the Wnt agonist is administered at a concentration of about 0.1 μM to 1000 mM.
Embodiment 193. The method of any of the above enumerated embodiments wherein the Wnt agonist is administered at a concentration of about 1 μM to 100 mM.
Embodiment 194. The method of any of the above enumerated embodiments wherein the Wnt agonist is administered at a concentration of about 10 μM to 10 mM.
Embodiment 195. The method of any of the above enumerated embodiments wherein the Wnt agonist is administered at a concentration of about 1 μM to 10 μM.
Embodiment 196. The method of any of the above enumerated embodiments wherein the Wnt agonist is administered at a concentration of about 10 μM to 100 μM.
Embodiment 197. The method of any of the above enumerated embodiments wherein the Wnt agonist is administered at a concentration of about 100 μM to 1000 μM.
Embodiment 198. The method of any of the above enumerated embodiments wherein the Wnt agonist is administered at a concentration of about 1 mM to 10 mM.
Embodiment 199. The method of any of the above enumerated embodiments wherein the Wnt agonist is administered at a concentration of about 10 mM to 100 mM.
Embodiment 200. The method of any of the above enumerated embodiments wherein the Wnt agonist is administered at a concentration of about 1 nM.
Embodiment 201. The method of any of the above enumerated embodiments wherein the Wnt agonist is administered at a concentration of about 5 nM.
Embodiment 202. The method of any of the above enumerated embodiments wherein the Wnt agonist is administered at a concentration of about 10 nM.
Embodiment 203. The method of any of the above enumerated embodiments wherein the Wnt agonist is administered at a concentration of about 15 nM.
Embodiment 204. The method of any of the above enumerated embodiments wherein the Wnt agonist is administered at a concentration of about 20 nM.
Embodiment 205. The method of any of the above enumerated embodiments wherein the Wnt agonist is CHIR99021.
Embodiment 206. The method of any of the above enumerated embodiments wherein the Wnt agonist is a GSK-3 inhibitor.
Embodiment 207. The method of any of the above enumerated embodiments wherein the Wnt agonist is a GSK-3α inhibitor.
Embodiment 208. The method of any of the above enumerated embodiments wherein the Wnt agonist is a GSK-3 β inhibitor.
Embodiment 209. The method of any of the above enumerated embodiments wherein the agent having activity as a Wnt agonist is a Wnt agonist listed in Table 3.
Embodiment 210. The method of any of the above enumerated embodiments wherein the agent having activity as a Wnt agonist is a Wnt agonist listed in Table 4.
Embodiment 211. The method of any of the above enumerated embodiments wherein the agent having activity as a Wnt agonist is AZD1080.
Embodiment 212. The method of any of the above enumerated embodiments wherein the agent having activity as a Wnt agonist is LY2090314.
Embodiment 213. The method of any of the above enumerated embodiments wherein the agent having activity as a Wnt agonist is GSK3 inhibitor XXII.
Embodiment 214. The method of any of the above enumerated embodiments wherein the agent having activity as a Wnt agonist is CHIR99021.
Embodiment 215. The method of any of the above enumerated embodiments wherein the Wnt agonist is an analogue of LY2090314 as known in the art.
Embodiment 216. The method of any of the above enumerated embodiments wherein the Wnt agonist is Wnt-l.
Embodiment 217. The method of any of the above enumerated embodiments wherein the Wnt agonist is Wnt-2/Irp.
Embodiment 218. The method of any of the above enumerated embodiments wherein the Wnt agonist is Wnt-2b/13.
Embodiment 219. The method of any of the above enumerated embodiments wherein the Wnt agonist is Wnt-3/Int-4.
Embodiment 220. The method of any of the above enumerated embodiments wherein the Wnt agonist is Wnt-3a.
Embodiment 221. The method of any of the above enumerated embodiments wherein the Wnt agonist is Wnt-4.
Embodiment 222. The method of any of the above enumerated embodiments wherein the Wnt agonist is Wnt-5a.
Embodiment 223. The method of any of the above enumerated embodiments wherein the Wnt agonist is Wnt-5b.
Embodiment 224. The method of any of the above enumerated embodiments wherein the Wnt agonist is Wnt-6.
Embodiment 225. The method of any of the above enumerated embodiments wherein the Wnt agonist is Wnt-7a.
Embodiment 226. The method of any of the above enumerated embodiments wherein the Wnt agonist is Wnt-7b.
Embodiment 227. The method of any of the above enumerated embodiments wherein the Wnt agonist is Wnt-8a/8d.
Embodiment 228. The method of any of the above enumerated embodiments wherein the Wnt agonist is Wnt-8b.
Embodiment 229. The method of any of the above enumerated embodiments wherein the Wnt agonist is Wnt-9a/14.
Embodiment 230. The method of any of the above enumerated embodiments wherein the Wnt agonist is Wnt-9b/14b/15.
Embodiment 231. The method of any of the above enumerated embodiments wherein the Wnt agonist is Wnt-10a.
Embodiment 232. The method of any of the above enumerated embodiments wherein the Wnt agonist is Wnt-10b/12.
Embodiment 233. The method of any of the above enumerated embodiments wherein the Wnt agonist is Wnt-11.
Embodiment 234. The method of any of the above enumerated embodiments wherein the Wnt agonist is Wnt-16.
Embodiment 235. The method of any of the above enumerated embodiments wherein the Wnt agonist is R-Spondin 1/2/3/4.
Embodiment 236. The method of any of the above enumerated embodiments wherein the Wnt agonist is Norrin.
Embodiment 237. The method of any of the above enumerated embodiments wherein the Wnt agonist is BML-284.
Embodiment 238. The method of any of the above enumerated embodiments wherein the Wnt agonist is IQ 1.
Embodiment 239. The method of any of the above enumerated embodiments wherein the Wnt agonist is DCA.
Embodiment 240. The method of any of the above enumerated embodiments wherein the Wnt agonist is QS 11.
Embodiment 241. The method of any of the above enumerated embodiments wherein the Wnt agonist is WASP-1.
Embodiment 242. The method of any of the above enumerated embodiments wherein the Wnt agonist is WAY 316606.
Embodiment 243. The method of any of the above enumerated embodiments wherein the Wnt agonist is (Dimethylamino)propyl)-2-ethyl-5-(phenylsulfonyl)benzenesulfonamide.
Embodiment 244. The method of any of the above enumerated embodiments wherein the Wnt agonist is Cyclosporine A (CsA).
Embodiment 245. The method of any of the above enumerated embodiments wherein the Wnt agonist is PSC833 (Valspodar).
Embodiment 246. The method of any of the above enumerated embodiments wherein the Wnt agonist is a Cyclosporine analog.
Embodiment 247. The method of any of the above enumerated embodiments wherein the Wnt agonist is WAY-262611.
Embodiment 248. The method of any of the above enumerated embodiments wherein the Wnt agonist is HLY78.
Embodiment 249. The method of any of the above enumerated embodiments wherein the Wnt agonist is SKL2001.
Embodiment 250. The method of any of the above enumerated embodiments wherein the Wnt agonist is Cpd1.
Embodiment 251. The method of any of the above enumerated embodiments wherein the Wnt agonist is Cpd2.
Embodiment 252. The method of any of the above enumerated embodiments wherein the Wnt agonist is ISX 9.
Embodiment 253. The method of any of the above enumerated embodiments wherein the Wnt agonist is Selumetinib.
Embodiment 254. The method of any of the above enumerated embodiments wherein the Wnt agonist is Radicicol.
Embodiment 255. The method of any of the above enumerated embodiments wherein the Wnt agonist is AT 7519.
Embodiment 256. The method of any of the above enumerated embodiments wherein the Wnt agonist is AZD1080.
Embodiment 257. The method of any of the above enumerated embodiments wherein the Wnt agonist is Tivantinib.
Embodiment 258. The method of any of the above enumerated embodiments wherein the Wnt agonist is 15.
Embodiment 259. The method of any of the above enumerated embodiments wherein the Wnt agonist is BRD4003 chiral.
Embodiment 260. The method of any of the above enumerated embodiments wherein the Wnt agonist is BRD1172.
Embodiment 261. The method of any of the above enumerated embodiments wherein the Wnt agonist is BRD1652.
Embodiment 262. The method of any of the above enumerated embodiments wherein the Wnt agonist is AR-A014418.
Embodiment 263. The method of any of the above enumerated embodiments wherein the Wnt agonist is Bikinin.
Embodiment 264. The method of any of the above enumerated embodiments wherein the Wnt agonist is Hymenialdisine.
Embodiment 265. The method of any of the above enumerated embodiments wherein the Wnt agonist is Aloisine A.
Embodiment 266. The method of any of the above enumerated embodiments wherein the Wnt agonist is Aloisine B.
Embodiment 267. The method of any of the above enumerated embodiments wherein the Wnt agonist is TWS119.
Embodiment 268. The method of any of the above enumerated embodiments wherein the Wnt agonist is CT20026.
Embodiment 269. The method of any of the above enumerated embodiments wherein the Wnt agonist is CHIR99021.
Embodiment 270. The method of any of the above enumerated embodiments wherein the Wnt agonist is CHIR98014.
Embodiment 271. The method of any of the above enumerated embodiments wherein the Wnt agonist is CHIR98023.
Embodiment 272. The method of any of the above enumerated embodiments wherein the Wnt agonist is CHIR98024.
Embodiment 273. The method of any of the above enumerated embodiments wherein the Wnt agonist is CGP60474.
Embodiment 274. The method of any of the above enumerated embodiments wherein the Wnt agonist is AZD2858 (AR28).
Embodiment 275. The method of any of the above enumerated embodiments wherein the Wnt agonist is CID 755673.
Embodiment 276. The method of any of the above enumerated embodiments wherein the Wnt agonist is TCS 2002.
Embodiment 277. The method of any of the above enumerated embodiments wherein the Wnt agonist is Dibromocantharelline.
Embodiment 278. The method of any of the above enumerated embodiments wherein the Wnt agonist is ML320.
Embodiment 279. The method of any of the above enumerated embodiments wherein the Wnt agonist is Flavopiridol.
Embodiment 280. The method of any of the above enumerated embodiments wherein the Wnt agonist is Hymenidin.
Embodiment 281. The method of any of the above enumerated embodiments wherein the Wnt agonist is 6-Bromoindirubin-3-acetoxime.
Embodiment 282. The method of any of the above enumerated embodiments wherein the Wnt agonist is Indirubin-3′-monoxime.
Embodiment 283. The method of any of the above enumerated embodiments wherein the Wnt agonist is 5-lodo-indirubin-3′-monoxime.
Embodiment 284. The method of any of the above enumerated embodiments wherein the Wnt agonist is Indirubin-5-sulfonic acid sodium salt.
Embodiment 285. The method of any of the above enumerated embodiments wherein the Wnt agonist is Indirubin.
Embodiment 286. The method of any of the above enumerated embodiments wherein the Wnt agonist is Lithium Chloride.
Embodiment 287. The method of any of the above enumerated embodiments wherein the Wnt agonist is Beryllium.
Embodiment 288. The method of any of the above enumerated embodiments wherein the Wnt agonist is Zinc.
Embodiment 289. The method of any of the above enumerated embodiments wherein the Wnt agonist is Tungstate.
Embodiment 290. The method of any of the above enumerated embodiments wherein the Wnt agonist is GF109203x.
Embodiment 291. The method of any of the above enumerated embodiments wherein the Wnt agonist is Ro318220.
Embodiment 292. The method of any of the above enumerated embodiments wherein the Wnt agonist is Bisindolylmaleimide X HCl.
Embodiment 293. The method of any of the above enumerated embodiments wherein the Wnt agonist is Enzastaurin.
Embodiment 294. The method of any of the above enumerated embodiments wherein the Wnt agonist is SB-216763.
Embodiment 295. The method of any of the above enumerated embodiments wherein the Wnt agonist is SB-415286.
Embodiment 296. The method of any of the above enumerated embodiments wherein the Wnt agonist is 3F8.
Embodiment 297. The method of any of the above enumerated embodiments wherein the Wnt agonist is TCS 21311.
Embodiment 298. The method of any of the above enumerated embodiments wherein the Wnt agonist is LY2090314.
Embodiment 299. The method of any of the above enumerated embodiments wherein the Wnt agonist is IM-12.
Embodiment 300. The method of any of the above enumerated embodiments wherein the Wnt agonist is KT 5720.
Embodiment 301. The method of any of the above enumerated embodiments wherein the Wnt agonist is Isogranulatimide.
Embodiment 302. The method of any of the above enumerated embodiments wherein the Wnt agonist is BIP-135.
Embodiment 303. The method of any of the above enumerated embodiments wherein the Wnt agonist is CP21R7.
Embodiment 304. The method of any of the above enumerated embodiments wherein the Wnt agonist is HB12.
Embodiment 305. The method of any of the above enumerated embodiments wherein the Wnt agonist is DW12.
Embodiment 306. The method of any of the above enumerated embodiments wherein the Wnt agonist is NP309.
Embodiment 307. The method of any of the above enumerated embodiments wherein the Wnt agonist is (RRu)-HB1229.
Embodiment 308. The method of any of the above enumerated embodiments wherein the Wnt agonist is (RRu)-NP549.
Embodiment 309. The method of any of the above enumerated embodiments wherein the Wnt agonist is Staurosporine.
Embodiment 310. The method of any of the above enumerated embodiments wherein the Wnt agonist is Manzamine A.
Embodiment 311. The method of any of the above enumerated embodiments wherein the Wnt agonist is TC-G 24.
Embodiment 312. The method of any of the above enumerated embodiments wherein the Wnt agonist is SU9516.
Embodiment 313. The method of any of the above enumerated embodiments wherein the Wnt agonist is AZD1080.
Embodiment 314. The method of any of the above enumerated embodiments wherein the Wnt agonist is Kenpaullone.
Embodiment 315. The method of any of the above enumerated embodiments wherein the Wnt agonist is Cmpd 17b.
Embodiment 316. The method of any of the above enumerated embodiments wherein the Wnt agonist is Azakenpaullone.
Embodiment 317. The method of any of the above enumerated embodiments wherein the Wnt agonist is Alsterpaullone.
Embodiment 318. The method of any of the above enumerated embodiments wherein the Wnt agonist is Alsterpaullone CN Ethyl.
Embodiment 319. The method of any of the above enumerated embodiments wherein the Wnt agonist is Cazpaullone.
Embodiment 320. The method of any of the above enumerated embodiments wherein the Wnt agonist is FRATtide.
Embodiment 321. The method of any of the above enumerated embodiments wherein the Wnt agonist is L803.
Embodiment 322. The method of any of the above enumerated embodiments wherein the Wnt agonist is L803-mts.
Embodiment 323. The method of any of the above enumerated embodiments wherein the Wnt agonist is AT 7519.
Embodiment 324. The method of any of the above enumerated embodiments wherein the Wnt agonist is NSC 693868.
Embodiment 325. The method of any of the above enumerated embodiments wherein the Wnt agonist is VP0.7.
Embodiment 326. The method of any of the above enumerated embodiments wherein the Wnt agonist is Palinurin.
Embodiment 327. The method of any of the above enumerated embodiments wherein the Wnt agonist is Tricantin.
Embodiment 328. The method of any of the above enumerated embodiments wherein the Wnt agonist is NP031115.
Embodiment 329. The method of any of the above enumerated embodiments wherein the Wnt agonist is NP031112 (Tideglusib).
Embodiment 330. The method of any of the above enumerated embodiments wherein the Wnt agonist is AR-A014418.
Embodiment 331. The method of any of the above enumerated embodiments wherein the Wnt agonist is A-1070722.
Embodiment 332. The method of any of the above enumerated embodiments wherein the subject is human.
Embodiment 333. The method of any of the above enumerated embodiments wherein the subject is an animal other than a human.
Embodiment 334. The method of any preceding embodiment, wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 335. The method of the preceding embodiment, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is selected from those disclosed in Table 4.
Embodiment 336. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione.
Embodiment 337. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile.
Embodiment 338. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 339. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 340. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is I-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde.
Embodiment 341. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 342. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 343. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(8-oxa-3-azabicyclo[3,2,1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 344. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione.
Embodiment 345. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide.
Embodiment 346. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 347. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 348. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-((1R4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 349. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(8-oxa-3-azabicyclo[3,2,1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile.
Embodiment 350. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile.
Embodiment 351. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile.
Embodiment 352. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 353. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(8-oxa-3-azabicyclo[3,2,1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 354. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 355. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 356. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile.
Embodiment 357. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 358. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 359. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 360. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[l1,2-a]pyridin-3-yl)-1H-pyrrole-2,5-dione.
Embodiment 361. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 362. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 363. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione.
Embodiment 364. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 365. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione.
Embodiment 366. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 367. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 368. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 369. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 370. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 371. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 372. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide,
Embodiment 373. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide,
Embodiment 374. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide,
Embodiment 375. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 376. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 377. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 378. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione.
Embodiment 379. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 380. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4-(dimethylamino)piperidine-1-carbonyl)-7-(4-(imidazo[1,2-a]pyridin-3-yl)-2,5-dioxo-2,5-dihydro-I H-pyrrol-3-yl)-1,2,3,4-tetrahydro-[1,4]diazepino[6,7,1-hi]indole-9-carbonitrile.
Embodiment 381. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide.
Embodiment 382. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile.
Embodiment 383. A valproic acid compound and a Wnt agonist for use in a method of treating or preventing a disease or disorder of the ear in a subject in need thereof, said method comprising:
   a) systemically administering the valproic acid compound to the subject; and
   b) locally administering the Wnt agonist to the middle or inner ear of the subject,
   wherein steps a) and b) can occur in any order or simultaneously.
Embodiment 384. A valproic acid compound for use in a method of treating or preventing a disease or disorder of the ear in a subject in need thereof, said method comprising systemically administering the valproic acid compound, wherein the subject has been, or will be, administered a Wnt agonist locally to the middle or inner ear of the subject.
Embodiment 385. A Wnt agonist for use in a method of treating or preventing a disease or disorder of the ear in a subject in need thereof, said method comprising locally administering the Wnt agonist to the middle or inner ear of the subject, wherein the subject has been, or will be, administered a valproic acid compound systemically.
Embodiment 386. A Wnt agonist for use in a method of treating or preventing a disease or disorder of the ear in a subject in need thereof, said method comprising:
   a) systemically administering a valproic acid compound to the subject; and
   b) locally administering the Wnt agonist to the middle or inner ear of the subject,
   wherein steps a) and b) can occur in any order or simultaneously.
Embodiment 387. A valproic acid compound for use in a method of treating or preventing a disease or disorder of the ear in a subject in need thereof, said method comprising:
   a) systemically administering the valproic acid compound to the subject; and
   b) locally administering a Wnt agonist to the middle or inner ear of the subject,
   wherein steps a) and b) can occur in any order or simultaneously.
Embodiment 388. The valproic acid and/or Wnt agonist for use as described in the preceding five embodiments, wherein the method of treating or preventing is as defined in any preceding method of treatment claim.
Embodiment 389. A method of treating or preventing a disease or disorder of the ear in a subject in need thereof, comprising:
   a) systemically administering a valproic acid compound to the subject; and
   b) locally administering a Wnt agonist to the middle or inner ear of the subject;
   wherein steps a) and b) can occur in any order or simultaneously.
Embodiment 390. A method of treating or preventing a disease or disorder of the ear in a subject in need thereof comprising a) systemically administering a valproic acid compound to the subject, wherein the subject has been, or will be, b) administered a Wnt agonist locally to the middle or inner ear of the subject.
Embodiment 391. A method of treating or preventing a disease or disorder of the ear in a subject in need thereof comprising b) locally administering a Wnt agonist to the middle or inner ear of the subject, wherein the subject has been, or will be, a) administered a valproic acid compound systemically.
Embodiment 392. The method of any one of embodiments 389-391, wherein said systemically administering of step (a) is 0.5-25000 mg/day of the valproic acid compound.
Embodiment 393. The method of any one of embodiments 389-392, wherein the systemic plasma concentration of valproic acid in the subject ranges from about 5 μg/mL to about 5000 μg/mL.
Embodiment 394. The method of any one of embodiments 389-393, wherein said systemically administering of step (a) is once or twice daily.
Embodiment 395. The method of embodiment 394, wherein said systemically administering of step (a) is once daily.
Embodiment 396. The method of embodiment 394, wherein said systemically administering of step (a) is twice daily.
Embodiment 397. The method of any one of embodiments 389-396, wherein said systemically administering of step (a) begins 1 to about 14 days before beginning said local administration of step (b).
Embodiment 398. The method of embodiment 397, wherein said systemically administering of step (a) begins 1, 2, 3, 4, 7, or 14 days before beginning said locally administering of step (b).
Embodiment 399. The method of any one of embodiments 389-398, wherein said locally administering of step (b) is at least once weekly.
Embodiment 400. The method of any one of embodiments 389-399, wherein said locally administering of step (b) is once weekly.
Embodiment 401. The method of any one of embodiments 389-399, wherein said locally administering of step (b) is twice weekly.
Embodiment 402. The method of any one of embodiments 389-399, wherein said locally administering of step (b) is at least once daily.
Embodiment 403. The method of any one of embodiments 389-399, wherein said locally administering of step (b) is once daily.
Embodiment 404. The method of any one of embodiments 389-399, wherein said locally administering of step (b) is twice daily.
Embodiment 405. The method of any one of embodiments 389-398, wherein said systemically administering of step (a) occurs at least daily until 389-8 of said topical administrations of step (b) occur.
Embodiment 406. The method of any one of embodiments 389-405, wherein the valproic acid compound is a pharmaceutically acceptable alkali or alkaline earth metal salt of valproic acid.
Embodiment 407. The method of embodiment 406, wherein the valproic acid compound is sodium valproate.
Embodiment 408. The method of any one of embodiments 389-407, wherein said systemically administering of step (a) comprises administering an oral dosage form comprising an amount of valproic acid compound equivalent to about 50 mg, about 100 mg, about 125 mg, about 250 mg, about 500 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid.
Embodiment 409. The method of any one of embodiments 389408, wherein the Wnt agonist is a GSK-3 inhibitor.
Embodiment 410. 22. The method of any one of embodiments 389-409, wherein the Wnt agonist is a GSK-3α inhibitor.
Embodiment 411. The method of any one of embodiments 410, wherein the GSK-3α inhibitor is selected from Table 2.
Embodiment 412. The method of embodiment 411, wherein the Wnt agonist is a GSK-3β inhibitor.
Embodiment 413. The method of embodiment 412, wherein the GSK-3β inhibitor is selected from Table 1.
Embodiment 414. The method of any one of embodiments 389-409, wherein the Wnt agonists is selected from Table 4.
Embodiment 415. The method of any one of embodiments 389-408, wherein the Wnt agonist is

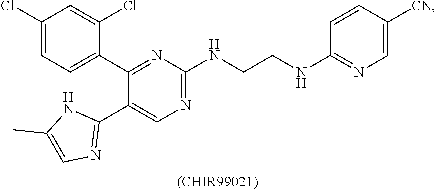 [SMILES: Cc1cnc(-c2cnc(NCCNc3ccc(C#N)cn3)nc2-c2ccc(Cl)cc2Cl)[nH]1]

or a pharmaceutically acceptable salt thereof.
Embodiment 416. The method of any one of embodiments 389-408, wherein the Wnt aonist is

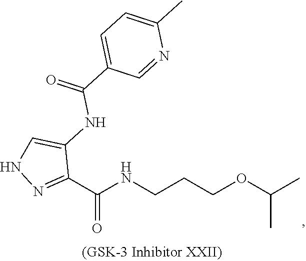 [SMILES: Cc1ccc(C(=O)Nc2c[nH]nc2C(=O)NCCCOC(C)C)cn1]

or a pharmaceutically acceptable salt thereof.
Embodiment 417. The method of any one of embodiments 389-408, wherein the Wnt agonist is a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof.
Embodiment 418. The method of any one of embodiments 389-408, wherein the Wnt agonist is

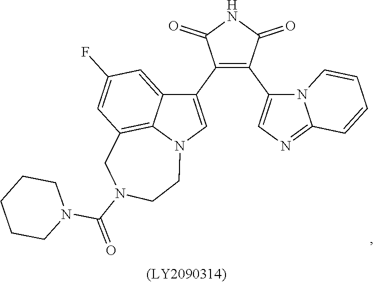 [SMILES: O=C1NC(=O)C(c2cnc3ccccn23)=C1c1cn2c3c(cc(F)cc13)CN(C(=O)N1CCCCC1)CC2]

or a pharmaceutically acceptable salt thereof.
Embodiment 419. The method of any one of embodiments 389408, wherein the Wnt agonist is

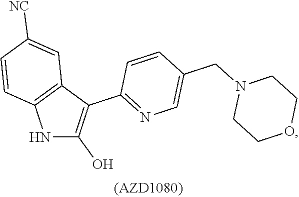 [SMILES: N#Cc1ccc2[nH]c(O)c(-c3ccc(CN4CCOCC4)cn3)c2c1]

or a pharmaceutically acceptable salt thereof.
Embodiment 420. The method of any one of embodiments 389-419, wherein said systemically administering in step (a) is an amount of valproic acid compound equivalent to about 10-60 mg/kg/day of valproic acid.
Embodiment 421. The method of embodiments 389-419, wherein said systemically administering in step (a) is an amount of valproic acid compound equivalent to about 10-150 mg/kg/day of valproic acid.
Embodiment 422. The method of any one of embodiments 389-419, wherein said systemically administering in step (a) is an amount of valproic acid compound equivalent to about 10-300 mg/kg/day of valproic acid.
Embodiment 423. The method of any one of embodiments 389-422, wherein the concentration of Wnt agonist locally administered in step (b) ranges from about 1 nM to about 1000 mM.
Embodiment 424. The method of any one of embodiments 389-408 and 420-423, wherein the Wnt agonist is CHIR99021 at a concentration of about 4 mM.
Embodiment 425. The method of any one of embodiments 389-408 and 419-423, wherein the Wnt agonist is LY2090314 at a concentration of about 10 μM.
Embodiment 426. The method of any one of embodiments 389-408 and 419-423, wherein the Wnt agonist is GSK-3 Inhibitor XXII at a concentration of about 500 μM.
Embodiment 427. The method of any one of embodiments 389408 and 419-423, wherein the Wnt agonist is AZD1080 at a concentration of about 3000 μM.
Embodiment 428. The method of any one of embodiments 389408, wherein said systemically administering in step (a) is an amount of sodium valproate equivalent to about 50 mg, about 100 mg, about 125 mg, about 250 mg, about 500 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid, and said locally administering in step (b) is an amount of CHIR99021 of about 125 μg to about 650 μg per day.
Embodiment 429. The method of any one of embodiments 389-408, wherein said systemically administering in step (a) is an amount of sodium valproate equivalent to about 125 mg, about 250 mg, about 500 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid, and said locally administering in step (b) is an amount of LY2090314 of about 0.1 μg to about 2.5 μg per day.
Embodiment 430. The method of any one of embodiments 389408, wherein said systemically administering in step (a) is an amount of sodium valproate equivalent to about 50 mg, about 100 mg, about 125 mg, about 250 mg, about 500 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid, and said locally administering in step (b) is an amount of GSK-3 Inhibitor XXII at about 15 μg to about 85 μg per day.
Embodiment 431. The method of any one of embodiments 389-408, wherein said systemically administering in step (a) is an amount of sodium valproate equivalent to about 50 mg, about 100 mg, about 125 mg, about 250 mg, about 500 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid, and said locally administering in step (b) is an amount of AZD1080 at about 115 μg to about 475 μg per day.
Embodiment 432. The method of any one of embodiments 389-431, further comprising step (c) locally administering a valproic acid compound to the middle or inner ear of the subject.
Embodiment 433. The method of embodiment 432, wherein said locally administering a valproic acid compound of step (c) provides a therapeutic effect and the locally administered valproic acid compound in step (c) is at a dose or concentration which is lower than the dose or concentration required to obtain a substantially similar therapeutic effect when locally administered in the absence of the systemically administered valproic acid compound in step (a).
Embodiment 434. The method of embodiment 433, wherein the dose or concentration of valproic acid compound administered in step (c) provides a valproic acid blood plasma concentration in a mammal of greater than about 90 μg/mL at about 3 h post administration.
Embodiment 435. The method of any one of embodiments 433434, wherein the dose of locally administered valproic acid compound required to obtain a therapeutic effect in the absence of the systemically administered valproic acid compound in step (a) is less than about 18 mg of valproic acid, and/or the concentration of locally administered valproic acid compound required to obtain a therapeutic effect in the absence of the systemically administered valproic acid compound in step (a) is less than about 88.6 mg/mL.
Embodiment 436. The method of any one of embodiments 433-435, wherein the daily dose of the valproic acid compound of step (a) is equivalent to about 0.5-25000 mg valproic acid, and the daily dose of the valproic acid compound of step (c) is equivalent to about 1 mg to about 18 mg of valproic acid, and/or the concentration of the daily dose of the valproic acid compound of step (c) is equivalent to about 90 mg/mL.
Embodiment 437. The method of any one of embodiments 433-436, wherein the daily dose of the valproic acid compound of step (a) is equivalent to about 0.5-25000 mg valproic acid, and the dose of valproic acid compound of step (c) is equivalent to an amount of valproic acid selected from the group consisting of about 1 mg, about 2 mg, about 4 mg, about 6 mg, about 8 mg, about 10 mg, about 12 mg, about 13 mg, about 14 mg, about 15 mg, about 16 mg, about 17 mg, and about 18 mg, and/or the concentration of the daily dose of the valproic acid compound of step (c) is selected from the group consisting of about 5 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, and 100 mg/mL, and about 110 mg/mL.
Embodiment 438. The method of embodiment 436, wherein the daily dose of the valproic acid compound of step (c) is equivalent to about 1 mg valproic acid.
Embodiment 439. The method of embodiment 436, wherein the daily dose of the valproic acid compound of step (c) is equivalent to about 2 mg valproic acid.
Embodiment 440. The method of embodiment 436, wherein the daily dose of the valproic acid compound of step (c) is equivalent to about 4 mg valproic acid.
Embodiment 441. The method of embodiment 436, wherein the daily dose of the valproic acid compound of step (c) is equivalent to about 6 mg valproic acid.
Embodiment 442. The method of embodiment 436, wherein the daily dose of the valproic acid compound of step (c) is equivalent to about 8 mg valproic acid.
Embodiment 443. The method of embodiment 436, wherein the daily dose of the valproic acid compound of step (c) is equivalent to about 10 mg valproic acid.
Embodiment 444. The method of embodiment 436, wherein the daily dose of the valproic acid compound of step (c) is equivalent to about 12 mg valproic acid.
Embodiment 445. The method of embodiment 436, wherein the concentration of the daily dose of the valproic acid compound of step (c) is about 5 mg/mL.
Embodiment 446. The method of embodiment 436, wherein the concentration of the daily dose of the valproic acid compound of step (c) is about 10 mg/mL.
Embodiment 447. The method of embodiment 436, wherein the concentration of the daily dose of the valproic acid compound of step (c) is about 20 mg/mL.
Embodiment 448. The method of embodiment 436, wherein the concentration of the daily dose of the valproic acid compound of step (c) is about 30 mg/mL.
Embodiment 449. The method of embodiment 436, wherein the concentration of the daily dose of the valproic acid compound of step (c) is about 40 mg/mL.
Embodiment 450. The method of embodiment 436, wherein the concentration of the daily dose of the valproic acid compound of step (c) is about 50 mg/mL.
Embodiment 451. The method of embodiment 436, wherein the concentration of the daily dose of the valproic acid compound of step (c) is about 60 mg/mL.
Embodiment 452. The method of embodiment 436, wherein the concentration of the daily dose of the valproic acid compound of step (c) is about 70 mg/mL.
Embodiment 453. The method of embodiment 436, wherein the concentration of the daily dose of the valproic acid compound of step (c) is about 80 mg/mL.
Embodiment 454. The method of embodiment 436, wherein the concentration of the daily dose of the valproic acid compound of step (c) is about 90 mg/mL.
Embodiment 455. The method of embodiment 436, wherein the concentration of the daily dose of the valproic acid compound of step (c) is about 100 mg/mL.
Embodiment 456. The method of embodiment 436, wherein the concentration of the daily dose of the valproic acid compound of step (c) is about 110 mg/mL.
Embodiment 457. The method of embodiment 436, wherein the concentration of the daily dose of the valproic acid compound of step (c) is about 88.6 mg/mL.
Embodiment 458. The method of embodiment 436, wherein the concentration of the daily dose of the valproic acid compound of step (c) is about 17.7 mg.
Embodiment 459. The method of any one of embodiments 432458, wherein the locally administering of steps (b) and (c) occur at about the same time.
Embodiment 460. The method of any one of embodiments 432-458, wherein the locally administered Wnt agonist and the locally administered valproic acid compound of step (c) are administered together in a fixed-dose combination.
Embodiment 461. The method of any one of embodiments 432-458, wherein the concentration of the valproic acid compound locally administered in step (c) is equivalent to about 5 to less than about 89 g/mL of valproic acid.
Embodiment 462. The method of any one of embodiments 432458, wherein the concentration of the valproic acid compound locally administered in step (c) is a concentration equivalent to a valproic acid concentration selected from the group consisting of about 5 mg/mL, about 10 mg/mL, about 20 mg/mL, about 30 mg/mL, about 40 mg/mL, about 50 mg/mL, about 60 mg/mL, about 80 mg/mL, and about 88.6 mg/mL.
Embodiment 463. The method of any one of embodiments 429459, wherein said systemically administering a valproic acid compound and said locally administering a valproic acid compound produce a valproic acid half-life in the perilymph of the subject that is about 1.1-fold to about 10-fold longer than a half-life achieved upon locally administering a valproic acid compound alone.
Embodiment 464. The method of any one of embodiments 389-463, wherein the disease or disorder of the ear is associated with a reduced level of sensory hair cells in the subject.
Embodiment 465. The method of any one of embodiments 389-463, wherein the disease or disorder is a reduction or loss of hearing in the subject.
Embodiment 466. The method of any one of embodiments 389-45, wherein the disease or disorder is selected from the group consisting of tinnitus, hyperacusis, vertigo and Meniere's disease.
Embodiment 467. The method of embodiment 389464, wherein after said treating, the population of stem cells of the sensory hair cells increases by at least 1.25-fold to about 20-fold.
Embodiment 468. A method of treating or preventing a disease or disorder of the ear in a subject in need thereof, comprising locally administering a Wnt agonist to the middle or inner ear of the subject, wherein the locally administering is at a time when the subject has a systemic plasma concentration of valproic acid of about 5 μg/mL to about 5000 μg/mL.
Embodiment 469. The method of embodiment 468, wherein the systemic plasma concentration of valproic acid is about 100 μg/mL.
Embodiment 470. The method of embodiment 468, wherein the systemic plasma concentration of valproic acid is maintained at about 5 μg/mL to about 500 μg/mL for at least about 1-10 hours prior to the locally administering.
Embodiment 471. The method of embodiment 468, wherein the systemic plasma concentration of valproic acid is maintained at about 5 μg/mL to about 500 μg/mL for at least about 1-10 hours after the locally administering.
Embodiment 472. A method of treating or preventing a disease or disorder of the ear in a subject in need thereof, comprising:
   a) systemically administering a valproic acid compound to the subject; and
   b) systemically administering a Wnt agonist to the subject,
   wherein steps a) and b) can occur in any order or simultaneously.
Embodiment 473. A method of treating or preventing a disease or disorder of the ear in a subject in need thereof, comprising:
   a) systemically administering a valproic acid compound to the subject; and
   b) systemically administering CHIR99021, or a pharmaceutically acceptable salt thereof, to the subject,
   wherein steps a) and b) can occur in any order or simultaneously.
Embodiment 474. A method of treating or preventing a disease or disorder of the ear in a subject in need thereof, comprising:
   a) systemically administering a valproic acid compound to the subject; and
   b) systemically administering GSK-3 Inhibitor XXII or a pharmaceutically acceptable salt thereof, to the subject,
   wherein steps a) and b) can occur in any order or simultaneously.
Embodiment 475. A method of treating or preventing a disease or disorder of the ear in a subject in need thereof, comprising:
   a) systemically administering a valproic acid compound to the subject; and
   b) systemically administering LY2090314 or a pharmaceutically acceptable salt thereof, to the subject,
   wherein steps a) and b) can occur in any order or simultaneously.
Embodiment 476. A method of treating or preventing a disease or disorder of the ear in a subject in need thereof, comprising:
   a) systemically administering a valproic acid compound to the subject; and
   b) systemically administering AZD1080 or a pharmaceutically acceptable salt thereof, to the subject,
   wherein steps a) and b) can occur in any order or simultaneously.
Embodiment 477. The method of any one of embodiments 472-476, wherein the Wnt agonist is an agent other than a valproic acid compound.
Embodiment 478. A method of treating or preventing a disease or disorder of the ear in a subject in need thereof, comprising:
   a) systemically administering a valproic acid compound to the subject; and
   b) locally administering CHIR99021, or a pharmaceutically acceptable salt thereof, to the middle or inner ear of the subject;
   wherein steps a) and b) can occur in any order or simultaneously.
Embodiment 479. The method of embodiment 478, wherein the valproic acid compound is administered in an amount equivalent to about 0.5-25000 mg valproic acid.
Embodiment 480. The method of any one of embodiments 479480, wherein the valproic acid compound comprises sodium valproate.
Embodiment 481. The method of any one of embodiments 478-480, wherein CHIR99021, or a pharmaceutically acceptable salt thereof, is administered in an amount of about 125 μg to about 650 μg per day.
Embodiment 482. The method of any one of embodiments 478-481, further comprising c) locally administering a valproic acid compound to the middle or inner ear of the subject,
   wherein steps a), b), and c) can occur in any order or any of steps a), b), or c) can occur simultaneously.
Embodiment 483. The method of any one of embodiments 482, wherein the valproic acid compound of step c) comprises sodium valproate.
Embodiment 484. The method of any one of embodiments 482483, wherein the daily dose of the valproic acid compound in step c) is equivalent to about 1 mg to about 12 mg valproic acid.
Embodiment 485. A method of treating or preventing a disease or disorder of the ear in a subject in need thereof, comprising:
   a) systemically administering a valproic acid compound to the subject; and
   b) locally administering GSK-3 Inhibitor XXII, or a pharmaceutically acceptable salt thereof, to the middle or inner ear of the subject:
   wherein steps a) and b) can occur in any order or simultaneously.
Embodiment 486. The method of embodiment 485, wherein the valproic acid compound is administered in an amount equivalent to about 0.5-25000 mg valproic acid.
Embodiment 487. The method of any one of embodiments 484-485, wherein the valproic acid compound comprises sodium valproate.
Embodiment 488. The method of any one of embodiments 484-487, wherein GSK-3 Inhibitor XXII, or a pharmaceutically acceptable salt thereof, is administered in an amount of about 15 μg to about 85 μg per day.
Embodiment 489. The method of any one of embodiments 484-488, further comprising c) locally administering a valproic acid compound to the middle or inner ear of the subject,
   wherein steps a), b), and c) can occur in any order or any of steps a), b), or c) can occur simultaneously.
Embodiment 490. The method of any one of embodiments 1489, wherein the valproic acid compound of step c) comprises sodium valproate.
Embodiment 491. The method of any one of embodiments 489-490, wherein the daily dose of the valproic acid compound in step c) is equivalent to about 1 mg to about 12 mg valproic acid.
Embodiment 492. A method of treating or preventing a disease or disorder of the ear in a subject in need thereof, comprising:
   a) systemically administering a valproic acid compound to the subject; and
   b) locally administering LY2090314, or a pharmaceutically acceptable salt thereof, to the middle or inner ear of the subject;
   wherein steps a) and b) can occur in any order or simultaneously.
Embodiment 493. The method of embodiment 492, wherein the valproic acid compound is administered in an amount equivalent to about 0.5-25000 mg valproic acid.
Embodiment 494. The method of any one of embodiments 492-493, wherein the valproic acid compound comprises sodium valproate.
Embodiment 495. The method of any one of embodiments 492-494, wherein LY2090314, or a pharmaceutically acceptable salt thereof, is administered in an amount of about 0.1 μg to about 2.5 μg per day.
Embodiment 496. The method of any one of embodiments 492-495, further comprising c) locally administering a valproic acid compound to the middle or inner ear of the subject,
   wherein steps a), b), and c) can occur in any order or any of steps a), b), or c) can occur simultaneously.
Embodiment 497. The method of any one of embodiments 496, wherein the valproic acid compound of step c) comprises sodium valproate.
Embodiment 498. The method of any one of embodiments 496-497, wherein the daily dose of the valproic acid compound in step c) is equivalent to about 1 mg to about 12 mg valproic acid.
Embodiment 499. A method of treating or preventing a disease or disorder of the ear in a subject in need thereof, comprising:
   a) systemically administering a valproic acid compound to the subject; and
   b) locally administering AZD1080, or a pharmaceutically acceptable salt thereof, to the middle or inner ear of the subject;
   wherein steps a) and b) can occur in any order or simultaneously.
Embodiment 500. The method of embodiment 499, wherein the valproic acid compound is administered in an amount equivalent to about 0.5-25000 mg valproic acid.
Embodiment 501. The method of any one of embodiments 499-500, wherein the valproic acid compound comprises sodium valproate.
Embodiment 502. The method of any one of embodiments 499-501, wherein AZD1080, or a pharmaceutically acceptable salt thereof, is administered in an amount of about 115 μg to about 475 μg per day.
Embodiment 503. The method of any one of embodiments 499-502, further comprising c) locally administering a valproic acid compound to the middle or inner ear of the subject,
   wherein steps a), b), and c) can occur in any order or any of steps a), b), or c) can occur simultaneously.
Embodiment 504. The method of any one of embodiments 503, wherein the valproic acid compound of step c) comprises sodium valproate.
Embodiment 505. The method of any one of embodiments 503-504, wherein the daily dose of the valproic acid compound in step c) is equivalent to about 1 mg to about 12 mg valproic acid.
Embodiment 506. A method of treating or preventing a disease or disorder of the ear in a subject in need thereof, comprising:
   a) systemically administering a valproic acid compound to the subject; and
   b) locally administering a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, to the middle or inner ear of the subject,
   wherein steps a) and b) can occur in any order or simultaneously.
Embodiment 507. The method of embodiment 506, wherein the valproic acid compound is administered in an amount equivalent to about 0.5-25000 mg valproic acid.
Embodiment 508. The method of any one of embodiments 506-507, wherein the valproic acid compound comprises sodium valproate.
Embodiment 509. The method of any one of embodiments 507-508, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, is administered in an amount of about 1 μg to about 1000 μg per day.
Embodiment 510. The method of any one of embodiments 499-502, further comprising c) locally administering a valproic acid compound to the middle or inner ear of the subject,
   wherein steps a), b), and c) can occur in any order or any of steps a), b), or c) can occur simultaneously.
Embodiment 511. The method of any one of embodiments 510, wherein the valproic acid compound of step c) comprises sodium valproate.
Embodiment 512. The method of any one of embodiments 510-511, wherein the daily dose of the valproic acid compound in step c) is equivalent to about 1 mg to about 12 mg valproic acid.
Embodiment 513. The method of any one of embodiments 506-512, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1-I-48, or a pharmaceutically acceptable salt thereof.
Embodiment 514. The method of any one of embodiments 506-512, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof.
Embodiment 515. The method of any of the preceding embodiments, wherein the subject has an inner ear hearing disorder.
Embodiment 516. The method of any of the preceding embodiments, wherein the subject has a balance disorder.
Embodiment 517. The method of any of the preceding embodiments, wherein the disease or disorder is sensorineural hearing loss.
Embodiment 518. The method of any of the preceding embodiments, wherein treatment results in improved auditory function when assessed by behavioral audiometry or auditory brainstem response (ABR) testing.
Embodiment 519. A valproic acid compound and a Wnt agonist for use in a method of treating or preventing a disease or disorder of the ear in a subject in need thereof, said method comprising:
   a) systemically administering the valproic acid compound to the subject; and
   b) locally administering the Wnt agonist to the middle or inner ear of the subject,
   wherein steps a) and b) can occur in any order or simultaneously.
Embodiment 520. The valproic acid compound of embodiment 519, wherein treatment is as defined in any of embodiments 489-518.
Embodiment 521. A valproic acid compound for use in a method of treating or preventing a disease or disorder of the ear in a subject in need thereof, said method comprising systemically administering the valproic acid compound, wherein the subject has been, or will be, administered a Wnt agonist locally to the middle or inner ear of the subject.
Embodiment 522. The valproic acid compound of embodiment 521, wherein treatment is as defined in any of embodiments 489-518.
Embodiment 523. A Wnt agonist for use in a method of treating or preventing a disease or disorder of the ear in a subject in need thereof, said method comprising locally administering the Wnt agonist to the middle or inner ear of the subject, wherein the subject has been, or will be, administered a valproic acid compound systemically.
Embodiment 524. The Wnt agonist of embodiment 135235, wherein treatment is as defined in any of embodiments 489-518.
Embodiment 525. A Wnt agonist for use in a method of treating or preventing a disease or disorder of the ear in a subject in need thereof, said method comprising:
   a) systemically administering a valproic acid compound to the subject; and
   b) locally administering the Wnt agonist to the middle or inner ear of the subject,
   wherein steps a) and b) can occur in any order or simultaneously.
Embodiment 526. The Wnt agonist of embodiment 525, wherein treatment is as defined in any of embodiments 489-518.
Embodiment 527. A valproic acid compound for use in a method of treating or preventing a disease or disorder of the ear in a subject in need thereof, said method comprising:
   a) systemically administering the valproic acid compound to the subject; and
   b) locally administering a Wnt agonist to the middle or inner ear of the subject,
   wherein steps a) and b) can occur in any order or simultaneously.
Embodiment 528. The valproic acid compound of embodiment 527, wherein treatment is as defined in any of embodiments 489-518.
Embodiment 529. A kit comprising:
   a first pharmaceutical composition comprising a valproic acid compound:
   a second pharmaceutical composition comprising a Wnt agonist; and
   a set of instructions directing a user to:
   a) systemically administer the first pharmaceutical composition to a subject; and
   b) locally administer the second pharmaceutical composition to the middle or inner ear of the subject,
   wherein steps a) and b) can occur in any order or simultaneously.
Embodiment 530. The kit of embodiment 529, further comprising a third pharmaceutical composition comprising a valproic acid compound,
   wherein the set of instructions directs the user to c) locally administer the third pharmaceutical composition to the middle or inner ear of the subject, and
   wherein steps a), b), and c) can occur in any order or simultaneously.
Embodiment 531. The kit of embodiment 529, wherein the second pharmaceutical composition further comprising a valproic acid compound.
Embodiment 532. A kit comprising:
   a first pharmaceutical composition comprising a valproic acid compound;
   a second pharmaceutical composition comprising a Wnt agonist; and
   a set of instructions directing a user to:
   a) locally administer the first pharmaceutical composition to the middle or inner ear of a subject; and
   b) locally administer the second pharmaceutical composition to the middle or inner ear of the subject,
   wherein steps a) and b) can occur in any order or simultaneously.
Embodiment 533. A kit comprising:
   a pharmaceutical composition comprising:
   a valproic acid compound, and
   a Wnt agonist; and
   a set of instructions directing a user to locally administer the pharmaceutical composition to the middle or inner ear of a subject.
Embodiment 534. A kit comprising:
   a first pharmaceutical composition comprising a valproic acid compound:
   a second pharmaceutical composition comprising CHIR99021, or a pharmaceutically acceptable salt thereof; and
   a set of instructions directing a user to:
   a) systemically administer the first pharmaceutical composition to a subject; and
   b) locally administer the second pharmaceutical composition to the middle or inner ear of the subject,
   wherein steps a) and b) can occur in any order or simultaneously.
Embodiment 535. The kit of embodiment 534, wherein the instructions direct the user to administer the valproic acid compound in an amount equivalent to about 0.5-25000 mg valproic acid per day.
Embodiment 536. The kit of any one of embodiments 534-535, wherein the first pharmaceutical composition comprises the valproic acid compound in an amount equivalent to about 0.5-25000 mg valproic acid per unit dose.
Embodiment 537. The kit of any one of embodiments 534-536, wherein the valproic acid compound comprises sodium valproate.
Embodiment 538. The kit of any one of embodiments 534-537, wherein the instructions direct the user to administer about 125 μg to about 650 μg CHIR99021, or a pharmaceutically acceptable salt thereof, per day.
Embodiment 539. The kit of any one of embodiments 534-538, wherein the second pharmaceutical composition comprises CHIR99021, or a pharmaceutically acceptable salt thereof, in an amount of about 125 μg to about 650 μg per unit dose.
Embodiment 540. The kit of any one of embodiments 534-539, wherein the second pharmaceutical composition further comprises a valproic acid compound.
Embodiment 541. The kit of embodiment 540, wherein the second pharmaceutical composition comprises the valproic acid compound in an amount equivalent to about 1 mg to about 12 mg valproic acid per unit dose.
Embodiment 542. The kit of any one of embodiments 534-541, further comprising a third pharmaceutical composition comprising a valproic acid compound,
   wherein the set of instructions directs the user to c) locally administer the third pharmaceutical composition to the middle or inner ear of the subject, and
   wherein steps a), b), and c) can occur in any order or simultaneously.
Embodiment 543. The kit of embodiment 542, wherein the instructions direct the user to locally administer the valproic acid compound in an amount equivalent to about 1 mg to about 12 mg valproic acid per day.
Embodiment 544. The kit of any one of embodiments 542-543, wherein the third pharmaceutical composition comprises the valproic acid compound in an amount equivalent to about 1 mg to about 12 mg valproic acid per unit dose.
Embodiment 545. The kit of any one of embodiments 542-544, wherein the third pharmaceutical composition comprises sodium valproate.
Embodiment 546. A kit comprising:
   a first pharmaceutical composition comprising a valproic acid compound;
   a second pharmaceutical composition comprising CHIR99021, or a pharmaceutically
   acceptable salt thereof; and
   a set of instructions directing a user to:
   a) locally administer the first pharmaceutical composition to the middle or inner ear of a subject; and
   b) locally administer the second pharmaceutical composition to the middle or inner ear of the subject,
   wherein steps a) and b) can occur in any order or simultaneously.
Embodiment 547. The kit of embodiment 546, wherein the instructions direct the user to administer the valproic acid compound in an amount equivalent to about 1 mg to about 12 mg valproic acid per day.
Embodiment 548. The kit of any one of embodiments 546-547, wherein the first pharmaceutical composition comprises the valproic acid compound in an amount equivalent to about 1 mg to about 12 mg valproic acid per unit dose.
Embodiment 549. The kit of any one of embodiments 546-548, wherein the first pharmaceutical composition comprises sodium valproate.
Embodiment 550. The kit of any one of embodiments 546-549, wherein the instructions direct the user to administer about 125 μg to about 650 μg CHIR99021, or a pharmaceutically acceptable salt thereof, per day.
Embodiment 551. The kit of any one of embodiments 546-550, wherein the second pharmaceutical composition comprises CHIR99021, or a pharmaceutically acceptable salt thereof, in an amount of about 125 μg to about 650 μg per unit dose.
Embodiment 552. A kit comprising:
   a pharmaceutical composition comprising:
   a valproic acid compound, and
   CHIR99021, or a pharmaceutically acceptable salt thereof; and
   a set of instructions directing a user to locally administer the pharmaceutical composition to the middle or inner ear of a subject.
Embodiment 553. The kit of embodiment 552, wherein the instructions direct the user to administer the valproic acid compound in an amount equivalent to about 1 mg to about 12 mg valproic acid per day, and to administer about 125 μg to about 650 μg CHIR99021, or a pharmaceutically acceptable salt thereof, per day.
Embodiment 554. The kit of any one of embodiments 552-553, wherein the pharmaceutical composition comprises per unit dose:
   the valproic acid compound in an amount equivalent to about 1 mg to about 12 mg valproic acid, and
   CHIR99021, or a pharmaceutically acceptable salt thereof, in an amount of about 125 μg to about 650 μg.
Embodiment 555. A kit comprising:
   a first pharmaceutical composition comprising a valproic acid compound;
   a second pharmaceutical composition comprising GSK-3 Inhibitor XXII, or a pharmaceutically acceptable salt thereof; and
   a set of instructions directing a user to:
   a) systemically administer the first pharmaceutical composition to a subject; and
   b) locally administer the second pharmaceutical composition to the middle or inner ear of the subject,
   wherein steps a) and b) can occur in any order or simultaneously.
Embodiment 556. The kit of embodiment 555, wherein the instructions direct the user to administer the valproic acid compound in an amount equivalent to about 0.5-25000 mg valproic acid per day.
Embodiment 557. The kit of any one of embodiments 555-556, wherein the first pharmaceutical composition comprises the valproic acid compound in an amount equivalent to about 0.5-25000 mg valproic acid per unit dose.
Embodiment 558. The kit of any one of embodiments 555-557, wherein the valproic acid compound comprises sodium valproate.
Embodiment 559. The kit of any one of embodiments 555-558, wherein the instructions direct the user to administer about 15 μg to about 85 μg GSK-3 Inhibitor XXII, or a pharmaceutically acceptable salt thereof, per day.
Embodiment 560. The kit of any one of embodiments 555-559, wherein the second pharmaceutical composition comprises GSK-3 Inhibitor XXII, or a pharmaceutically acceptable salt thereof, in an amount of about 15 μg to about 85 μg per unit dose.
Embodiment 561. The kit of any one of embodiments 555-560, wherein the second pharmaceutical composition further comprises a valproic acid compound.
Embodiment 562. The kit of embodiment 561, wherein the second pharmaceutical composition comprises the valproic acid compound in an amount equivalent to about 1 mg to about 12 mg valproic acid per unit dose.
Embodiment 563. The kit of embodiment 555, further comprising a third pharmaceutical composition comprising a valproic acid compound,
   wherein the set of instructions directs the user to c) locally administer the third pharmaceutical composition to the middle or inner ear of the subject, and
   wherein steps a), b), and c) can occur in any order or simultaneously.
Embodiment 564. The kit of embodiment 563, wherein the instructions direct the user to locally administer the valproic acid compound in an amount equivalent to about 1 mg to about 12 mg valproic acid per day.
Embodiment 565. The kit of any one of embodiments 563-564, wherein the third pharmaceutical composition comprises the valproic acid compound in an amount equivalent to about 1 mg to about 12 mg valproic acid per unit dose.
Embodiment 566. The kit of any one of embodiments 563-565, wherein the third pharmaceutical composition comprises sodium valproate.
Embodiment 567. A kit comprising:
   a first pharmaceutical composition comprising a valproic acid compound;
   a second pharmaceutical composition comprising GSK-3 Inhibitor XXII, or a pharmaceutically acceptable salt thereof; and
   a set of instructions directing a user to:
   a) locally administer the first pharmaceutical composition to the middle or inner ear of a subject; and
   b) locally administer the second pharmaceutical composition to the middle or inner ear of the subject,
   wherein steps a) and b) can occur in any order or simultaneously.
Embodiment 568. The kit of embodiment 556, wherein the instructions direct the user to administer the valproic acid compound in an amount equivalent to about 1 mg to about 12 mg valproic acid per day.
Embodiment 569. The kit of any one of embodiments 567-568, wherein the first pharmaceutical composition comprises the valproic acid compound in an amount equivalent to about 1 mg to about 12 mg valproic acid per unit dose.
Embodiment 570. 182. The kit of any one of embodiments 567-569, wherein the first pharmaceutical composition comprises sodium valproate.
Embodiment 571. The kit of any one of embodiments 567-570, wherein the instructions direct the user to administer about 15 μg to about 85 μg GSK-3 Inhibitor XXII, or a pharmaceutically acceptable salt thereof, per day.
Embodiment 572. The kit of any one of embodiments 567-571, wherein the second pharmaceutical composition comprises GSK-3 Inhibitor XXII, or a pharmaceutically acceptable salt thereof, in an amount of about 15 μg to about 85 μg per unit dose.
Embodiment 573. A kit comprising:
   a pharmaceutical composition comprising:
   a valproic acid compound, and
   GSK-3 Inhibitor XXII, or a pharmaceutically acceptable salt thereof, and
   a set of instructions directing a user to locally administer the pharmaceutical composition to the middle or inner ear of a subject.
Embodiment 574. The kit of embodiment 573, wherein the instructions direct the user to administer the valproic acid compound in an amount equivalent to about 1 mg to about 12 mg valproic acid per day, and to administer about 15 μg to about 85 μg GSK-3 Inhibitor XXII, or a pharmaceutically acceptable salt thereof, per day.
Embodiment 575. The kit of any one of embodiments 573-574, wherein the pharmaceutical composition comprises per unit dose:
   the valproic acid compound in an amount equivalent to about 1 mg to about 12 mg valproic acid, and
   GSK-3 Inhibitor XXII, or a pharmaceutically acceptable salt thereof, in an amount of about 15 μg to about 85 μg.
Embodiment 576. A kit comprising:
   a first pharmaceutical composition comprising a valproic acid compound:
   a second pharmaceutical composition comprising LY2090314, or a pharmaceutically acceptable salt thereof; and
   a set of instructions directing a user to:
   a) systemically administer the first pharmaceutical composition to a subject; and
   b) locally administer the second pharmaceutical composition to the middle or inner ear of the subject,
   wherein steps a) and b) can occur in any order or simultaneously.
Embodiment 577. The kit of embodiment 576, wherein the instructions direct the user to administer the valproic acid compound in an amount equivalent to about 0.5-25000 mg valproic acid per day.
Embodiment 578. The kit of any one of embodiments 576-577, wherein the first pharmaceutical composition comprises the valproic acid compound in an amount equivalent to about 0.5-25000 mg valproic acid per unit dose.
Embodiment 579. The kit of any one of embodiments 576-578, wherein the valproic acid compound comprises sodium valproate.
Embodiment 580. The kit of any one of embodiments 576-579, wherein the instructions direct the user to administer about 0.1 μg to about 2.5 μg LY2090314, or a pharmaceutically acceptable salt thereof, per day.
Embodiment 581. The kit of any one of embodiments 576-580, wherein the second pharmaceutical composition comprises LY2090314, or a pharmaceutically acceptable salt thereof, in an amount of about 0.1 μg to about 2.5 μg per unit dose.
Embodiment 582. The kit of any one of embodiments 576-581, wherein the second pharmaceutical composition further comprises a valproic acid compound.
Embodiment 583. The kit of embodiment 582, wherein the second pharmaceutical composition comprises the valproic acid compound in an amount equivalent to about 1 mg to about 12 mg valproic acid per unit dose.
Embodiment 584. The kit of embodiment 576, further comprising a third pharmaceutical composition comprising a valproic acid compound,
   wherein the set of instructions directs the user to c) locally administer the third pharmaceutical composition to the middle or inner ear of the subject, and
   wherein steps a), b), and c) can occur in any order or simultaneously.
Embodiment 585. The kit of embodiment 584, wherein the instructions direct the user to locally administer the valproic acid compound in an amount equivalent to about 1 mg to about 12 mg valproic acid per day.
Embodiment 586. The kit of any one of embodiments 584-585, wherein the third pharmaceutical composition comprises the valproic acid compound in an amount equivalent to about 1 mg to about 12 mg valproic acid per unit dose.
Embodiment 587. The kit of any one of embodiments 584-586, wherein the third pharmaceutical composition comprises sodium valproate.
Embodiment 588. A kit comprising:
   a first pharmaceutical composition comprising a valproic acid compound;
   a second pharmaceutical composition comprising LY2090314, or a pharmaceutically acceptable salt thereof; and
   a set of instructions directing a user to:
   a) locally administer the first pharmaceutical composition to the middle or inner ear of a subject; and
   b) locally administer the second pharmaceutical composition to the middle or inner ear of the subject,
   wherein steps a) and b) can occur in any order or simultaneously.
Embodiment 589. The kit of embodiment 588, wherein the instructions direct the user to administer the valproic acid compound in an amount equivalent to about 1 mg to about 12 mg valproic acid per day.
Embodiment 590. The kit of any one of embodiments 588-589, wherein the first pharmaceutical composition comprises the valproic acid compound in an amount equivalent to about 1 mg to about 12 mg valproic acid per unit dose.
Embodiment 591. The kit of any one of embodiments 588-590, wherein the first pharmaceutical composition comprises sodium valproate.
Embodiment 592. The kit of any one of embodiments 588-591, wherein the instructions direct the user to administer about 0.1 μg to about 2.5 μg LY2090314, or a pharmaceutically acceptable salt thereof, per day.
Embodiment 593. The kit of any one of embodiments 588-592, wherein the second pharmaceutical composition comprises LY2090314, or a pharmaceutically acceptable salt thereof, in an amount of about 0.1 μg to about 2.5 μg per unit dose.
Embodiment 594. A kit comprising:
   a pharmaceutical composition comprising:
   a valproic acid compound, and
   LY2090314, or a pharmaceutically acceptable salt thereof; and
   a set of instructions directing a user to locally administer the pharmaceutical composition to the middle or inner ear of a subject.
Embodiment 595. The kit of embodiment 594, wherein the instructions direct the user to administer the valproic acid compound in an amount equivalent to about 1 mg to about 12 mg valproic acid per day, and to administer about 0.1 μg to about 2.5 μg LY2090314, or a pharmaceutically acceptable salt thereof, per day.
Embodiment 596. The kit of any one of embodiments 594-595, wherein the pharmaceutical composition comprises per unit dose:
   the valproic acid compound in an amount equivalent to about 1 mg to about 12 mg valproic acid, and
   LY2090314, or a pharmaceutically acceptable salt thereof, in an amount of about 0.1 μg to about 2.5 μg.
Embodiment 597. A kit comprising:
   a first pharmaceutical composition comprising a valproic acid compound;
   a second pharmaceutical composition comprising AZD1080, or a pharmaceutically acceptable salt thereof; and
   a set of instructions directing a user to:
   a) systemically administer the first pharmaceutical composition to a subject; and
   b) locally administer the second pharmaceutical composition to the middle or inner ear of the subject,
   wherein steps a) and b) can occur in any order or simultaneously.
Embodiment 598. The kit of embodiment 597, wherein the instructions direct the user to administer the valproic acid compound in an amount equivalent to about 0.5-25000 mg valproic acid per day.
Embodiment 599. The kit of any one of embodiments 597-598, wherein the first pharmaceutical composition comprises the valproic acid compound in an amount equivalent to about 0.5-25000 mg valproic acid per unit dose.
Embodiment 600. The kit of any one of embodiments 597-599, wherein the valproic acid compound comprises sodium valproate.
Embodiment 601. The kit of any one of embodiments 597-600, wherein the instructions direct the user to administer about 115 μg to about 475 μg AZD1080, or a pharmaceutically acceptable salt thereof, per day.
Embodiment 602. The kit of any one of embodiments 597-601, wherein the second pharmaceutical composition comprises AZD1080, or a pharmaceutically acceptable salt thereof, in an amount of about 115 μg to about 475 μg per unit dose.
Embodiment 603. The kit of any one of embodiments 597-602, wherein the second pharmaceutical composition further comprises a valproic acid compound.
Embodiment 604. The kit of embodiment 603, wherein the second pharmaceutical composition comprises the valproic acid compound in an amount equivalent to about 1 mg to about 12 mg valproic acid per unit dose.
Embodiment 605. The kit of embodiment 597, further comprising a third pharmaceutical composition comprising a valproic acid compound,
   wherein the set of instructions directs the user to c) locally administer the third pharmaceutical composition to the middle or inner ear of the subject, and
   wherein steps a), b), and c) can occur in any order or simultaneously.
Embodiment 606. The kit of embodiment 605, wherein the instructions direct the user to locally administer the valproic acid compound in an amount equivalent to about 1 mg to about 12 mg valproic acid per day.
Embodiment 607. The kit of any one of embodiments 605-606, wherein the third pharmaceutical composition comprises the valproic acid compound in an amount equivalent to about 1 mg to about 12 mg valproic acid per unit dose.
Embodiment 608. The kit of any one of embodiments 605-607, wherein the third pharmaceutical composition comprises sodium valproate.
Embodiment 609. A kit comprising:
   a first pharmaceutical composition comprising a valproic acid compound;
   a second pharmaceutical composition comprising AZD1080, or a pharmaceutically acceptable salt thereof; and
   a set of instructions directing a user to:
   a) locally administer the first pharmaceutical composition to the middle or inner ear of a subject; and
   b) locally administer the second pharmaceutical composition to the middle or inner ear of the subject,
   wherein steps a) and b) can occur in any order or simultaneously.
Embodiment 610. The kit of embodiment 609, wherein the instructions direct the user to administer the valproic acid compound in an amount equivalent to about 1 mg to about 12 mg valproic acid per day.
Embodiment 611. The kit of any one of embodiments 609-610, wherein the first pharmaceutical composition comprises the valproic acid compound in an amount equivalent to about 1 mg to about 12 mg valproic acid per unit dose.
Embodiment 612. The kit of any one of embodiments 609-611, wherein the first pharmaceutical composition comprises sodium valproate.
Embodiment 613. The kit of any one of embodiments 609-612, wherein the instructions direct the user to administer about 115 μg to about 475 μg AZD1080, or a pharmaceutically acceptable salt thereof, per day.
Embodiment 614. The kit of any one of embodiments 609-613, wherein the second pharmaceutical composition comprises AZD1080, or a pharmaceutically acceptable salt thereof, in an amount of about 115 μg to about 475 μg per unit dose.
Embodiment 615. A kit comprising:
   a pharmaceutical composition comprising:
   a valproic acid compound, and
   AZD1080, or a pharmaceutically acceptable salt thereof; and
   a set of instructions directing a user to locally administer the pharmaceutical composition to the middle or inner ear of a subject.
Embodiment 616. The kit of embodiment 615, wherein the instructions direct the user to administer the valproic acid compound in an amount equivalent to about 1 mg to about 12 mg valproic acid per day, and to administer about 115 μg to about 475 μg AZD1080, or a pharmaceutically acceptable salt thereof, per day.
Embodiment 617. The kit of any one of embodiments 615-616, wherein the pharmaceutical composition comprises per unit dose:
   the valproic acid compound in an amount equivalent to about 1 mg to about 12 mg valproic acid, and
   AZD1080, or a pharmaceutically acceptable salt thereof, in an amount of about 115 μg to about 475 μg.
Embodiment 618. A kit comprising:
   a first pharmaceutical composition comprising a valproic acid compound:
   a second pharmaceutical composition comprising a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof; and
   a set of instructions directing a user to:
   a) systemically administer the first pharmaceutical composition to a subject; and
   b) locally administer the second pharmaceutical composition to the middle or inner ear of the subject,
   wherein steps a) and b) can occur in any order or simultaneously.
Embodiment 619. The kit of embodiment 618, wherein the instructions direct the user to administer the valproic acid compound in an amount equivalent to about 0.5-25000 mg valproic acid per day.
Embodiment 620. The kit of any one of embodiments 618-619, wherein the first pharmaceutical composition comprises the valproic acid compound in an amount equivalent to about 0.5-25000 mg valproic acid per unit dose.
Embodiment 621. The kit of any one of embodiments 618-620, wherein the valproic acid compound comprises sodium valproate.
Embodiment 622. The kit of any one of embodiments 618-621, wherein the instructions direct the user to administer about 1 μg to about 1000 μg substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, per day.
Embodiment 623. The kit of any one of embodiments 618-622, wherein the second pharmaceutical composition comprises a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, in an amount of about 1 μg to about 1000 μg per unit dose.
Embodiment 624. The kit of any one of embodiments 618-623, wherein the second pharmaceutical composition further comprises a valproic acid compound.
Embodiment 625. The kit of embodiment 624, wherein the second pharmaceutical composition comprises the valproic acid compound in an amount equivalent to about 1 mg to about 12 mg valproic acid per unit dose.
Embodiment 626. The kit of embodiment 618, further comprising a third pharmaceutical composition comprising a valproic acid compound,
   wherein the set of instructions directs the user to c) locally administer the third pharmaceutical composition to the middle or inner ear of the subject, and
   wherein steps a), b), and c) can occur in any order or simultaneously.
Embodiment 627. The kit of embodiment 626, wherein the instructions direct the user to locally administer the valproic acid compound in an amount equivalent to about 1 mg to about 12 mg valproic acid per day.
Embodiment 628. The kit of any one of embodiments 626-627, wherein the third pharmaceutical composition comprises the valproic acid compound in an amount equivalent to about 1 mg to about 12 mg valproic acid per unit dose.
Embodiment 629. The kit of any one of embodiments 626-627, wherein the third pharmaceutical composition comprises sodium valproate.
Embodiment 630. The kit of any one of embodiments 618-629, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1-I-48, or a pharmaceutically acceptable salt thereof.
Embodiment 631. The kit of any one of embodiments 618-629, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43. I-44, and I-48, or a pharmaceutically acceptable salt thereof.
Embodiment 632. A kit comprising:
   a first pharmaceutical composition comprising a valproic acid compound,
   a second pharmaceutical composition comprising a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof; and
   a set of instructions directing a user to:
   a) locally administer the first pharmaceutical composition to the middle or inner car of a subject; and
   b) locally administer the second pharmaceutical composition to the middle or inner ear of the subject,
   wherein steps a) and b) can occur in any order or simultaneously.
Embodiment 633. The kit of embodiment 632, wherein the instructions direct the user to administer the valproic acid compound in an amount equivalent to about 1 mg to about 12 mg valproic acid per day.
Embodiment 634. The kit of any one of embodiments 632-633, wherein the first pharmaceutical composition comprises the valproic acid compound in an amount equivalent to about 1 mg to about 12 mg valproic acid per unit dose.
Embodiment 635. The kit of any one of embodiments 632-634, wherein the first pharmaceutical composition comprises sodium valproate.
Embodiment 636. The kit of any one of embodiments 632-635, wherein the instructions direct the user to administer about 1 μg to about 1000 μg substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, per day.
Embodiment 637. The kit of any one of embodiments 632-636, wherein the second pharmaceutical composition comprises a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, in an amount of about 1 μg to about 1000 μg per unit dose.
Embodiment 638. The kit of any one of embodiments 632-637, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1-I-48, or a pharmaceutically acceptable salt thereof.
Embodiment 639. The kit of any one of embodiments 632-637, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof.
Embodiment 640. A kit comprising:
   a pharmaceutical composition comprising:
   a valproic acid compound, and
   a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof; and
   a set of instructions directing a user to locally administer the pharmaceutical composition to the middle or inner ear of a subject.
Embodiment 641. The kit of embodiment 640, wherein the instructions direct the user to administer the valproic acid compound in an amount equivalent to about 1 mg to about 12 mg valproic acid per day, and to administer about 1 μg to about 1000 μg substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, per day.
Embodiment 642. The kit of any one of embodiments 640-641, wherein the pharmaceutical composition comprises per unit dose: the valproic acid compound in an amount equivalent to about 1 mg to about 12 mg valproic acid, and
   a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, in an amount of about 1 μg to about 1000 μg.
Embodiment 643. The kit of any one of embodiments 640-642, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1-I-48, or a pharmaceutically acceptable salt thereof.
Embodiment 644. The kit of any one of embodiments 640-642, wherein a substituted 3-imidazo[1,2-a]pyridin-3-yl-4-(1,2,3,4-tetrahydro-[1,4]diazepino-[6,7,1-hi]indol-7-yl)pyrrole-2,5-dione is selected from Compounds I-1, I-2, I-3, I-6, I-7, I-8, I-9, I-12, I-16, I-18, I-20, I-22, I-23, I-24, I-25, I-26, I-27, I-29, I-30, I-31, I-33, I-36, I-39, I-43, I-44, and I-48, or a pharmaceutically acceptable salt thereof.

The following non-limiting examples illustrate various aspects of the present disclosure.

### EXAMPLES

To assess cochlear PK properties, four sets of experiments were conducted to generate data that were used to model human cochlear PK with an established computer model (Salt et al. Laryngoscope. 118, 1793-800 (2008)). These consisted of 1) assessing drug levels and residence time in the inner ear by sampling perilymph from lateral semicircular canal after delivery directly into the perilymph or 2) delivery to the round window membrane, 3) assessing drug distribution along the cochlear length using sequential sampling from the cochlear apex, and 4) assessing drug level and residence time in the middle ear. Data from these experiments were then used to model human cochlear PK using an established computer modeling system.

### Example 1a: General Methods

Guinea pigs were treated with systemically delivered VPA with or without a local delivery to the middle ear of a Wnt agonist, CHIR99021, with VPA to assess the efficacy of different routes of administration and delivery of said compounds. Guinea pigs were treated and sampled according to protocols below.

Samples were analyzed using high-pressure liquid chromatography with mass spectrometry detection (HPLC-MS) methods that were validated under matrix matched conditions, e.g. perilymph and plasma matrices. Analysts were blinded to the dose levels used in the animal experiments.

Pharmacokinetic studies were performed as non-recovery procedures in pigmented, NIH-strain guinea pigs weighing 400-600 g. Animal use followed policies in accordance with the United States Department of Agriculture and National Institutes of Health guidelines for the handling and use of laboratory animals. Animals were anesthetized with 100 mg/kg sodium thiobutabarbital (Inactin®, Sigma, St Louis, Mo.) after which a polyethylene tracheal cannula was placed. The animal was then maintained on 0.8% to 1.2% isofluorane in oxygen using a mechanical ventilator. End-tidal CO2 level was held close to 5% by adjustment of the ventilator tidal volume. Heart rate and O2 saturation were monitored, and core body temperature was maintained at 38° C. with a thermistor-controlled heating blanket.

Local drug delivery to the middle ear was performed by first exposing the auditory bulla using the ventral approach. After the cochlear apex or LSCC had been prepared for fluid sampling a 20 μL bolus of poloxamer containing 6.75 mM CHIR99021 and 533 mM VPA was applied to the round window (RW) niche using a positive displacement hand pipettor (Eppendorf Biomaster 4830). The applied volume was sufficient to fill the RW niche with excess flowing over the stapes footplate towards the apical bulla.

### Example 1B: Lateral Canal Sampling

For lateral canal drug delivery and perilymph sampling, the lateral portion of the auditory bulla was exposed through a post-auricular incision and opened. Bone over the lateral semicircular canal (LSCC) was thinned with a dental burr. A branch of the facial nerve was removed where it ran parallel to the LSCC. When the canal lumen was visible through the translucent thinned bone, the bone was dried and thin cyanoacrylate glue (PermaBond™ 101; Permabond, Pottstown, Pa.) was applied followed by layers of two-part silicone adhesive (Kwik-Cast, World Precision Instruments, Sarasota, Fla.). The silicone was applied thinly over the translucent bone region but multiple layers were built up at the edges to form a hydrophobic cup. The canal wall was fenestrated (30-40 μm diameter) using a 300 House stapes pick (N1705 80, Bausch and Lomb Inc.). A blunt-tipped drug-injection pipette was inserted through the fenestration. Drug injections were performed with a 100 μL gas-tight syringe (1710TLL Hamilton) mounted on a digitally-controlled Ultrapump (World Precision Instruments, Sarasota, Fla.) held in a manipulator. A plexiglass coupler (MPH6S10, World Precision Instruments, Sarasota, Fla.) was used to attach a 1 mm diameter glass pipette to the syringe. The injection pipette had been pulled in an electrode puller and broken with forceps to produce a blunt tip with a diameter of 15-20 μm. The pipette tip was inserted into the fenestration and sealed in place with a droplet of thin cyanoacrylate glue. Injections into the perilymphatic space were performed at a rate of 1 μL/min for 60 minutes. The injected solution contained 3.14 mg/ml CHIR99021 and 88.6 mg/ml valproic acid in a bicarbonate-buffered artificial perilymph.

If drug was not being injected at the LSCC, the LSCC was prepared and fenestrated as described above. If drug injection was taking place at the LSCC, the injection pipette was removed and the cyanoacrylate glue at the fenestration site was broken away with a pick. In either case, the perilymph emerging from the LSCC perforation accumulated on the hydrophobic surface of the silicone where it was collected in hand-held, blunt tipped capillary tubes (VWR 53432-706), marked to a nominal volume of 1 μL. Twenty individual samples were collected from each animal, each taking 1-2 minutes to collect. The precise volume of each sample was determined by measuring its length under a calibrated dissecting microscope immediately after collection. The 20 samples were paired consecutively and each pair added to 25 μL of 1:1 acetonitrile:water diluent, resulting in 10 diluted samples, each containing 2 μL perilymph in 25 μL diluent. Samples were stored frozen at −80° C. until analysis.

### Example 2: Apical Sampling

The concentration of compound/drug (i.e. VPA and/or Wnt agonist) along the length of the guinea pig cochlea when VPA is delivered systemically with or without a local delivery to the middle ear of a Wnt agonist, CHIR99021, with VPA, was assessed as described herein.

Gradients of drug along the perilymphatic spaces were directly measured from multiple samples obtained by a technique called “sequential sampling.” When the cochlear apex (apex) is perforated, perilymph is driven out by cerebrospinal fluid (CSF) entering the basal turn of scala tympani (ST) through the cochlear aqueduct, pushing perilymph in an apical direction along the scala tympani. The first sample collected originates from perilymph near the apex of the cochlea and each following sample from perilymph that originated from a cochlear location progressively closer to the cochlear base (base). After approximately 4 samples, all perilymph has been pushed out, and subsequent samples contain CSF that has passed through the cochlea. Samples collected in this manner allow drug gradients along the length of the cochlea to be quantified. The cochlear apex was prepared for sampling by removing the mucosa with a cotton swab and allowing the bone to dry. A thin layer of cyanoacrylate glue (Permabond 101; Permabond, Pottstown, Pa.) was applied to the dry bone, followed by layers of two-part silicone adhesive (Kwik-Cast. World Precision Instruments, Sarasota, Fla.), built up at the edges to form a hydrophobic cup. At the time of sampling a 30-40 μm fenestration was made at the apex through the adhesives using a 30° House stapes pick (N1705 80, Bausch and Lomb Inc.). Clear fluid flowed from the fenestration, accumulating on the hydrophobic surface. Fluid was collected into hand-held, blunt tipped capillary tubes (VWR 53432-706; VWR Radnor, Pa.), each marked for a nominal volume of 1 μL and taking 1-2 min to collect. The length of each sample in its capillary tube was immediately measured with a calibrated dissecting microscope, to establish the exact sample volume. Ten individual samples were collected in this manner and each expelled into 25 μL of 1:1 acetonitrile:water diluent. Samples were stored frozen at −80° C. until analysis.

### Example 3: Systemic Valproic Acid Administration

The concentration of valproic acid in the guinea pig cochlea following systemic administration was assessed to determine if therapeutically relevant levels could be reached.

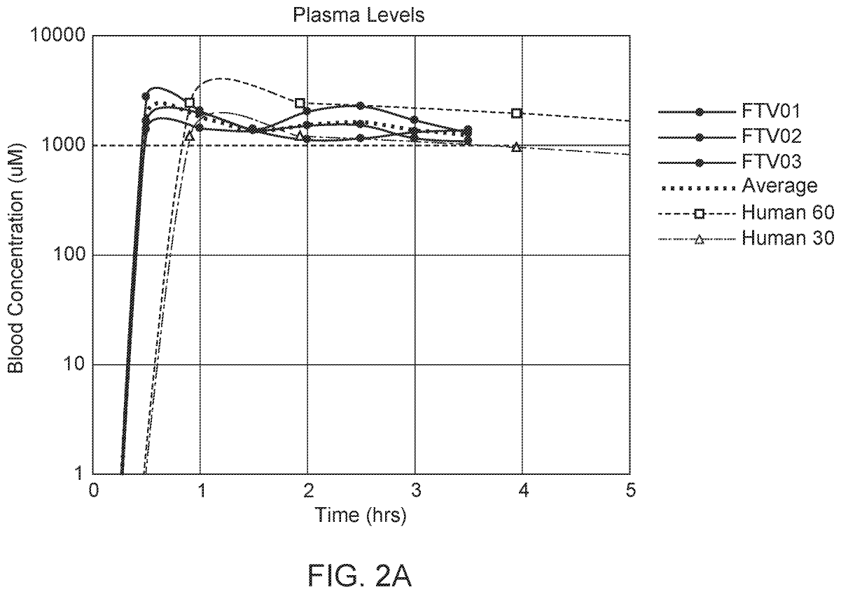

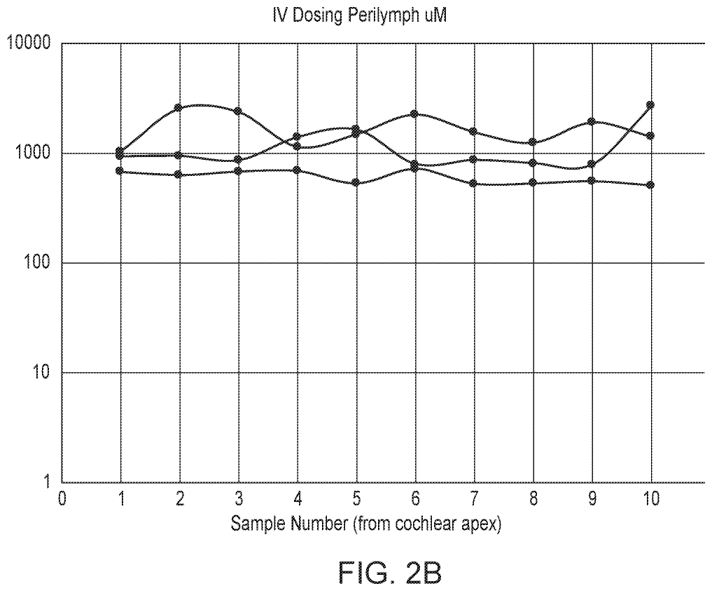

Three guinea pigs weighing 340 g (FTV01), 370 g (FTV02), and 370 g (FTV03), respectively, were injected intravenously with 100 mg/mL (601.7 mM) sodium valproate at 20 μL/min for 60 minutes, and compounds were delivered to the middle ear 30 minutes after the systemic administration had started. Each animal systemically received 120 mg (1.2 mL), i.e. averaging about 330 mg/kg. The dosage equates to about 70 mg/kg in humans according to the Federal Drug Administration scaling factor of 4.6. Blood samples were taken every thirty minutes and blood valproic acid concentrations in the samples were assayed and compared to human doses of 60 mg/kg (Human 60) and 30 mg/kg (Human 30). As shown in FIG. 2A, plasma levels generally peaked within 1 hour, consistent with published human data (Georgoff et al. Clin Pharmacokinet. 2018:57(2):209-219). Guinea pig perilymph samples were obtained and assayed as described in Example 2. Uniform distribution of valproate was observed throughout the cochlea at 3 hours (apex to base; serial samples 1-4), and at concentrations correlated with therapeutic levels determined in vitro using primary cochlear Lgr5 cells (FIG. 2B).

These data indicate that systemic administration of valproate can be used to achieve therapeutic levels in the cochlea and systemic administration can be useful for treating or preventing hearing disorders responsive to valproate treatment.

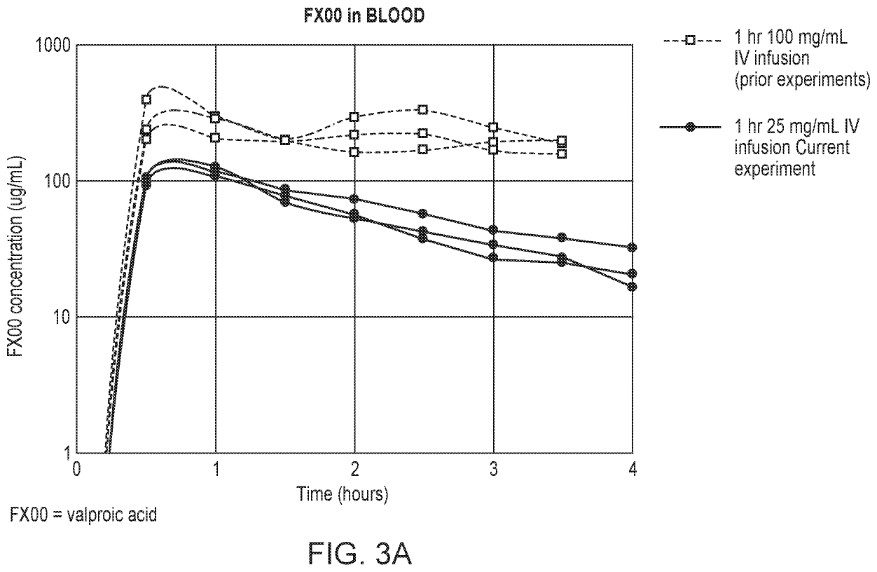

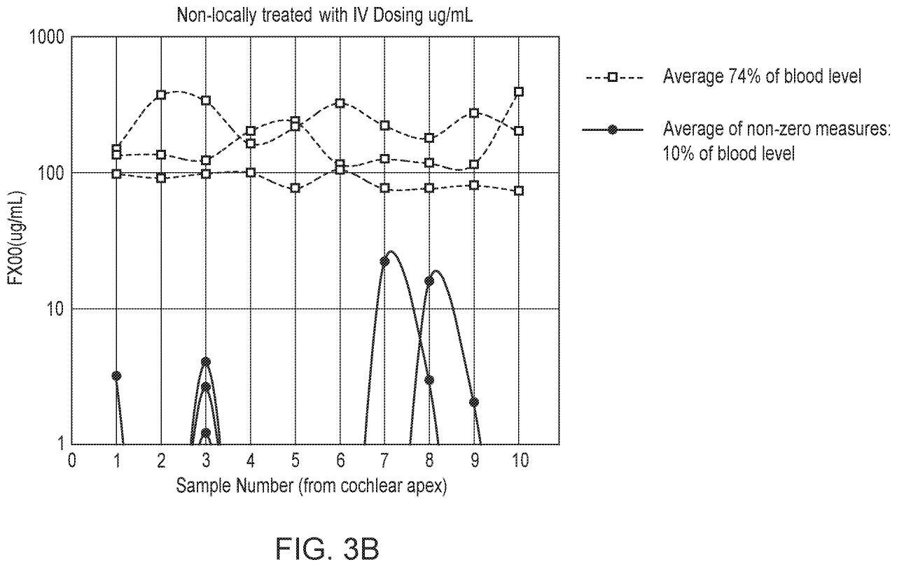

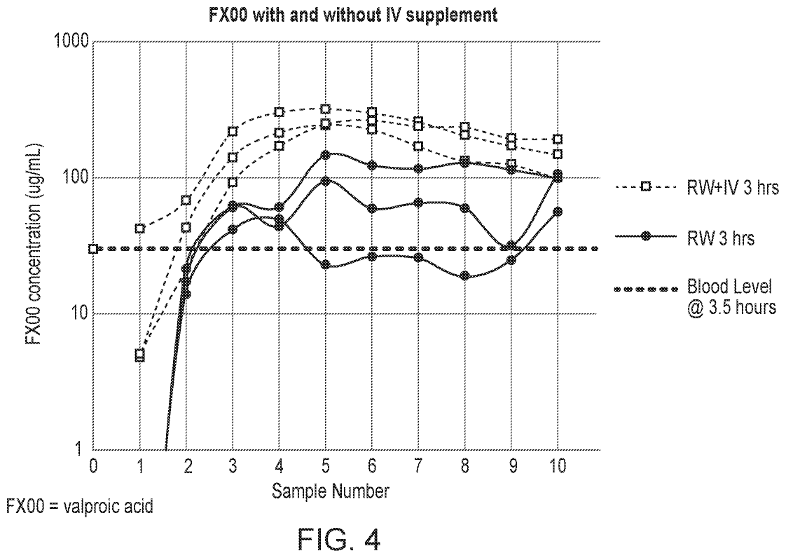

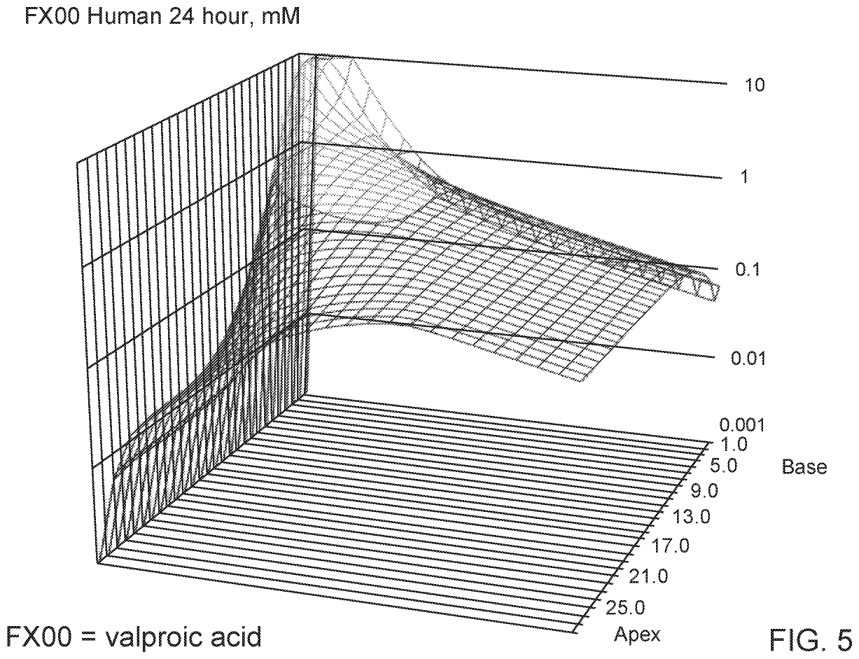

Three guinea pigs were injected intravenously with 25 mg/mL sodium valproate solution (21.69 mg/mL valproate) at 20 μL/min for 60 min, and compounds were delivered to the middle ear 30 minutes after the systemic administration had started. Each animal received about 26 mg valproate (1.2 mL). FIG. 3A compares the blood valproic acid concentrations at 100 mg/mL and 25 mg/mL administrations every 30 minutes. Perilymph samples from the animals were tested to determine whether levels of valproate predicted to be therapeutic were achieved in the cochlea after systemic administration of the lower dose. FIG. 3B compares perilymph valproic acid concentrations at 100 mg/mL and 25 mg/mL administrations at 3 hours. The results indicate that the 100 mg/mL dosage shows uniform distribution and therapeutic levels, whereas the 25 mg/mL dosage did not achieve therapeutic levels as determined in vitro on primary cochlear Lgr5 cells.

### Example 4: Systemic and Local Administration of Valproic Acid Improves Distribution and Retention in Cochlea Compared to Local-Only Administration

The cochlear distribution and temporal change of CHIR99021 and/or VPA concentrations were assessed following systemic administration of valproic acid combined with local administration of CHIR99021 and VPA.

Three guinea pigs were injected intravenously with 25 mg/mL sodium valproate solution (21.69 mg/mL valproate) at 20 μL/min for 60 minutes, with local application to the RW occurring 30 minutes after the systemic administration had started. Each animal systemically received about 26 mg valproate (1.2 mL). Each animal was locally administered 20 μL of a poloxamer formulation including sodium valproate (88.63 mg/mL) and CHIR99021 (3.14 mg/mL) to the round window niche. Perilymph valproic acid concentrations were measured at 3 hours.

Three other guinea pigs were locally administered 20 μL of a poloxamer formulation including sodium valproate (88.63 mg/mL) and CHIR99021 (3.14 mg/mL) to the round window niche. Perilymph valproic acid concentrations were measured at 3 hours.

The perilymph valproic acid concentrations from the two groups of guinea pigs were compared. As illustrated in FIG. 4, the combination of systemic and local administration improved the drug distribution and retention in guinea pig cochlea as compared to local-only administration.

These data indicate that a combination of systemic and local administration can facilitate treatment in the cochlea.

### Example 5: Human In Vivo Modeling—Systemic and Local Administration

Human in vivo modeling studies were conducted to study the effects of systemic valproic acid delivery combined with local administration of compound formulations comprising valproic acid and Wnt agonists.

The established finite-element inner ear fluids simulation program (FluidSim®) was configured to replicate all aspects of the experiments performed on animals to predict PK kinetics in humans. For intratympanic applications, calculations included entry from the middle ear at the round window and the stapes. For LSCC drug injections, the induced flows from the injection site to the cochlear aqueduct were simulated. Drug distribution through the fluid and tissue spaces of the entire inner ear were calculated based on defined diffusion coefficients and elimination rates. Simulation of the sequential sampling procedure accounted for the associated volume flows, replicating the flow rates necessary to account for the specific volumes, and collection times for each of the samples. FIG. 5 illustrates the model simulated by FluidSim.

Data extrapolation predicted that systemic sodium valproate administration (60 mg/kg) combined with local administration of a poloxamer formulation of sodium valproate and CHIR99021 will improve valproic acid retention and distribution in the cochlea in humans and that the valproic acid will travel to the apical portion of the cochlea at therapeutic levels.

The embodiments described herein and illustrated by the foregoing examples should be understood to be illustrative of the present disclosure, and should not be construed as limiting. On the contrary, the present disclosure embraces alternatives and equivalents thereof, as embodied by the appended claims. Each reference disclosed herein is incorporated by reference herein in its entirety for all purposes.

What is claimed is:

1. A method of treating or preventing a disease or disorder of the ear in a subject in need thereof, comprising:
a) systemically administering a valproic acid compound to the subject; and
b) locally administering a Wnt agonist to the middle or inner ear of the subject;
wherein a) and b) can occur in any order or simultaneously.

2. The method of claim 1, further comprising (c) locally administering a valproic acid compound to the middle or inner ear of the subject wherein a), b), and c) can occur in any order or any of a), b), or c) can occur simultaneously.

3. The method of claim 1 or 2, wherein the locally administered Wnt agonist and the locally administered valproic acid compound of (c) are administered together in a fixed-dose combination.

4. A method of treating or preventing a disease or disorder of the ear in a subject in need thereof, comprising:
a) systemically administering a valproic acid compound to the subject; and
b) systemically administering a Wnt agonist to the subject,
wherein a) and b) can occur in any order or simultaneously.

5. The method of any of the preceding claims, wherein the disease or disorder of the ear is associated with a reduced level of sensory hair cells in the subject.

6. The method of any of the preceding claims, wherein the disease or disorder is a reduction or loss of hearing in the subject.

7. The method of any of the preceding claims, wherein the disease or disorder is selected from the group consisting of tinnitus, hyperacusis, vertigo, and Meniere's disease.

8. The method of any of the preceding claims, wherein the subject has an inner ear hearing disorder.

9. The method of any of the preceding claims, wherein the subject has a balance disorder.

10. The method of any of the preceding claims, wherein the disease or disorder is sensorineural hearing loss.

11. The method of any of the preceding claims, wherein treatment results in improved auditory function when assessed by behavioral audiometry, auditory brainstem response (ABR) testing, sound intelligibility assessments, pure tone audiometry, Distortion product otoacoustic emissions (DPOAEs), or extended high frequency (EHF) audiometry.

12. The method of any one of claims 1-3 and 5-11, wherein the systemically administering of (a) begins 1 day to about 14 days before beginning the local administration of step (b).

13. The method of any of the preceding claims, wherein the systemically administering of (a) is once or twice daily.

14. The method of any one of claims 1-3 and 5-13, wherein said locally administering of step (b) is at least once weekly.

15. The method of any one of claims 1-3 and 5-14, wherein said locally administering of step (b) is at least once daily.

16. The method of any one of claims 1-3 and 5-15, wherein the locally administering is at a time when the subject has a systemic plasma concentration of valproic acid of about 5 μg/mL to about 5000 μg/mL.

17. The method of claim 16, wherein the systemic plasma concentration of valproic acid is maintained at about 5 μg/mL to about 500 μg/mL for at least about 1-10 hours prior to the locally administering.

18. The method of any of the preceding claims, wherein the valproic acid compound is a pharmaceutically acceptable alkali or alkaline earth metal salt of valproic acid.

19. The method of claim 18, wherein the valproic acid compound is sodium valproate.

20. The method of any of the preceding claims, wherein the Wnt agonist is a GSK-3 inhibitor.

21. The method of claim 20, wherein the GSK-3 inhibitor is a GSK-3α inhibitor or GSK-3β inhibitor.

22. The method of any of the preceding claims, wherein the Wnt agonist is:
a) 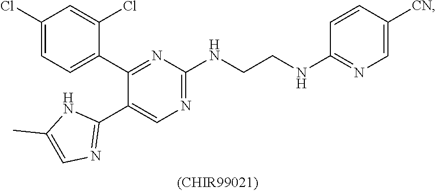 [SMILES: Cc1cnc(-c2cnc(NCCNc3ccc(C#N)cn3)nc2-c2ccc(Cl)cc2Cl)[nH]1]
or a pharmaceutically acceptable salt thereof;
b) 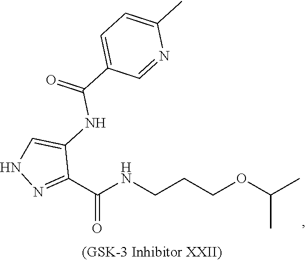 [SMILES: Cc1ccc(C(=O)Nc2c[nH]nc2C(=O)NCCCOC(C)C)cn1]
or a pharmaceutically acceptable salt thereof;
c) 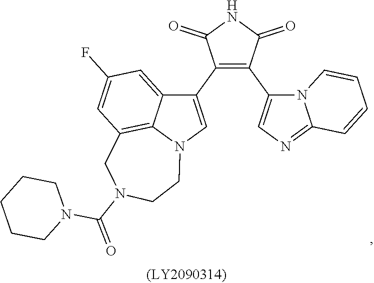 [SMILES: O=C1NC(=O)C(c2cnc3ccccn23)=C1c1cn2c3c(cc(F)cc13)CN(C(=O)N1CCCCC1)CC2]
or a pharmaceutically acceptable salt thereof;
d) 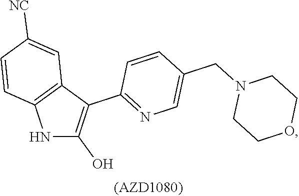 [SMILES: N#Cc1ccc2[nH]c(O)c(-c3ccc(CN4CCOCC4)cn3)c2c1]
or a pharmaceutically acceptable salt thereof; or
e) a substituted 3-imidazo[1,2-a]pyridin-3-yl-4-(1,2,3,4-tetrahydro-[1,4]diazepino-[6,7,1hi]indol-7-yl)pyrrole-2,5-dione, or a pharmaceutically acceptable salt thereof.

23. The method of any of the preceding claims, wherein the systemic administration of step (a) is 0.5-25000 mg/day of the valproic acid compound.

24. The method of any of the preceding claims, wherein said systemically administering of step (a) comprises administering an oral dosage form comprising an amount of valproic acid compound equivalent to about 50 mg, about 100 mg, about 125 mg, about 250 mg, about 500 mg, about 1000 mg, about 2000 mg, about 3000 mg, about 4000 mg, about 5000 mg, about 6000 mg, about 7000 mg, about 8000 mg, about 9000 mg, about 10000 mg, about 15000 mg, about 20000 mg, or about 25000 mg of valproic acid.

25. The method of any one of claims 1-3 and 5-24, wherein the concentration of Wnt agonist locally administered in step (b) ranges from about 1 nM to about 1000 mM.

26. The method claim 25, wherein the Wnt agonist is CHIR99021 at a concentration of about 4 mM.

27. The method of claim 25, wherein the Wnt agonist is LY2090314 at a concentration of about 10 μM.

28. The method claim 25, wherein the Wnt agonist is GSK-3 Inhibitor XXII at a concentration of about 500 μM.

29. The method of claim 25, wherein the Wnt agonist is AZD1080 at a concentration of about 3000 μM.

30. The method of claim 2, wherein said systemically administering a valproic acid compound and said locally administering a valproic acid compound produces a valproic acid half-life in the perilymph of the subject that is about 1.1-fold to about 10-fold longer than a half-life achieved upon locally administering a valproic acid compound alone.

31. A kit comprising:
a first pharmaceutical composition comprising a valproic acid compound;
a second pharmaceutical composition comprising a Wnt agonist; and
a set of instructions directing a user to:
a) systemically administer the first pharmaceutical composition to a subject; and
b) locally administer the second pharmaceutical composition to the middle or inner ear of the subject,
wherein steps a) and b) can occur in any order or simultaneously.

32. The kit of claim 31, further comprising a third pharmaceutical composition comprising a valproic acid compound,
wherein the set of instructions directs the user to c) locally administer the third pharmaceutical composition to the middle or inner ear of the subject, and
wherein steps a), b), and c) can occur in any order or simultaneously.

33. The kit of claim 31, wherein the second pharmaceutical composition further comprising a valproic acid compound.

34. The kit of any one of claims 31-33, wherein the valproic acid compound is a pharmaceutically acceptable alkali or alkaline earth metal salt of valproic acid.

35. The kit of claim 34, wherein the valproic acid compound is sodium valproate.

36. The kit of any one of claims 31-35, wherein the first pharmaceutical composition comprises the valproic acid compound in an amount equivalent to about 0.5-25000 mg valproic acid per unit dose.

37. The kit of any one of claims 33-36, wherein the second or third pharmaceutical composition comprises the valproic acid compound in an amount equivalent to about 1 mg to about 12 mg valproic acid per unit dose.

38. The kit of any one of claims 31-37, wherein the Wnt agonist is a GSK-3 inhibitor.

39. The kit of claim 38, wherein the GSK-3 inhibitor is a GSK-3α inhibitor or GSK-3β inhibitor.

40. The kit of any one of claims 31-39, wherein the Wnt agonist is:
a) 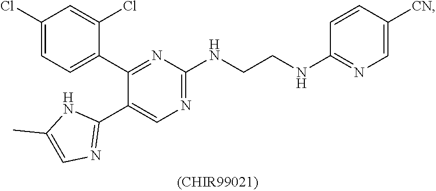 [SMILES: Cc1cnc(-c2cnc(NCCNc3ccc(C#N)cn3)nc2-c2ccc(Cl)cc2Cl)[nH]1]
or a pharmaceutically acceptable salt thereof;
b) 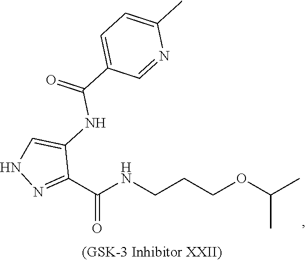 [SMILES: Cc1ccc(C(=O)Nc2c[nH]nc2C(=O)NCCCOC(C)C)cn1]
or a pharmaceutically acceptable salt thereof;
c) 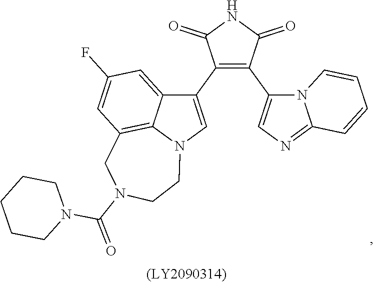 [SMILES: O=C1NC(=O)C(c2cnc3ccccn23)=C1c1cn2c3c(cc(F)cc13)CN(C(=O)N1CCCCC1)CC2]
or a pharmaceutically acceptable salt thereof;
d) 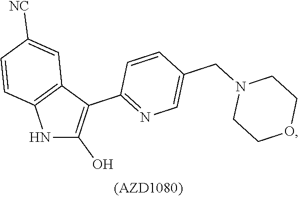 [SMILES: N#Cc1ccc2[nH]c(O)c(-c3ccc(CN4CCOCC4)cn3)c2c1]
or a pharmaceutically acceptable salt thereof; or
e) a substituted 3-imidazo[1,2-a]pyridin-3-yl-4-(1,2,3,4-tetrahydro-[1,4]diazepino-[6,7,1hi]indol-7-yl)pyrrole-2,5-dione, or a pharmaceutically acceptable salt thereof.

41. The kit of any one of claims 31-37, wherein the second pharmaceutical composition comprises CHIR99021, or a pharmaceutically acceptable salt thereof, in an amount of about 125 μg to about 650 μg per unit dose.

42. The kit of any one of claims 31-37, wherein the second pharmaceutical composition comprises GSK Inhibitor XXII, or a pharmaceutically acceptable salt thereof, in an amount of about 15 μg to about 85 μg per unit dose.

43. The kit of any one of claims 31-37, wherein the second pharmaceutical composition comprises LY2090314, or a pharmaceutically acceptable salt thereof, in an amount of about 0.1 μg to about 2.5 μg per unit dose.

44. The kit of any one of claims 31-37, wherein the second pharmaceutical composition comprises AZD1080, or a pharmaceutically acceptable salt thereof, in an amount of about 115 μg to about 475 μg per unit dose.

45. The kit of any one of claims 31-37, wherein the second pharmaceutical composition comprises a substituted 3-imidazo[1,2-a]pyridin-3-yl-4-(1,2,3,4-tetrahydro-[1,4]diazepino-[6,7,1-hi]indol-7-yl)pyrrole-2,5-dione, or a pharmaceutically acceptable salt thereof, in an amount of about 1 μg to about 1000 μg per unit dose.
